(19) United States
(12) Patent Application Publication
(10) Pub. No.: US 2024/0261332 A1
(43) Pub. Date: Aug. 8, 2024

(54) INHIBITORY CHIMERIC ANTIGEN RECEPTOR PREVENTS ON-TARGET OFF-TUMOR EFFECTS OF ADOPTIVE CELL THERAPY

(71) Applicant: BOARD OF REGENTS, THE UNIVERSITY OF TEXAS SYSTEM, Austin, TX (US)

(72) Inventors: Katy REZVANI, Houston, TX (US); Ye LI, Houston, TX (US)

(73) Assignee: BOARD OF REGENTS, THE UNIVERSITY OF TEXAS SYSTEM, Austin, TX (US)

(21) Appl. No.: 18/559,146

(22) PCT Filed: May 25, 2022
(86) PCT No.: PCT/US2022/030948

§ 371 Date: Nov. 6, 2023

Related U.S. Application Data
(60) Provisional application No. 63/296,785, filed on Jan. 5, 2022.
(60) Provisional application No. 63/193,363, filed on May 26, 2021.

(51) Int. Cl. A61K 35/17; A61K 39/00; C07K 14/725; C07K 16/28; C12N 5/0783

(52) U.S. Cl. CPC A61K 35/17; A61K 39/4613; A61K 39/4631; A61K 39/464412; C07K 14/7051; C07K 16/2803; C12N 5/0646; A61K 2239/22; A61K 2239/28; A61K 2239/29; C07K 2317/622; C07K 2319/03; C12N 2510/00

(57) ABSTRACT

Embodiments of the disclosure encompass methods and compositions that enhance adoptive cell therapy by preventing or at least reducing on-target off-tumor effects of adoptive cell therapy. The disclosure concerns an immune effector cells of any type that are engineered to express two separate chimeric molecules: an activating chimeric antigen receptor that activates the immune effector cell through costimulatory domains following binding to a first antigen, and an inhibitory chimeric antigen receptor that inhibits cell-mediated activation upon binding to a second antigen. In specific cases, the inhibitory chimeric antigen receptor prevents fratricide and exhaustion by inhibiting activation of the cell through the activating chimeric antigen receptor when the inhibitory chimeric antigen receptor binds a particular antigen, including one adopted by sibling engineered immune effector cells through trogocytosis.

This application claims priority to U.S. Provisional Patent Application Ser. No. 63/193,363, filed May 26, 2021, and also claims priority to U.S. Provisional Patent Application Ser. No. 63/296,785, filed Jan. 5, 2022, both of which applications are incorporated by reference herein in their entirety.

### SEQUENCE LISTING

The instant application contains a Sequence Listing which has been submitted electronically in ASCII format and is hereby incorporated by reference in its entirety. Said ASCII copy, created on May 13, 2022, is named MDACP1275WO_ST25.txt and is 13,050 bytes in size.

### TECHNICAL FIELD

The present disclosure relates generally at least to the fields of immunology, cell biology, molecular biology, and medicine.

### BACKGROUND

Immune effector cells with CAR engineering have shown their revolutionary therapeutic effect against refractory malignancies (see, e.g., Rohaan et al., 2019; June & Sadelain 2018; and Rafiq et al., 2020). The CAR molecule redirects the cytotoxicity of immune effectors toward target cells expressing the cognate antigen. By equipping them with different antigen specificity, CAR-T cells and CAR-NK cells can mediate cytotoxicity against various types of tumor. The most promising clinical data have been reported with CD19 CART (see, e.g., June & Sadelain 2018; and Rafiq et al., 2020) and CD19 CAR NK cells (see, e.g., Liu et al., 2020). In addition to CD19, the antigen specificity of CARs can be directed against other surface molecules (see, e.g., Sadelain et al., 2013), such HER-2, ERBB-2, Mesothelin (MSLN), etc. (see, e.g., Rohaan et al., 2019; Sadelain 2015; and Morgan et al., 2010). An ideal tumor targeted antigen should be exclusively expressed on tumor cells. However, most tumor antigens are also expressed on normal tissue, albeit often at lower levels. As a result, the curative gains of using cellular therapy with CAR-T cells or CAR-NK cells may be hampered by the induction of on-target off-tumor effects in clinic (see, e.g., Kalos et al., 2011; Brentjens et al., 2011; Brentjens et al., 2013; and Parker et al., 2020). Indeed, infusion of CAR-T cells against HER-2, ERBB-2, or MSLN were thought to contribute to acute severe side effects such as acute respiratory failure as a result of cross-reactivity with the lung epithelium (see, e.g., Morgan et al., 2010; Hegde et al., 2020; and Haas et al., 2019). Though the use of immunosuppressive regimens such as corticosteroids can curb some of these deleterious toxicities, it can also compromise the therapeutic benefit of the cells (see, e.g., Lupo-Stanghellini et al., 2010; Vogler et al., 2010; and Kieback et al., 2008). More recently, CAR mediated trogocytosis (trogo) has been reported to transfer the targeted antigen to the CAR-T cells (see, e.g., Hamieh et al., 2019) and CAR-NK cells, resulting in fratricide of the trogo-positive CAR T and CAR NK cells, thereby limiting their in vivo persistence, and their capacity for anti-tumor activity in the clinic. Therefore, these on-target off-tumor effects are impeding clinical usage of CAR-T cells and CAR-NK cells.

### BRIEF SUMMARY

Embodiments of the disclosure include methods and compositions that enhance adoptive cell therapy. In particular embodiments, the methods and compositions enhance adoptive cell therapy by enhancing persistence of the cells of the adoptive cell therapy and/or by protecting cells that are not the target of the cell therapy. The disclosure provides for improvement of the efficacy of cellular therapies against cancers, including solid tumors, while minimizing their side effects. In specific embodiments, the efficacy of the cellular therapy is improved because it allows the engineered cells of the therapy to recognize and spare “off-target” normal or other cells from the “on-target” cancer cells without abrogating the function of the cellular therapy in tumor rejection.

The disclosure concerns genetically engineered immune effector cells of any kind with an inhibitory chimeric antigen receptor (iCAR) in addition to the cells being engineered to have one or more other engineered antigen receptors, including a chimeric antigen receptor (activating CAR, or aCAR) that comprises at least one activation signaling domain. The iCAR is configured to comprise one or more intracellular inhibitory domains (e.g., inhibitory signaling endodomains) that prevent cytotoxicity against cells that are not intended to be a target, including normal tissue and/or their sibling cells (other engineered immune effector cells). In specific embodiments, the iCARs utilize the natural inhibitory signaling of NK cell inhibitory receptors, such as KIRs and other NK cell inhibitory receptors. Thus, upon binding of the iCAR to the antigen to which it is targeted, the iCAR sends a signal to the cell expressing the iCAR not to kill the cell that expresses the antigen to which the iCAR is targeted. In specific embodiments, this dual system includes an NK self-recognizing iCAR that, upon binding of the iCAR to a cell expressing the antigen targeted by the iCAR, transfers a “don't kill me” signal to the NK cells expressing the iCAR, thereby stopping the engineered NK cells that express the iCAR from killing the cell (via the aCAR) to which the iCAR has targeted. Thus, upon engagement with the antigen that the iCAR binds, the cell expressing both the iCAR and aCAR does not kill the cell expressing the antigen targeted by the iCAR, because the iCAR inhibits the aCAR from killing the cell expressing the antigen targeted by the iCAR. In some cases, the antigen targeted by the iCAR happens to be expressed by normal cells or by sibling cells of the engineered immune effector cells (other engineered immune effector cells of the same kind), and the iCAR prevents their fratricide.

As noted, in specific embodiments, the iCAR of a particular engineered immune effector cell prevents killing through the aCAR of other engineered immune effector cells of the same kind. In specific embodiments, the immune effector cell expressing both the iCAR and aCAR expresses the antigen targeted by the iCAR because of trogocytosis (a process in which lymphocytes bound to antigen-expressing cells extract surface molecules from these antigen-expressing cells and then express them on their own surface). That is, in some cases an antigen targeted by the engineered immune effector cells is taken up by the engineered immune effector cells themselves, but the iCAR prevents one engineered immune effector cell from killing another immune effector cell that happens to express the antigen as a result of trogocytosis.

In specific embodiments, methods of the disclosure enhance the persistence and anti-tumor activity of CAR NK cells while preventing fratricide, off-target toxicity, and exhaustion, thus enhancing or improving anti-tumor cellular therapy.

Embodiments of the disclosure include compositions comprising an engineered immune effector cell, comprising: (a) at least one inhibitory chimeric antigen receptor (iCAR) comprising: (1) at least one extracellular antigen binding domain, wherein a first extracellular antigen binding domain binds a first antigen; (2) a first transmembrane domain; and (3) at least one natural killer (NK) cell inhibitory signaling domain and/or at least one co-inhibitory domain; and (b) at least one activating chimeric antigen receptor (aCAR) comprising: (1) at least one extracellular antigen binding domain, wherein a second extracellular binding domain binds a second antigen; (2) a second transmembrane domain; and (3) at least one activating endodomain with or without a costimulatory signaling domain. The cell may be an NK cell or a T cell. The first antigen and the second antigen may be different antigens. In specific cases, the first extracellular antigen binding domain of (a)(1) binds an antigen on an NK cell. The cell may be an NK cell and the first extracellular antigen binding domain of (a)(1) may bind an antigen on an NK cell, including KIR2DL1, KIR2DL2, KIR2DL3, KIR2DL5, KIR3DL1, KIR3DL2, KIR3DL3, KIR2DL4, KIR2DS1, KIR2DS2, KIR2DS3, KIR2DS4, KIR2DS5, KIR3DS1, DAP10, DAP12, CD56, CD57, CD25, CD122, NKP30, NKP44, NKP46, NKG2C, NKG2D, NKG2A, CRTAM, TIGIT, CD96, 2B4, CD16, CD27, CD100, CD160, ILT2, ILT4, KLRG1, LAIR1, CD161, CS1, an (Natural Cytotoxicity Receptor) NCR, KIR, and/or other NK-related antigen. In some cases, the iCAR has two antigen binding domains that each target different antigens. The second extracellular antigen binding domain of (b)(1) may bind a cancer antigen or a pathogen antigen. The second extracellular antigen binding domain of (b)(1) may bind a cancer antigen on a solid tumor or on a hematological malignancy. In specific embodiments, the NK cell inhibitory signaling domain and/or co-inhibitory domain is from an NK cell inhibitory receptor, and the NK cell inhibitory signaling domain and/or co-inhibitory domain may be from leukocyte immunoglobulin-like receptor (LIR-1), CD300A, NKG2A, Siglec-7, CD96, TIM3, TIGIT, LAIR-1, KIR2DL1, KIR2DL2, KIR2DL3, KIR2DL4, KIR2DL5, KIR3DL1, KIR3DL2, KIR3DL3, KIR2DL5A, and/or KIR2DL5B. The first transmembrane domain and the inhibitory signaling domain may or may not be from the same molecule; for example, the first transmembrane domain and the inhibitory signaling domain may be from LIR-1 or KIR2DL1. In specific embodiments, the iCAR comprises at least one co-inhibitory domain, including one from LAIR-1, NKG2A, CD300A, or a combination thereof. The first and/or second extracellular antigen binding domain may comprise an scFv or a natural ligand. In a specific embodiment, the second extracellular antigen binding domain of (b)(1) comprises an scFv that binds an antigen selected from the group consisting of CD19, EBNA, CD123, HER2, CA-125, TRAIL/DR4, CD20, CD70, HLA-G, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases, MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, GAGE-2, GAGE-3, GAGE-4, GAGE-5, GAGE-6, GAGE-7A, GAGE-7B, GAGE-8, NA88-A, MCIR, MDA-7, gp75, Gp100, PSA, PSM, Tyrosinase, tyrosinase-related protein, TRP-1, TRP-2, ART-4, CAMEL, CEA, Cyp-B, hTERT, hTRT, ICE, MUC1, MUC2, a Phosphoinositide 3-kinase, a TRK receptor, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1), TACSTD2, a receptor tyrosine kinase, Epidermal Growth Factor receptor (EGFR), EGFRvIII, platelet derived growth factor receptor (PDGFR), vascular endothelial growth factor receptor (VEGFR), VEGFR2, a cytoplasmic tyrosine kinase, integrin-linked kinase (ILK), signal transducers and activators of transcription STAT3, STATS, and STATE, HIF-1, HIF-2, Nuclear Factor-Kappa B (NF-B), a Notch receptor NY ESO 1, c-Met, mammalian targets of rapamycin (mTOR), WNT, extracellular signal-regulated kinases (ERKs), PMSA, PR-3, MDM2, Mesothelin, renal cell carcinoma-5T4, SM22-alpha, carbonic anhydrases I (CAI), CAIX), STEAD, TEL/AML1, GD2, proteinase3, hTERT, sarcoma translocation breakpoints, EphA2, ML-IAP, EpCAM, TMPRSS2 ETS fusion gene, ERG, NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, and a combination thereof.

In particular embodiments, the composition of the iCAR comprises: a first extracellular antigen binding domain of (a)(1) that comprises an scFv that binds CS1; an IgG1 hinge; a first transmembrane domain of (a)(2) from KIR2DL1; and an inhibitory signaling domain of (a)(3) from KIR2DL1.

In particular embodiments, the composition of the iCAR comprises: a first extracellular antigen binding domain of (a)(1) that comprises an scFv that binds CD19; an IgG1 hinge; a first transmembrane domain of (a)(2) from KIR2DL1; and an inhibitory signaling domain of (a)(3) from KIR2DL1.

In particular embodiments, the composition of the iCAR comprises: a first extracellular antigen binding domain of (a)(1) that comprises an scFv that binds CD19; an IgG1 hinge; a first transmembrane domain of (a)(2) from LIR-1; and an inhibitory signaling domain of (a)(3) from LIR-1.

In particular embodiments, the composition of the iCAR comprises: a first extracellular antigen binding domain of (a)(1) that comprises an scFv that binds CD19; an IgG1 hinge; a first transmembrane domain of (a)(2) from KIR2DL1; an inhibitory signaling domain of (a)(3) from KIR2DL1; and a co-inhibitory domain from LAIR-1.

In particular embodiments, the composition of the iCAR comprises: a first extracellular antigen binding domain of (a)(1) that comprises an scFv that binds CD19; an IgG1 hinge; a first transmembrane domain of (a)(2) from KIR2DL1; an inhibitory signaling domain of (a)(3) from KIR2DL1; and a co-inhibitory domain from NKG2A.

In particular embodiments, the composition of the iCAR comprises: a first extracellular antigen binding domain of (a)(1) that comprises an scFv that binds CD19; an IgG1 hinge; a first transmembrane domain of (a)(2) from KIR2DL1; an inhibitory signaling domain of (a)(3) from KIR2DL1; and a co-inhibitory domain from CD300A.

In specific embodiments, the iCAR comprises SEQ ID NO:2, SEQ ID NO:4, SEQ ID NO:6, SEQ ID NO:8, or SEQ ID NO: 10.

In specific embodiments, at least part of the iCAR is encoded by sequence of SEQ ID NO:1, SEQ ID NO:3, SEQ ID NO:5, SEQ ID NO:7, or SEQ ID NO:9.

Compositions comprising pluralities of any cells encompassed herein are encompassed, including any composition housed in a pharmaceutically acceptable carrier.

Embodiments of the disclosure include methods of enhancing an adoptive cell therapy for an individual in need thereof, comprising administering to the individual a therapeutically effective amount of engineered immune effector cells, each comprising: (a) at least one inhibitory chimeric antigen receptor (iCAR) comprising: at least one extracellular antigen binding domain, wherein a first extracellular antigen binding domain binds a first antigen; and at least one natural killer (NK) cell inhibitory signaling domain and/or at least one co-inhibitory domain; and (b) at least one activating chimeric antigen receptor (aCAR) comprising: at least one extracellular antigen binding domain, wherein a second antigen binding domain binds a second antigen; and an activating endodomain and at least one costimulatory signaling domain, and wherein: (I) when the first antigen and the second antigen are the same and when the engineered immune effector cell binds through the second extracellular antigen binding domain a cell that expresses the antigen, the iCAR inhibits the killing by the engineered immune effector cell of the cell that expresses the antigen; or (II) when the first and second antigen are non-identical and are both expressed on a fellow engineered immune effector cell or on a non-engineered immune effector cell of the same type or on a non-diseased cell, when the engineered immune effector cell binds through the second extracellular antigen binding domain to the second antigen on the fellow engineered immune effector cell or the non-engineered immune effector cell of the same type or the non-diseased cell, respectively, the iCAR inhibits the killing by the engineered immune effector cell of the fellow engineered immune effector cell or the non-engineered immune effector cell of the same type or the non-diseased cell, respectively. In particular cases, in (I) the cell that expresses the antigen is a non-cancerous cell. In some cases, in (I) the cell that expresses the antigen is a fellow engineered immune effector cell. In particular cases, in (II), the engineered immune effector cell binds through the second extracellular antigen binding domain to the second antigen on the fellow engineered immune effector cell. The second antigen may or may not be expressed by the fellow engineered immune effector cell as a result of trogocytosis.

In any method of the disclosure, engineered immune effector cells may be obtained from storage or they may be produced without storage. The engineered immune effector cells may or may not be engineered to express the iCAR prior to being engineered to express the aCAR. The engineered immune effector cells may or may not be engineered to express the iCAR subsequent to being engineered to express the aCAR. In specific embodiments, the engineered immune effector cells are NK cells and are engineered to express an iCAR that targets an NK cell self antigen. The engineered immune effector cells may be engineered to express an iCAR that targets an NK cell self antigen and are then engineered to express an aCAR that targets an antigen on cancer cells of the individual.

In any method of the disclosure, the individual has cancer (including metastatic) and is administered a therapeutically effective amount of a second cancer therapy, such as surgery, radiation, chemotherapy, drug therapy, hormone therapy, immunotherapy or a combination thereof. The second cancer therapy may be delivered prior to, during, or after the engineered immune effector cells.

In certain embodiments, the engineered immune effector cells are derived from cells that are autologous with respect to the individual or that are allogeneic with respect to the individual. Any engineered immune effector cells may be NK cells or T cells.

In specific embodiments, the first extracellular antigen binding domain binds an antigen on an NK cell. The engineered immune effector cells may be NK cells and the first extracellular antigen binding domain binds an antigen on an NK cell, including KIR2DL1, KIR2DL2, KIR2DL3, KIR2DL5, KIR3DL1, KIR3DL2, KIR3DL3, KIR2DL4, KIR2DS1, KIR2DS2, KIR2DS3, KIR2DS4, KIR2DS5, KIR3DS1, DAP10, DAP12, CD56, CD57, CD25, CD122, NKP30, NKP44, NKP46, NKG2C, NKG2D, NKG2A, CRTAM, TIGIT, CD96, 2B4, CD16, CD27, CD100, CD160, ILT2, ILT4, KLRG1, LAIR1, CD161, CS1, an (Natural Cytotoxicity Receptor) NCR, KIR, and/or other NK-related antigen.

In particular embodiments of the method, the iCAR has two antigen binding domains that each target different antigens. The second extracellular antigen binding domain may or may not bind a cancer antigen or a pathogen antigen. The second extracellular antigen binding domain may bind a cancer antigen on a solid tumor or on a hematological malignancy. In specific cases, the NK cell inhibitory signaling domain and/or co-inhibitory domain is from an NK cell inhibitory receptor. The first transmembrane domain and the inhibitory signaling domain may or may not be from the same molecule. In specific cases, the first transmembrane domain and the inhibitory signaling domain are from LIR-1 or KIR2DL1. The iCAR may comprise at least one co-inhibitory domain, including one from LAIR-1, NKG2A, CD300A, or a combination thereof.

In certain aspects, the first and/or second extracellular antigen binding domain comprises an scFv or a natural ligand, and the second extracellular antigen binding domain may comprise an scFv that binds an antigen selected from the group consisting of CD19, EBNA, CD123, HER2, CA-125, TRAIL/DR4, CD20, CD70, HLA-G, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases, MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, GAGE-2, GAGE-3, GAGE-4, GAGE-5, GAGE-6, GAGE-7A, GAGE-7B, GAGE-8, NA88-A, MCIR, MDA-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, a Phosphoinositide 3-kinase, a TRK receptor, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1), TACSTD2, a receptor tyrosine kinase, Epidermal Growth Factor receptor (EGFR), EGFRvIII, platelet derived growth factor receptor (PDGFR), vascular endothelial growth factor receptor (VEGFR), VEGFR2, a cytoplasmic tyrosine kinase, integrin-linked kinase (ILK), signal transducers and activators of transcription STAT3, STATS, and STATE, HIF-1, HIF-2, Nuclear Factor-Kappa B (NF-B), a Notch receptor NY ESO 1, c-Met, mammalian targets of rapamycin (mTOR), WNT, extracellular signal-regulated kinases (ERKs), PMSA, PR-3, MDM2, Mesothelin, renal cell carcinoma-5T4, SM22-alpha, carbonic anhydrases I (CAI), CAIX), STEAD, TEL/AML1, GD2, proteinase3, hTERT, sarcoma translocation breakpoints, EphA2, ML-IAP, EpCAM, TMPRSS2 ETS fusion gene, ERG, NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, and a combination thereof.

In particular cases of the method, the iCAR comprises: a first extracellular antigen binding domain of (a)(1) that comprises an scFv that binds CS1; an IgG1 hinge; a first transmembrane domain of (a)(2) from KIR2DL1; and an inhibitory signaling domain of (a)(3) from KIR2DL1.

In particular cases of the method, the iCAR comprises: a first extracellular antigen binding domain of (a)(1) that comprises an scFv that binds CD19; an IgG1 hinge; a first transmembrane domain of (a)(2) from KIR2DL1; and an inhibitory signaling domain of (a)(3) from KIR2DL1.

In particular cases of the method, the iCAR comprises: a first extracellular antigen binding domain of (a)(1) that comprises an scFv that binds CD19; an IgG1 hinge; a first transmembrane domain of (a)(2) from LIR-1; and an inhibitory signaling domain of (a)(3) from LIR-1.

In particular cases of the method, the iCAR comprises: a first extracellular antigen binding domain of (a)(1) that comprises an scFv that binds CD19; an IgG1 hinge; a first transmembrane domain of (a)(2) from KIR2DL1; an inhibitory signaling domain of (a)(3) from KIR2DL1; and a co-inhibitory domain from LAIR-1.

In particular cases of the method, the iCAR comprises: a first extracellular antigen binding domain of (a)(1) that comprises an scFv that binds CD19; an IgG1 hinge; a first transmembrane domain of (a)(2) from KIR2DL1; an inhibitory signaling domain of (a)(3) from KIR2DL1; and a co-inhibitory domain from NKG2A.

In particular cases of the method, the iCAR comprises: a first extracellular antigen binding domain of (a)(1) that comprises an scFv that binds CD19; an IgG1 hinge; a first transmembrane domain of (a)(2) from KIR2DL1; an inhibitory signaling domain of (a)(3) from KIR2DL1; and a co-inhibitory domain from CD300A.

In some cases of the method, the iCAR comprises SEQ ID NO:2, SEQ ID NO:4, SEQ ID NO:6, SEQ ID NO:8, or SEQ ID NO:10. In specific cases of the method, at least part of the iCAR is encoded by sequence of SEQ ID NO:1, SEQ ID NO:3, SEQ ID NO:5, SEQ ID NO:7, or SEQ ID NO:9.

Other objects, features and advantages of the present disclosure will become apparent from the following detailed description. It should be understood, however, that the detailed description and the specific examples, while indicating particular embodiments of the disclosure, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Expression of CAR in NK cells. Representative FACS plots showing the percentage (%) of CAR+ transduced primary human NK cells expressing either CAR19, 19scFv, or five different iCAR19 constructs (n=5 donors). The inset numbers are the percentages (%) of CAR-NK cells within the indicated gated regions.

FIG. 2. Dual-CAR expression in NK cells. Schematic representation of retrovirus vector encoding the aCAR19 and/or iCAR1-CS1. Following the expression of aCAR19 and iCAR1-CS1 in engineered NK cells, the surface expression was assessed using a tag-tandem protein CD19 and CS1 flow cytometric analysis; inset numbers are the percentages (%) of cells within the indicated gated quadrants. TM: transmembrane; SE: signaling endodomain.

FIGS. 3A-B. CAR mediated phosphorylation in NK cells. Representative flow cytometry histograms (left) and pooled data (right) summarizing the mean fluorescence intensity (MFI) of phospho-SHP1 (pSHP1) (A) and phospho-Syk/Zap70 (pSyk/pZap70) (B) in NK cells expressing CAR19, 19scFv, or the different iCAR19 constructs (n=5 donors) after co-culture with Raji CD19+ cells. P values were determined by two-tailed one-way ANOVA. ***P<0.0001. Data were assessed by flow cytometry and shown by mean+s.e.m. Each dot represents an individual donor.

FIGS. 4A-D. iCAR19 NK cells show reduced cytotoxicity against CD19+ targets. Plots summarizing the flow cytometry data on cytokine production (IFN-γ and TNF-α) and degranulation (CD107a) by CAR19 and each iCAR19 NK cells after co-culture with K562^{gCD19+} cells (A), Raji cells (B), K562 cells (C) or Raji^{CD19KO} cells (D) (n=5 donors per each condition). The statistical significance is represented as *P<0.01, **P<0.001, ***P<0.0001. Bars represent mean values+s.e.m. Each dot represents an individual donor.

FIGS. 5A-B. iCAR mediated NK cytotoxicity. Cytotoxicity (Incucyte analysis of percentage (%) Caspase-3/7+ events) of NK cells transduced with CAR19, 19scFv, or the different iCAR19 constructs, against Raji^{CD19+} cells (A) and autoNK^{gCD19+} target cells (B) over 30 hours as measured by Incucyte live imaging cell killing assay (representative of three donors). ***P<0.0001. Bars represent mean values+s.e.m. P values were determined by two-tailed two-way ANOVA.

FIG. 6. Dual-CAR mediated NK cytotoxicity against MM1S cells. Cytotoxicity (Caspase-3/7+) of CAR19/iCAR-CS1 ((ii); bottom) and CAR19/CS1scFv ((i); top) NK cells against MM1S^{CD19-/CS1+} targets over 30 hours as measured by Incucyte live imaging cell killing assay (n=3). **P<0.0001. Bars represent mean values+s.e.m.

FIGS. 7A-E. iCAR- and aCAR-expressing NK cells discern targets in vitro, (A-C) Incucyte analysis of the percentage (%) cytotoxicity (caspase-3/7+ events) of NK cells expressing 19scFv/CS1scFv (scFv; (i)), 19scFv/iCARCS1 (iCAR1; (ii)), aCAR19/CS1scFv (aCAR19; (iii)), or aCAR19/iCARCS1 (aCAR/iCAR; (iv)) (representative of 3 donors) against Raji^{CD19+/CS1-} cells, SKOV3^{gCD19+/CS1-} cells, Raji^{CD19-/CS1-} (e.g., CD19^{KO}/CS1⁻) cells and SKOV3^{CD19-/CS1-} cells (A), self-targets-NK^{CD19+/CS+} cells that overexpressed CD19 on their surface (B), and normal CD19+/CS1-low B cells (C), at different time points over 30 hours as measured by Incucyte live imaging cell killing assay (n=3). (D-E) CD107a (left) IFN-γ (middle), and TNF-α (right) production by each CAR-expressing NK cell in response to 6-hr stimulation with Raji cells (D) or K562 target cells (E) (n=3 donors). P values were determined by two-tailed two-way ANOVA in FIGS. 7A, B, and C, or two-tailed one-way ANOVA in FIGS. 7 D and E; *P<0.01, **P<0.001, ***P<0.0001, n.s. not significant. Data were shown by mean+s.e.m.

FIGS. 8A-B. Impact of iCAR signaling on NK cell expansion and proliferation. Graphs showing the cumulative population doublings (PDs) of NK cells expressing CAR19, 19scFv, or the different iCAR19 constructs over 70 days of culture with IL-2 alone (A), or IL-2 plus weekly uAPC stimulation (B) (n=5 donors). Bars represent mean values+s.e.m.

FIG. 9. Impact of iCAR signaling on CAR-NK proliferation. Graph showing the cumulative population doublings (PDs) of NK cells with dual-CAR expression ((i) CAR19/CS1scFv-NK cells; (ii) CAR19/iCAR1-CS1-NK cells) cultured with IL-2 only, (n=5 donors for each). Bars represent mean values+s.e.m.

FIGS. 10A-G. CAR19-mediated trogocytosis in NK cells co-cultured with CD19⁺ tumor targets. (A) Representative flow cytometric analysis (FACS) showing expression of CD19 on the CAR NK cell product, gated on the CAR positive vs the CAR-negative fractions, and in NK cells expressing 19scFv (no intracellular signaling domain), 5 min co-culture with Raji cells in conditions of pre-treatment with Latrunculin A (LatA) or vehicle control (representative for 3 donors). All NK cell populations were pre-gated on live, single cells, characterized as GFP⁻CD56⁺CD3⁻. The CD19⁺ gate was determined both based on fluorescent minus one (FMO) control and by referring to the negative control of NK cells cultured alone. Inset numbers indicate the percentages of cells within the indicated gated regions. (B) Trogocytic (TROG)-CD19 (tCD19) expression on singlet cells of CAR19-NK ((i) or (iv) with LatA), 19scFv-NK ((ii) or (v) with LatA), and NT-NK ((iii) or (vi) with LatA), shown as the ratio of TROG⁺/TROG⁻ NK cells at different time points after co-culture with Raji cells in conditions of pre-treatment with LatA or vehicle control (n=3 donors per condition). TROG⁺ fractions comprised NK cells expressing CAR⁺CD19⁺ (from FIG. 10A, Q2), while TROG⁻ fractions included NK cells expressing CAR⁺CD19⁻ (FIG. 10A, Q1). (C) tCD19 expression on singlet CAR 19-NK cells (light gray symbols; top) or 19scFv-NK cells (black symbols; bottom), shown as the ratio of TROG⁺/TROG⁻ cell populations at different time points after co-culture with patient-derived B-CLL cells (n=4) (circle, square, triangle, and diamond symbols) or patient-derived B-ALL cells (n=4) (X, plus, vertical line, or star symbols), respectively. Each symbol indicates a different patient. (D) Flow cytometric analysis representing the expression of CD107a and IFN-γ in TROG⁺ vs. TROG⁻ fractions of CAR19-NK cells after 6 hrs stimulation with Raji cells (representative of 3 donors); inset numbers indicate the percentages of cells within the indicated gated regions. Bar graphs show the percentage (%) of CD107a⁺ cells and IFN-γ⁺ cells in each fraction normalized to the expression level in CAR19-NK cells cultured alone (non-stimulated; n=5 donors per condition). (E) Schematic of engineering CD19-mCherry fusion protein expression on CD19-knockout Raji (Raji^{CD19-KO}) cells, controlled by Raji cells genetically modified for intracellular mCherry expression (Raji^{mCherry}). (F) Incucyte analyses showing the percentage (%) of CAR19-NK cells or 19scFv-NK cells acquiring mCherry expression (% NK^{TROG+} cells) after co-culture with engineered CD19-KO Raji^{CD19-mCherry} cells or Raji^{mCherry} in conditions of LatA pre-treatment or vehicle control (representative of 3 donors). (G) Incucyte analyses showing the percentage (%) of caspase 3/7 events in the TROG⁺ fraction of CAR19-NK cells vs. 19scFv-NK cells in conditions of culture alone, or co-culture with autologous fresh CAR19-NK cells. For CAR19-NK^{TROG+} cells, an anti-human CD19 blocking antibody (αCD19) was added to the culture media during incubation to block CD19-antigen, controlled by an antigen-mismatched scFv antibody (representative of 3 donors). P values were determined by two-tailed two-way ANOVA in FIGS. 10 B, C, F, and G, or two-sided Student's paired-t test in FIG. 10 D; ***P<0.0001; n.s.: not significant. Data were assessed by flow cytometry in FIGS. 10A-F, and shown by mean+s.e.m. Each circle represents an individual donor of NK cells or an experimental replication of Raji cells.

FIGS. 11A-H. Impact of antigen-induced self-engagement on CAR-NK effector cell phenotype and function. (A) Schematic illustrating a re-challenge assay in which CAR19-NK cells are repeatedly challenged (chlg) by autologous NK cells that were genetically engineered to express CD19 antigen (autoNK^{gCD19+}) at an E:T ratio of 1:3, controlled by their co-culture with autoNK cells (lacking CD19 expression). Both autoNK^{gCD19+} and autoNK cells were genetically modified to express intracellular GFP to facilitate their identification when co-cultured with effector CAR19-NK cells (GFP-negative). (B) tSNE analysis of live hCD45⁺GFP⁻CD56⁺CD3⁻ CAR19-NK cells 4 days after the second round of antigen challenge with autoNK^{gCD19+/gGFP+} cells; controls included CAR19-NK cells cultured alone or after 4 days of co-culture with autoNK^{gGFP+} cells. Cells from each condition were evaluated by mass cytometry, and their phenotypic signatures were merged to create a single t-SNE map (10,000 cells from 3 pooled donors per condition). Each t-SNA cluster (EC1-EC5) is represented in different colors, and frequencies of each cluster are indicated in contour plots generated for each condition. (C) Heat map of mass cytometry data representing expression levels of key NK cell phenotypic and functional markers in each cluster. The expression level for each marker is represented by the color light gray (low)-orange (high; positive Z scores are marked with a positive sign in the upper right corner of the respective circles, while negative Z scores are left unmarked) and the size of the circle shows expression of the marker, the larger the circle the greater the proportion of cells expressing the noted marker. TFs: transcription factors, Grm: granzymes. (D) Schematic illustrating the single-cell time-lapse imaging cytotoxicity assay. Time was recorded over 6 hours (T₀-T₃₆₀ₘᵢₙ) from the start of co-culture, where a single CAR-NK cell was incubated with a single targeted tumor cell. For the duration of the assay, the amount of time taken to detect Annexin V influx in the tumor cell was determined as the time taken to induce cell apoptosis. (E and F) Kaplan-Meier curves showing the percent (%) of apoptosis in the targeted cell when CAR-NK effector cells ((i): fresh CAR-NK cells; (ii): CAR-NK cells isolated after 4 days co-culture with autoNK cells (CD19 negative); (iii): CAR-NK cells isolated after the second round antigen challenge by autoNK^{gCD19+/GFP+} cells) were co-cultured with (E) K562 cells (comparisons: top (i) vs. (ii) P=0.4359; middle (i) vs. (iii) P<0.0001; bottom (ii) vs. (iii) P=0.0001) or (F) Raji cells; (comparisons: top (i) vs. (ii) P=0.1090; middle (i) vs. (iii) P<0.0001; bottom (ii) vs. (iii) P<0.0001). (G) Ex vivo analysis of CAR-NK cell glycolytic fitness as measured by ECAR (extracellular acidification rate); the following bar graphs to the right show their basal glycolysis ECAR level, determined by culturing cells in basal medium with 10 mM glucose (left), and their maximum ECAR glycolysis capacity after exposing cells to 1 mM oligomycin (a mitochondrial ATP synthase inhibitor, right). (H) Oxidative metabolism (OXPHOS) of CAR-NK cells as measured by OCR (oxygen consumption rate); the following bar graphs to the right show their basal OCR level determined at the steady state (left), and their maximal OCR in response to 0.5 mM FCCP exposure, which uncouples the mitochondrial OXPHOS chain (right). P value was determined by log-rank test in FIGS. 11 E and F, or two-sided Student's t test in FIGS. 11 G and H. Data were shown by mean+s.e.m. Each symbol represents an individual donor-derived CAR-NK cell sample.

FIGS. 12A-L. Impact of TROG-antigen acquisition on CAR-NK cell phenotype and function in vivo. (A) tSNE analysis of live hCD45⁺GFP⁻CD56⁺CD3⁻ NK cells collected from different organs (blood, bone marrow, spleen, and liver) of mice at different points during the treatment course. Phenotypic signatures of all collected NK cells were evaluated by mass cytometry and merged to create a single tSNE map, where the analysis generated four distinct color-coded clusters (C1-C4). (B) Contour plots showing the tSNE cluster prevalence in the pre-infusion product, 2 weeks after infusion (day 13-15), 3-4 weeks after infusion (day 20-27), or at the endpoint (day 29-34). The number of cell objects for each condition was indicated. (C) Frequencies of NK cells expressing the TROG-antigen (tCD19) in the CAR19-positive cells (upper, CAR-NK cell) or CAR-negative (lower, NT-NK cell) fractions at different time points during treatment are presented, controlled by their counterparts in the pre-infusion product. (D) Heat map representing the expression levels of phenotypic and functional markers on CAR-NK cells within each cluster. The expression level for each marker is represented by the color gray (low)—orange (high; positive Z scores are marked with a positive sign in the upper right corner of the respective circles, while negative Z scores are left unmarked) and the size of the circle shows expression of the marker, the larger the circle the greater the proportion of cells expressing the noted marker. (E) FlowSOM analysis of post-infusion NK cell populations where each metacluster is mapped using a self-organizing mapping strategy. Each colored region corresponds to a metacluster with inserted pie chart representing the frequency of NK cells expressing CAR and TROG-antigen (tCD19) on clustered cells; the size of each chart represents the number of clustered cells. (F and G) Violin plots showing the expression of (F) CAR19, and (G) tCD19 on CAR19-NK cells in each cluster, determined based on their level in pre-infused NT-NK cells shown as the gray line, (H) Violin plot showing cisplatin levels in post-infusion CAR19-NK^{TROG+} cell (expressing CD19) or their CAR19-NK^{TROG-} counterparts for each cluster. Cisplatin level represents the cellular viability of each population. (I-L) Violin plots showing the expression of (I) c-Kit, EOMES and Tbet; (J) ZAP70, Syk, and 2B4; (K) Granzyme (Gr) A, GrB, and perforin; (L) PD-1, TIM3, and TIGIT in TROG⁺ and TROG⁻ fractions of CAR19-NK cells. The median expression strength for each marker in CAR19-NK cells prior to infusion (in C1) is indicated by the gray line. P values were determined by two-tailed Wilcoxon matched pairs test; *P<0.001, **P<0.0001, ***P<0.00001.

FIGS. 13A-D. A lower level of CAR-mediated TROG-antigen expression was associated with improved clinical response to CAR-NK cell-based immunotherapy. (A) tCD19 expression on singlet cells of donor-derived CAR-expressing NK cells (NK^{CAR+}), donor-derived non-CAR expressing NK cells (NK^{CAR-}), and patient-derived NK cells at different time points after receiving CAR19-NK cell immunotherapy. Geometric mean fluorescent intensity (gMFI) of tCD19 expression was assessed by flow cytometry. Samples from individual patients at different times after CAR-NK cell infusion are presented. (B) Non-linear regression analyses using polynomial models show tCD19 expression on donor-derived NK^{CAR+} cells over time after CAR-NK cell infusion. The normalized mean tCD19 gMFI on CAR19-NK cells for the whole patient population was 6.29 (range of 0.61-35.77). Patients with a high (>mean) versus low (≤mean) normalized tCD19-gMFI at more than one time point were defined as group of TROG^{high} (ii) (n=4 patients) vs. TROG^{low} (i) (n=7 patients), respectively. (C) CD19 expression (upper), and cell counts (lower) for singlet CD19⁺ B cells in the TROG^{low} vs. TROG^{high} patient groups at different time points after CAR-NK cell infusion. (D) Pie charts showing the number of responders (res, upper) vs. non-responders (non-res, lower) after receiving CAR19-NK cell infusion in the TROG^{low} vs. TROG^{high} groups. P value was determined by two-tailed two-way ANOVA in FIG. 13 B, two-tailed one-way ANOVA in FIG. 13 C, two-tailed Fisher's exact test in FIG. 13 D; *P<0.1. Data were assessed by mass cytometry and shown by mean+s.e.m. Each circle represents an individual patient.

FIGS. 14A-I. Genetic modification of NK cells to express an iCAR reduced fratricide and exhaustion induced by aCAR-NK cells. (A) Diagram illustrating the engagement of AI-CAR-expressing NK cells with potential targets; “−” symbol indicates inhibitory signal, “+” symbol indicates activating signal. (B) Flow cytometric analyses representing the expression of phos-CD3ζ (pCD3z, left) and phos-Syk/Zap70 (right) in NK cell expressing 19scFv/CS1scFv (scFv only—no intracellular signaling; (i)), 19scFv/iCAR1-CS1 (iCAR/scFv; (ii)), aCAR19/CS1scFv (aCAR/scFv; (iii)), or aCAR19/iCAR1-CS1 (aCAR/iCAR; (iv)) after stimulation with Raji^{CD19+/CS1-} cells; the following bar graphs show their expression, determined by the fold change (FC) of their gMFI after normalization to isotope control (n=5 donors per condition). (C) Incucyte analyses showing the percentage (%) of caspase 3/7 events in CD19⁺CS1⁻ primary tumor cells derived from patients with CLL after co-culture with CAR-expressing NK cells, controlled by scFv-expressing NK cells (representative of 3 donors). (D) Flow cytometric analyses representing the expression of phos-CD3ζ (pCD3z, left) and phos-Syk/Zap70 (right) in NK cell expressing 19scFv/CS1scFv (scFv only—no intracellular signaling; (i)), 19scFv/iCAR1-CS1 (iCAR/scFv; (ii)), aCAR19/CS1scFv (aCAR/scFv; (iii)), or aCAR19/iCAR1-CS1 (aCAR/iCAR; (iv)) after stimulation with genetically-modified CD19⁺ autoNK^{CSI+} cells; the following bar graphs showing their expression, determined by the FC of their gMFI after normalization to isotope control (n=5 donors per condition) (E) Incucyte analysis showing the percentage (%) of caspase 3/7 events in gCD19⁺CS1⁺ autoNK cells after co-culture with CAR-expressing NK cells, controlled by scFv-expressing NK cells (representative of 3 donors); 19scFv/CS1scFv (scFv only—no intracellular signaling; (i)), 19scFv/iCAR1-CS1 (iCAR/scFv; (ii)), aCAR19/CS1scFv (aCAR/scFv; (iii)), or aCAR19/iCAR1-CS1 (aCAR/iCAR; (iv)). (F and G) (F) Co-expression of PD1, TIM3, and TIGIT, and (G) the expression of TFs, shown as the ratio of EOMES over Tbet expression in singlet CAR-expressing NK cell populations after the second round of antigen challenge with autoNK^{gCD19+/CS1+/GFP+} cells (n=5 donors per condition). Representative flow cytometry histogram expression for EOMES and Tbet are shown (left panel). (H) Cumulative population doublings (PDs) of each CAR-expressing NK cells (n=3 donors per condition) over 70 days of culture with IL-2; 19scFv/CS1scFv ((i)), 19scFv/iCAR1-CS1 ((ii)), aCAR19/CS1scFv ((iii)), or aCAR19/iCAR1-CS1 ((iv)). (I) tCD19 expression on singlet CAR-expressing NK cells (n=3 donors per condition) indicated as the ratio of TROG⁺/TROG⁻ cell populations at different time points after co-culture with Raji cells, controlled by scFv-expressing NK cells (representative of 3 donors); 19scFv/CS1scFv ((i)), 19scFv/iCAR1-CS1 ((ii)), aCAR19/CS1scFv ((iii)), or aCAR19/iCAR1-CS1 ((iv)). P values were determined by two-tailed two-way ANOVA in FIGS. 14 C, E, H, and I, or two-sided one-way ANOVA in FIGS. 14 B, D, F, and G; ***P<0.0001; n.s: not significant. Data were assessed by flow cytometry in FIGS. 14 B, D, F, G, and I, and shown as mean±s.e.m. Each circle represents an individual donor.

FIGS. 15A-Q. AI-CAR expressing NK cells showed superior in vivo anti-tumor activity. (A) Schematic illustration of the timeline using a mouse model engrafted with Raji cells. Luciferase/GFP-expressing CD19⁺ Raji cells (0.2×10⁵) were infused in mice, followed by a single infusion of NK cells expressing 19scFv/CS1scFv, 19scFv/iCAR-CS1, aCAR19/CS1scFv, or aCAR19/iCAR-CS1. trt.: treatment, n=5 mice per group. (B.1, B.2 and C) Tumor burden was assessed weekly by bioluminescence imaging (BLI), (B.1 and B.2) representative images for specific time points (Day 0, Day 7, Day 14, and Day 21) are shown for Raji cells alone (B.1 and B.2 left column) 19scFv/CS1scFv treated animals (B.1, middle column), 19scFv/iCAR-CS1 treated animals (B.1, right column), aCAR19/CS1scFv treated animals (B.2, middle column), or aCAR19/iCAR-CS1 treated animals (B.2, right column). trt.: treatment, n=5 mice per group; and (C) the normalized intensity of BLI in mice after infusion with the different NK cell groups. Untreated mice were used as the control; dashed lines indicate data for each mouse. (D) Kaplan-Meier curves showing the percent survival of mice after infusion with the different NK cell treatment groups. (E) CD19 expression on Raji cells, shown as the count of molecules per cell, in peripheral blood (left) and bone marrow (BM, right), harvested from mice at different time points after infusion of CAR-expressing NK cells, with the non-treated tumor only group as control. (F) tCD19 expression on singlet NKCAR¹⁹⁺ cells, indicated as TROG⁺/TROG⁻ ratio cell populations (left graph), and the percentage (%) viability in TROG⁺ fraction (NK^{tCD19+}, right graph) of NK cells expressing aCAR19/CS1scFv ((i)) or aCAR19/iCAR-CS1 ((ii)), harvested from the peripheral blood of mice at different time points after aCAR 19-expressing NK cell infusion (n=5 mice per group). (G) The percentage (%) of live GFP⁻CD3⁻CD56⁺ aCAR19⁺ NK cells in the peripheral blood of mice at different time points after infusion of cells expressing aCAR19/iCAR1-CS1 ((i), left) vs. aCAR19/CS1scFv ((ii), right; n=5 mice per group). (H) Cell count of live NK^{aCAR19+} cells in blood and spleen at days 3, 10, and 20 after infusion; cells expressing aCAR19/iCAR1-CS1 ((ii); top clusters) vs. aCAR19/CS1scFv ((i); bottom clusters) are shown (n=5 mice per group). (I) Schematic illustration of the timeline using mice engrafted with SKOV3^{ROR1+} ovarian cancer cells, and treated with a single dose infusion of NK cell expressing aCAR-ROR1/CS1scFv, or aCAR-ROR1/iCAR-CS1, with a non-treated tumor engrafted group as control (n=5 mice per group). (J-L) Tumor burden in SKOV3 engrafted mice without NK cell treatment (left column; (i)), with aCAR-ROR1/CS1scFv NK cell treatment (middle column; (ii)), or with aCAR-ROR1/iCAR-CS1 NK cell treatment (right column; (iii)) was determined weekly by BLI, (J) representative images from specific time points are shown (Day 0, Day 7, Day 21, and Day 28); (K) graph showing the normalized intensity of BLI over time during the treatment course, dashed lines indicate data for each mouse, and (L) the BLI intensity for each group at day 28. (M and N) (M) tROR1 expression on singlet NK^{aCAR-ROR1+} cells, shown as the ratio of TROG⁺/TROG⁻ cell populations, and (N) the percentage (%) viability in TROG⁺ fraction (NK^{tROR1+}) of NK cells expressing aCAR-ROR1/CS1scFv ((i), left bars), or aCAR-ROR1/iCAR-CS1 ((ii), right bars), harvested from the peripheral blood of mice at different time points after NK cell infusion (n=5 mice per group). (O) The percentage (%) of live GFP-CD3⁻CD56⁺aCAR-ROR1⁺ NK cells in the peripheral blood of mice at different time points after infusion of NK cells expressing aCAR-ROR1/CS1scFv ((i), left) or aCAR-ROR1/iCAR-CS1 ((ii), right; n=5 mice per group). (P) Cell count of live NK^{aCAR-ROR1+} cells in blood at days 5, 15, and 30 after infusion of NK cells expressing aCAR-ROR1/CS1scFv ((i), lower clusters), or aCAR-ROR1/iCAR-CS1 ((ii), upper clusters; n=5 mice per group). (Q) Charts the levels of IFN-γ and TNF-α in sera of mice at days 3, 10, and 20 after infusion of CAR-expressing NK cells; Raji only ((i)), Raji+19scFv/iCAR-CS1-NK ((ii)), Raji+19scFv/iCAR-CS1-NK ((iii)), Raji+aCAR19/CS1scFv-NK ((iv)), and Raji+aCAR19/iCAR-CS1-NK ((v)); (n=5 mice per group). P value was determined by two-tailed two-way ANOVA in FIGS. 14 C, E, K, and I or two-tailed one-way ANOVA in FIGS. G, O, and L, log-rank test in FIG. 14 D, or two-tailed Student's t test in FIGS. 14 F, H, M, N, P, and Q; ***P<0.0001. Data were pooled from two independent experiments in FIGS. 14 C, D, Q, and L, where NK cells were derived from different donors, or assessed by flow cytometry in FIGS. 14 E, F, G, H, M, N, O, and P, and shown as mean+s.e.m. Each symbol represents an individual mouse sample.

FIGS. 16A-G. CAR-mediated CD19 trogocytosis on NK cells in vitro. (A) Gating strategy to distinguish NK cells and Raji cells in co-culture experiments. Expression of GFP, CD56, and CAR was used to identify CAR-NK cells (GFP⁻CD56⁺CAR⁻), Raji cells genetically modified to express GFP (CD19⁺GFP⁺CD56⁻CAR⁻), and CAR-NK/Raji doublets (CD19⁺GFP⁺CD56⁺CAR⁺). Expression of CD19 on CAR-NK cells after co-culture with Raji cells was compared to that of control CAR-NK cells cultured alone. Inset numbers indicate the percentage of cells within the indicated gated regions. (B) Representative AMNIS® images showing surface protein expression of CD56, CAR, CD19, and intracellular expression of F-actin and GFP in CAR-NK cells cultured alone, Raji cells cultured alone, or CAR-NK/Raji doublets (CAR-NK cell engaged with Raji cell). Cells were identified by nuclear stain with DAPI; scale bar indicates 7 μm. Representative images show trogocytic CD19 (tCD19) expression on CAR-NK cells following engagement with Raji tumor targets. (C) AMNIS® imaging flow cytometry analysis on the surface of singlet CAR-NK cells cultured alone (negative control), CAR-NK cells engaged with Raji cells (d: CAR-NK/Raji doublet), or the singlet CAR-NK cells after 5 min co-culture with Raji cells. The geometric mean fluorescent intensity (gMFI) of CD19 was indicated for each condition. Inserted image shows representative cells for each culture condition. (D) Quantification of the AMNIS® imaging flow cytometry analysis showing the percentage (%) of tCD19 expression on singlet CD56⁺ CAR-NK cells cultured alone, CAR-NK cells engaged with Raji cells (d: CAR-NK/Raji doublet), or the tCD19⁺ fractions of singlet CAR-NK cells after 5 min co-culture with Raji cells (left); and percentage (%) of co-localized tCD19 and CAR molecules in singlet NK cells (right) (n=25 objects per culture condition). (E-F) Flow cytometric analyses representing the expression of CD19, CD20, and CD22 at (E) the protein level and (F) the mRNA level in CAR-NK cells cultured alone, Raji cells cultured alone, and CAR-NK cells after 5 min of co-culture with Raji cells (representative of 3 donors). Inset numbers indicate the percentages of cells within the indicated gated regions. (G) Displays bar graphs showing the summary data for each of the markers displayed in FIGS. 16 E and F. P values were determined by two-tailed one-way ANOVA in FIG. 16 D. or two-tailed student t-test in FIG. 16 G; **P<0.001, ***P<0.0001. Data were shown by mean+s.e.m. Each circle represents an individual cell.

FIGS. 17A-F. CAR 19-mediated TROG-CD19 transfer on NK cells is associated with reciprocal CD19 antigen reduction on tumor targets. (A) tCD19 expression on singlet CAR19-NK ((i)), NT-NK ((ii)), CAR19-T ((iii)), or T ((iv)) cells, shown as the ratio of TROG⁺/TROG⁻ cell populations at different time points after co-culture with RajiCD19+ cells (n=3 donors). (B) The flow cytometry histograms showing different levels of CD19 expression on Raji clones after CD19 gene knockout using CRISPR-Cas9 (left); five clones were selected that expressed CD19 at high (H; (i)), medium high (MH; (ii)), medium (M; (iii)), medium low (ML; (iv)), or low (L; (v)) levels. The gMFI for CD19 is indicated for each Raji cell clone. Acquisition of tCD19 expression on singlet CAR19-NK cells co-cultured with the different Raji clones is presented as the ratio of TROG⁺/TROG⁻ cell populations (right graph) (n=3 donors). (C) CD19 expression, determined as the number of molecules per cell, on singlet cells of Raji at different time points after co-culture with CAR19-NK cells or 19scFv-NK cells in conditions of pre-treatment with LatA or vehicle control (n=3 donors). (D) tCD19 expression on singlet cells of CAR19-NK, 19scFv-NK, or NT-NK, shown as the ratio of TROG/TROG cell populations, at different time points after co-culture with CD19⁺ target cells, including NALM-6, Ramos, healthy B cells, or SKOV3 genetically modified to express CD19 (SKOV3^{gCD19+}) (n=3 NK cell donors per tumor condition). (E) CD19 expression, determined as the number of molecules per cell, on singlet cells of NALM-6, Ramos, healthy B cells, or SKOV3^{gCD19+}, respectively, at different time points after co-culture with CAR19-NK cells or 19scFv-NK cells (n=3 donors per co-culture condition). (F) CD19 expression, determined as the number of molecules per cell on singlet CD19⁺ primary B cells derived from patients with either CLL (left panel) or ALL (right panel) (n=5 patients per each experimental condition), respectively, at different time points after co-culture with CAR19-NK cells or 19scFv-NK cells. P values were determined by two-tailed two-way ANOVA; ***P<0.0001. Data were assessed by flow cytometry and shown by mean+s.e.m.

FIGS. 18A-E. CAR-NK cell-mediated trogocytosis occurs with several targets and tumor cell types. Cognate TROG-antigen expression on singlet NK cells transduced with (A) CAR5-NK cells co-cultured with CD5⁺ CCRF tumor targets, (B) CAR70-NK cells co-cultured with CD70⁺ THP-1 tumor targets, (C) CAR123-NK cells co-cultured with CD123⁺ MOLM14 tumor targets, (D) CAR-BCMA-NK co-cultured with BCMA MM1-S tumor targets, or (E) CAR-ROR1-NK cells co-cultured with ROR1⁺ SKOV3 tumor targets. TROG-antigen expression is shown as the ratio of TROG⁺/TROG⁻ NK cell populations at different time points during co-culture (left panel for FIGS. 18A-E). Expression levels of cognate antigens, based on the number of surface molecules per cell, are presented for tumor cell lines before and after co-culture with the relevant CAR-NK cells (right panel for FIGS. 18A-E). n=3 NK cell donors per tumor condition. CAR19-NK cells (antigen-mismatched) and NT-NK cells were used as control. P values were determined by two-tailed two-way ANOVA; ***P<0.0001. Data were assessed by flow cytometry and shown by mean+s.e.m.

FIGS. 19A-I. NK cell-mediated trogocytosis that occurs with CAR-NK cells is affected by the affinity of the CAR for its cognate antigen and is determined by the different activating signaling endodomains. (A) Binding affinity of NK cells expressing CARs with different NK binding affinity for CD70, including the extracellular domain of CD27 and scFv70s derived from two different antibody clones (ARGX-110 or LB #14) (left panel), and TROG-antigen expression on singlet CD70-targeting NK cells, shown as the ratio of TROG⁺/TROG⁻ cell populations at different time points during co-culture with Raji^{CD70+} cells (right panel) (n=3 donors per condition). (B-I) TROG-antigen expression on singlet NK cells expressing anti-CD5 CARs with different intracellular signaling endodomains: (B) CD28/CD3ζ, (C) CD3ζ, (D) DAP10/CD3ζ, (E) DAP10 only (no CD3ζ), (F) NKG2D/CD3ζ, (G) 4-1BB/CD3ζ, (H) DAP12/CD3ζ, or (I) DAP12 only (no CD3ζ), respectively, shown as the ratio of TROG⁺/TROG⁻ cell populations at different time points during co-culture with Jurkat^{CD5+} cells (n=3 donors per condition). NT-NK cells were used as control. P values were determined by two-tailed two-way ANOVA; ***P<0.0001. Data were assessed by flow cytometry and shown by mean+s.e.m.

FIGS. 20A-E. CAR19-mediated acquisition of tCD19 on NK cells from targeted Raji cells was associated with decreased anti-tumor cytotoxicity. (A) UMAP analyses of CAR-NK cells collected after co-culture with Raji^{CD19+} cells. Phenotypic signatures of all collected CAR-NK cells were evaluated by mass cytometry, and data from 10,000 cells derived from 3 donors were merged to create a single UMAP map, where the analysis generated seven distinct color-coded clusters ((i) is CD2⁺KIR⁺NKG2A⁻NKG2C⁺CD94⁻; (ii) is CD2⁻NKG2A⁻NKG2C⁺CD94⁻; (iii) is CD2⁻KIR⁺NKG2A⁺CD94⁺; (iv) is CD2⁺KIR⁺NKG2A⁺NKG2C⁻CD94⁺; (v) is CD2⁺NKG2A⁺NKG2C⁻CD94⁺KLRG1⁺; (vi) is CD2⁺KIR⁺NKG2A⁺CD94⁺CD16⁻; and (vii) is CD2⁺KIR⁺NKG2A⁺CD94⁺CD16⁺) that represented the different subsets of NK cells. Marker expression for each NK cell subset was shown in UMAP plots (2B4, CD2, CD94, DAP12, KIR, KLRG-1, TRAIL, NKG2D, NKG2A, NKG2C, NKp30, NKp44, NKp46, and CD16, respectively). (B) Contour plots showing the UMAP cluster prevalence of CAR-NK cell alone, or after 30 min, 1 hr, 3 hrs, or 6 hrs of co-culture with Raji cells. The percentage of TROG⁺ CAR-NK cells, and tCD19 expression are presented for each subset for the different conditions. (C) Real-time images representing the co-culture of CAR19-NK cells (green) with Raji^{CD19-mCherry} cells (red). Black arrows (top row) indicate events of cell apoptosis; yellow arrows (second and bottom rows, 4 min and 20 min (upper two arrows) conditions) indicate immunologic synapse-like structures; white arrows (second and bottom rows, 12 min, 20 min, (bottom left arrow), 34 min, 44 min, and 54 min conditions) indicate CAR19-NK cells with evidence of mCherry transfer; scale bar indicates 10 μm. (D) Flow cytometric analyses representing co-expression of CD19 and mCherry on singlet Raji^{CD19-mCherry} cells cultured either alone ((i), representative for 3 samples) or CAR19-NK cells after co-culture with Raji^{CD19-mCherry} cells for 5 mins only ((ii), representative for 5 donors); the following graph shows the correlation between mCherry (as determined as gMFI) and CD19 (as determined as the number of molecules per cell) expression for each singlet cell. (E) CD19 expression, determined as the number of molecules per cell, and gMFI of mCherry expression on singlet Raji^{CD19-mCherry} cells at different time points during co-culture with CAR19-NK cells (n=3 donors). P values were determined by Pearson's correlation coefficient in FIG. 20 D, or two-tailed Student's t-test in FIG. 20 E; ***P<0.0001. Data were assessed by flow cytometry in FIGS. 20 D and E, and shown by mean+s.e.m.

FIGS. 21A-J. Impact of repeated antigen-induced CAR activation on CAR-NK cell phenotype. (A) Tumor rechallenge assay where CAR-19 NK cells were repeatedly challenged with Raji^{CD19-mCherry} at different E:T ratios of 3:1 ((i)), 1:1 ((ii)), or 1:3 ((iii)), respectively. Tumor cells were added every 2 days (n=5 donors) and tumor cell killing was measured by the tumor cell index and percentage (%) of caspase 3/7 events in Raji^{CD19-mCherry} (B-D). (B-D) Incucyte analyses showing the percentage (%) of caspase 3/7 events in Raji^{CD19-mCherry} after co-culture with CAR 19-NK cells at E:T ratios of (B) 3:1, (C) 1:1, or (D) 1:3. Tumor cell were added every 2 days, data were normalized to tumor cell alone. (E) tCD19-mCherry expression on singlet CAR19-NK cells is shown as the ratio of TROG⁺/TROG⁻ cells at different time points after re-challenge with Raji^{CD19-mCherry} cells. (F) Incucyte analyses showing the percentage (%) of caspase 3/7 events in Raji^{CD19-mCherry} cells after co-culture with CAR19-NK cells, 6 days after rechallenge at E:T ratios of 3:1, 1:1, or 1:3 respectively compared to co-culture with fresh CAR-NK cells. (G) Schematic illustrating the re-challenge assay in which CAR19-NK cells are repeatedly challenged with CD19-expressing Raji cells, or autologous NK cells genetically modified to express CD19 (autoNK^{gCD19-/GFP}) at an E:T ratio of 1:3. Raji cells and autoNK^{gCD19+} target cells were modified to express GFP to facilitate their identification when co-cultured with effector CAR19-NK cells (GFP-negative). (H) Flow cytometric analysis of CD19 expression on CAR19-NK cells cultured alone, on the TROG⁺ (tCD19⁺) vs. TROG⁻ fractions of CAR19-NK cells after 1 hr of co-culture with Raji cells, and on autoNK^{gCD19+} cells cultured alone. (I) Number of CD19 molecules per cell for CAR19-NK cells cultured alone, the TROG⁺ (tCD19⁺) vs. TROG⁻ fractions of CAR19-NK cells after co-culture with Raji cells, and autoNK^{gCD19+} cells cultured alone (n=3 donors). (J) The percentage (%) of PD1, TIM3, and TIGIT co-expression (lower left panel), and ratio of EOMES vs Tbet (lower right panel) in CAR19-NK cells after several rounds of antigen challenge with Raji^{CD19+/gGFP+} cells (circles: tumor targets) or autologous NK cells genetically modified to express both CD19 and intracellular GFP (NK^{gCD19+/GFP+} cells; squares: self-targets). CAR19-NK cells were evaluated after days (D) of co-culture with rounds (R) of antigen challenge (e.g., D2R1 is day 2, with 1 round of antigen challenge). Representative histograms for each marker expression are shown. TT: tumor targets (Raji^{CD19+}), ST: self targets (autoNK^{gCD19+}) (n=5 donors per condition). P values were determined by two-tailed two-way ANOVA in FIGS. 21 A-F or Student's t test in FIGS. 21 I and J; *P<0.01, **P<0.001, ***P<0.0001, n.s. not significant. Data were assessed by flow cytometry and shown by mean+s.e.m. Each symbol indicates an individual donor.

FIGS. 22A-F. Repeated challenge of CAR19-NK cells with autoNK^{CD19+} cells result in CAR-NK cell hyporesponsiveness. (A) tSNE analysis of live hCD45⁺GFP⁻CD56⁺CD3⁻ CAR19-NK cells 4 days following a second round of antigen challenge with autoNK^{gCD19+gGFP+} cells; controls include CAR19-NK cells cultured alone or co-cultured with autoNK^{gGFP+} cells for 4 days (lacking CD19 expression). The phenotypic cell signature for each condition was evaluated by mass cytometry and merged to create a single t-SNE map (10,000 cells from 3 pooled donors per condition). All cells were marked (blue (i)), and expression of CAR19 (green; (ii)) and tCD19 (orange; (iii)) was determined based on their expression on NT-NK cell controls. Insert numbers indicate the percentages (%) of CAR and tCD19 expression on CAR19-NK cells for each condition. (B) Kaplan-Meier plots showing the percentage (%) of apoptotic Raji cells following co-culture with CAR-NK cell isolated after the 1^{st} round (2 days, left) or 3^{rd} round (6 days, right) of re-challenge with autoNK^{gCD19+/GFP+} cells when compared to fresh CAR-NK cells. Assays were performed in microwells with E:T ratio of 1:1; data were pooled from two donors. (C) Schematic representation of single-cell time-lapse imaging cytotoxicity assay, where a single CAR-NK cell was cultured with two Raji cells. Annexin V influx in Raji cells defined cell apoptosis. The bar plots on the right show the percentage of CAR-NK cells isolated at different time points after re-challenge with autoNK^{gCD19+/GFP+} cells that succeeded in lysing one (light) or two (dark) Raji cells. Fresh CAR-NK cells (pre) were used as control (n=2-4 donors). (D-F) Incucyte analyses showing the percentage (%) of caspase 3/7 events in Raji cells co-cultured with CAR19-NK cells isolated after (D) the 1^{st} round, (E) 2^{nd} round, or (F) 3^{rd} round of re-challenge with autoNK^{gCD19+/GFP+} cells. Fresh CAR-NK cells and CAR-NK cells that were isolated after each round of re-challenge and cultured for 24 hrs in complete media supplemented with 100 U/mL IL-2 (referred to as ‘rested’ cells) were used as controls (representative of 3 donors). P value was determined by log-rank test in FIG. 22 B, two-tailed paired Student t test in FIG. 22 C, or two-tailed two-way ANOVA in FIGS. 22 D, E, and F; *P<0.01, **P<0.001, ***P<0.0001. Data were shown by mean+s.e.m.

FIGS. 23A-E. CAR19-NK cell trogocytosis and reciprocal reduction in CD19 antigen expression on tumor cells in vivo. (A) Schematic illustration of timeline using a mouse model engrafted with Raji cells. Mice received three dose levels of Luc/GFP-expressing CD19⁺ Raji cells (0.2×10⁵, 1×10⁵, or 5×10⁵), respectively, followed by a single infusion of CAR19-NK cells (1×10⁷) or NT-NK cells alone (1×10⁷) as control. (B) Graphs showing the intensity of bioluminescence imaging (BLI) over time for Raji cells only (black); Raji cells with NT-NK cell infusion (dashed blue or light gray); and Raji cells with CAR19-NK cell infusion (solid green or darker gray); shown are the BLI for each of the timepoints of days 0-7 vs days 7-14 vs days 14-21. (C) tCD19 expression (indicated as TROG⁺/TROG⁻ ratio) on singlet hCD45⁺GFP⁻CD56⁺CD3⁻ on CAR-NK cell products gated on CAR19 (dark gray; left bars) vs. CAR-negative fractions (light gray; right bars) in peripheral blood samples collected at different time points after infusion (n=15 mice per group). (D) CD19 expression on Raji cells, shown as the count of molecules per cell in peripheral blood (left, n=15 mice), and organs [spleen, liver, bone marrow (BM), and blood] (right, n=24 mice) of mice at the end time point after CAR19-NK cell infusion. (E) CD19 expression on Raji cells, shown as the count of molecules per cell in organs harvested at the end time point after NT-NK cell infusion (n=10 mice). P values were determined by two-tailed one-way ANOVA for analyses. Data were assessed by flow cytometry in samples with cell objects of interest >20 counts, and shown by mean+s.e.m. Each circle represents an individual mouse sample and outliers are indicated as dark dots.

FIGS. 24A-B. CD19 expression in Raji cells retrieved from Raji-bearing mice treated with CAR19-NK cells was reversible after short-term in vitro culture. (A) CD19 expression on Raji cells, shown as the count of CD19 molecules per cell, performed serially on ex vivo cultured Raji cells harvested from liver of Raji-bearing mice treated with CAR19-NK cells (n=10 mice). Raji cells from untreated mice were used as control. (B) Incucyte analysis showing the percentage (%) of caspase 3/7 events in Raji cells harvested from CAR19-treated Raji-bearing mice and cocultured with fresh CAR19-NK cells vs. NT-NK cells either immediately after being harvested (DO) or three days (D3) after in vitro culture, controlled by the co-culture with fresh Raji cells (representative for 3 experiments). P values were determined by two-tailed one-way ANOVA in FIG. 24A, or two-tailed two-way ANOVA in FIG. 24 B; ***P<0.0001. Data were assessed by flow cytometry, and shown by mean+s.e.m. Each dot represents an individual mouse-derived Raji cell sample.

FIGS. 25A-D. In vivo trogocytosis was associated with limited persistence of CAR-NK cells. (A) The proportion of CAR19-expressing vs CAR-negative live NK cells in the peripheral blood of mice at different time points after CAR-NK cell infusion (left, n=15 mice), and in different organs at the end time point (right, n=24 mice). (B.1 and B.2) tCD19 expression on NK cells in the CAR19-expression fraction vs. the CAR-negative NK cell fractions and their viability based on TROG-positivity (Q2: CAR19-NK^{TROG+}, Q3: CAR-negative NK^{TROG+}) in cells harvested from different organs (B.1 comprises Spleen and Liver results; B.2 comprises BM and blood results) of mice at the end time point (representative of n=24 mice). Inset numbers indicate the percentages of cells within the indicated gated regions. (C) tCD19 expression (indicated as TROG⁺/TROG⁻ ratio) on singlet hCD45⁺GFP⁻CD56⁺CD3⁻CAR19-NK (dark gray, left bars) vs CAR-negative NK (light gray, right bars) cells harvested from organs of mice at end time point after CAR-NK cell infusion (left graph, n=24 mice) and in mice treated with NT-NK cells alone (right graph: dark gray, n=10 mice). (D) Percentage (%) of viable NK^{TROG+} (tCD19″, left graph, (i) left bars or (ii) right bars) and NK^{TROG-} cells (middle graph, (iii) left bars or (iv) right bars) in CAR19-NK cells ((i) or (iii)) vs. CAR-negative NK cells ((ii) or (iv)) harvested from organs of mice at end time point after infusion of CAR-NK cells (n=24 mice), or in mice treated with NT-NK cells (right graph: dark gray (v), n=10 mice). P values were determined by two-tailed one-way ANOVA in FIG. 25A (right panel), or two-sided Student's paired-t test in FIGS. 25A (left panel), C, and D. Data were assessed by flow cytometry in samples with cell objects of interest >20 counts, and shown by mean+s.e.m. Each circle represents an individual mouse sample, and outliers were indicated as dark dots.

FIGS. 26A-F. CAR5-NK cell trogocytosis and reciprocal reduction in CD5 antigen on CCRF^{CD5+} tumor cells in vivo. (A) Tumor burden was assessed weekly by BLI after infusion of CAR5-NK cell (gray). Mice engrafted with CCRF tumor only were used as the control (black); data were pooled from two independent experiments (n=5 mice per group). (B) CD5 expression, shown as gMFI, on engrafted CCRF cells in the peripheral blood of mice at different time points (left), and in organs harvested from mice at the end time point (right) after CAR5-NK cell infusion. (C-D) Ratio of CAR5-expressing vs. non-CAR expressing live NK cells in the peripheral blood of mice at different time points after CAR5-NK cell infusion (C) and in organs harvested at the end time point (D); data were pooled from two independent experiments (n=5 mice per group). (E) tCD5 expression on singlet hCD45⁺GFP⁻CD56⁺CD3⁻ NK cells in the CAR5-expressing ((i), left bars) vs. non-CAR expressing ((ii), right bars) NK cell fractions, indicated as TROG⁺/TROG⁻ ratio in organs of mice harvested at the end time point after CAR5-NK cell infusion. (F) Percentage (%) of viable NK^{TROG+} (tCD5⁺; left), NK^{TROG-} (right) and cells in the CAR5-expressing (dark gray) vs. non-CAR expressing (light gray) NK cell fractions harvested from organs of mice at the end time point after CAR5 NK cell infusion. P values were determined by two-tailed one-way ANOVA in FIGS. 26 B and D, or two-sided Student's t test in FIGS. 26 C, E, and F. Data were assessed by flow cytometry in samples with cell objects of interest >20 counts, pooled from two independent experiments, where NK cells were derived from different donors, and shown by mean+s.e.m. Each circle represents an individual mouse sample, outliers were indicated in dark dots.

FIGS. 27A-E. CAR123-NK cell trogocytosis and reciprocal reduction in CD123 antigen on MOLM-14^{CD123+} tumor cells in vivo. (A) Kaplan-Meier curves showing the percent survival of MOLM14-engrafted mice after infusion of CAR123-NK cells vs no treatment (n=5 mice per group). Data were pooled from two independent experiments. (B) CD123 expression on engrafted MOLM-14 cells in the peripheral blood of mice at different time points (left), and in organs harvested from mice at the end time point (right) after CAR123-NK cell infusion. (C) CAR123 expression on live hCD45⁺GFP⁻CD56⁺CD3⁻ NK cells, shown as ratio of CAR123-expressing vs. non-CAR expressing NK cells in the peripheral blood of mice at different time points after CAR123-NK cell infusion (left) and in organs harvested at the end time point (right). (D) tCD123 expression on singlet hCD45⁺GFP⁻CD56⁺CD3⁻ NK cells, gated on CAR123-expressing (dark gray, left bars) vs. non-CAR expressing (light gray, right bars) NK cells, indicated as TROG⁺/TROG⁻ ratio in organs of mice harvested at the end time point. (E) Percentage (%) of viable NK^{TROG+} (tCD123⁺; left) and NK^{TROG-} (right) cells in the CAR123 expressing (dark gray, left bars) vs. non-CAR expressing (light gray, right bars) NK cell fractions. P values were determined by log-rank test in FIG. 27A, two-tailed one-way ANOVA in FIGS. 27 B and C, or two-sided Student's t test in FIGS. 27 D and E. Data were assessed by flow cytometry in samples with cell objects of interest >20 counts, pooled from two independent experiments, where NK cells were derived from different donors, and shown by mean+s.e.m. Each circle represents an individual mouse sample.

FIGS. 28A-G. In vivo trogocytosis was associated with poor viability of CAR-NK cells. (A) Schematic illustration of the timeline using a mouse model of lymphoma, engrafted with 0.2×10⁵ luc/GFP-expressing CD19⁺ Raji cells and treated with a single infusion of CAR19-NK cells or NT-NK cells as control. Blood, BM, spleen and liver were harvested for analysis at two weeks (day 13-15), three to four weeks (day 20-27), or at the end time point (day 29-34) after infusion. (B) Tumor burden was assessed weekly by BLI. The BLI intensity is shown for each mouse after infusion with CAR-NK cells (solid green or dark gray) or NT-NK cells (dashed blue or light gray). Untreated mice were used as controls (black). (C) Heat map representing the expression levels of phenotypic and functional markers on fractions of TROG⁺ (tCD19⁺) and TROG⁻ live hCD45⁺GFP⁻CD56⁺CD3⁻ NK cells at different timepoints post-infusion. The expression level for each marker is represented by the color gray (low)—orange (high; positive Z scores are marked with a positive sign in the upper right corner of the respective circles, while negative Z scores are left unmarked) and the size of the circle shows expression of the marker, the larger the circle the greater the proportion of cells expressing the noted marker. (D) The phenotypic cell signature for each condition was evaluated by mass cytometry and merged to create a single t-SNE map. Expression of tCD19 (orange, (ii)) and CAR19 (green; (iii)) on hCD45⁺GFP⁻CD56⁺CD3⁻ NK cells was determined based on their expression on NT-NK cell controls. (E) Violin plots showing expression of tCD19 on NK cells within each cluster harvested from mice treated with CAR19-NK cells (left); cisplatin levels within the TROG⁺ vs. TROG⁻ fractions for each cluster (right) are shown. (F) Violin plots showing expression of tCD19 on NT-NK cells within each cluster (left); cisplatin levels within the TROG⁺ vs. TROG⁻ fractions for each cluster (right) are shown. (G) Gene signature for total hCD45⁺ cells at different time points during the treatment course. The t-SNE maps, generated with the Seurat package in R, show color-coded expression levels for (D)19 and MS4A1 (Raji cells), NKG7 and FCGR3A (NK cells) for each cluster (NK-C1; NK-C2; NK-C3; and Raji-cells). P values were determined by two-tailed Wilcoxon matched pairs test in FIGS. 28 E and F; **P<0.001, ***P<0.0001. Data were assessed by mass cytometry and shown in violin graph with the indicated median.

FIG. 29. Gating strategy for the immunophenotyping of human PBMCs after CAR19-NK cell-based immunotherapy. PBMCs from patients who received CAR-NK cell therapy were isolated and prepared for flow cytometry analysis as described herein in the Methods. Single live cells were determined based on FSC/SSC selection, and Live/Dead separation. Hematopoietic cells within the live population were then selected by gating on hCD45⁺CD33⁻CD14⁻ cells. Differential expressions of CD56, CD3, CD16 and CD19 were used to discern NK cell populations (CD56⁺CD16⁺CD3⁻), T cell populations (CD56⁻CD16⁻CD3⁺), or B cell populations (CD19⁺CD56⁻CD16⁻CD3⁻). Within the CD3⁻CD56⁺ subset, cord blood-derived donor NK cells were identified based on expression of the donor-specific HLA-antigen. Expression of CAR on donor NK cells was further determined by expression of CAR, determined using an antibody (109606088/Jackson Immuno Rsch) directed against the CH2-CH3 domain of the human IgG hinge.

FIGS. 30A-B. iCAR design, and impact on primary human NK cell trogocytosis.
(A) Schematic diagram of a viral vector encoding different anti-CD19 iCARs; TM: transmembrane; SE: signaling endodomain. (B) tCD19 expression on NK cell transduced with CAR19, 19scFv or the different iCAR19 constructs, presented as ratio of TROG⁺/TROG⁻ cells at different time points during co-culture with Raji^{CD19+} cells (n=5 donors). P values were determined by two-tailed two-way ANOVA; *P<0.01, **P<0.001, ***P<0.0001. Data were assessed in flow cytometry and shown by mean+s.e.m.

FIGS. 31A-F. CS1 expression on Raji cells and NK cell populations. (A) CS1 expression on healthy human B cells (hCD45⁺CD56⁻CD3⁻CD19⁺CD20⁺), primary human NK cells (hCD45⁺CD56⁺CD3⁻CD19⁻CD20⁻), resting T cells (hCD45⁺CD56⁻CD3⁺CD19⁻CD20⁻), or T cells activated with CD3/CD28 beads (n=3 donors). (B) Flow cytometric analysis of CS1 expression on Raji cells and primary human NK cells from five individual donors. (C) CS1 expression on Raji cells cultured alone, Raji cells co-cultured with CAR19-NK cells for one hr, and on the TROG⁺ (tCD19⁺) vs. the TROG⁻ fractions of CAR19-expressing vs. non-CAR expressing NK cells after co-culture with Raji cells for one hr (n=5 donors). (D) Tumor burden in mice engrafted with Raji cells was assessed by BLI weekly; the intensity of BLI is shown for each mouse after infusion with CAR19-NK cells (light gray (ii)). Untreated mice were used as control (black (i); n=5 mice). (E) CS1 expression on Raji cells (i), CAR-negative NK cells (ii), or CAR19-NK cells (iii) in blood of mice at different time points after infusion (n=10 mice). (F) CS1 expression on Raji cells, and on the TROG⁺ (tCD19⁺) vs. the TROG⁻ fractions of CAR19-NK expressing (left) or non-CAR expressing (right) NK cell fractions harvested from organs of mice at the end time point after CAR19 NK cell infusion; Raji (i), NK^{TROG-} (ii), NK^{TROG+} (iii), CAR19-NK^{TROG-} (iv), and CAR19-NK^{TROG+} (v); (n=10 mice). CS1 expression was assessed by flow cytometry, determined by gMFI, and normalized to isotope control; fold change of 1 was indicated by the gray dashed line. P values were determined by two-tailed one-way ANOVA. Data were shown by mean+s.e.m. Each dot represents an individual mouse sample.

FIGS. 32A-F. AI-CAR expressing NK cells exert superior anti-tumor activity in vivo. (A and B) tCD19 expression (indicated as TROG⁺/TROG⁻ population) and viability of singlet NK cells harvested from (A) peripheral blood and (B) BM of mice at different time points after infusion of 19scFv/CS1scFv (darker gray; left bars) or 19scFv/iCAR-CS1 (light gray; right bars) NK cells (n=5 mice per group). (C) tCD19 (indicated as TROG⁺/TROG⁻ population) and viability of singlet NK cells harvested from the BM of mice at different time points after infusion of aCAR19/CS1scFv (darker gray; left bars) or aCAR19/iCAR-CS1 (light gray; right bars) NK cells (n=5 mice per group). (D) Live NK cell counts in the blood, BM, spleen and liver collected from mice at days 3, 10, and 20 after infusion of 19scFv/CS1scFv (light gray) or 19scFv/iCAR-CS1 (black with dark lines for averages) NK cells (n=5 mice per group). (E) Live NK cell counts collected from the BM and liver of mice at days 3, 10, and 20 after infusion of aCAR19/CS1scFv (darker gray; (i)) or aCAR19/iCAR-CS1 (light gray; (ii)) NK cells (n=5 mice in per group). (F) Levels of IL-15, granzyme A (GrA), GrB, and perforin in serum collected from mice at days 3, 10, and 20 after infusion of CAR-expressing NK cells; Raji alone ((i)), Raji+19scFv/CS1-scFv-NK ((ii)), Raji+19scFv/iCAR-CS1-NK ((iii)), Raji+aCAR19/CS1scFv-NK ((iv)), and Raji+aCAR19/iCAR-CS1-NK ((v)); (n=5 mice per group). P value was determined by two-tailed Student's t test in FIGS. 32A-E, or two-tailed two-way ANOVA in FIG. 32 F; **P<0.001, ***P<0.0001, n.d.: not detected. Data were assessed by flow cytometry in FIGS. 32A-E, and shown by mean+s.e.m. Each circle represents an individual mouse sample.

FIGS. 33A-I. AI-CAR expressing NK cells exert superior in vivo anti-tumor activity in a SKOV3^{gCD19+} ovarian cancer model. (A) Schematic illustration of the timeline using a mouse model of ovarian cancer, engrafted with 0.5×10⁶ of SKOV3 tumor cells genetically modified to express CD19 (SKOV3^{gCD19+}). Seven days later, mice received a single infusion of 1×10⁷ NK cells expressing aCAR19/CS1scFv (dark gray, middle column), or aCAR19/iCAR-CS1 (light gray, right column), or no NK cells (black; left column, tumor only control group) (n=5 mice per group). (B and C) Tumor burden was determined by weekly BLI, (B) representative images at select time points are shown; (C) normalized intensity of BLI for each treatment group over the treatment course; the dashed lines represent the data for each mouse. (D) Kaplan-Meier curves showing the survival of mice after NK cell infusion. (E and F) (E) tCD19 (indicated as TROG⁺/TROG⁻ population) and (F) viability of the TROG⁺ fraction (NK^{gCD19+}) of NK cells in the peripheral blood of mice at Day 5, 15, and 30 after infusion of aCAR19/CS1scFv ((i)) or aCAR19/iCAR-CS1 ((ii)) NK cells (n=5 mice per group). (G) Percentage (%) of live GFP⁻CD3⁻CD56⁺CAR19⁺ NK cells in the peripheral blood of mice at Day 5, 15, and 30 after infusion of aCAR19/CS1scFv ((i), left) or aCAR19/iCAR-CS1 ((ii), right) NK cells (n=5 mice per group). (H) Live NK^{CAR19+} NK cell counts in the peripheral blood of mice at Day 5, 15, and 30 after infusion of aCAR19/CS1scFv ((i)) or aCAR19/iCAR-CS1 ((ii)) NK cells (n=5 mice per group). (I) Representative images showing H&E and IHC staining with anti-Luciferase, anti-hCD45, or anti-hROR1 antibodies on sections from the mesentery tissue of SKOV3^{ROR1+} grafted mice treated with aCAR-ROR1/CS1-scFv NK cells or aCAR-ROR1/iCAR-CS1 NK cells. Inserted numbers indicate hCD45⁺ cell count per 0.1 mm². Black arrows (right panel) indicate ROR1 expression on tumor cells; light gray arrows (right panel) indicate ROR1 expression on NK cells; scale bar indicates 100 μm. P value was determined by two-tailed one-way ANOVA in FIG. 33 C, or log-rank test in FIG. 33 D, or two-tailed Student's t test in FIGS. 33 E, F, H, and I, or two-tailed one-way ANOVA in FIG. 33 G; *P <0.01, **P<0.001, ***P<0.0001, n.s: not significant. Data was pooled from two independent experiments in FIGS. 33 C and D, where NK cells were derived from different donors, or assessed by flow cytometry in FIGS. 33 E, F, G, and H, and shown by mean+s.e.m. Each circle represents an individual mouse sample.

FIG. 34A-B. Model of AI-CAR NK cell function. (A) aCAR-NK cell-mediated trogocytosis results in a decrease in antigen density on tumor cells, and promotes CAR-NK cell fratricide and hyporesponsiveness; (B) Engineering NK cells to express both an aCAR against a tumor antigen and a KIR-based inhibitory CAR (iCAR) against an NK self-antigen prevents TROG-induced self-recognition and TROG-antigen mediated fratricide of CAR-NK cells, while preserving their on-target tumor recognition and cytotoxicity.

FIG. 35A-D. Self-engagement of TROG⁺ CAR-NK cells resulted in NK cell fratricide. (A and B) Schematic (left panel) illustrating the subsequent single-cell time-lapse imaging cytotoxicity/survival assay (right panel). Time was recorded over 5 hours (T₀-T₃₀₀ₘᵢₙ) from the start of co-culture, (A) where one single cell, or (B) two cells of non-TROG-antigen expressing fresh CAR-NK cells (control; gray (i)) or CAR-NK^{TROG+} cells (dark gray (ii)) were incubated in each nanowell. For the duration of the assay, the amount of time taken to detect Annexin V influx in the sorted CAR-NK cell was determined as the time taken to induce cell apoptosis. The Kaplan-Meier curves (right panel) show the percent (%) of apoptosis in CAR-NK cells during incubation. (C) Schematic representation (top left illustration) of the experimental plan: TROG⁺ CAR19-NK cells were purified and their phenotypic signature evaluated before and 5 hrs post-culture by mass cytometry. Data from 10,000 cells from 3 donors per condition were merged to create a single UMAP map with eight distinct color-coded clusters that represented the different subsets of CAR-NK cells. Marker expression (NKp30, NKp44, TRAIL, CD94, CD2, CD16, 2B4, NKp46, DAP12, KIR, KLRG-1, NKG2A, NKG2C, and NKG2D) for each NK cell subset is shown. (D) UMAP plots showing the expression of TROG-antigen (tCD19) on CAR-NK cells before (left panel) and after 5 hrs of culture with their sibling cells (right panel); the contour plots show the prevalence of each CAR-NK cell subset before and after 5 hrs of culture, with the percentage of each subset also indicated. Changes in CD2⁺CD16⁺NKG2A⁺NKG2C⁺CD94⁺ cell populations, CD2⁻KIR⁺NKG2C⁺CD94^{′} cell populations, and CD2⁻KIR⁺NKG2A⁺NKG2C⁻CD94⁺ cell populations were observed. P value was determined by log-rank test in FIGS. 35A and B.

### DESCRIPTION OF ILLUSTRATIVE EMBODIMENTS

Trogocytosis is a well-described phenomenon by which lymphocytes extract plasma membrane fragments from their targets following immunological synapse formation (see, e.g., Joly & Hundrisier 2003; Dance 2019; and Ahmed et al., 2008). Throughout this disclosure, evidence is provided of a unique mechanism for tumor escape following CAR-effector cell therapy, particularly that activation of activating CAR (aCAR) drives the transfer of target antigens from target cells, that are engaged by but not killed by an effector cell, to effector cells. In some embodiments, an effector cell that acquires a target antigen via trogocytosis (TROG), referred to herein as a TROG-antigen, in turn, becomes a target cell (antigen-induced self-recognition) and such cells are lysed by other aCAR-effector cells while those that are not killed by fratricide become hyporesponsive, similar to reports with CAR-T cells (see e.g., Hamieh et al., 2019; and Wang et al., 2021). In some embodiments, this phenomenon is associated with a concurrent loss of target antigen on the cancer cells, rendering them less susceptible to aCAR-mediated killing, and thus, increasing the risk of tumor relapse in a subject. Also described and shown herein are novel inhibitory CAR (iCAR) systems that combine at least one extracellular domain targeting an effector cell specific antigen with one or more signaling endodomains from an inhibitory killer Ig-like receptor (KIR). These novel iCAR systems successfully achieved antigen-specific suppression of aCAR-mediated effector cell fratricide and hyporesponsiveness, while retaining the effector cells on-target anti-tumor activity with improved persistence and/or activity in multiple in vitro and in vivo models.

Intercellular protein transfer is mediated through multiple pathways (see, e.g., Dance 2019; Griffiths & Tsun 2010; Hochreiter-Hufford & Ravichandran 2013; and Kalluri & LeBleu 2020). Among these pathways, trogocytosis is a well-described mechanism for exchange of surface protein(s) between NK cells and their targets (see, e.g., Miyake & Karasuyama 2021; Tabiasco et al., 2002; and Tabiasco et al., 2003). Additionally, trogocytosis has been shown to significantly impact NK cell function (see, e.g., Caumartin et al., 2007; Nakamura et al., 2013(b); Domaica et al., 2009; and Nakayama et al., 2011). For instance, NKG2D-mediated trogocytosis of NKG2D-ligands has been associated with NK cell hyporesponsiveness and fratricide (see, e.g., Nakamura et al., 2013(a); Miner et al., 2015; and Nakamura et al., 2013(b)). Similarly, trogocytosis triggered by the engagement of the CD16 receptor on NK cells with monoclonal antibodies leads to target antigenic modulation and compromised therapeutic efficacy (see, e.g., Carlsten et al., 2016; Taylor & Lindorfer 2015; and Beum et al., 2008). A recent study reported how trogocytosis promotes antigen density reduction and T-cell exhaustion and fratricide after CAR-T cell therapy (see, e.g., Hamieh et al., 2019). This disclosure is the first to show that human CAR-NK cells acquire cognate-antigen targets from tumor cells through antigen-specific trogocytosis that requires CAR-activation and signaling. This phenomenon was observed with CARs expressing different CAR-signaling endodomains and targeting multiple antigens across multiple cancer types. In some embodiments, the degree of trogocytosis is influenced by the affinity of the CAR for its cognate ligand and/or by the density of the antigen on tumor cells. Given that TROG-antigen expressing NK cells are susceptible to aCAR-NK cell cytotoxicity by induced-self recognition, in some embodiments, preventing trogocytosis can be a beneficial approach to improving the efficacy and/or in vivo persistence of aCAR-NK cells.

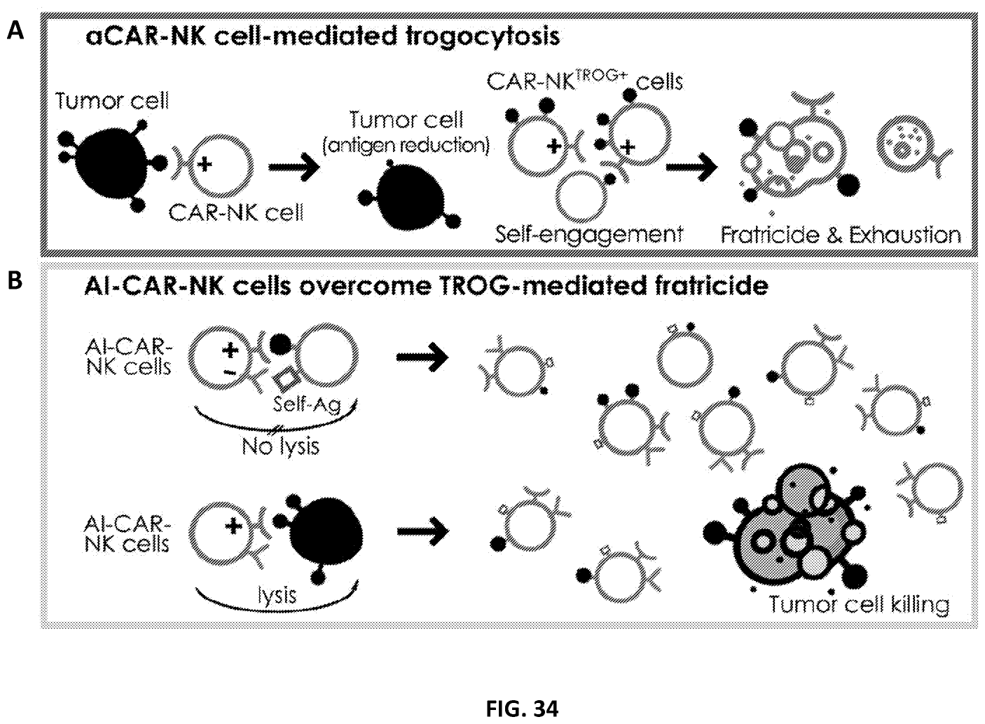

To date, there are no strategies that can be applied therapeutically to regulate trogocytosis in a specific manner. Described and shown herein is a novel engineering approach that leveraged NK cell biology and employed an ITIM-containing iCAR to suppress aCAR-mediated recognition of TROG-antigen-expressing NK cells (on-target/off-tumor recognition of TROG⁺ NK cells), while retaining the aCAR activity against tumor targets. In some embodiments, by combining the activity of two chimeric receptors, one of which generated a dominant negative signal upon recognition of an NK-specific antigen and one that induced an activating signal upon engagement with the tumor target, the response of the counteracting aCAR-activation against the TROG-antigen on NK cells can successfully be switched off in an antigen-specific manner, while sparing the effector cells activity against the tumor target. In some embodiments, NK cells transduced with this AI-CAR system were less susceptible to TROG-antigen-mediated fratricide and exhaustion, and mediated a superior anti-tumor response both in vitro and in multiple in vivo models (see FIGS. 34A and B for a graphical reproduction).

This disclosure describes and shows how aCAR-mediated trogocytosis, which contributes to a reduction in target antigen density, and effector cell fratricide and hyporesponsiveness, contributes as a novel mechanism of disease relapse after aCAR cell therapy. Herein are provided multiple working examples and proof of concepts suitable for use in the rescue of aCAR effector cell (e.g., aCAR-NK cells) function from TROG-antigen induced immunomodulatory consequences using antigen-specific iCARs that successfully inhibit aCAR-mediated TROG-antigen induced effector cell fratricide and exhaustion, while retaining critical effector function against tumor cells expressing the same antigen. This dynamic modulation of AI-CAR-signaling can find useful applications for at least improvement of the in vivo persistence and therapeutic efficacy of a range of adoptive effector cell therapies for various maladies including but not limited to: cancer (e.g., hematological and/or solid tumor), infections (e.g., viral, bacterial, fungal, parasitic, etc.), autoimmune disorders, and genetically inherited disorders.

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The term “engineered” as used herein refers to an entity that is generated by the hand of man, including a cell, nucleic acid, polypeptide, vector, a combination thereof, and so forth. In at least some cases, an engineered entity is synthetic and comprises elements that are not naturally present or configured in the manner in which it is utilized in the disclosure. With respect to cells, the cells may be engineered because they express one or more heterologous genes (such as synthetic antigen receptors and/or cytokines) and/or they have reduced expression of one or more endogenous genes, in which case(s) the engineering is all performed by the hand of man. With respect to an antigen receptor, the antigen receptor may be considered engineered because it comprises multiple components that are genetically recombined to be configured in a manner that is not found in nature, such as in the form of a fusion protein of components not found in nature so configured.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” or “individual” refer to either a human or non-human, such as primates, mammals, and vertebrates. In particular embodiments, the subject is a human.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, horses, goats, sheep, and chimpanzees. Mammals may be referred to as “patients” or “subjects” or “individuals”.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “heterologous” as used herein refers to being derived from a different cell type or a different species than the recipient. In specific cases, it refers to a gene or protein that is synthetic and/or not from an NK cell. The term also refers to synthetically derived genes or gene constructs. The term also refers to synthetically derived genes or gene constructs. For example, a cytokine may be considered heterologous with respect to a NK cell even if the cytokine is naturally produced by the NK cell because it was synthetically derived, such as by genetic recombination, including provided to the NK cell in a vector that harbors nucleic acid sequence that encodes the cytokine.

The disclosure provides cell therapy methods and compositions in which the cell therapy is cytotoxic to cells in need of being killed, such as cancer cells. The cells of the cell therapy include a mechanism that acts as an inhibitory signal for the cell therapy when the cytoxicity of the cells needs to be deterred. In particular embodiments, the inhibitory signal acts under conditions in which the cytotoxic cell therapy will kill cells that are not their intended target, such as cells that are not desired to be killed. In specific embodiments, the cells that are not their intended target are non-cancerous cells. In specific embodiments, the cells that are not their intended target have acquired through trogocytosis an antigen that otherwise would not have been expressed by the cells, at least to a detectable extent. In some cases, cells of the cell therapy have acquired an antigen through trogocytosis that earmarks those cells for destruction by other cells of the cell therapy, which may or may not also have acquired the antigen through trogocytosis. The disclosure provides methods and compositions that prevent fratricide and/or self-engagement induced hyporesponsiveness among cells of the cell therapy by use of this inhibitory signal. In particular embodiments, the inhibitory signal is an inhibitory CAR (iCAR).

Trogocytosis is an active cellular process that involves the transfer of surface material from one cell to another, mediated by a constitutive ligand-induced and receptor-mediated antigen endocytosis and recycling process. CAR-mediated trogocytosis has been reported to suppress CAR-T cell anti-tumor cytotoxicity by mediating fratricide and exhaustion (see, e.g., Hamich et al., 2019). The disclosure provides engineered CAR-NK cells (e.g., as one example of a cell therapy) to express a dual CAR system that includes at least one activation CAR (aCAR) and at least one iCAR that recognizes an antigen on other cells and that, as a result of that antigen recognition, instructs the iCAR to inhibit the cytotoxic activity of the antigen-expressing cells through the activity of the CAR.

This technology can also be applied to reduce the on-target off-tumor toxicity of CAR T cells or CAR NK cells targeting a solid tumor antigen with shared expression on normal tissue (e.g., mesothelin, also expressed on lung epithelium). That is, because the iCARs exert their inhibitory function in an antigen-specific manner, the iCAR can target an antigen specifically expressed on normal tissue. When combined with iCAR engineering, the CAR-expressing immune effector cells can discern off-tumor targets from on-tumor targets that could largely improve their curative efficacies and/or reduce undesirable side effects.

### II. Inhibitory CARs and Constructs and Uses Thereof

In some embodiments, the iCARs of the disclosure encompass single polypeptides comprising one or more extracellular antigen-binding domains, a transmembrane domain, and one or more intracellular inhibitory signaling domains. In some embodiments, an inhibitory signaling domain imparts inhibition of the cell that expresses the iCAR upon binding of the antigen to which the iCAR is targeted, including imparting inhibition of the cell through inhibiting an activating CAR also expressed by the cell. In some embodiments, the inhibitory signaling domain(s) are from the natural inhibitory signaling of NK cells. That is, in some cases the inhibitory signaling domains are inhibitory signaling domains found in natural NK cell inhibitory receptors (that act through immunoreceptor tyrosine-based inhibitory motif). In some embodiments, upon binding of the iCAR to the antigen to which it is targeted, an inhibitory signaling domain from the NK cell receptors inhibits the activity of the corresponding activating CAR (aCAR), regardless of whether or not the dual CAR system is in an NK cell or other type of cell, such as a T cell. Examples of inhibitory signaling domains include, but are not limited to, the signaling domains of any of the killer Ig-like receptor (KIRs), leukocyte immunoglobulin-like receptor (LIR-1; also referred to as LILRB1), CD300A, NKG2A, Siglec-7, CD96, TIM3, TIGIT, and/or LAIR-1. Examples of KIRs include at least KIR2DL1, KIR2DL2, KIR2DL3, KIR2DL4, KIR2DL5, KIR3DL1, KIR3DL2, KIR3DL3, KIR2DL5A, and KIR2DL5B. In some embodiments, one or more co-inhibitory domains are utilized in addition to one or more inhibitory signaling domains or as an alternative to one or more inhibitory signaling domains. In specific embodiments, the co-inhibitory domain is from LAIR-1, NKG2A, CD300A, or a combination thereof. In some embodiments, when more than one inhibitory signaling domain is utilized, the order from N-terminus to C-terminus in the iCAR may be of any order.

In specific embodiments, the iCAR targets one or more antigens. In some embodiments, when the iCAR targets more than one antigen, the two or more antigens may be non-identical. In specific embodiments, selection of the antigen to which the antigen binding domain of the iCAR is targeted is determined by cells that are in need of protection from immune effector cells expressing the iCAR and the aCAR. In some embodiments, cells in need of protection may or may not be of the same type of cells as the immune effector cells (e.g., both NK cells, both T cells, or one type of each). In specific embodiments, the antigen is selected because it is a self antigen for NK cells, such that the iCAR is an NK self-recognizing inhibitory iCAR that recognizes a self antigen and transfers a “don't′ kill me” signal to the NK immune effector cells, e.g., upon engagement with their sibling cell. Such an inhibitory mechanism prevents or reduces fratricide and/or exhaustion, while allowing the aCAR still to target and kill cancer cells expressing the tumor antigen. Examples of NK self antigens include, but are not limited to, at least KIR2DL1, KIR2DL2, KIR2DL3, KIR2DL5, KIR3DL1, KIR3DL2, KIR3DL3, KIR2DL4, KIR2DS1, KIR2DS2, KIR2DS3, KIR2DS4, KIR2DS5, KIR3DS1, DAP10, DAP12, CD56, CD57, CD25, CD122, NKP30, NKP44, NKP46, NKG2C, NKG2D, NKG2A, CRTAM, TIGIT, CD96, 2B4, CD16, CD27, CD100, CD160, ILT2, ILT4, KLRG1, LAIR1, CD161, CS1, an (Natural Cytotoxicity Receptor) NCR, KIR, and/or other NK-related antigen.

In a specific case, the iCAR comprises two antigen binding domains each specific for a different NK cell self antigen, such as an antigen binding domain that targets CS1 and an antigen binding domain that targets CD56. In any case in which two antigen binding domains are utilized in the iCAR, the order of a first antigen binding domain and a second antigen binding domain may be in any order in an N-terminus to C-terminus direction. In specific embodiments, when two antigen binding domains are utilized in the iCAR, there may be a spacer of suitable length and content between the two antigen binding domains. In any event, the antigen binding domain of the iCAR may comprise an antibody or functional fragment thereof (e.g., scFv or single-domain antibody) that binds the antigen, or the antigen binding domain may be a natural ligand in some cases. The extracellular antigen binding domain may be associated with a hinge of any kind, such as from IgG1, CD28, CD8alpha, and so forth.

With respect to the transmembrane domain (TM) of the iCAR, the TM may be of any suitable kind. In specific embodiments, the TM may or may not be from the same molecule as the inhibitory signaling domain or co-inhibitory domain. In specific embodiments, the TM is from KIR2DL1 or LIR-1, although it may also be from the alpha, beta or zeta chain of the T-cell receptor, CD28, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, and DAP molecules.

Examples of Constructs of iCAR signaling: By harnessing the natural inhibitory pathways that prevent NK cell from cytotoxicity against normal antigens, the inventors have designed a dual CAR system that includes at least one iCAR. In specific embodiments, the scFv against a normal or self antigen is selectively conjugated to the transmembrane domains (TMs or TMDs), intracellular inhibitory domains (IDs; also referred to as inhibitory signaling domains), and intracellular co-inhibitory domains (CIDs) of KIR2DL1, LIR1, LAIR-1, NKG2A, and CD300A. Examples of combinations of TMDs and intracellular domains (ICD that may also encompass IDs and CIDs) are as follows:
KIR2DL1 (TMD)—KIR2DL1 (ICD)
KIR2DL (TMD)—KIR2DL1 (ICD)−Siglec-7 (ICD)
KIR2DL1 (TMD)—KIR2DL1 (ICD)−LAIR-1 (ICD)
KIR2DL1 (TMD)—KIR2DL1 (ICD)−NKG2A(ICD)
KIR2DL1 (TMD)—KIR2DL1 (ICD)—CD300A(ICD)
KIR2DL2 (TMD)—KIR2DL2 (ICD)
KIR2DL2 (TMD)—KIR2DL2 (ICD)−Siglec-7 (ICD)
KIR2DL2 (TMD)—KIR2DL2 (ICD)−LAIR-1 (ICD)
KIR2DL2 (TMD)—KIR2DL2 (ICD)−NKG2A(ICD)
KIR2DL2 (TMD)—KIR2DL2 (ICD)—CD300A(ICD)
KIR2DL3 (TMD)—KIR2DL3 (ICD)
KIR2DL3 (TMD)—KIR2DL3 (ICD)−Siglec-7 (ICD)
KIR2DL3 (TMD)—KIR2DL3 (ICD)−LAIR-1 (ICD)
KIR2DL3 (TMD)—KIR2DL3 (ICD)−NKG2A(ICD)
KIR2DL3 (TMD)—KIR2DL3 (ICD)—CD300A(ICD)
KIR3DL1 (TMD)—KIR3DL1 (ICD)
KIR3DL1 (TMD)—KIR3DL1 (ICD)−Siglec-7 (ICD)
KIR3DL1 (TMD)—KIR3DL1 (ICD)−LAIR-1 (ICD)
KIR3DL1 (TMD)—KIR3DL1 (ICD)−NKG2A(ICD)
KIR3DL1 (TMD)—KIR3DL1 (ICD)—CD300A(ICD)
KIR3DL2 (TMD)—KIR3DL2 (ICD)
KIR3DL2 (TMD)—KIR3DL2 (ICD)−Siglec-7 (ICD)
KIR3DL2 (TMD)—KIR3DL2 (ICD)−LAIR-1 (ICD)
KIR3DL2 (TMD)—KIR3DL2 (ICD)−NKG2A(ICD)
KIR3DL2 (TMD)—KIR3DL2 (ICD)—CD300A(ICD)
KIR3DL3 (TMD)—KIR3DL3 (ICD)
KIR3DL3 (TMD)—KIR3DL3 (ICD)−Siglec-7 (ICD)
KIR3DL3 (TMD)—KIR3DL3 (ICD)−LAIR-1 (ICD)
KIR3DL3 (TMD)—KIR3DL3 (ICD)−NKG2A(ICD)
KIR3DL3 (TMD)—KIR3DL3 (ICD)—CD300A(ICD)
KIR2DL4 (TMD)—KIR2DL4 (ICD)
KIR2DL4 (TMD)—KIR2DL4 (ICD)−Siglec-7 (ICD)
KIR2DL4 (TMD)—KIR2DL4 (ICD)−LAIR-1 (ICD)
KIR2DL4 (TMD)—KIR2DL4 (ICD)−NKG2A(ICD)
KIR2DL4 (TMD)—KIR2DL4 (ICD)—CD300A(ICD)
KIR2DL5 (TMD)—KIR2DL5 (ICD)
KIR2DL5 (TMD)—KIR2DL5 (ICD)−Siglec-7 (ICD)
KIR2DL5 (TMD)—KIR2DL5 (ICD)−LAIR-1 (ICD)
KIR2DL5 (TMD)—KIR2DL5 (ICD)−NKG2A(ICD)
KIR2DL5 (TMD)—KIR2DL5 (ICD)—CD300A(ICD)
NKG2A (TMD)—NKG2A(ICD)
CD300A (TMD)—CD300A(ICD)
LAIR-1 (TMD)—LAIR-1 (ICD)
Siglec-7(TMD)—Siglec-7 (ICD)
CD96(TMD)—CD96 (ICD)
TIM-3(TMD)—TIM3(ICD)
TIGIT(TMD)—TIGIT(ICD)
NKG2A (TMD)—NKG2A(ICD)−LAIR-1 (ICD)
NKG2A (TMD)—NKG2A(ICD)−CD300A(ICD)
NKG2A (TMD)—NKG2A(ICD)−LAIR-1 (ICD)
NKG2A (TMD)—NKG2A(ICD)−Siglec-7 (ICD)
NKG2A (TMD)—NKG2A(ICD)−CD96 (ICD)
NKG2A (TMD)—NKG2A(ICD)—TIM-3(ICD)
NKG2A (TMD)—NKG2A(ICD)−TIGIT (ICD)
LAIR-1 (TMD)—Siglec-7 (ICD)
LAIR-1 (TMD)—CD96 (ICD)
LAIR-1 (TMD)—TIM-3 (ICD)
LAIR-1 (TMD)—TIGIT (ICD)
Siglec-7(TMD)—CD96 (ICD)
Siglec-7(TMD)—TIM-3 (ICD)
Siglec-7(TMD)—TIGIT (ICD)
CD96(TMD)—TIM3 (ICD)
CD96 (TMD)—TIGIT(ICD)

The sequences of parts of examples of iCARs are listed below.

SEQ ID NO: 1 - iCAR1: KIR2DL1 (TM)- KIR2DL1 (ID)
Nucleotide sequence:
(SEQ ID NO: 1)
attctgattgggacctcagtggtcatcatcctcttcatcctcctcttctttctccttcatcgc
tggtgctccaacaaaaaaaatgctgcggtaatggaccaagagtctgcaggaaacagaacagcg
aatagcgaggactctgatgaacaagaccctcaggaggtgacatacacacagttgaatcactgc
gttttcacacagagaaaaatcactcgcccttctcagaggcccaagacacccccaacagatatc
atcgtgtacacggaacttccaaatgctgagtccagatccaaagttgtctcctgccca
SEQ ID NO: 2 - iCAR1: KIR2DL1 (TM)- KIR2DL1 (ID)
Peptide sequence:
(SEQ ID NO: 2)
ILIGTSVVIILFILLFFLLHRWCSNKKNAAVMDQESAGNRTANSEDSDEQDPQEVTYTOLNHC
VFTQRKITRPSQRPKTPPTDIIVYTELPNAESRSKVVSCP
SEQ ID NO: 3 - iCAR2: LIR-1 (TM)- LIR-1 (ID)
Nucleotide sequence:
(SEQ ID NO: 3)
gttgtgatcggcatcttggtggccgtcatcctactgctcctcctcctcctcctcctcttcctc
atcctccgacatcgacgtcagggcaaacactggacatcgacccagagaaaggctgatttccaa
catcctgcaggggctgtggggccagagcccacagacagaggcctgcagtggaggtccagccca
gctgccgatgcccaggaagaaaacctctatgctgccgtgaagcacacacagcctgaggatggg
gtggagatggacactcggcagagcccacacgatgaagacccccaggcagtgacgtatgccgag
gtgaaacactccagacctaggagagaaatggcctctcctccttccccactgtctggggaattc
ctggacacaaaggacagacaggcggaagaggacaggcagatggacactgaggctgctgcatct
gaagccccccaggatgtgacctacgcccagctgcacagcttgaccctcagacgggaggcaact
gagcctcctccatcccaggaagggccctctccagctgtgcccagcatctacgccactctggcc
atccac
SEQ ID NO: 4 - iCAR2: LIR-1 (TM)- LIR-1 (ID)
Peptide sequence:
(SEQ ID NO: 4)
VIGILVAVILLLLLLLLLFLILRHRRQGKHWTSTQRKADFQHPAGAVGPEPTDRGLQWRSSPA
ADAQEENLYAAVKHTQPEDGVEMDTRSPHDEDPQAVTYAEVKHSRPRREMASPPSPLSGEFLD
TKDRQAEEDRQMDTEAAASEAPQDVTYAQLHSLTLRREATEPPPSQEGPSPAVPSIYATLAIH
SEQ ID NO: 5 - iCAR3: KIR2DL1 (TM)- KIR2DL1 (ID)-
LAIR-1 (CID) Nucleotide sequence:
(SEQ ID NO: 5)
attctgattgggacctcagtggtcatcatcctcttcatcctcctcttctttctccttcatcgc
tggtgctccaacaaaaaaaatgctgcggtaatggaccaagagtctgcaggaaacagaacagcg
aatagcgaggactctgatgaacaagaccctcaggaggtgacatacacacagttgaatcactgc
gttttcacacagagaaaaatcactcgcccttctcagaggcccaagacacccccaacagatatc
atcgtgtacacggaacttccaaatgctgagtccagatccaaagttgtctcctgcccacatcgc
cagaatcagataaagcaggggccccccagaagcaaggacgaggagcagaagccacagcagagg
cctgacctggctgttgatgttctagagaggacagcagacaaggccacagtcaatggacttcct
gagaaggacagagagacggacacctcggccctggctgcagggagttcccaggaggtgacgtat
gctcagctggaccactgggccctcacacagaggacagcccgggctgtgtccccacagtccaca
aagcccatggccgagtccatcacgtatgcagccgttgccagacac
SEQ ID NO: 6 - iCAR3: KIR2DL1 (TM)- KIR2DL1 (ID)-
LAIR-1 (CID) Peptide sequence:
(SEQ ID NO: 6)
ILIGTSVVIILFILLFFLLHRWCSNKKNAAVMDQESAGNRTANSEDSDEQDPQEVTYTQLNHC
VFTQRKITRPSQRPKTPPTDIIVYTELPNAESRSKVVSCPHRQNQIKQGPPRSKDEEQKPQQR
PDLAVDVLERTADKATVNGLPEKDRETDTSALAAGSSQEVTYAQLDHWALTQRTARAVSPQST
KPMAESITYAAVARH
SEQ ID NO: 7 - iCAR4: KIR2DL1 (TM)- KIR2DL1 (ID)-
NKG2A(CID) Nucleotide sequence:
(SEQ ID NO: 7)
attctgattgggacctcagtggtcatcatcctcttcatcctcctcttctttctccttcatcgc
tggtgctccaacaaaaaaaatgctgcggtaatggaccaagagtctgcaggaaacagaacagcg
aatagcgaggactctgatgaacaagaccctcaggaggtgacatacacacagttgaatcactgc
gttttcacacagagaaaaatcactcgcccttctcagaggcccaagacacccccaacagatatc
atcgtgtacacggaacttccaaatgctgagtccagatccaaagttgtctcctgcccaaaagaa
ccggcgagcccgctggataaatgccattataccaaagataacggccagtttgatcagagcgcg
aaacagctgaacctggaagcgtataccattgaacaggaaaccgcgctgattagcaacaaaaac
ggcaaaccgaaacgccagcagcgcaaaccgaacccgccgctgaacctggatagctatattgtg
ggccagaacgatatg
SEQ ID NO: 8 - iCAR4: KIR2DL1 (TM)- KIR2DL1
(ID)- NKG2A(CID) Peptide sequence:
(SEQ ID NO: 8)
ILIGTSVVIILFILLFFLLHRWCSNKKNAAVMDQESAGNRTANSEDSDEQDPQEVTYTQLNHC
VFTQRKITRPSQRPKTPPTDIIVYTELPNAESRSKVVSCPKEPASPLDKCHYTKDNGQFDQSA
KQLNLEAYTIEQETALISNKNGKPKRQQRKPNPPLNLDSYIVGQNDM
SEQ ID NO: 9 - iCAR5: KIR2DL1 (TM)- KIR2DL1 (ID)-
CD300A(CID) Nucleotide sequence:
(SEQ ID NO: 9)
attctgattgggacctcagtggtcatcatcctcttcatcctcctcttctttctccttcatcgc
tggtgctccaacaaaaaaaatgctgcggtaatggaccaagagtctgcaggaaacagaacagcg
aatagcgaggactctgatgaacaagaccctcaggaggtgacatacacacagttgaatcactgc
gttttcacacagagaaaaatcactcgcccttctcagaggcccaagacacccccaacagatatc
atcgtgtacacggaacttccaaatgctgagtccagatccaaagttgtctcctgcccaaggatg
tttcagaaatggatcaaagctggtgaccattcagagctgtcccagaaccccaagcaggctgcc
acgcagagtgagctgcactacgcaaatctggagctgctgatgtggcctctgcaggaaaagcca
gcaccaccaagggaggtggaggtggaatacagcactgtggcctcccccagggaagaacttcac
tatgcctcggtggtgtttgattctaacaccaacaggatagctgctcagaggcctcgggaggag
gaaccagattcagattacagtgtgataaggaagaca
SEQ ID NO: 10 - iCAR5: KIR2DL1 (TM)- KIR2DL1 (ID)-
CD300A(CID) Peptide sequence:
(SEQ ID NO: 10)
ILIGTSVVIILFILLFFLLHRWCSNKKNAAVMDQESAGNRTANSEDSDEQDPQEVTYTQLNHC
VFTQRKITRPSQRPKTPPTDIIVYTELPNAESRSKVVSCPRMFQKWIKAGDHSELSQNPKQAA
TQSELHYANLELLMWPLQEKPAPPREVEVEYSTVASPREELHYASVVEDSNINRIAAQRPREE
EPDSDYSVIRKT

In some embodiments, the immediate disclosure demonstrates use of CD19-targeting iCARs to prevent recognition and killing of a CD19-expressing cell, and one can utilize this approach when targeting other antigens shared between healthy and cancer cells. In some embodiments, this approach is useful for solid tumors when the antigens are often shared with normal cells. So, in certain embodiments, the iCAR will signal not to kill a cell if the antigen is shared between the normal and healthy cells. Although the iCAR and aCAR may target the same antigen, in particular embodiments, the iCAR and the aCAR target different antigens.

In particular embodiments, a single immune effector cell expresses one or more iCARs and one or more aCARs. In situations where more than one iCAR per cell is utilized, the respective antigen binding domains of the different iCARs may target different antigens, including different NK self antigens, for example. In specific embodiments, when the cells are desired to be engineered to express multiple engineered receptors of any kind, they may or may not be expressed from the same vector. When they are expressed from the same vector, they may be ultimately produced as separate polypeptides, such as through separation by a cleavage site, 2A element (e.g., T2A, P2A, E2A, F2A, etc.), or IRES element comprised in the vector.

In particular embodiments, an aCAR and/or an iCAR may be tailored to suit a particular need. For example, immune effector cells may be engineered to express a particular iCAR and later tailored to express a particular aCAR. In other cases, immune effector cells may be engineered to express a particular aCAR and later tailored to express a particular iCAR. Between such modifications, the immune effector cells may or may not be stored, such as cryogenically stored. In some embodiments, NK cells are engineered to express a particular iCAR, such as an iCAR that targets an NK self antigen (e.g., CS1), and then later (such as following cryogenic storage) it is modified to express a certain aCAR based on an individual's need, such as based on the type of cancer of an individual. In some embodiments, NK cells are engineered to express a particular aCAR, such as an aCAR that targets a cancer antigen, and then later (such as following cryogenic storage) it is modified to express a certain iCAR.

Embodiments of the disclosure include use of any of the engineered immune effector cells encompassed herein. Methods include enhancing cell therapies, including adoptive cell therapies, for individuals in need, such as individuals that have cancer and for which the engineered immune effectors cells are to be used for cancer therapy. In specific embodiments, the cell therapies employ aCARs that target one or more antigens present on the cancer cells. In specific cases, the iCAR inhibits killing of cells expressing the antigen to which the aCAR is directed when that antigen is not present on a cancer cell.

Embodiments of the disclosure include methods of administering to the individual a therapeutically effective amount of engineered immune effector cells, each comprising: (a) at least one inhibitory chimeric antigen receptor (iCAR) comprising at least one extracellular antigen binding domain, wherein a first extracellular antigen binding domain binds a first antigen; and at least one natural killer (NK) cell inhibitory signaling domain and/or at least one co-inhibitory domain; and (b) at least one activating chimeric antigen receptor (aCAR) comprising: at least one extracellular antigen binding domain, wherein a second antigen binding domain binds a second antigen; and an activating endodomain and at least one costimulatory signaling domain, and wherein:
(I) when the first antigen and the second antigen are the same and when the engineered immune effector cell binds through the second extracellular antigen binding domain a cell that expresses the antigen, the iCAR inhibits the killing by the engineered immune effector cell of the cell that expresses the antigen; or
(II) when the first and second antigen are non-identical and are both expressed on a fellow engineered immune effector cell or on a non-engineered immune effector cell of the same type or on a non-diseased cell, when the engineered immune effector cell binds through the second extracellular antigen binding domain to the second antigen on the fellow engineered immune effector cell or the non-engineered immune effector cell of the same type or the non-diseased cell, respectively, the iCAR inhibits the killing by the engineered immune effector cell of the fellow engineered immune effector cell or the non-engineered immune effector cell of the same type or the non-diseased cell, respectively.

In specific embodiments, the cell that expresses the antigen is a fellow engineered immune effector cell. In specific embodiments, the engineered immune effector cell binds through the second extracellular antigen binding domain to the second antigen on the fellow engineered immune effector cell. In specific embodiments, wherein the second antigen is expressed by the fellow engineered immune effector cell as a result of trogocytosis.

### III. NK Cells

Although it is contemplated that any type of immune effector cells may be utilized in the methods and compositions of the disclosure, including any type of T cell, in particular embodiments the present disclosure concerns processes of modifying NK cells, as opposed to other types of immune cells. NK cells are a subpopulation of lymphocytes that have spontaneous cytotoxicity against a variety of tumor cells, virus-infected cells, and some normal cells in the bone marrow and thymus. NK cells differentiate and mature in the bone marrow, lymph nodes, spleen, tonsils, and thymus. NK cells can be detected by specific surface markers, such as CD16 and/or, CD56 in humans. NK cells do not express T cell antigen receptors, the pan T marker CD3, or surface immunoglobulin B cell receptors.

In certain embodiments, NK cells are derived from human peripheral blood mononuclear cells (PBMC), unstimulated leukapheresis products (PBSC), human embryonic stem cells (hESCs), induced pluripotent stem cells (iPSCs), human hematopoietic stem cells, bone marrow, or umbilical cord blood or NK cell lines by methods well known in the art. Particularly, umbilical CB may be used to derive NK cells. In certain embodiments, precursor cells are engineered as described herein prior to differentiation and/or derivation into effector cells such as NK cells. In certain aspects, the NK cells are isolated and expanded by the previously described method of ex vivo expansion of NK cells (see, e.g., Spanholtz et al., 2011; and Shah et al., 2013). In this method, CB mononuclear cells are isolated by ficoll density gradient centrifugation and cultured in a bioreactor with IL-2 and artificial antigen presenting cells (aAPCs). After 7 days, the cell culture may be depleted of any cells expressing CD3 and re-cultured for an additional 7 days. The cells may again be CD3-depleted and characterized to determine the percentage of CD56⁺/CD3⁻ cells or NK cells. In other methods, umbilical CB is used to derive NK cells by the isolation of CD34⁺ cells and differentiation into CD56⁺/CD3⁻ cells by culturing in medium contain SCF, IL-7, IL-15, and/or IL-2.

As described elsewhere herein, in some embodiments, the NK cells and/or their precursor cells are expanded once or twice during their preparation. As noted above, in specific cases expansion of the NK cells comprises: stimulating mononuclear cells (MNCs) from cord blood in the presence of antigen presenting cells (APCs) and IL-2; and at some point in the process re-stimulating the cells with APCs to produce expanded NK cells. In at least some cases, the method is performed in a bioreactor. Multiple steps of the process may occur in the same vessel, including the same bioreactor. The stimulating step can direct the MNCs towards NK cells. The re-stimulating step may or may not comprise the presence of IL-2. In particular aspects, the method does not comprise removal or addition of any media components during a stimulating step. In particular aspects, the method is performed within a certain time frame, such as in less than 15 days, for example in 14 days.

As detailed elsewhere herein, in a certain embodiment, the NK cells are expanded by an ex vivo method for the expansion comprising: (a) obtaining a starting population of mononuclear cells (MNCs) from cord blood; (b) stimulating the MNCs in the presence of antigen presenting cells (APCs) and IL-2; and (c) re-stimulating the cells with APCs to produce expanded NK cells, wherein the method is performed in a bioreactor and is good manufacturing practice (GMP) compliant. The stimulating of step (b) can direct the MNCs towards NK cells. Step (c) may or may not comprise the presence of IL-2, in some cases. In particular aspects, the method does not comprise removal or addition of any media components during step (b). In particular aspects, the method is performed in less than 15 days, such as in 14 days.

As described, in certain embodiments, the method further comprises depleting cells positive for one or more particular markers, such as CD3, CD14, and/or CD19, for example. In certain aspects, the depleting step is performed prior to transduction or transfection and/or after gene editing. In some aspects, the cells are removed from the bioreactor for CD3, CD14, and/or CD19 depletion and placed in the bioreactor for subsequent steps.

In certain aspects, obtaining the starting population of MNCs from cord blood comprises thawing cord blood in the presence of dextran, human serum albumin (HSA), DNAse, and/or magnesium chloride. In particular aspects, obtaining the starting population of MNCs from cord blood comprises thawing cord blood in the presence of dextran and/or DNase. In specific aspects, the cord blood is washed in the presence of 5-20%, such as 10%, dextran. In certain embodiments, the cord blood is washed in the presence of 5-20%, such as 5%, 6%, 7%, 8%, 9%, 10%, 11%, 12%, 13%, 14%, 15%, 16%, 17%, 18%, 19%, or 20% dextran. In certain aspects, the cord blood is suspended in the presence of magnesium chloride, such as at a concentration of 100-300 mM, particularly 200 mM. In certain aspects, the cord blood is suspended in the presence of magnesium chloride, such as at a concentration of 100-300 mM, including 100-120 mM, 120-140 mM, 140-160 mM, 160-180 mM, 180-200 mM, 190-210 mM, 200-220 mM, 220-240 mM, 240-260 mM, 260-280 mM, or 280-300 mM. In some aspects, obtaining comprises performing ficoll density gradient centrifugation to obtain mononuclear cells (MNCs). In certain aspects, the cord blood from which the NK cells are derived is frozen cord blood. In particular aspects, the frozen cord blood has been tested for one or more infectious diseases, such as hepatitis A, hepatitis B, hepatitis C, Trypanosoma cruzi, HIV, Coronavirus, Human T-Lymphotropic virus, syphilis, Zika virus, and so forth. In some aspects, the cord blood is pooled cord blood, such as from 3, 4, 5, 6, 7, or 8 individual cord blood units.

In certain aspects, the method does not comprise human leukocyte antigen (HLA) matching. In some aspects, the starting population of NK cells are not obtained from a haploidentical donor.

In some aspects, the expanded NK cells produced from the process comprise a clinically relevant dose. In some aspects, the NK cells are autologous with respect to a recipient individual. In certain aspects, the NK cells are allogeneic with respect to a recipient individual.

In some embodiments, the NK cells express one or more heterologous antigen receptors, one or more antibodies, and/or one or more bispecific, trispecific, or multispecific engager/antibody.

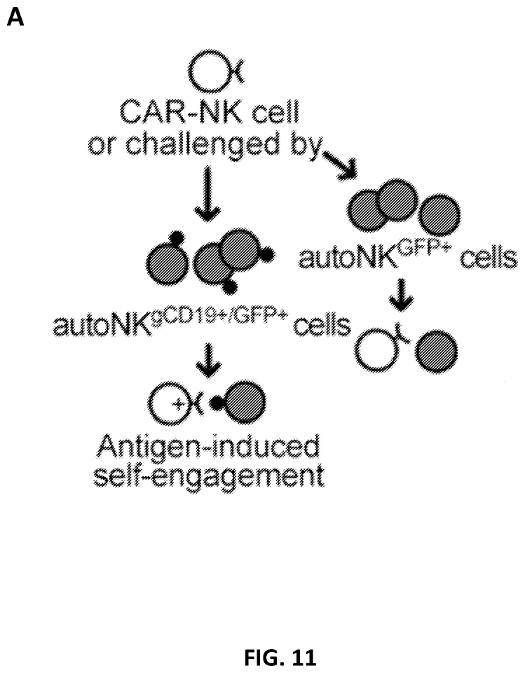

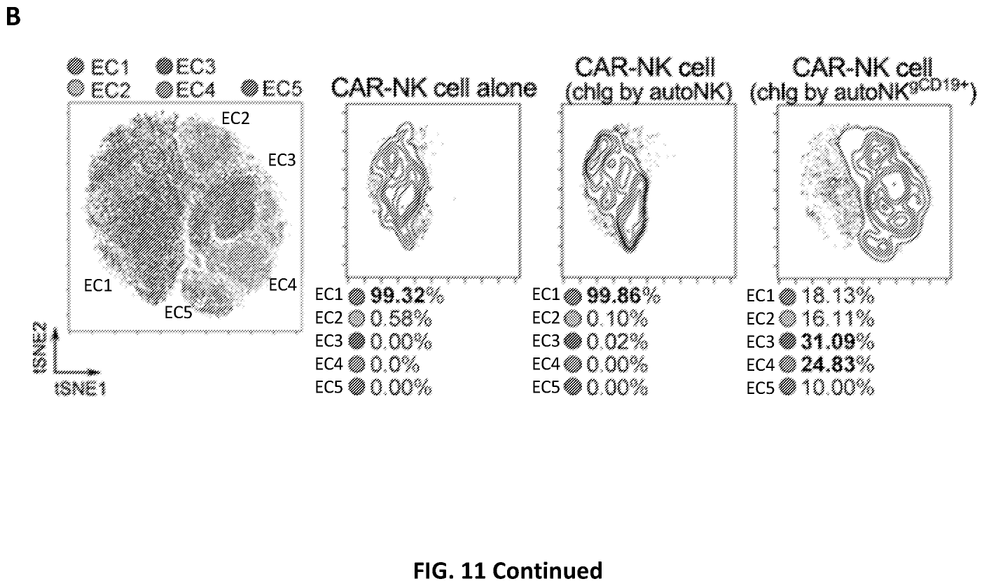

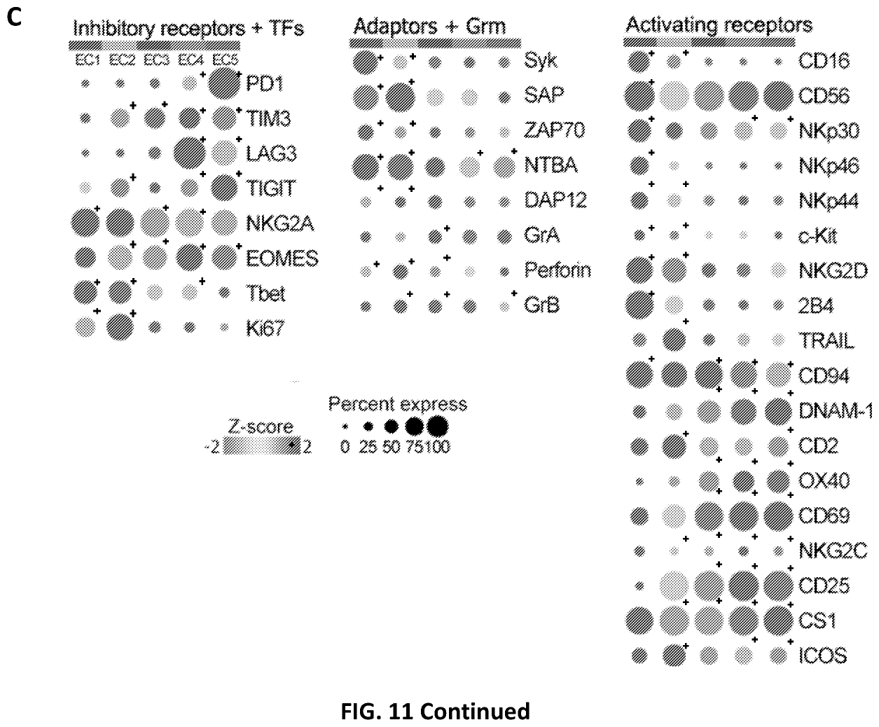

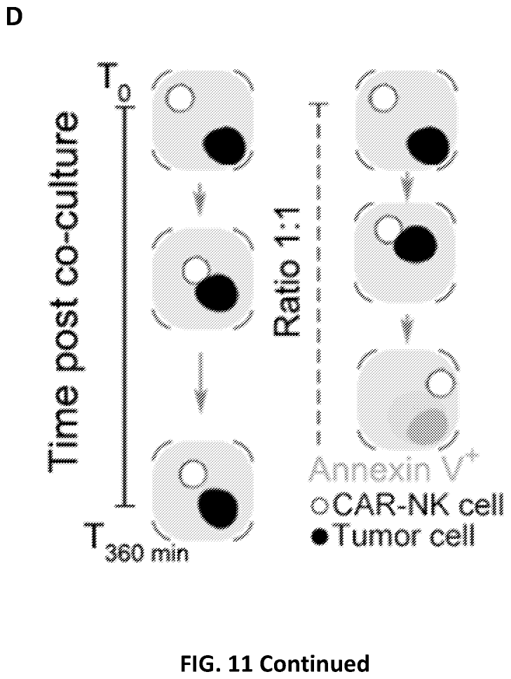

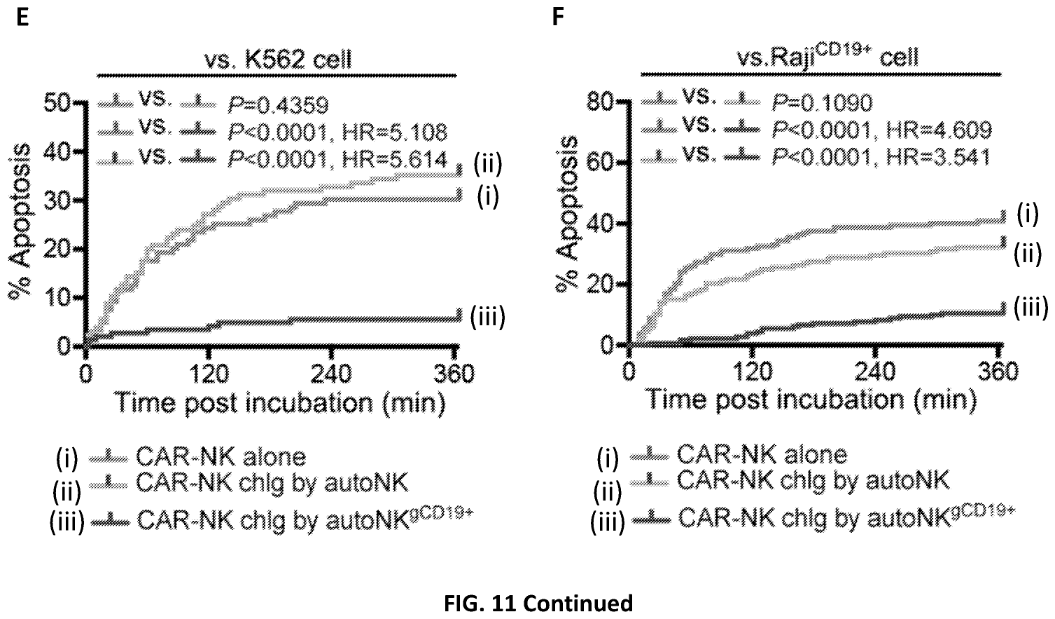

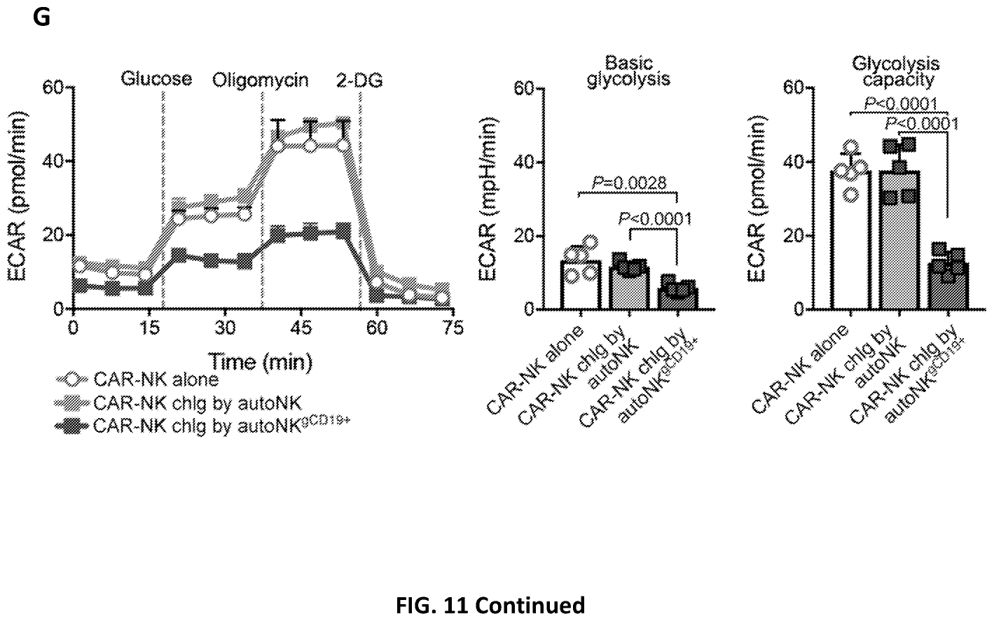

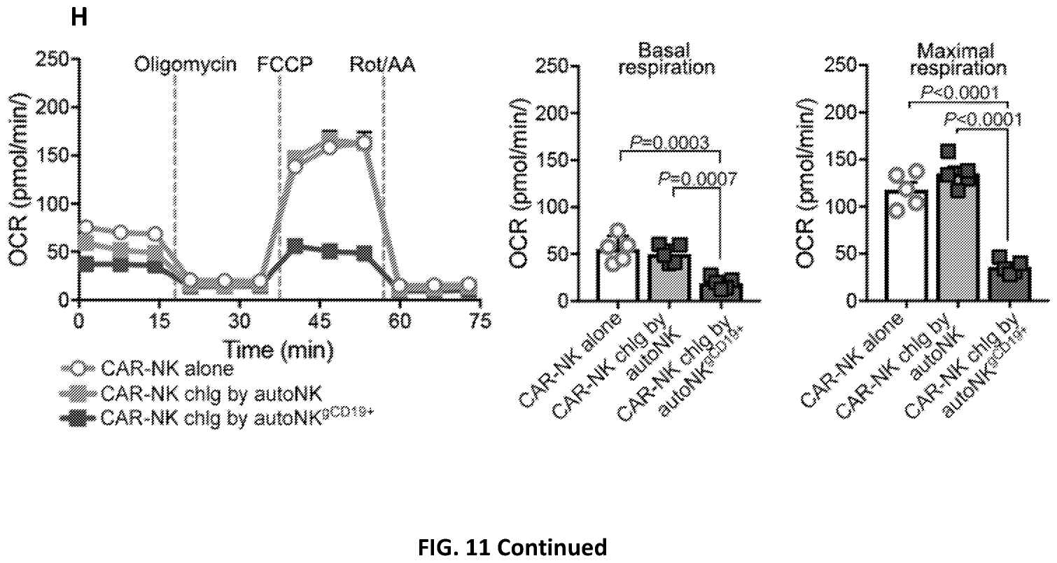

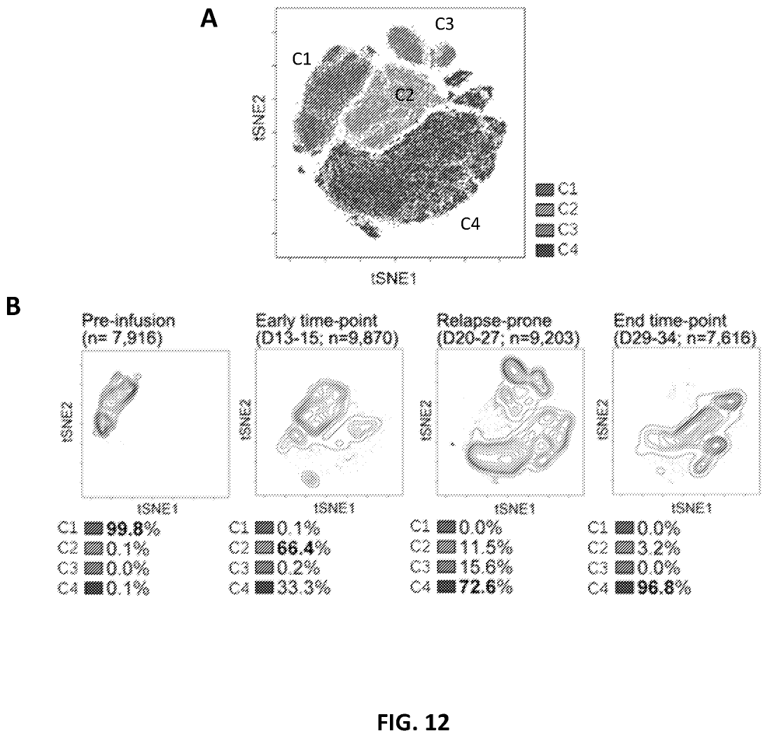

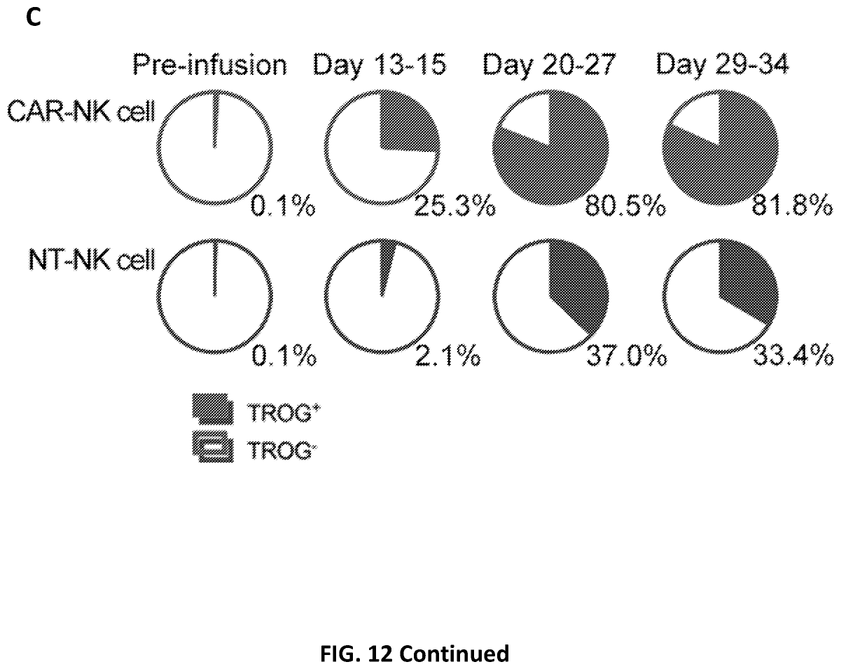

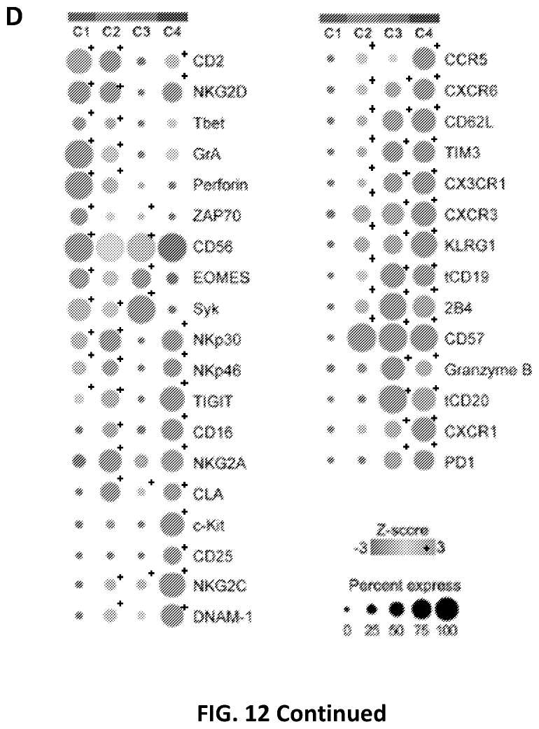

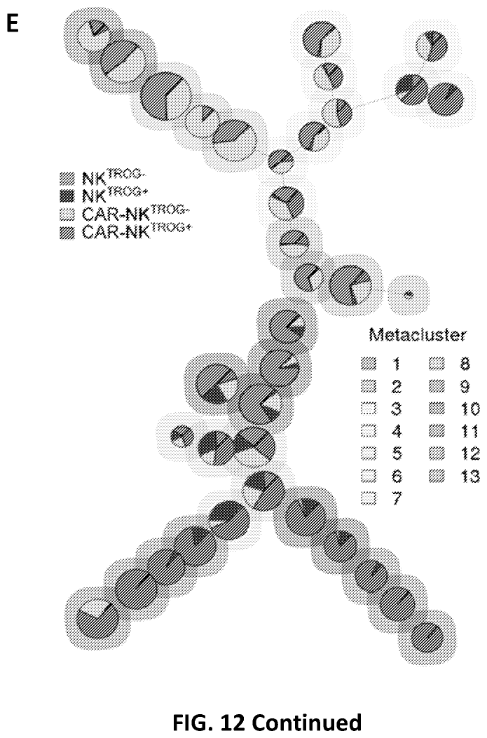

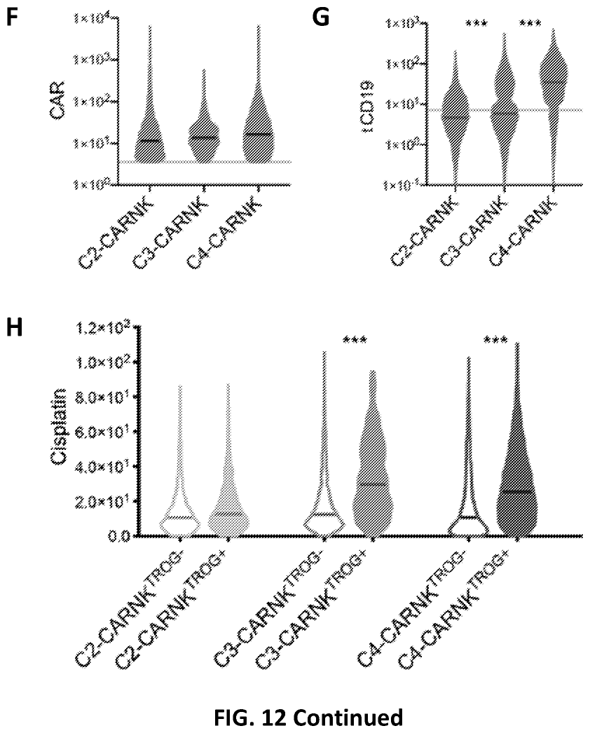

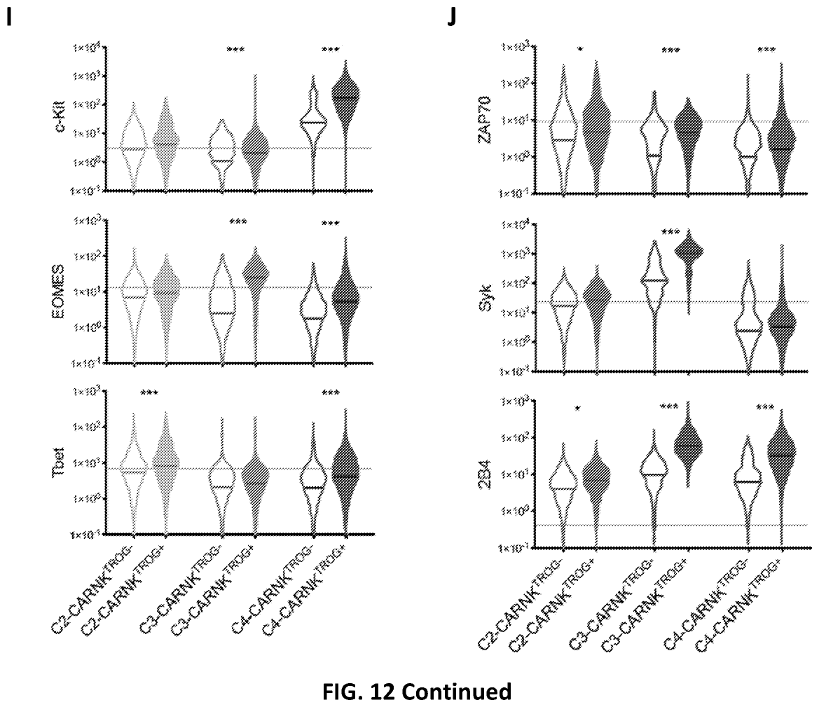

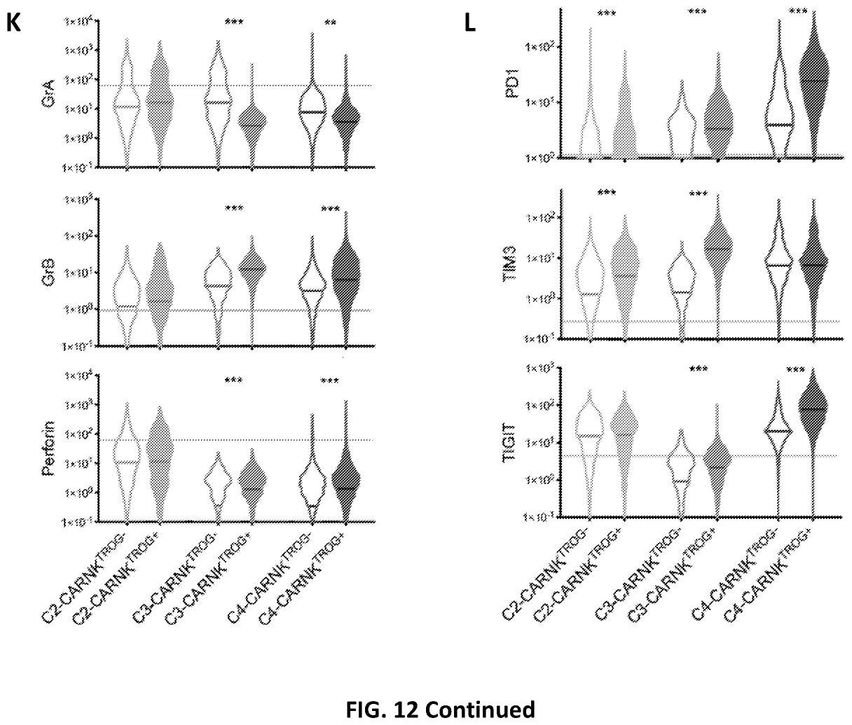

In some embodiments, an NK cell can be characterized by its cellular phenotype. In some embodiments, an NK cell can be described as hyporesponsive and/or exhausted when it expresses certain cell markers as described herein (see e.g., FIGS. 11 B-C, and FIGS. 12A-D). In some embodiments, technologies provided herein reduce the rate of NK cell hyporesponsiveness and/or exhaustion. For example, in some embodiments, technologies provided herein reduce the percentage of hyporesponsive and/or exhausted NK cells in a population by 5% to 100% when compared to an appropriate control. Including for example, reduction in hyporesponsiveness and/or exhaustion of NK cells in a population by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% when compared to an appropriate control.

In some embodiments, an NK cell can be characterized by its propensity to commit fratricide. In some embodiments, the likelihood of an NK cell to commit fratricide can be described as a function of the percentage of fraternal cells that have acquired a target antigen coupled with any NK cell specific negative inhibitory factors, e.g., presence of an iCAR as described herein, and can be measured through assays known in the art and/or described herein. In some embodiments, an NK cell can be characterized by its propensity to be a victim of fratricide. In some embodiments, the likelihood of an NK cell to undergo fratricide can be described as a function of the percentage of NK cells that have acquired a target antigen coupled with any fraternal cell specific negative inhibitory factors, e.g., presence of an iCAR as described herein. In some embodiments, technologies provided herein reduce the rate of NK cell fratricide, and can be measured through assays known in the art and/or described herein. For example, in some embodiments, technologies provided herein reduce the percentage of fratricide in an NK cell population by 5% to 100% when compared to an appropriate control. Including for example, reduction of fratricide in an NK cell population by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% when compared to an appropriate control.

In some embodiments, an NK cell can be characterized by its in vivo viability. In some embodiments, NK cell in vivo viability can be determined by analyzing checkpoint markers such as TIGIT, PD1, and/or TIM3. In some embodiments, NK cell in vivo viability can be increased using technologies provided herein. In some embodiments, in vivo viability can be increased by 1.1 to 10 fold (e.g., 1.1× to 10×) when compared to an appropriate control. In some embodiments, in vivo viability can be increased by 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 40×, 50×, 60×, 70×, 80×, 90×, 100×, 1000×, or up to 10000× when compared to an appropriate control.

In some embodiments, an NK cell can be characterized by its effector function. In some embodiments, effector function can be measured by determining the cytotoxicity of NK cells against target cells when compared to an appropriate control. In some embodiments, NK cell effector function can be increased using technologies provided herein. In some embodiments, NK cell effector function can be increased. In some embodiments, NK cell effector function can be increased by 1.1 to 10 fold (e.g., 1.1× to 10×) when compared to an appropriate control. In some embodiments, NK cell effector function can be increased by 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 40×, 50×, 60×, 70×, 80×, 90×, 100×, 1000×, or up to 10000× when compared to an appropriate control.

In some embodiments, an NK cell can be characterized by its in vivo persistence. In some embodiments, NK cell in vivo persistence can be determined by analyzing cell levels at appropriate timepoints after administration. In some embodiments, NK cell in vivo persistence can be increased using technologies provided herein. In some embodiments, in vivo persistence can be increased by 1.1 to 10 fold (e.g., 1.1× to 10×) when compared to an appropriate control. In some embodiments, in vivo persistence can be increased by 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 40×, 50×, 60×, 70×, 80×, 90×, 100×, 1000×, or up to 10000x when compared to an appropriate control.

In some embodiments, an NK cell can be characterized by its ability to shrink and/or inhibit growth of a target cell population (e.g., a tumor cell population). In some embodiments, an NK cells ability to shrink and/or inhibit growth of a target cell population can be measured using known methods such as ultrasound, x-ray, CT scan, MRI, biopsy, etc. In some embodiments, an NK cells ability to shrink and/or inhibit growth of a target cell population can be increased using technologies provided herein. In some embodiments, an NK cells ability to shrink and/or inhibit growth of a target cell population can be increased by 1.1 to 10 fold (e.g., 1.1× to 10×) when compared to an appropriate control. In some embodiments, an NK cells ability to shrink and/or inhibit growth of a target cell population can be increased by 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 40×, 50×, 60×, 70×, 80×, 90×, 100×, 1000×, or up to 10000× when compared to an appropriate control.

### IV. Heterologous Antigen Receptors

The immune effector cells of the present disclosure are genetically engineered to express one or more iCARs and also one or more heterologous antigen receptors, such as one or more engineered TCRs, one or more CARs, a combination thereof, and so on. The heterologous antigen receptors are synthetically generated by the hand of man. In particular embodiments, the immune effector cells are modified to express one or more CARs and/or one or more TCR, each having antigenic specificity for a different cancer antigen, and in addition to the iCAR. In some aspects, the immune effector cells are engineered to express the CAR(s) and/or TCR(s) by knock-in of the CAR or TCR at a particular gene locus, such as by using CRISPR.

Although in particular cases the NK cells are edited using CRISPR, where applicable, alternative suitable methods of modification are known in the art. See, for instance, Sambrook and Ausubel, supra. For example, the cells may be transduced to express a CAR or TCR having antigenic specificity for a cancer antigen using transduction techniques described in Heemskerk et al., 2008 and Johnson et al., 2009. In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; and/or Wu et al., 2012. In some aspects, the genetically engineered antigen receptors include a CAR as described in U.S. Pat. No. 7,446,190, and those described in International Patent Application Publication No.: WO/2014055668 A1.

### A. Chimeric Antigen Receptors

In some embodiments, the CAR comprises: a) one or more intracellular signaling domains, b) a transmembrane domain, and c) an extracellular domain comprising one or more antigen binding domains. The extracellular antigen binding domain may be associated with a hinge of any kind, such as from IgG1, CD28, CD8alpha, and so forth.

In some embodiments, the engineered antigen receptors include CARs, including activating or stimulatory CARs, costimulatory CARs (see WO2014/055668), and/or inhibitory CARs (iCARs, see e.g., Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

In some embodiments, an antigen-specific CAR polypeptide may comprise one or more epitope recognition domains that do not comprise an scFv. In some embodiments, an antigen-specific CAR polypeptide may comprise an antigen binding domain derived from one or more proteins selected from adnectins, affibodies, affillins, anticalins, atrimers, avimers, bicyclie peptides, centyrins, cys-knots, DARPins, FN3, Fynomers, Kunitz domains, Obodies, pronectins, and Tn3. In some embodiments, such antigen binding domains may be modified and/or optimized for human codon usage and/or expression in human cells.

In some embodiments, a CAR arrangement can comprise polypeptides and/or proteins that could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, 4-1BB (CD137), or a combination thereof. In addition to a primary signal initiated by CD3ζ, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of antigens include CD19, CD70, HLA-G, CD38, CD123, CLL1, EBNA, CD123, HER2, CA-125, TRAIL/DR4, CD20, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MCIR, mda-7, gp75, Gp100, PSA, PSM, Tyrosinase, tyrosinase-related protein, TRP-1, TRP-2, ART-4, CAMEL, CEA, Cyp-B, hTERT, hTRT, ICE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mber-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1, or a combination thereof.

In certain embodiments, the CAR may be co-expressed with one or more cytokines to improve persistence when there is a low amount of tumor-associated antigen. For example, CAR may be co-expressed with one or more cytokines, such as IL-7, IL-2, IL-15, IL-12, IL-18, IL-21, or a combination thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR″ immune cells (see, e.g., Singh et al., 2008; and Singh et al., 2011).

### B. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail. For example, in some aspects, each chain of the TCR can possess one N-terminal immunoglobulin variable domain, one immunoglobulin constant domain, a transmembrane region, and a short cytoplasmic tail at the C-terminal end (see, e.g., Janeway et al., 1997). In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; and Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Cₐ, typically amino acids 117 to 259 based on Kabat, B-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009; and Cohen et al., 2005). In some embodiments, phage display is used to isolate TCRs against a target antigen (see, e.g., Varela-Rohena et al., 2008; and Li et al., 2005). In some embodiments, the TCR or antigen-binding portion thereof can be synthetically generated from knowledge of the sequence of the TCR.

### C. Antigens

Among the antigens targeted by the genetically engineered iCARs and/or aCARs are those expressed in the context of a disease, condition, syndrome, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (see, e.g., Linnemann et al., 2015). In particular aspects, the antigens include CD19, EBNA, CD123, HER2, CA-125, TRAIL/DR4, CD20, CD70, HLA-G, CD3δ, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MCIR, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1. Examples of sequences for antigens are known in the art, for example, in the GENBANK® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRVIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, LAGE (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, instead of a cancer antigen (tumor antigen), an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), coronavirus, cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (see, e.g., Ryckman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see, e.g., Bennekov et al., 2004; Loewendorf & Benedict 2010; and Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 2009).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Antigens derived from coronavirus (e.g., SARS-CoV-2) that are contemplated for use in certain embodiments include: membrane (M) protein, envelope (E) protein, spike (S) protein, nucleocapsid (N) protein, coronavirus RNA, non-structural proteins Nsp1-Nsp16, and/or accessory proteins (3a, 3b, 6, 7a, 7b, 8, 9b, 9c, and/or 10). In a preferred embodiment, antigens derived from coronavirus are from the S protein.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, see, e.g., Snyder et al., 2009; and Snyder et al., 2010). As would be understood by the skilled person, Staphylococcus proteins for use as antigens may also be identified in other public databases such as GENBANK®, SWISS-PROT®, and TREMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### D. Suicide Genes

In some cases, any cells of the disclosure are modified to produce one or more agents other than heterologous cytokines, engineered receptors, and so forth. In specific embodiments, the cells, such as NK cells, are engineered to harbor one or more suicide genes, and the term “suicide gene” as used herein is defined as a gene which, upon administration of a prodrug, effects transition of a gene product to a compound which kills its host cell. In some cases, the NK cell therapy may be subject to utilization of one or more suicide genes of any kind when an individual receiving the NK cell therapy and/or having received the NK cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Examples of suicide genes include engineered nonsecretable (including membrane bound) tumor necrosis factor (TNF)-alpha mutant polypeptides (see PCT/US19/62009, which is incorporated by reference herein in its entirety), and they may be targeted by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP), as examples.

### E. Methods of Delivery

In specific embodiments, any composition may be delivered to the recipient immune effector cells by any suitable methods. The compositions may be delivered to the cells by electroporation or by a vector, for example. In specific embodiments, for example, one or more compositions for introduction of at least one or more heterologous antigen receptors are delivered to the immune effector cells in a vector. In some embodiments, one or more compositions for gene editing are delivered to the cells in a vector. One of skill in the art would be well-equipped to construct a vector through standard recombinant techniques (see, e.g., Sambrook et al., 2001; and Ausubel et al., 1996) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV, etc.), lentiviral vectors (e.g. derived from HIV-1, HIV-2, SIV, BIV, FIV, etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

In specific embodiments, the vector is a multicistronic vector, such as is described in PCT/US19/62014, which is incorporated by reference herein in its entirety. In such cases, a single vector may encode one or more CAR and/or TCR (and the expression construct may be configured in a modular format to allow for interchanging parts of the CAR or TCR), a suicide gene, and/or one or more cytokines. In some embodiments, at least one activating CAR and at least one inhibitory CAR are included in a single vector.

### 1. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, e.g., U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

### a Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

b. Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 to 110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. In some embodiments, a promoter and/or an enhancer may be an endogenous gene's promoter and/or enhancer. In some embodiments, a promoter and/or an enhancer may be associated with a “safe harbor” locus as known in the art. Alternatively, in certain embodiments, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the Blactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, e.g., Sambrook et al. 2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GAPDH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

c. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

d. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

e. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### 2. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### V. Gene Editing and CRISPR

The NK cell production process of the disclosure may include gene editing of the NK cells to remove 1, 2, 3, 4, 5, 6, 7, 8, 9, 10, or more endogenous genes in the NK cells. In some cases the gene editing occurs in NK cells expressing one or more heterologous antigen receptors, whereas in other cases the gene editing occurs in NK cells that do not express a heterologous antigen receptor but that ultimately will express one or more heterologous antigen receptors, in at least some cases. In particular embodiments, the NK cells that are gene edited are expanded NK cells. In certain embodiments, NK cells that are gene edited were derived from precursor cells that were previously gene edited.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. In specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD3δ, LAG3, TCR, beta2-microglobulin, HLA, CD73, and CD39. In specific embodiments, the TGFBR2 gene is knocked out or knocked down in the NK cells. In specific embodiments, the CISH gene is knocked out or knocked down in the NK cells. In specific embodiments, the CD38 gene is knocked out or knocked down in the NK cells. In specific embodiments, the CISH gene and the CD38 gene are knocked out or knocked down in the cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502.

### VI. Methods of Treatment

In some embodiments, the immune effector cells produced by the methods of the disclosure are utilized for methods of treatment for an individual in need thereof. Embodiments of the disclosure include methods of treating an individual for cancer, infections of any kind, and/or any immune disorder, as examples. The individual may utilize the treatment method of the disclosure as an initial treatment or after (and/or with) another treatment. In cancer embodiments, the immunotherapy methods may be tailored to the need of an individual with cancer based on the type and/or stage of cancer, and in at least some cases the immunotherapy may be modified during the course of treatment for the individual.

In specific cases, examples of treatment methods are as follows: 1) adoptive cellular therapy with the produced immune effector cells (ex vivo expanded or expressing CARs or TCRs) to treat cancer patients with any type of hematologic malignancy, (2) adoptive cellular therapy with the produced immune effector cells (ex vivo expanded or expressing CARs or TCRs) to treat cancer patients with any type of solid cancers, (3) adoptive cellular therapy with the produced immune effector cells (ex vivo expanded or expressing CARs or TCRs) to treat patients with infectious diseases and/or immune disorders.

In some embodiments, the present disclosure provides methods for immunotherapy comprising administering an effective amount of the immune effector cells produced by methods of the present disclosure. In one embodiment, a medical disease or disorder is treated by one or more transfers of immune effector cell populations produced by methods herein and that elicit an immune response, in at least particular cases. In certain embodiments of the present disclosure, cancer or infection is treated by delivery of one or more immune effector cell populations produced by methods of the disclosure and that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and/or viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of hematological malignancies, such as lymphoma or leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days, or 1, 2, 3, or 4 or more weeks, or 1, 2, 3, 4, 5, 6, 7, 8, 9, 10, 11, 12, or more months.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m2 fludarabine is administered for five days.

In certain embodiments, one or more growth factors that promotes the growth and activation of the NK cells is administered to the subject either concomitantly with the NK cells or subsequently to the NK cells. The growth factor can be any suitable growth factor that promotes the growth and activation of the NK cells. Examples of suitable immune cell growth factors include interleukin (IL)-2, IL-7, IL-12, IL-15, IL-18, and IL-21, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of the produced NK cells can be administered by a number of routes, including parenteral administration, for example, intravenous, intraperitoneal, intramuscular, intrasternal, intratumoral, intrathecal, intraventricular, through a reservoir, intraarticular injection, or infusion.

The therapeutically effective amount of the produced immune effector cells for use in adoptive cell therapy is that amount that achieves a desired effect in a subject being treated. For instance, this can be the amount of NK cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The produced immune effector cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of immune effector cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune effector cells/m². In a certain embodiment, the dose used in the treatment of human subjects ranges from about 3.8×10⁹ to about 3.8×10¹⁰ immune effector cells/m². In additional embodiments, a therapeutically effective amount of immune effector cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune effector cells is readily determined by one of skill in the art based on the age, weight, sex, and physiological condition of the subject. Effective doses can be extrapolated from dose-response curves derived from in vitro or animal model test systems.

The immune effector cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

In some embodiments, provided herein are methods for increasing effector cell (e.g., a CAR-NK cell) viability in a subject. For example, in some embodiments, utilization of an iCAR as described herein can increase effector cell viability in a subject by 1.1 to 10 fold (e.g., 1.1× to 10×) when compared to an appropriate control. In some embodiments, effector cell viability in a subject can be increased by 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 40×, 50×, 60×, 70×, 80×, 90×, 100×, 1000×, or up to 10000× when compared to an appropriate control.

In some embodiments, provided herein are methods for increasing effector cell (e.g., a CAR-NK cell) persistence in a subject. For example, in some embodiments, utilization of an iCAR as described herein can increase effector cell persistence in a subject by 1.1 to 10 fold (e.g., 1.1× to 10×) when compared to an appropriate control. In some embodiments, effector cell persistence in a subject can be increased by 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 40×, 50×, 60×, 70×, 80×, 90×, 100×, 1000×, or up to 10000× when compared to an appropriate control.

In some embodiments, provided herein are methods for increasing effector cell (e.g., a CAR-NK cell) efficacy in a subject. For example, in some embodiments, utilization of an iCAR as described herein can increase effector cell efficacy in a subject by 1.1 to 10 fold (e.g., 1.1× to 10×) when compared to an appropriate control. In some embodiments, effector cell efficacy in a subject can be increased by 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 40×, 50×, 60×, 70×, 80×, 90×, 100×, 1000×, or up to 10000× when compared to an appropriate control.

In some embodiments, provided herein are methods for increasing subject survival when compared to a suitable control subject or population. For example, in some embodiments, utilization of an effector cell comprising an iCAR as described herein can increase a subjects survival by 1 week, 2 weeks, 3 weeks, 4 weeks, 5 weeks, 6 weeks, 7 weeks, 8 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or more than 10 years when compared to a suitable control subject or population.

In some embodiments, provided herein are methods for inhibiting tumor growth and/or initiating tumor shrinkage. For example, in some embodiments, utilization of an effector cell comprising an iCAR as described herein can inhibit tumor growth by 5% to 100% when compared to an appropriate control. For example, in some embodiments, technologies provided herein inhibit tumor growth by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% when compared to an appropriate control. In some embodiments, utilization of an effector cell comprising an iCAR as described herein can shrink a tumor by 5% to 100% when compared to an appropriate control. For example, in some embodiments, technologies provided herein shrink a tumor by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% when compared to an appropriate control.

In some embodiments, provided herein are methods suitable for increasing the rate of subjects classified as responding to an effector cell based therapy. For example, in some embodiments, utilization of an effector cell comprising an iCAR as described herein can increase the percentage of subjects classified as responding to an effector cell based therapy from 5% to 100% when compared to an appropriate control. For example, in some embodiments, technologies provided herein increase the percentage of subjects classified as responding to an effector cell based therapy by 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% when compared to an appropriate control.

In some embodiments, provided herein are methods for increasing circulating serum levels of effector cell associated proteins, for example but not limited to, proteins such as GrA, GrB, Perforin, IFNγ, TNFα, or combinations thereof. In some embodiments, utilization of an effector cell comprising an iCAR as described herein can increase the circulating levels of one or more effector cell associated proteins by 1.1 to 10 fold (e.g., 1.1× to 10×) when compared to an appropriate control. In some embodiments, circulating levels of one or more effector cell associated proteins can be increased by 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 40×, 50×, 60×, 70×, 80×, 90×, 100×, 1000×, or up to 10000x when compared to an appropriate control.

### A. Pharmaceutical Compositions

Also provided herein are pharmaceutical compositions and formulations comprising immune effector cells produced by the processes encompassed herein and a pharmaceutically acceptable carrier.

Pharmaceutical compositions and formulations as described herein can be prepared by mixing the active ingredients (e.g., cells as described herein,) having the desired degree of purity with one or more optional pharmaceutically acceptable carriers (see, e.g., Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEXR, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### B. Combination Therapies

In certain embodiments, the compositions and methods of the present embodiments involve an immune effector cell population in combination with at least one additional therapy. For cancer embodiments, the additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. For pathogenic conditions, the additional therapy may comprise one or more antibiotics, antivirals, and so forth.

In some cancer embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune effector cell therapy of the disclosure may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates (ADCs) comprise monoclonal antibodies (mAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs include ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL I to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and YERVOY®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (e.g., Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. Articles of Manufacture or Kits

An article of manufacture or a kit is provided comprising immune effector cells, and/or one or more reagents for generating them. The immune effector cells may be from any source, and in specific embodiments the immune effector cells have been produced by methods encompassed herein. In specific embodiments, the immune effector cells have been gene edited and may be provided in the kit so that they may be further modified to express one or more iCARs and one or more heterologous antigen receptors. In specific embodiments, the immune effector cells have been modified to express one or more iCARs and one or more heterologous antigen receptors and may be provided in the kit so that they may be further modified to be gene edited. In specific embodiments, one or more reagents for generating the immune effector cells are provided in the kit, such as a vector encoding an iCAR or reagents to produce same, a vector encoding a CAR or reagents to produce same; reagents that target a specific NK cell gene, or a combination thereof. In general embodiments, the reagents may comprise nucleic acid including DNA or RNA, primers, protein, media, buffers, salts, co-factors, and so forth. In specific cases, the kit comprises one or more CRISPR-associated reagents, including for targeting a specific desired NK cell gene.

The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (e.g., such as polyvinyl chloride or polyolefin), or metal alloy (e.g., such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes, etc.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### METHODS

Unless otherwise stated, assays and experiments described in the following examples were performed as described herein.

### Cell Lines, Primary Cells and Culture Conditions

CD19⁺ cell lines of Raji (CCL-86), NALM-6 (CRL-3273) and Ramos (CRL-1596), CD5⁺ cell line CCRF (CRM-CCL-119), CD70⁺ cell line THP-1 (TIB-202), CD123⁺ cell line MOLM-14 (ACC 777), BCMA″ cell line MM1S (CRL-2974), SKOV3 cell line (HTB-77), K562 cell line (CRL-3344) and 293T cell line (CRL-3216) were obtained from the American Type Culture Collection (ATCC). Cells of Raji, NALM-6, Ramos, CCRF, MOLM-14, K562 were cultured in RPMI-1640 (Invitrogen) supplemented with 10% fetal bovine serum (FBS; HyClone), 1% penicillin-streptomycin, and 1% GLUTAMAX™; cells of THP-1 and 293T cells were cultured in DMEM (Invitrogen) supplemented with 10% FBS, 1% penicillin-streptomycin and 1% GLUTAMAX™; SKOV3 cells were cultured in McCoy's 5a Medium (Invitrogen) supplemented with 10% FBS, 1% penicillin-streptomycin and 1% GLUTAMAX™. K562 cells were retrovirally transduced to co-express 4-1BBL, CD48, and membrane-bound interleukin (IL)-21 and served as universal antigen presenting cells (uAPC) for in vitro NK cell expansion (see, e.g., Liu et al., 2021). For modeling trogocytosis detection by using fluoresecent-traceable marker, the CD19 gene in Raji cells was deleted using the CRISPR-Cas9 system; (crRNA1: CTAGGTCCGAAACATTCCAC-CGG (SEQ ID NO: 11), crRNA2: CGAGGAACCTCTAGTGGTGA-AGG (SEQ ID NO: 12) CD19-knockout Raji (Raji^{CD19-KO}) cells were purified by MoFlo Astrios (Beckmen Coulter) and then retrovirally transduced to express CD19-mCherry fusion protein with or without GFP co-expression (Raji^{CD19+mtCherry/GFP} and Raji^{CD19+mCherry}). Raji cells were transduced with firefly luciferase-GFP to allow in vivo tumor burden examination using the IVIS Spectrum imaging system (Caliper). To model solid tumors in vivo, SKOV3 cells were retrovirally transduced to express CD19 (SKOV3^{gCD19+}) and firefly luciferase-GFP. All cells were maintained in a 37° C. incubator with 5% CO₂, and regularly tested for mycoplasma contamination using the MycoAlert Mycoplasma Detection Kit (Lonza).

PBMC from Patients Treated with CAR19/IL-15 NK Cells

Clinical samples used in this study were collected from patients treated on a clinical trial of iC9/CAR19/IL-15 (CAR19/IL-15) transduced cord blood (CB)-NK cells as previously reported (NCT03056339, see, e.g., Liu et al., 2020). Primary cells of peripheral blood monocytes (PBMC) from 11 patients with chronic lymphocytic leukemia (CLL) or non-Hodgkin lymphoma (NHL) were collected at different time points during their treatment at MD Anderson Cancer Center. The normalized mean TROG-CD19 (tCD19) gMFI on CAR19-NK cells for the whole patient cohort was 6.29 (range of 0.61-35.77). Patients with a high (>mean) versus low (≤mean) normalized tCD19-gMFI at more than one time point were defined as TROG^{hi} (n=4 patients) vs TROG^{low} (n=7 patients), respectively. In addition, circulating leukemia cells from four patients with CLL and four patients with B cell-acute lymphocytic leukemia (ALL) enrolled on laboratory protocols were isolated after density-gradient centrifugation for in vitro studies of trogocytosis. All patients gave informed consent per the Institutional Review Board (IRB). All studies were performed in accordance with the Declaration of Helsinki.

### Vector Constructs and Retrovirus Production

The retroviral vector encoding iCas9, 19scFv.CD28.zeta.2A.IL-15 (CAR19/IL15), was kindly provided by Dr. Gianpietro Dotti (University of North Carolina—Chapel Hill) (see, e.g., Hoyos et al., 2010). The iCas9.19scFv.2A.IL-15 (19scFv/IL15), iCas9.CD5scFv (XZ-CD5 (see, e.g., Przepiorka et al., 1994)). CD28.zeta.2A.IL-15 (CAR5/IL15), iCas9.CD5scFv.DAP10.zeta.2A.IL-15 (CAR(DAP10z)/IL15), iCas9.CD5scFv.zeta.2A.IL-15 (CAR(3z)/IL15), iCas9.CD5scFv.DAP12.zeta.2A.IL-15 (CAR(DAP12z)/IL15), iCas9.CD5scFv.NKG2D.zeta.2A.IL-15 (CAR(2Dz)/IL15), iCas9.CD5scFv.41BB.zeta.2A.IL-15 (CAR(BBz)/IL15), iCas9.CD5scFv.DAP10.2A.IL-15 (CAR(DAP10)/IL15), iCas9.CD5scFv.DAP12.2A.IL-15 (CAR(DAP12)/IL15), iCas9.CD70scFv (ARGX-110 or LB #14 (see, e.g., Silence et al., 2014)).CD28.zeta.2A.IL-15 (CAR70/IL15), iCas9,CD27(ECD).CD28.zeta.2A.IL-15 (CAR27s/IL15), iCas9.CD123scFv (26292 (see, e.g., Du et al., 2007)).CD28.zeta.2A.IL-15 (CAR123/IL15), and iCas9.BCMAscFv (huc11D5.3-Luc90 (see, e.g., Zah et al., 2020)).CD28.zeta.2A.IL-15 (CAR-BCMA/IL15) constructs were cloned into the SFG retroviral backbone to generate additional viral vectors. For the construction of inhibitory CARs (iCARs), transmembrane domains of KIR2DL1 and LIR-1, cytoplasmic signaling domains of KIR2DL1, LIR-1, LAIR-1, NKG2A, and CD300A were used as inhibitory signals. Extracellular domains comprising either 19scFv or CS1scFv (HuLuc63 (see, e.g., Tai et al., 2008)), along with IgG hinge, were used to fuse and generate iCAR19 or iCAR-CS1 constructs, respectively. CS1-scFv, iCAR-CS1, and iCAR19/IL15 constructs were then each cloned into the SFG retroviral backbone. The whole CD19 coding sequence was fused to an mCherry reporter gene at the 3′ end to generate the CD19-mCherry construct. CD19-mCherry and GFP were then linked using the 2A peptide resulting in the bicistronic CD19-mCherry/GFP construct. mCherry, CD19-mCherry, CD19-mCherry/GFP were also each cloned into the SFG retroviral backbone. All construct syntheses and molecular cloning were performed by GeneArt Gene Synthesis (Thermo Fisher Scientific). Transient retroviral supernatants were produced from transfected 293T cells as previously described (see, e.g., Vera et al., 2006).

### Cord Blood NK Cell Transduction and Expansion

Cord blood (CB) units were provided by the MD Anderson Cancer Center Cord Blood Bank. CB-derived NK cells (CB-NK) were isolated and expanded as previously described (see, e.g., Liu et al., 2018). In brief, lymphocytes were collected by density-gradient centrifugation using Ficoll-Histopaque solution (Sigma-Aldrich). CD56⁺CD3⁻ NK cells were then purified using an NK negative isolation kit (Miltenyi Biotec), and co-cultured with irradiated (100 Gy) uAPC at a 2:1 ratio in complete stem cell growth medium (SCGM), supplemented with 200 U/ml recombinant human IL-2 (Proleukin). On Day 4 post uAPC stimulation, fresh NK cells were purified again and transduced with retroviral vectors expressing CAR-constructs. A second retroviral transduction of iCAR constructs was performed on Day 6 to then generate NK cells expressing AI-CAR. Following the same approach, CD19-mCherry or CD19-mCherry/GFP expressing cells (both primary NK cells and tumor cells) were prepared. CAR transduction efficiency was measured by flow cytometry. Irradiated uAPC were added weekly to the NK cell culture to support NK cell expansion.

### Flow Cytometry

CAR expression was measured by detection of IgG hinge using conjugated goat anti-human lgG (H+L) (Jackson ImmunoResearch). For AI-CAR detection, anti-CD19 aCAR expression was measured using the CD19-CAR detection reagent (Miltenyi Biotec). Expression of anti-CS1 iCAR was measured by binding of CAR to CS1 his-tag fusion protein (ACRO Biosystems). GHOST DYE™ Violet 450 (TONBO Biosciences) was used to determine viability, and aqua fixable viability dye (eBioscience) was used for assessing viability when fixation protocols were applied. Human Fc receptor blocking solution (Miltenyi Biotec) was used to block Fc receptors to minimize non-targeted specific staining. For intracellular staining, cells were fixed and permeabilized using Intracellular Fixation and Permeabilization Buffer Kit (eBioscience) according to the manufacturer's protocol. For phosphoflow staining, cells were prepared and fixed using the Perfix Expose Kit from Beckman Coulter, according to the manufacturer's protocol. Phycoerythrin Fluorescence Quantitation Kit (BD Biosciences) was used according to the manufacturer's protocol to determine the number of molecules of CD19, CD5, CD70, CD123, and BCMA per cell. AccuCheck Counting Beads (ThermoFisher) were used to determine the cell concentration in each tested population. Amnis Imagestream-X MarkII (Millipore) was used to visualize fixed cells at 60× magnification with a pixel size of 0.1 μm², data were analyzed using IDEAS (Millipore). Flow cytometry analysis was performed on LSRFORTESSA™ X-20 (BD Bioscience), and data were analyzed using FlowJo (BD Bioscience). Cell sorting was performed using MoFlo XDP cell sorter (Beckman Coulter).

### Trogocytosis Assay

NK cells were co-cultured with designated GFP″ target cells at an effector:target (E:T) ratio of 1:1. Co-cultured cells were washed with FACS buffer and then subjected to surface staining of anti-hCD56 (Biolegend, HCD56) and anti-hCD3 (Biolegend, SK7) antibodies at 4ºC for 20 minute in the dark. Following staining, cells were washed and assessed by flow cytometry. The TROG⁺ population was defined by the detection of TROG-antigen on the surface of singlet NK (CD56⁺CD3⁻GFP⁻) cells; also, the cognate antigen expression on the co-cultured tumor cells (CD56⁻GFP⁺) was evaluated. The IncuCyte Live-Cell Analysis System (ESSEN Bioscience) was used for the mCherry-based trogocytosis assay, where Raji^{CD19+mCherry} cells or Raji^{mCherry} cells were co-cultured with CFSE (ThermoFisher) labeled NK cells at a 1:1 ratio. Image scanning of the mCherry signal in NK cells was recorded in real-time and cells that showed both mCherry and CFSE signals were identified as the NK^{TROG+} population. To block trogocytosis, NK cells were pre-treated with 1 μM latrunculin A (Sigma-Aldrich) at 37° C. for 20 min before co-culture with target cells.

### NK Activation Assay

NK cells were stimulated by target cells at an E:T ratio of 1:1 for 6 hrs. To inhibit protein transport, GolgiStop and GolgiPlug (BD Bioscience) were added to the culture at the second hour post-co-culture according to the manufacturer's protocol. Anti-CD107a (Biolegend, H4A3) was also added at this time point to capture CD107a as a marker of NK cell degranulation. When examining NK^{TROG+} cell populations, GolgiStop and GolgiPlug were not added to allow the trogocytosis. After incubation, cells were washed with FACS buffer (BD Bioscience) and stained with anti-hCD56, anti-hCD3. GHOST DYE™ Violet 450 (Tonbo Biology) was used to identify the viability of NK populations. Intracellular staining with interferon-gamma (IFN-γ) (BD Bioscience, B27) and tumor necrosis factor-alpha (TNF-α) (BD Bioscience), MAb11 antibodies were subsequently applied. Expression of CD107a, IFN-γ, and TNF-α was measured and expressed as a percentage of CD56⁺CD3⁻ NK cells when compared with un-stimulated NK cells.

### Cytotoxicity Assay in IncuCyte System

NK cells were co-cultured at an E:T ratio of 1:1 with tumor cells either labeled with Vybrant DyeCycle Ruby Stain (ThermoFisher) or expressing mCherry signal. The IncuCyte Caspase-3/7 green apoptosis assay reagent (SAETORIUS) was added to each well to label apoptotic cells. Images of each well were captured in real-time during the period of 6-30 hrs post addition. Data were analyzed using the IncuCyte Live-Cell Analysis System that assessed the number of apoptotic cells (green) and target cells (red) in a real-time manner. The percentage (%) of Caspase-3/7 expression was measured in cells showing both green and red signals, and computed as an expression of the total detected target cells (red). CD19-scFv antibody (200 ng/ml, Invivogen) was pre-incubated with NK^{TROG-} populations for 30 min to block CD19-antigen exposure, and anti-β-Gal scFv antibody (Invivogen) was used as the negative control.

### Single-Cell Cytotoxicity Assay

Time-lapse imaging microscopy in nanowell grids (TIMING) was used to test NK-mediated cytotoxicity at a single-cell scale as previously described (see, e.g., Liadi et al., 2015). In brief, the sorted NK cell populations and target cells (K562 or Raji) were labeled with lipophilic PKH dyes, respectively, and loaded onto nanowell arrays. The array was incubated with media that was pre-mixed with Annexin V (BD Bioscience), and monitored in real-time for 5-6 hrs by a Carl Zeiss Axio Observer fitted with a Hamamatsu Orca-Flash sCMOS camera using a 20×0.8 NA objective. Images of ˜5,000 wells were collected and processed using an in-house algorithm for cell tracking and segmentation (see, e.g., Merouane et al., 2015).

### NK Population Doubling Assay

CB-NK cells were subcultured every week, with or without uAPC feeder cells, after the initial transduction and expansion. Using the equation for Population Doubling (PD)=log 10[(A/B)/2], where A is the number of harvested cells and B the number of plated cells from each subculture, the weekly PD was measured, then, the sum of each PD over time was determined as the accumulative PDs. Assays were terminated three weeks after the cell count of harvested cells from the subculture failed to achieve at least an equal amount of seeded cells. Data were obtained from three different CB-derived NK populations for each condition.

### Mass Cytometry (CyTOF)

Mass cytometry was performed as previously described (see, e.g., Li et al., 2019; and Daher et al., 2021(b)). Primary antibodies were conjugated in-house with corresponding metal tags using MaxparX8 polymer antibody labeling kit per manufacturer's protocol (Fludigm). NK cells were washed with cell staining buffer (0.5% bovine serum albumin/PBS), and incubated with human Fc receptor blocking solution (Miltenyi Biotec) before antibody mix was added. Cells were then incubated with 2.5 μM cisplatin (Sigma Aldrich), followed by fixation and permeabilization using BD CYTOFIX/CYTOPERM™ solution according to the manufacturer's protocol. For intracellular staining, cells were washed twice with perm/wash buffer and incubated directly with antibody master mix against intracellular markers. Cells were then stored overnight in 500 μl of 1.6% paraformaldehyde (EMD Milipore)/PBS with 125 nM iridium nucleic acid intercalator (Fluidigm). On the days that cells were assessed, they were washed in 1 ml of MilliQ dH₂O, and filtered through a 35 μm nylon mesh (cell strainer cap tubes, BD Bioscience). The cells were then resuspended in MilliQ dH₂O supplemented with EQTM 4-element calibration beads, and subsequently acquired at 300 events/second on a Helios instrument (Fluidigm). Antibodies used with the corresponding metal tag isotopes in vitro experiments: CD45 (Fluidigm, HI30, 89Y), GFP (Biolegend, FM264G, ¹⁴⁴Nd), DAP12 (R&D, 406288, 146Nd), NKG2C (Biolegend, 134591, ¹⁴⁷Sm), TRAIL (Miltenyi, REA1113, ¹⁴⁸Nd), CD25 (Miltenyi, REA570, ¹⁴⁹Sm), CD69 (Biolegend, FN50, ¹⁵⁰Nd), CD2 (Miltenyi, REA972, ¹⁵¹Eu), CAR (Jackson immune research, polyclonal, 152Sm), TIGIT (ThermoFisher, MBSA43, ¹⁵⁴Sm), OX40 (Miltenyi, REA621, ¹⁵⁸Gd), Perforin (Miltenyi, REA1061, ¹⁵⁹Tb), PD1 (Miltenyi, PD1.3.1.3, ¹⁶⁰Gd), Tbet (Miltenyi, 4B10, ¹⁶¹Dy), EOMES (ThermoFisher, WD1928, ¹⁶²Dy), c-Kit (Miltenyi, REA787, ¹⁶³Dy), SAP (Biolegend, 1A9, ¹⁶⁴Dy), TIM3 (R&D, 344823, ¹⁶⁵Ho), NKG2D (Miltenyi, REA797, ¹⁶⁶Er), 2B4 (ThermoFisher, C1.7, ¹⁶⁷Er), Ki67 (Biolegend, Ki67, ¹⁶⁸Er), NKG2A (Miltenyi, REA110, ¹⁶⁹Tm), DNAM-1 (Miltenyi, REA1040, ¹⁷⁰Er), CS1 (Biolegend, 162.1, ¹⁷²Yb), Granzyme B (Miltenyi, REA226, ¹⁷³Yb), CD94 (Miltenyi, REA113, ¹⁷⁴Yb), LAG3 (Miltenyi, REA351, ¹⁷⁵Lu), ICOS (Miltenyi, REA192, ¹⁷⁶Yb), CD16 (Fluidigm, 3G8, ²⁰⁹Bi), CD3 (Biolegend, UCHT1, ¹⁹⁴Pt), Cisplatin L/D (Fluidigm, ¹⁹⁸Pt), CD56 (BD Bioscience, NCAM16.2, ¹⁰⁶Cd), CD19 (Biolegend, HIB19, ¹¹⁰Cd), Granzyme A (Miltenyi, REA162, ¹¹¹Cd), Syk (Biolegend, 4D10.2, ¹¹²Cd), NKp30 (Miltenyi, AF29-4D12, ¹¹³Cd), NKp46 (Miltenyi, REA808, ¹¹¹Cd), NKp44 (Miltenyi, REA1163, ¹¹⁶Cd).

Antibodies used with the corresponding metal tag isotopes in vivo experiments: CD45 (Biolegend, HI30, ⁸⁹Y), CD2 (Biolegend, TS1/8, ¹⁴¹Pr), CD62L (BD Biosciences, DREG-56, ¹⁴³Nd), CD27 (Biolegend, M-T271, ¹⁴⁴Nd), CD56 (Biolegend, HCD56, ¹⁴⁶Nd), NKG2C (R&D, MAB138, ¹⁴⁷Sm), CXCR6 (R&D, MAB699, ¹⁴⁸Nd), CXCR3 (R&D, MAB160, ¹⁴⁹Sm), Granzyme B (R&D, polyclonal, ¹⁵⁰Nd), Tbet (Biolegend, 4B10, ¹⁵¹Eu), TIGIT (Biolegend, A15153G, ¹⁵²Sm), Granzyme A (Biolegend, CB9, ¹⁵⁴Sm), NKG2A (R&D, MAB1059, ¹⁵⁵Gd), TIM3 (Biolegend, F38-2E2, ¹⁵⁶Gd), 2B4 (Biolegend, 2-69, ¹⁵⁸Gd), CLA (Bioledend, KPL-1, ¹⁵⁹Tb), CD20 (Biolegend, RIK-2, ¹⁶⁰Gd), DNAM-1 (Miltenyi, DX11, ¹⁶¹Dy), EOMES (Thermo Fisher, WD1928, ¹⁶²Dy), NKp30 (Biolegend, P30-15, ¹⁶³Dy), c-Kit (BD Biosciences, YB5.B8, ¹⁶⁴Dy), CD25 (BD Biosciences, 2A3, ¹⁶⁵Ho), NKG2D (R&D, MAB139, ¹⁶⁶Er), Perforin (BD Biosciences, 8G9, ¹⁶⁷Er), ZAP70 (ThomoFisher, 1E7.2, ¹⁶⁸Er), CCR5 (Biolegend, J418F1, ¹⁶⁹Tm), CAR (Jackson immune research, polyclonal, ¹⁷⁰Er), CX3CR1 (Bioledend, 2A9-1, ¹⁷¹Yb), CXCR1 (Biolegend, 8f1, ¹⁷²Yb), PD1 (Biolegend, EH12.2H7, ¹⁷³Yb), Syk (Bioledend, 4D10.2, ¹⁷⁴Yb), NKp46 (R&D, MAB1850, ¹⁷⁵Lu), KLRG1 (ThermoFisher, 13F12F2, ¹⁷⁶Yb), CD57 (Biolegend, HNK-1, ¹⁹⁴Pt), Cisplatin L/D (Fluidigm, ¹⁹⁸Pt), CD16 (Fluidigm, 3G8, ²⁰⁹Bi).

### Mass Cytometry Data Analysis

Mass cytometry data were analyzed using Cytobank. NK cell populations were identified by using the strategy of gating singlets in Pt195(cisplatin)^{low} hCD45⁺CD56⁺CD3⁻, and were applied to all files. CAR⁺ and CD19⁺ expression was determined based on either isotype control or NK cells culture alone as controls. Data from 10,000 identified NK cells per each in vitro sample were randomly subsampled in FlowJo. Normalized data from each sample were pooled and analyzed to acquire their variability in signals. A t-Distribution Stochastic Neighbor Embedding (t-SNE) map was generated by the t-SNE analysis that performed a pairwise comparison of cellular phenotypes to optimally plot clusters and reduce dimensions from multiple parameters. Subsequently FlowSOM analysis was performed to determine metaclusters in optimized grouping distance between the empirically expected node and to build a minimum spanning tree by connecting nodes hierarchically. The expression of each marker was transformed and normalized locally, then hierarchically clustered, and plotted as a heat map using Morpheus matrix visualization and analysis software (Broad Institute).

### Metabolism Assays

Extracellular acidification rate (ECAR) and oxygen consumption rate (OCR) were measured in GFP-negative CAR-NK effector cells using Seahorse XF Cell Mito Stress Test Kit (Agilent), and Seahorse XF Glycolysis Stress Test Kit (Agilent) in Agilent Seahorse XFe96 Analyzer according to the manufacturer's protocol. The assay was performed in phenol red/carbonate free RPMI media (Agilent) containing 2 nM L-glutamine (Agilent), 25 mM glucose and 2 mM pyruvate (Agilent, but excluded in the glycolysis assay). Cell mito stress test was performed by examining OCRs after administering 1.5 μM oligomycin, 0.5 μM fluorocarbonyl cyanide phenylhydrazone (FCCP), 0.5 μM rotenone, and antimycin A. Glycolysis test was measured as the ECAR following injection with 10 mM glucose, 1 μM oligomycin, and 50 mM 2-Deoxy-D-glucose (2-DG).

### CAR-NK Cell Affinity Experiments

Experiments were performed using Poly-L-Lysine (Sigma-Aldrich) coated z-MOVI® chips. MM1S^{CD70+} cells were seeded onto Z-MOVI® chip, creating a monolayer. The Z-MOVI® chip was then sealed and incubated in a dry incubator for 30 minutes. Effector cells were stained with Cell Trace Far Red (ThermoFisher) and their flow measured onto the monolayer, 200-500 cells at a time. Effectors were then incubated with the target cell monolayer for five minutes before the start of the force ramp. Force ramp was set at 1000 pN over 90 seconds for each run. Affinity measurements were conducted on a Z-MOVI® Cell Avidity Analyzer using the Oceon software.

### Luminex Assays

The MILLIPLEX RMAP magnetic bead (Millipore) kit was used to measure human granzyme A, granzyme B, perforin, TNF-α, and IFN-γ in serum collected from mice at different time points after receiving NK cell infusion as per the manufacturer's protocol. Measurements were performed on the Luminex 200 System.

### Xenogeneic Tumor-Grafted Mouse Models

NOD/SCID IL-2Rγnull (NSG) mice grafted with aggressive, NK-resistant tumor cells were used to examine the anti-tumor activity of different NK populations as previously described (see, e.g., Liu et al., 2018; and Daher et al., 2021(b)). Tumor models included CD19⁺ Raji lymphoma, CD5⁺ CCRF T-ALL, CD123⁺ MOML14 acute myeloid leukemia (AML), and SKOV3 ovarian cancer. All experiments were performed in accordance with American Veterinary Medical Association (AVMA) and NIH recommendations under protocols approved by the Institutional Animal Care and Use Committee. Seven-week-old female NSG mice (Jackson Laboratories) were irradiated (300 cGy) on Day −1. On Day 0 firefly luciferase-GFP labeled Raji cells were injected intravenously (i.v.) at three escalating dose levels of Raji cells (0.2×10⁵, 1×10⁵, or 5×10⁵), respectively; for the other blood tumor models, one dose of CCRF^{CD5+/Luci+/GFP+} cells (0.5×10⁵), or MOML14^{CD123+/Luci+/GFP+} cells (0.5×10⁵) were injected i.v., respectively. The mice were then serially treated with the indicated NK cell populations. For ovarian cancer models, 7-week old female NSG mice (Jackson Laboratories) were injected intraperitoneally (i.p.) with luciferase-GFP labeled SKOV3 cells (1×10⁶ of SKOV3^{ROR1+} or 0.5×10⁶ of SKOV3^{gCD19+}) seven days (Day −7) before the treatment; at Day −1 the mice were irradiated (300 cGy), and then received AI-CAR expressing NK cells (1-1.5×10⁷) via i.p. injection on Day 0. Bioluminescence imaging (Xenogen-IVIS 200 Imaging system; Caliper) was performed regularly to examine the engraftment of Raji cells and SKOV3 cells. Signal quantitation in photons/second was measured using IVIS Living Image software (Caliper Life Sciences).

### Statistics

Statistical analyses were performed and plotted using Prism 7 software (GraphPad). The Student's t-test was used to test for significance; one-way ANOVA was applied to determine the comparison among groups at a certain condition; two-way ANOVA was applied to determine the comparison among groups in a time course; P value for pairwise comparisons was conservatively adjusted for multiple comparisons using Bonferroni correction. Mean values+s.e.m. are shown. The non-linear regression model with the least trimmed sum of squares was selected as the robust goodness-of-fit (see, e.g., Andreas et al., 2013). Overall survival (OS) analysis was calculated using Kaplan-Meier methods and compared to the treatment group using log-rank tests with 95% confidence intervals (CI).

### Example 1

Inhibitory Chimeric Antigen Receptor (iCAR) Prevents on-Target Off-Tumor Effects of Anti-Tumor Cellular Therapy by Using CAR-T Cells and CAR-NK Cells

The inventors have successfully engineered five examples of constructs of iCARs [KIR2DL1(TM)-KIR2DL1(CDs); LIR-1(TM)-LIR-1(CDs); KIR2DL1(TM)-KIR2DL 1(CDs)-LAIR-1(CD); KIR2DL1(TM)-KIR2DL1(CDs)-NKG2A(CDs); KIR2DL1(TM)-KIR2DL1(CDs)-CD300A(CD)], and expressed them in natural killer (NK) cells derived from cord blood stored in cord blood banks. The inhibitory signaling induced by each iCAR upon engagement with its cognate ligand results in increased phosphorylation of ITIMs but not the activating signals such as Syk and Zap70. The function of engagement of iCARs with the cognate ligand inhibits NK mediated cytokine secretion (IFN-gamma, TNF-alpha), degranulation (CD107a), and cytotoxicity in an antigen-specific manner. NK cells transduced with an iCAR that recognizes the NK-self antigen CS1 fused with an inhibitory signaling endodomain prevents fratricide of CAR-NK cells without compromising their cytotoxicity against “on-target” tumors. Moreover, expression of the iCAR did not negatively impact the proliferation and expansion of CAR-NK cells. Thus, engineering immune effectors with a dual CAR system that includes an NK self-recognizing inhibitory CARs (iCAR) that transfers a “don't kill me” signal to NK cells upon engagement with a normal cell prevents their off-target on tumor activity, while sparing their on-target on-tumor signal of an activating CAR (aCAR) against the tumor antigen.

### Example 2

Expression of iCARS in NK Cells Derived from Cord Blood

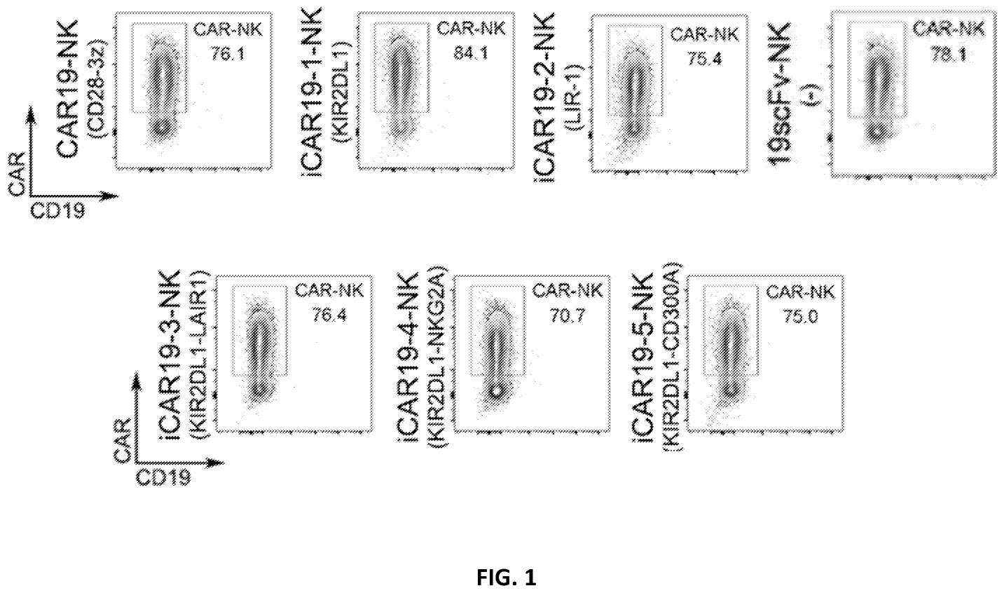

The inventors first designed iCAR against CD19 as a well-studied model. The anti-CD19 (single chain fragment variable (19scFv with lgG1 hinge) was ligated with the inhibitory KIR signals listed in Example 1. Each construct was then cloned into an SFG retroviral backbone, which allowed engineering of the NK cells by retrovirus-mediated transduction. Using goat anti-mouse lgG(H+L) antibody, the surface expression of the iCAR in NK cells was confirmed by flow cytometry. As shown in FIG. 1, there was successful and stable engineering of NK cells with each of the five CD19 iCARs (19scFv-iCAR1, 19scFv-iCAR2, 19scFv-iCAR3, 19scFv-iCAR4, and 19scFv-iCAR5) with 75% efficiency after transduction.

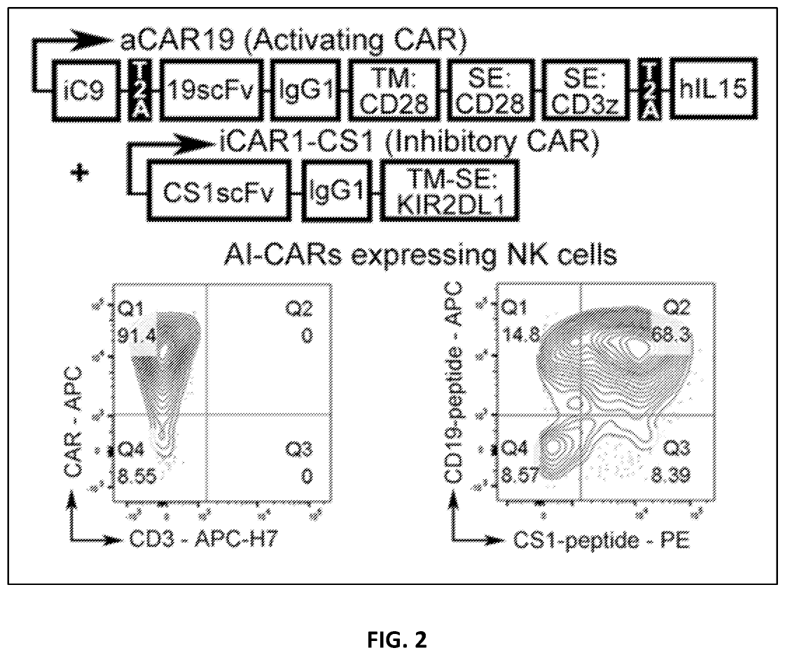

Alternatively, iCAR1 was also fused with anti-CS1 scFv, which recognizes CS1 that is expressed on normal NK cells and T cells (Tai et al., 2008), and fused with IgG1 hinge, and then cloned into an SFG retroviral backbone. Anti-CS1-iCAR1 (iCAR-CS1) was successfully introduced into NK cells together with the activating anti-CD19 CAR (CAR19-CD3zeta) in NK cells (FIG. 2). Fluorescent labeled immunogens (CS1 and CD19 peptide) were utilized to assess the expression levels of iCAR-CS1 and aCAR19, respectively. More than 80% of NK cells expressed iCAR-CS1 and more than 70% expressed the aCAR19. Importantly, around 70% engineered NK cell co-expressed both iCAR-CS1 and aCAR19.

### Example 3

Effects of iCAR Signaling in Inhibiting NK Activated Phosphorylation

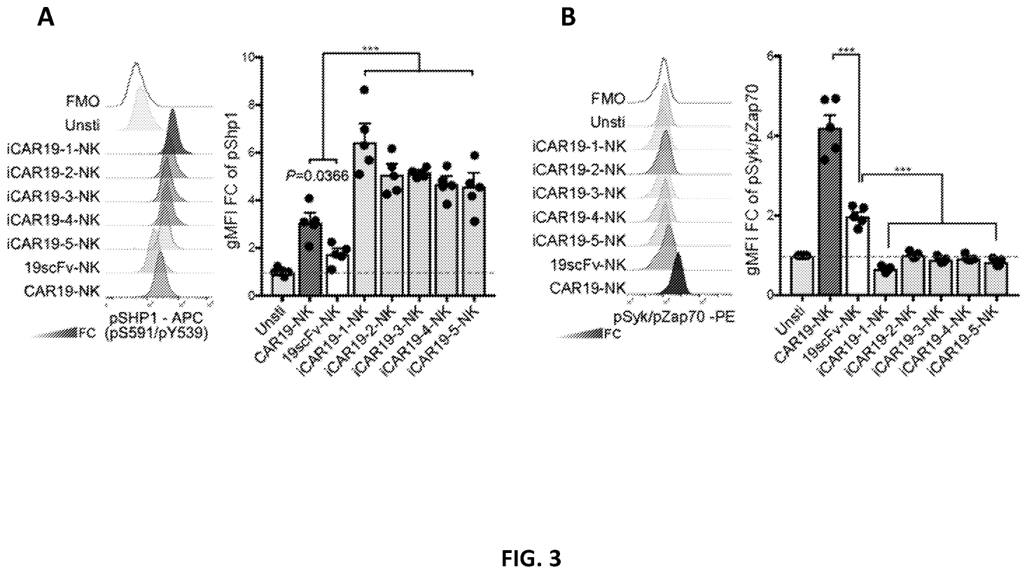

NK cell activation is tightly determined by changes in the phosphorylation status of signaling molecules rather than by transcriptional modulation (see, e.g., Bryceson & Long 2008; Vivier et al., 2004; and Long et al., 2013). Engagement of cognate ligands by inhibitory receptors results in phosphorylation of their ITIM domains that compete with the activating signals. Since the inventors combined the iCAR signaling endodomain with the CD19 recognition ectodomain, which allows for an inhibitory signal to be transmitted upon engagement with CD19 expressing cells, iCAR mediated phosphorylation signaling in ITIM-enriched adaptor (SHP1), but did not result in phosphorylation of ITAM-enriched adaptors (Syk/Zap70) after testing with co-culture of Raji^{CD19+} cells. aCAR NK cells transmitting an activating signal (CD19ScFv linked to CD3 zeta) and NK cells expressing the CD19 recognition domain (scFv only (without a signaling endodomain) were included as controls. Co-culture of iCAR-expressing NK cells with Raji cells resulted in a prompt increase in pSHP 1 that also limited the phosphorylation of Syk and ZAP70 (FIG. 3), supporting the strong inhibitory function of iCAR signaling in preventing NK cells from activation.

### Example 4

Negative Effect of iCAR Signaling on NK Mediated Cytotoxicity

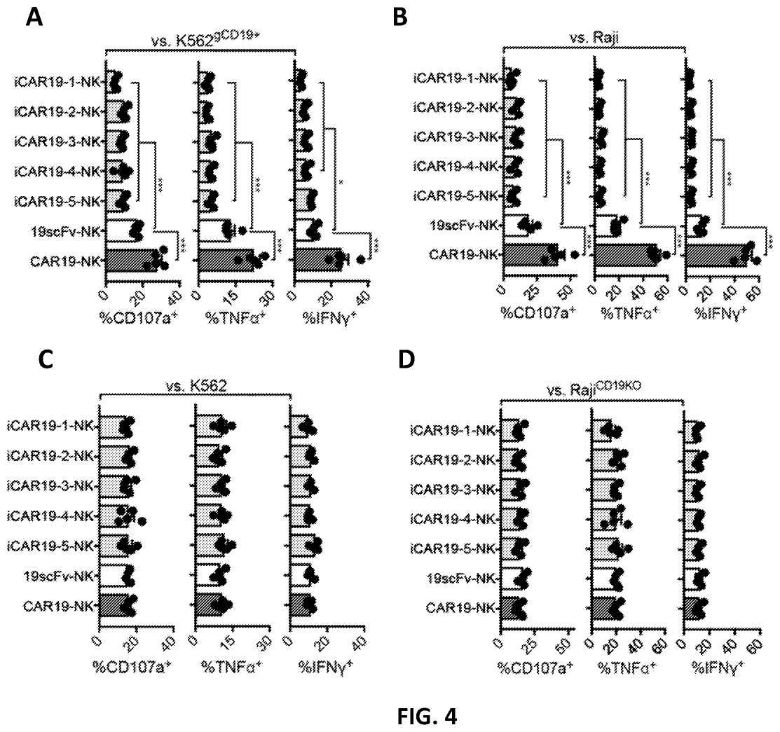

Having transduced NK cells with iCAR19s, their function against CD19+ targets was examined. For each iCAR19-NK cell, stimulation with CD19 expressing targets (K562^{CD19+} and Raji, FIGS. 4A and B) resulted in reduced degranulation levels (CD107a) and cytokine secretion (TNF-alpha, IFN-gamma) when compared to control 19scFv-NK cells or CAR19-CD3zeta NK cells. In contrast, this inhibitory effect of the iCAR19 on NK cell cytotoxicity was not observed when the cells were stimulated with non-CD19 target antigen expressing cells (K562 and Raji^{CD19-} (also known as Raji^{CD19-KO}), FIGS. 4 C and D). These findings indicate that iCARs inhibit NK cell activation in an antigen-specific manner.

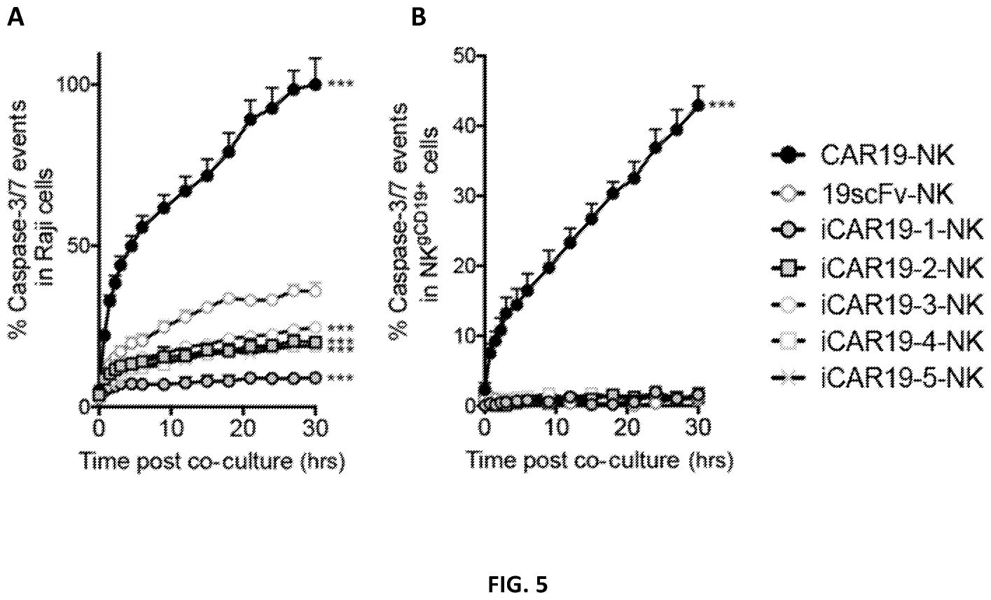

Since the signaling of iCAR significantly inhibits NK cell activation, the impact of iCAR on NK cell-mediated cytotoxicity was examined against cognate antigen expressing tumor targets. iCAR-expressing NK cells were co-cultured with Raji cells or NK^{CD19+} cells (e.g., NK cells engineered to express CD19), both of which express the cognate antigen CD19 (FIG. 5). When tested against Raji^{CD19+} cells, CAR19-CD3zeta-NK cells displayed greater cytotoxicity against targets than did 19scFv-NK cells (no signaling endodomain) or iCARs (inhibitory signaling endodomain) NK cells (FIG. 5A). When tested against NK^{CD19+} cells, only CAR19-CD3zeta-NK cells induced fratricide and apoptosis of NK^{CD19+} cells target cells (FIG. 5B). Together, these data indicate that iCAR signaling inhibits NK cell effector function and cytotoxicity in an antigen-dependent manner.

### Example 5

The Role of iCAR Signaling in Modulating CAR-NK Cell Mediated Cytotoxicity

Having confirmed that the iCAR signal can inhibit NK effector function in an antigen-dependent manner, a dual CAR system was designed that combined an activating CAR against a tumor antigen (e.g., CD19) with an inhibitory signal toward a ‘self antigen’ expressed on normal cells, e.g., CS1 expressed on all NK cells. Specifically, the inventors sought to address if the iCAR can suppress the aCAR signaling.

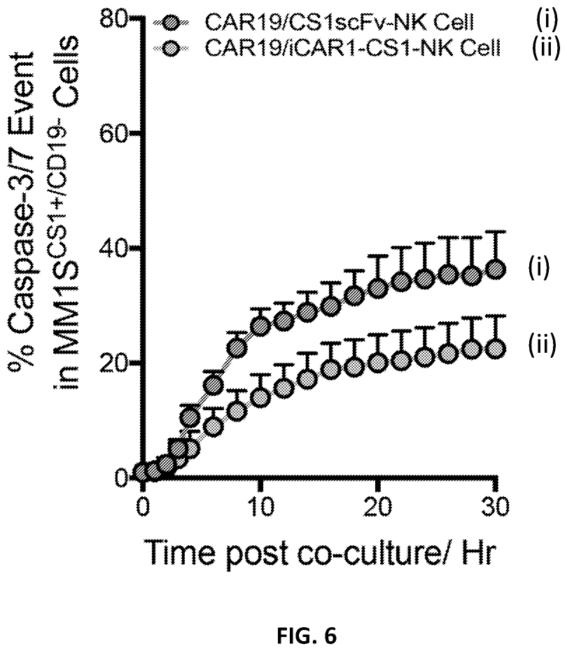

First, NK cells were transduced with constructs expressing CAR19-CD3zeta (aCAR) together with an iCAR1-CS1 or CS1scFv (without a signaling endodomain) as controls, and they were co-cultured with the MM1S myeloma cell line that expresses CS1 but not CD19 (FIG. 6). NK cells expressing aCAR19/iCAR-CS1 killed significantly fewer MM1S cells compared to aCAR19/CS1scFv-NK cells, indicating that the inhibitory signaling of iCAR1 suppresses NK cell mediated cytotoxicity after engagement with the self surface antigen CS1 on the target cells.

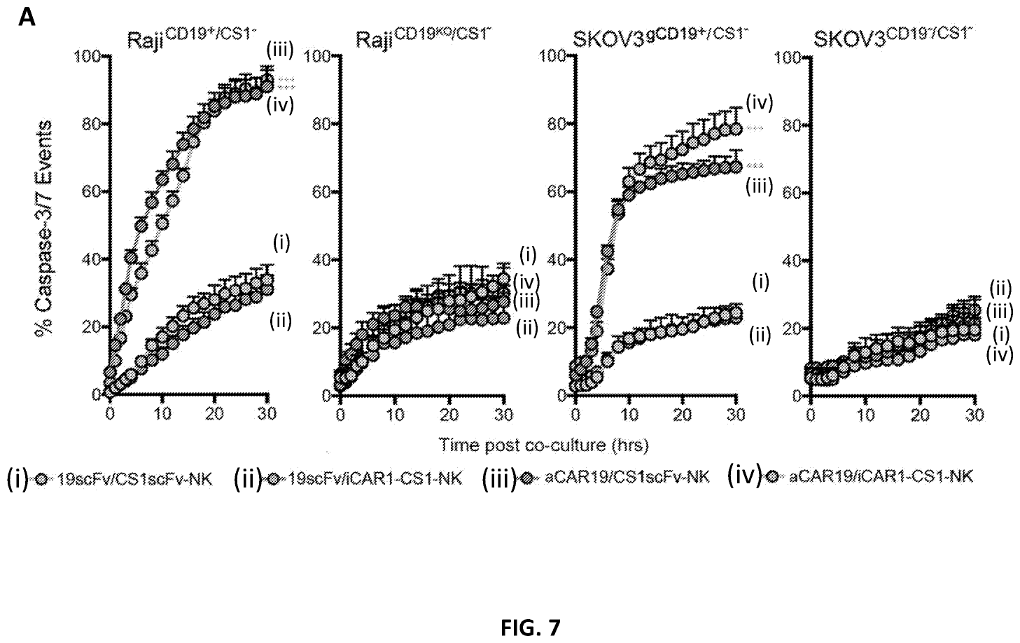

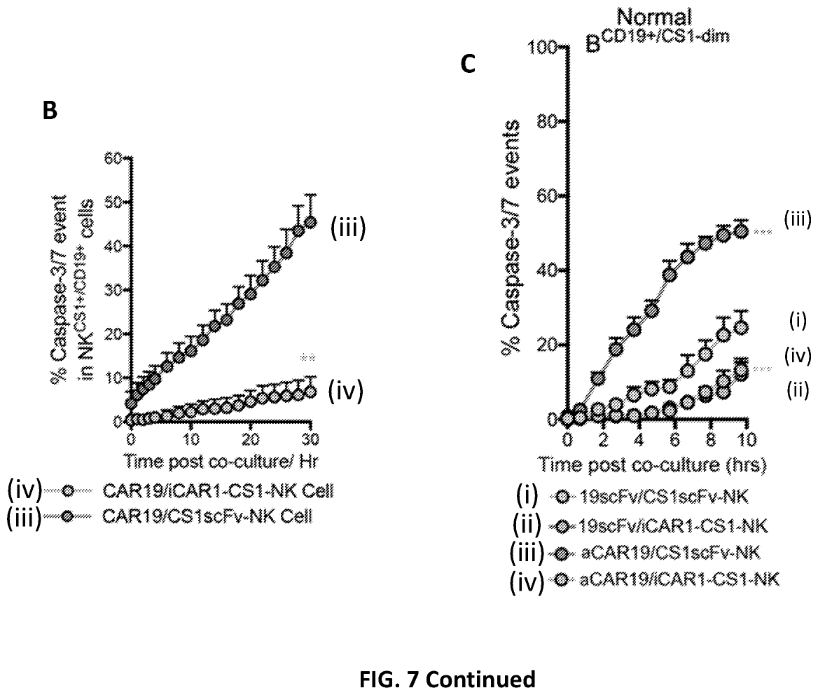

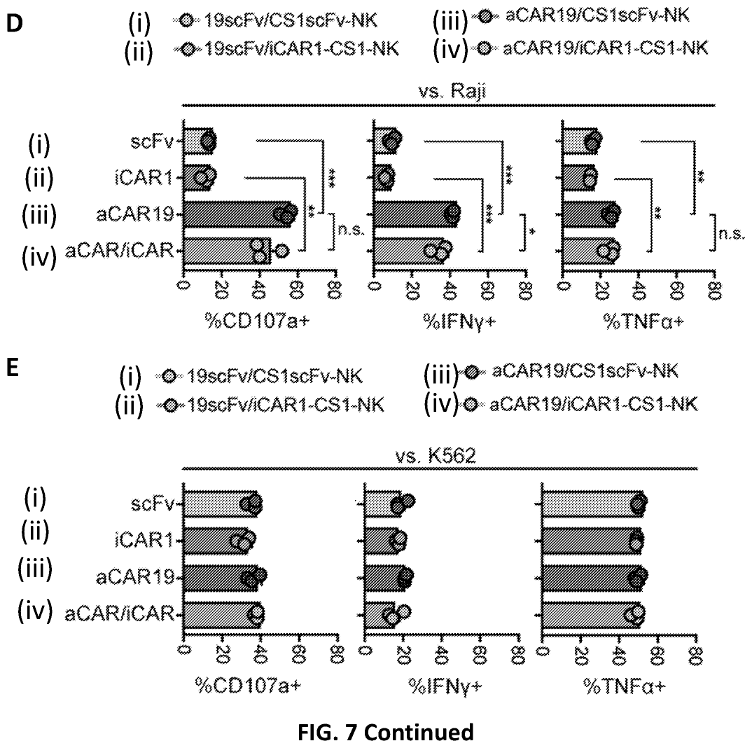

Next, it was examined if the iCAR can suppress aCAR19-mediated cytotoxicity. Along with CAR19/CS1scFv-NK cells, dual-CAR NK cells expressing both CAR19 and iCAR1-CS1 were evaluated for their cytotoxicity against CD19⁺ targets (e.g., Raji^{CD19+/CS1-} cells, SKOV3^{gCD19+/CS1-} cells), CD19− targets (e.g., Raji^{CD19+/CS1-} cells, SKOV3^{CD19-/CS1-} cells), and the self-target CS1 expressing targets (e.g., NK^{CD19+/CS1+}, FIG. 7). In the presence of the cognate antigen CD19, the aCAR19 mediated strong cytotoxicity that resulted in rapid cell death after co-culture with Raji^{CD19+/CS1-} cells and SKOV3^{gCD19+/CS1-} cells. In contrast, no significant difference was observed when compared to NK that also expressed iCAR1-CS1 or CS1scFv (FIG. 7A). Also, when tested against tumor targets not expressing the cognate self-antigen CS1, co-expression of iCAR1-CS1 or CS1scFv had little impact on CAR19-NK mediated cytotoxicity, again pointing to the antigen-specificity of the inhibitory CAR function (FIG. 7A). Importantly, while NK cells expressing an activating CAR19 mediated a strong cytotoxicity against their CD19+ siblings (fratricide), the co-expression of iCAR1-CS1 on NK cells prevented and reduced the scale of self-killing (FIG. 7B), suggesting that engagement of the iCAR-CS1 with the self-antigen CS1 on NK cells results in an inhibitory signal that can overcome NK cell activation mediated by the activating CAR19 signal. These data confirm that the additional expression of the iCAR1-CS1 in NK cells can suppress the anti-self activation signal and prevent their on-target off-tumor activity.

### Example 6

Effects of iCAR Signaling on Proliferation and Expansion of CAR-NK Cells and NK Cells

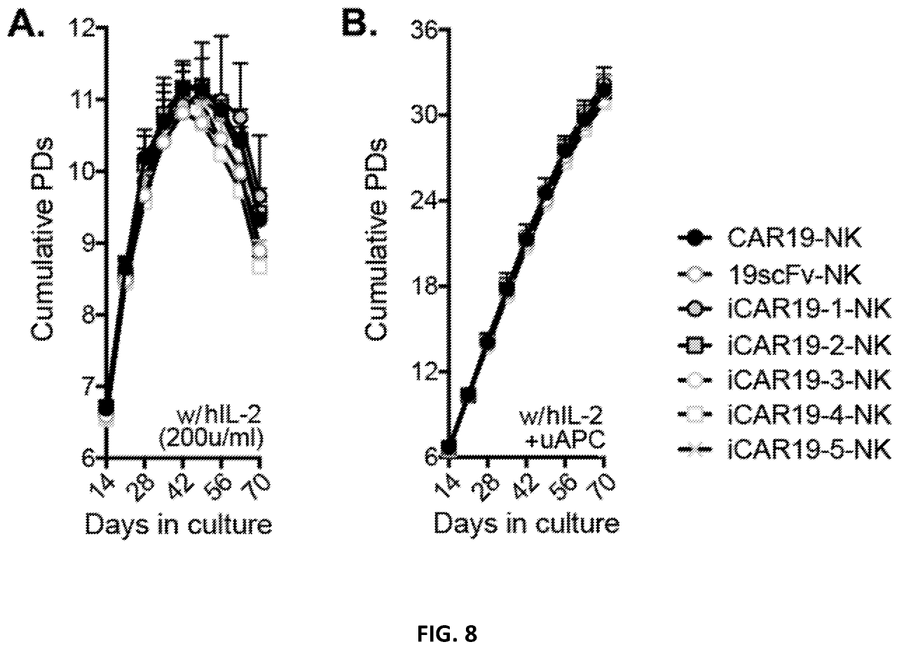

The effects of the iCARs on cell proliferation and expansion were determined. Ex vivo expansion of NK cells expressing iCAR19, CAR19 or 19scFv was performed with uAPC and IL-2, resulting in a similar kinetic of proliferation and expansion over a 4 week culture (FIG. 8), indicating that the expression of the iCAR signaling does not negatively impact the proliferation or expansion of NK cells in vitro.

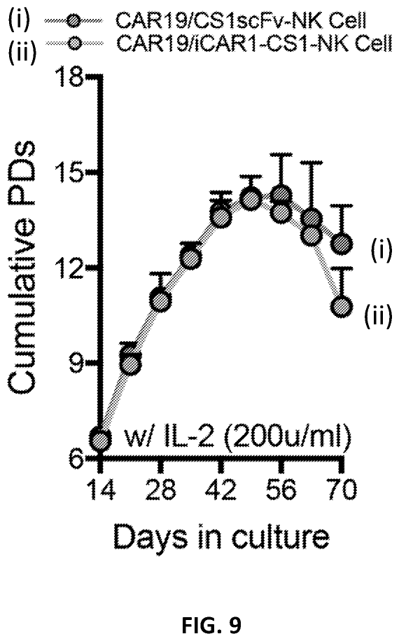

The impact of iCAR targeting CS1 on NK cells on cell proliferation was examined (FIG. 9). In keeping with the observation with iCAR19, the expression of iCAR1-CS1 on NK cells did not negatively alter their proliferative capacity. Altogether, the findings indicate that expression of an iCAR does not impair the proliferation of NK cells and CAR-NK cells and does not impair their on target antitumor activity.

In summary, the data confirm the efficacy of iCAR signaling in suppressing the effector function of immune cells. By ligating the iCAR signaling endodomain with an scFv that selectively binds to a self-antigen recognition domain, the iCAR can prevent an on-target and off-tumor response without negatively impacting an on-target on-tumor response through the aCAR signal.

### Example 7

### Car-Mediated TROG-Antigen Transfer to NK Cells

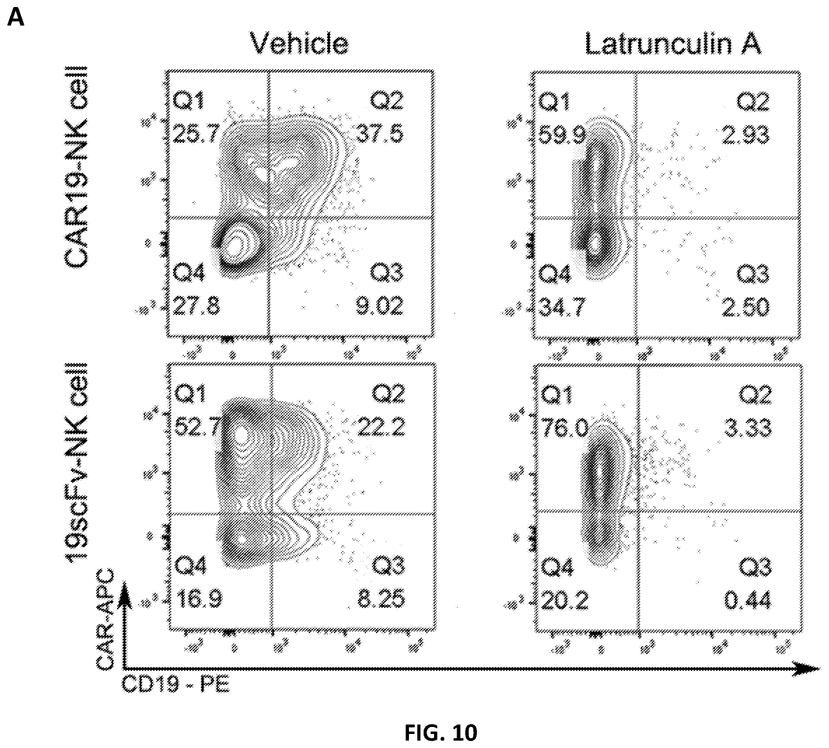

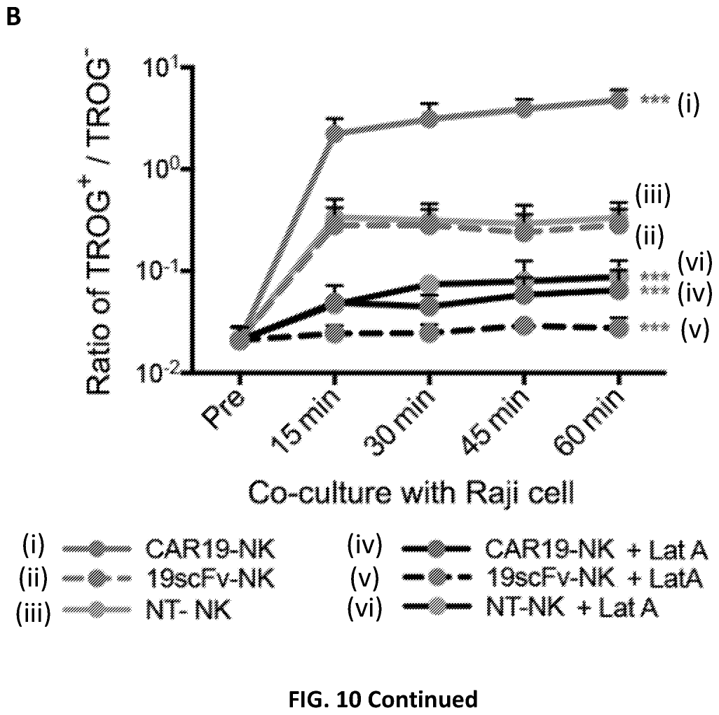

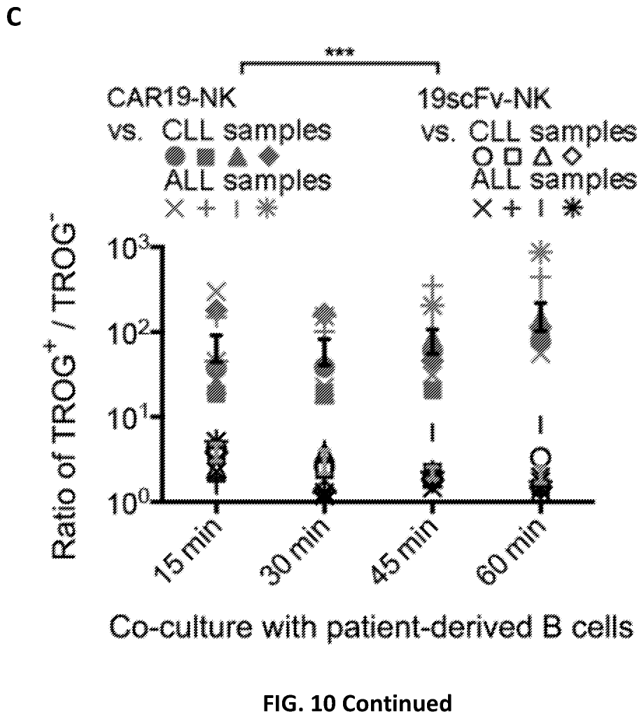

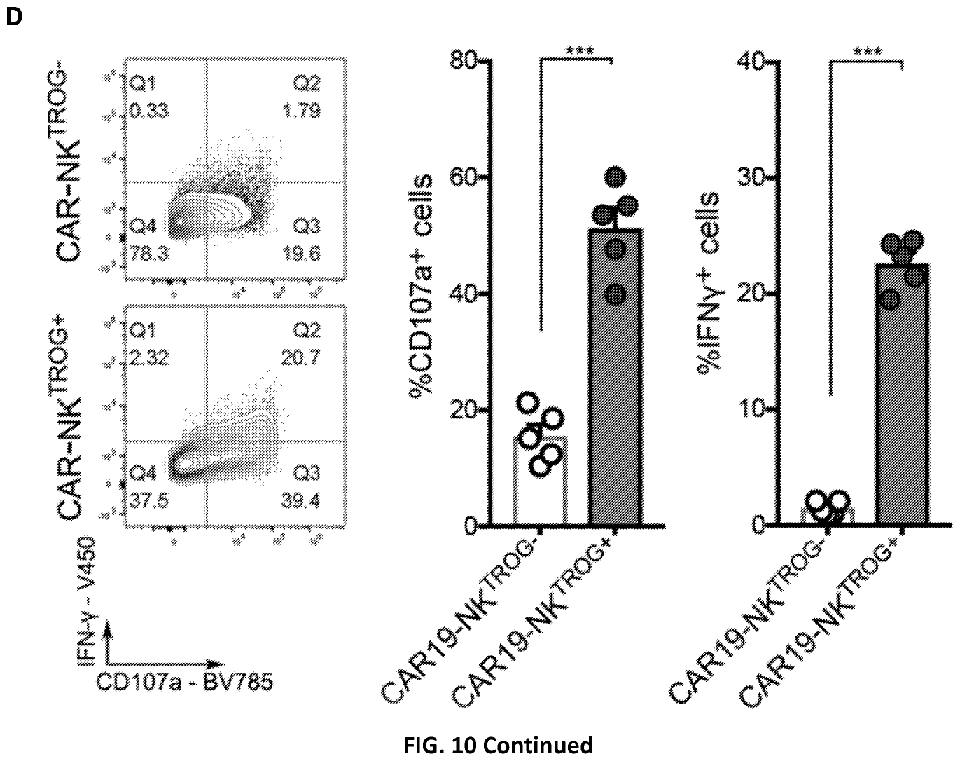

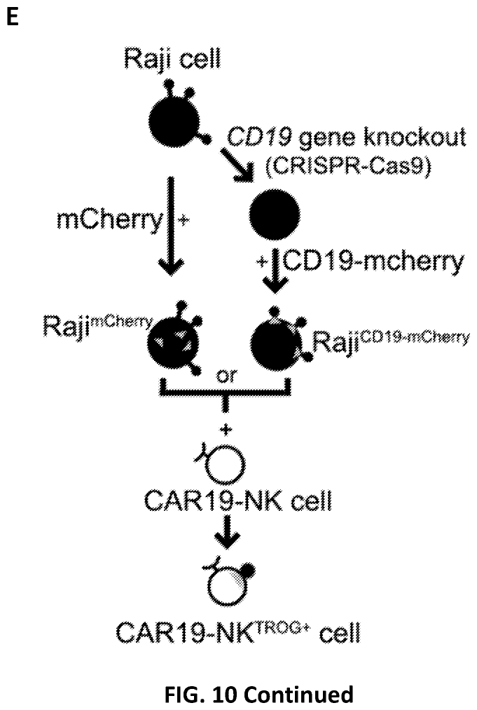

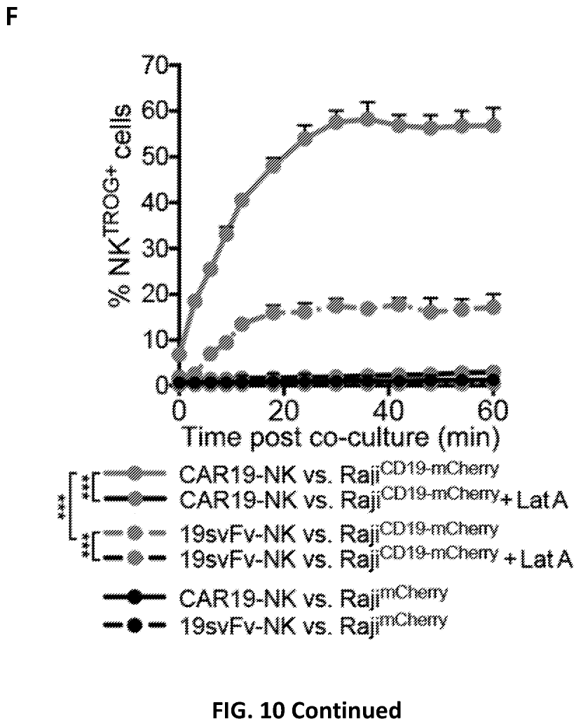

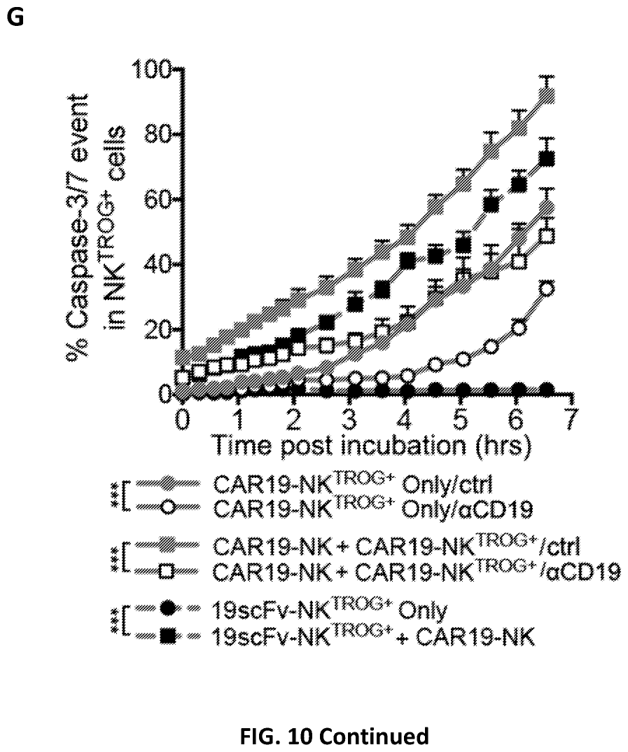

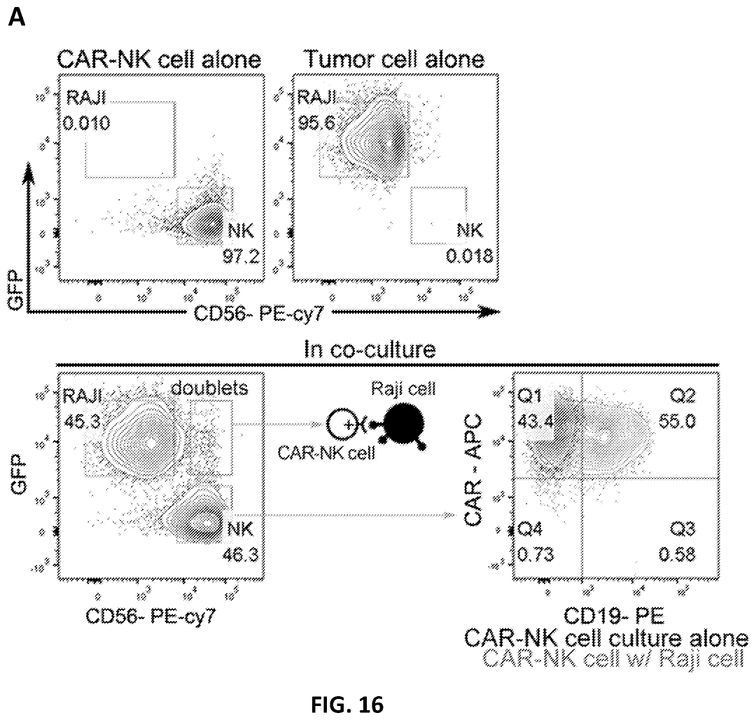

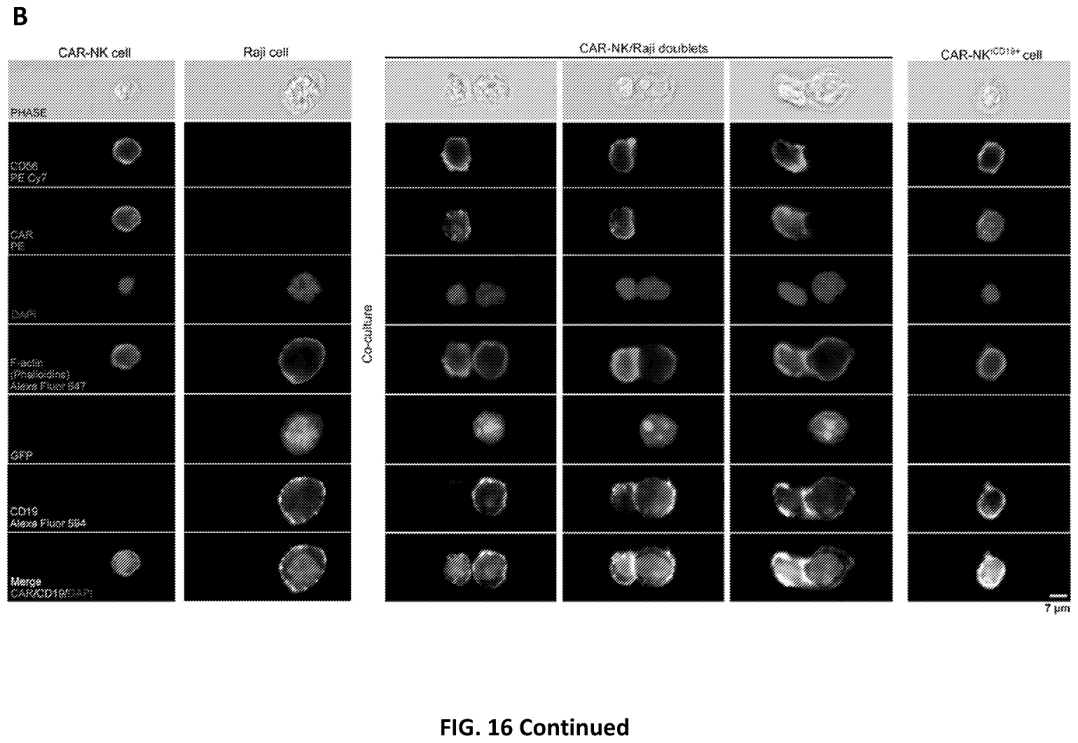

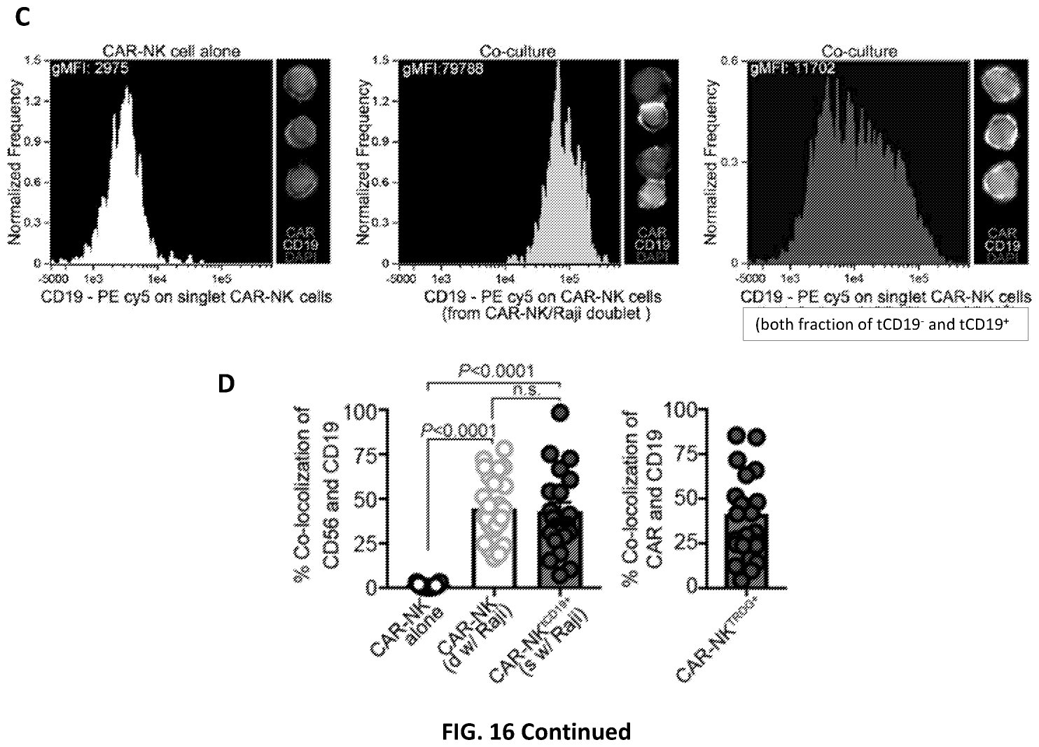

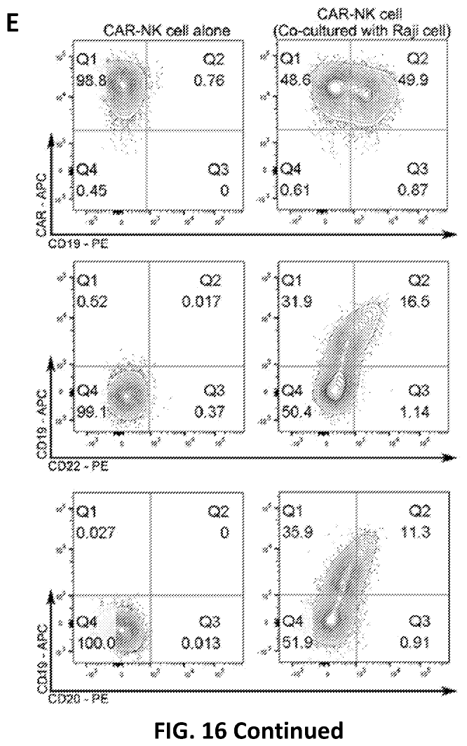

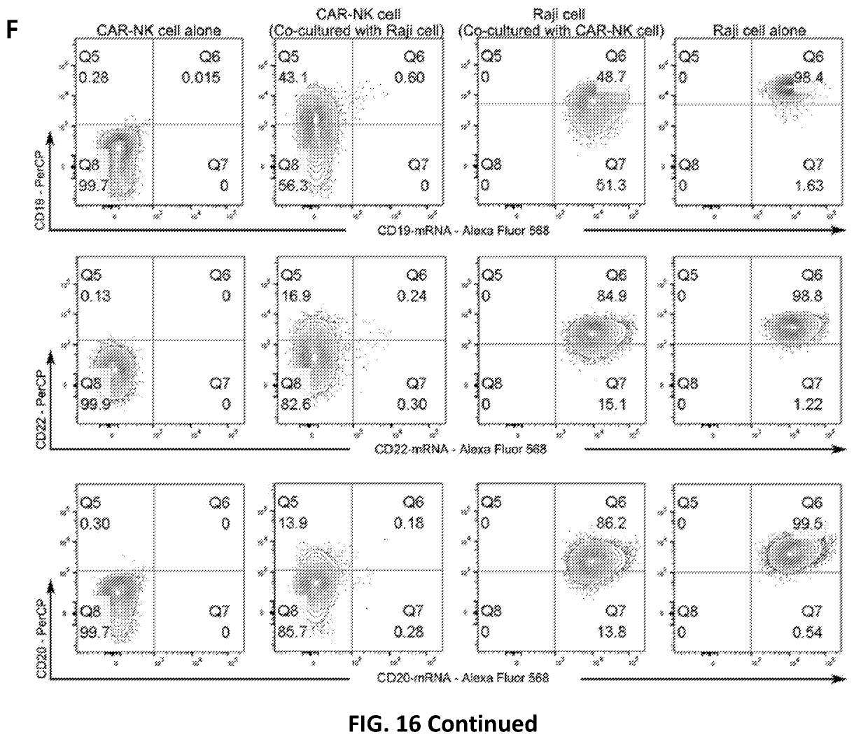

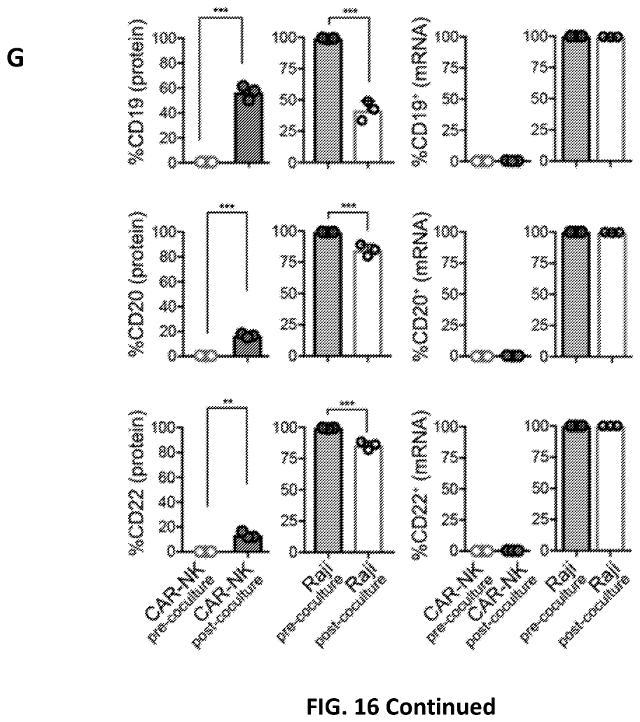

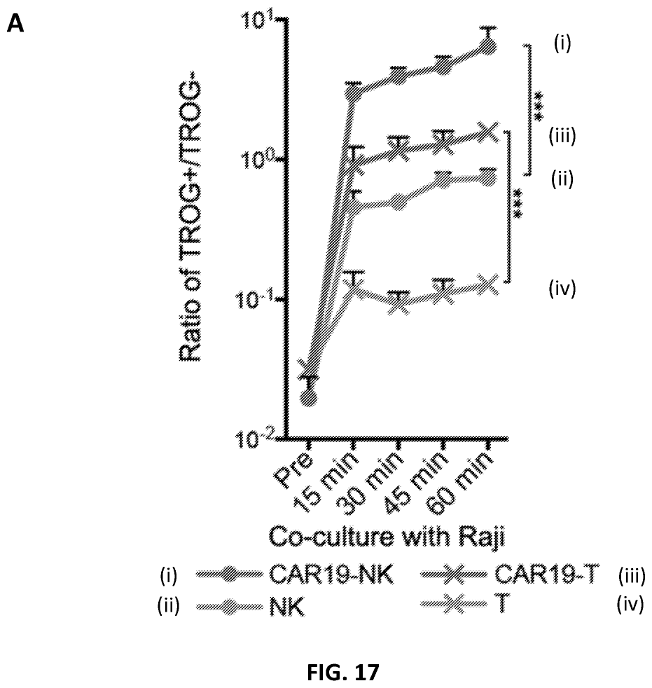

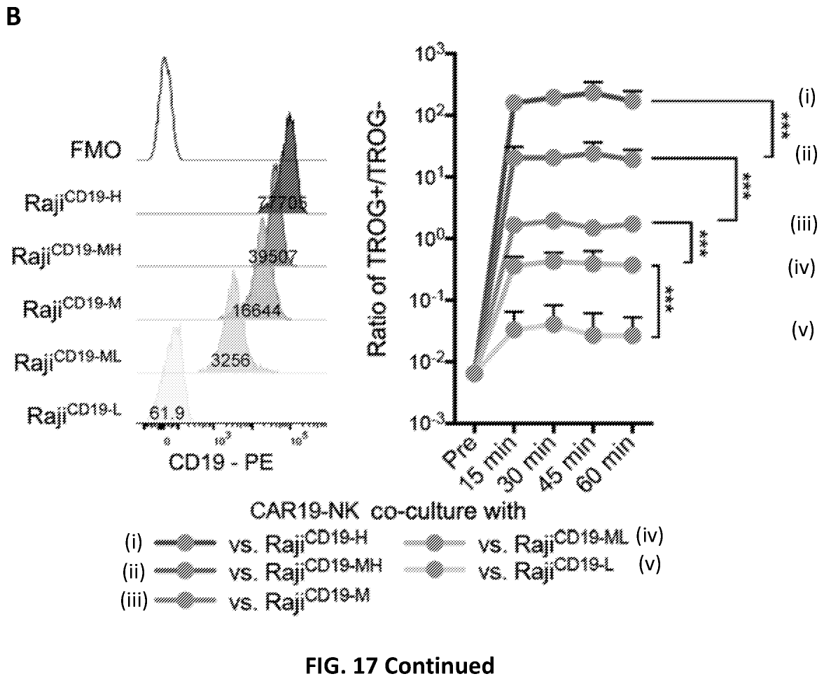

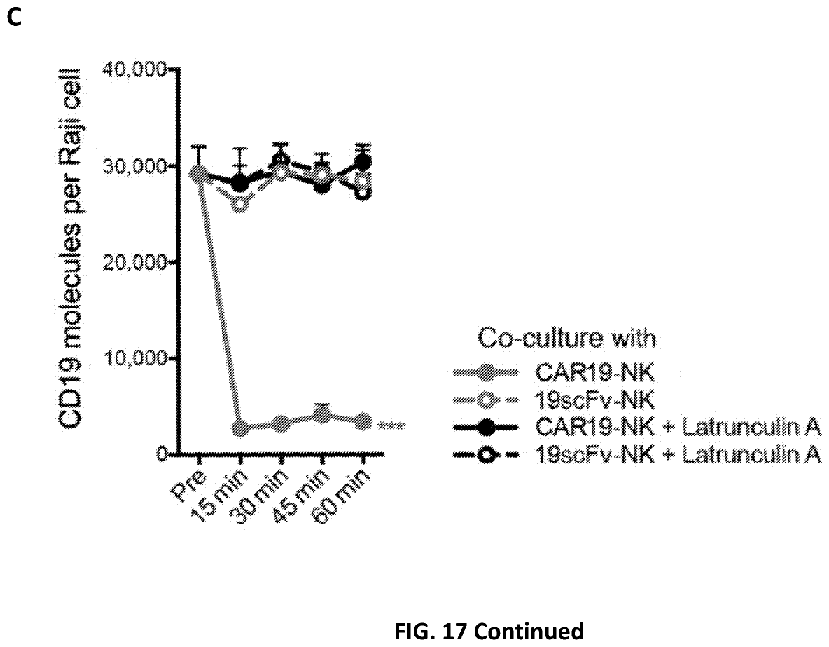

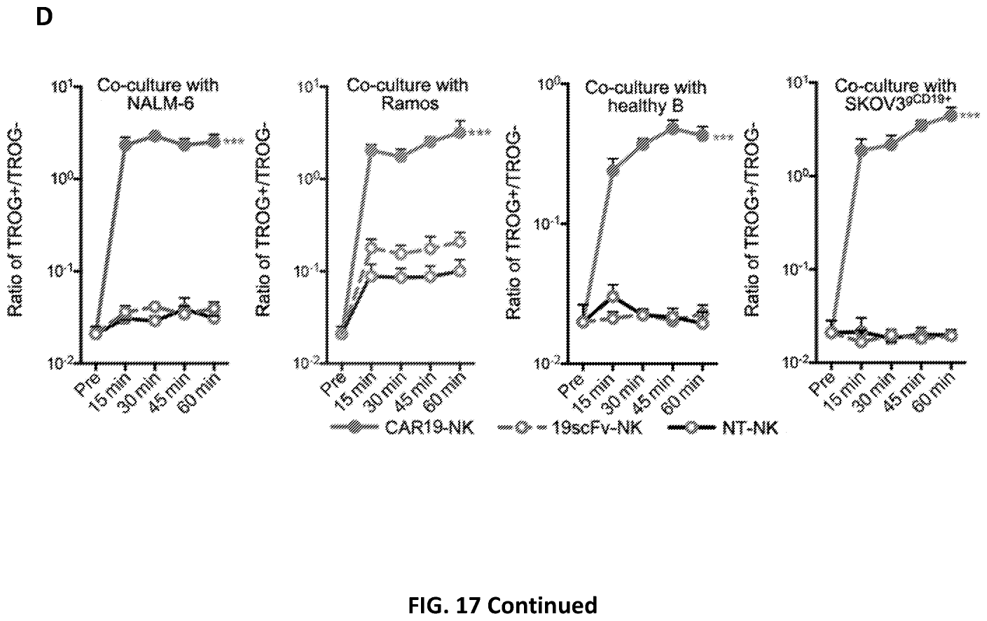

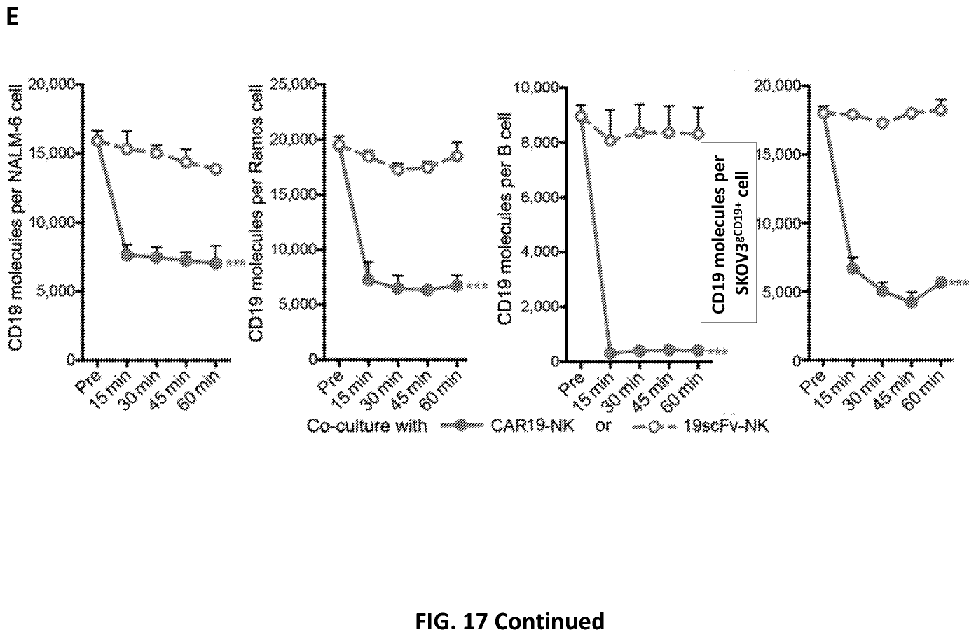

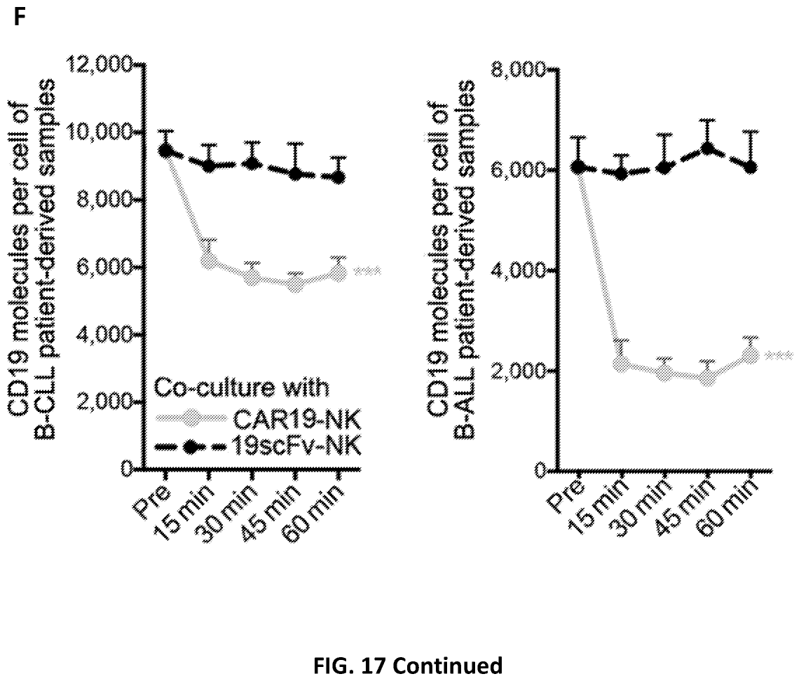

Trogocytosis triggered by receptor-ligand interactions is a well-described phenomenon in NK cells (see, e.g., Joly & Hundrisier 2003; Dance 2019; and Ahmed et al., 2008). But whether an engineered activating receptor such as anti-CD19 CAR (CAR19) can also mediate the transfer of its cognate antigen CD19 to NK cells following co-culture with Raji^{CD19+} lymphoma cells was unknown. Non-transduced NK (NT-NK) cells and those expressing 19scFv (anti-CD19 CAR lacking all intracellular domains) were used as parental control and receptor control, respectively. Within minutes of co-culture with Raji targets, a significantly greater portion (75%±10%) of CAR19-NK cells acquired CD19 expression as well as other B-cell associated markers such as CD20 and CD22 on their surface compared with NT-NK or 19scFv-NK controls (FIGS. 10A and B, and FIGS. 16A-G). These results suggested that trogocytosis is a rapid process and that CAR-signaling is necessary for the robust transfer of a cognate antigens to CAR-NK cells. A similar phenomenon, but to a lesser extent, was observed with CAR19 T cells (FIG. 17A, as also previously described (see e.g., Hamieh et al., 2019). Pre-treatment of CAR-NK cells with Latrunculin A (LatA), an F-actin inhibitor that blocks immunologic synapse formation (see, e.g., Hudrisier et al., 2007), prevented the transfer of cognate CD19-antigen from Raji cells to CAR19-NK cells (FIGS. 10A and B), supporting the importance of synapse formation in driving trogocytosis. Thus, CAR-NK cells that acquired surface expression of target-derived cognate antigen following trogocytosis (Raji-derived CD19 in this case) were termed as the TROG⁺ fraction. Trogocytic antigen (TROG-antigen) acquisition on CAR-NK cells coincided with a substantial reduction in CD19 expression on the targeted Raji cells, which could also be prevented by LatA pre-treatment of CAR-NK cells (FIG. 17 C). In contrast, CD19 was not selectively lost in co-culture of Raji with NT-NK cells or 19scFv-NK cells (FIG. 17 C).

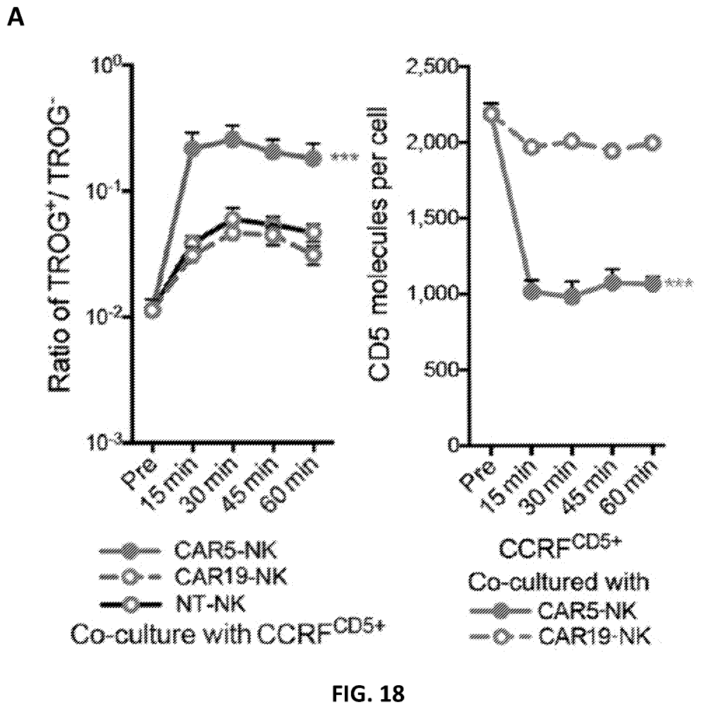

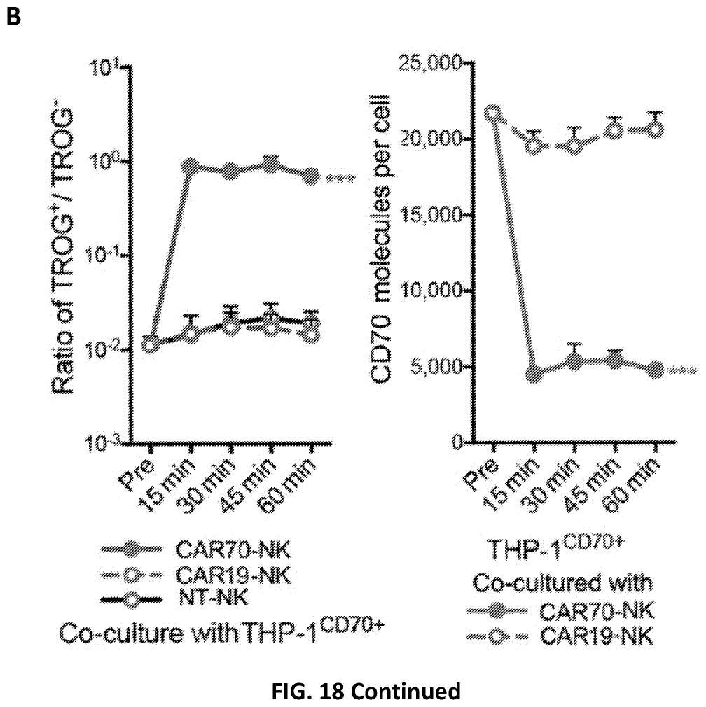

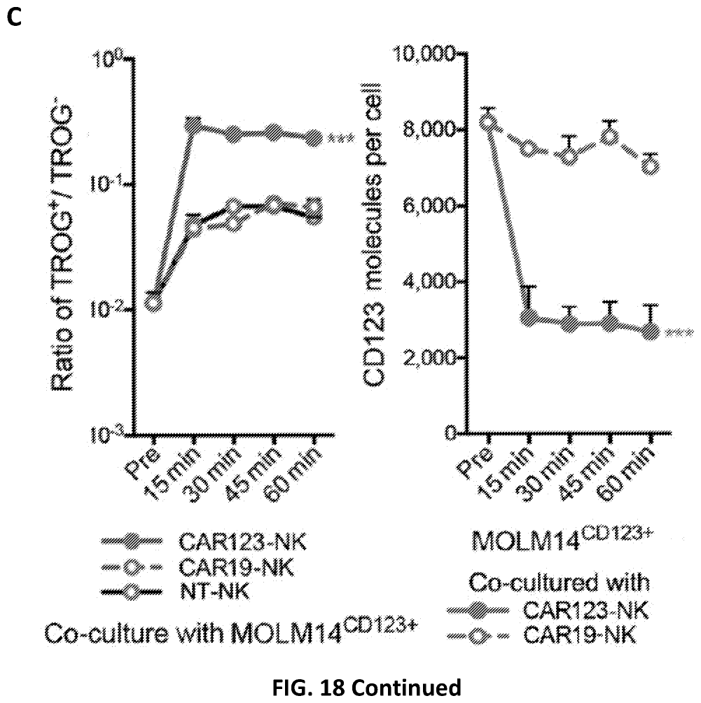

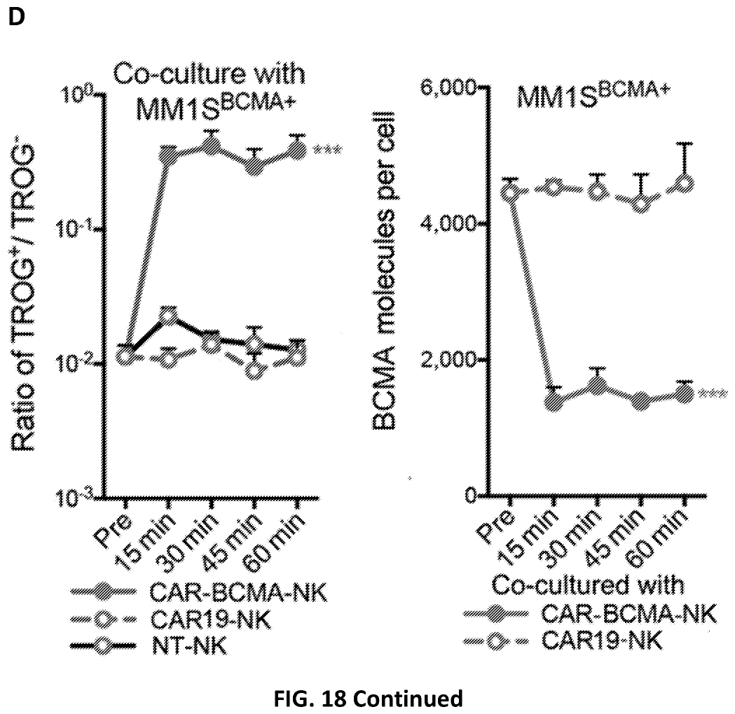

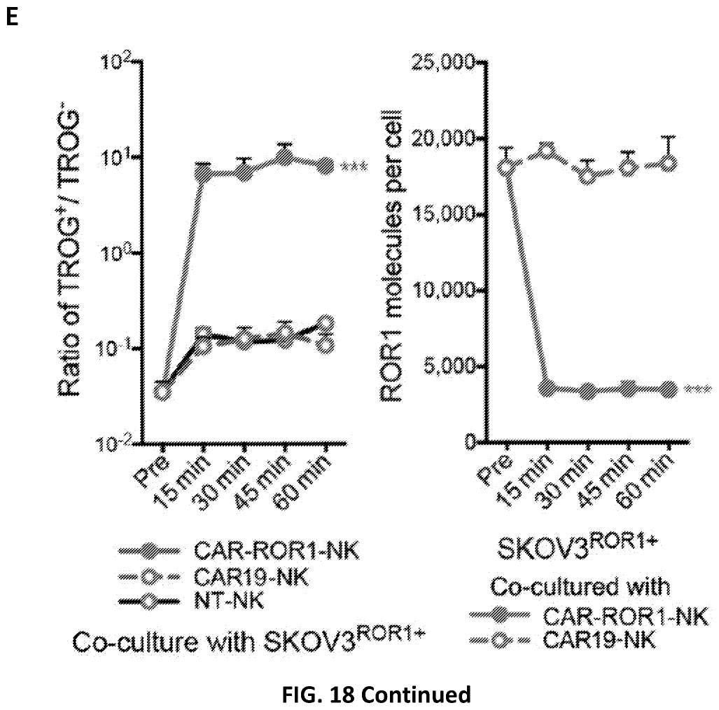

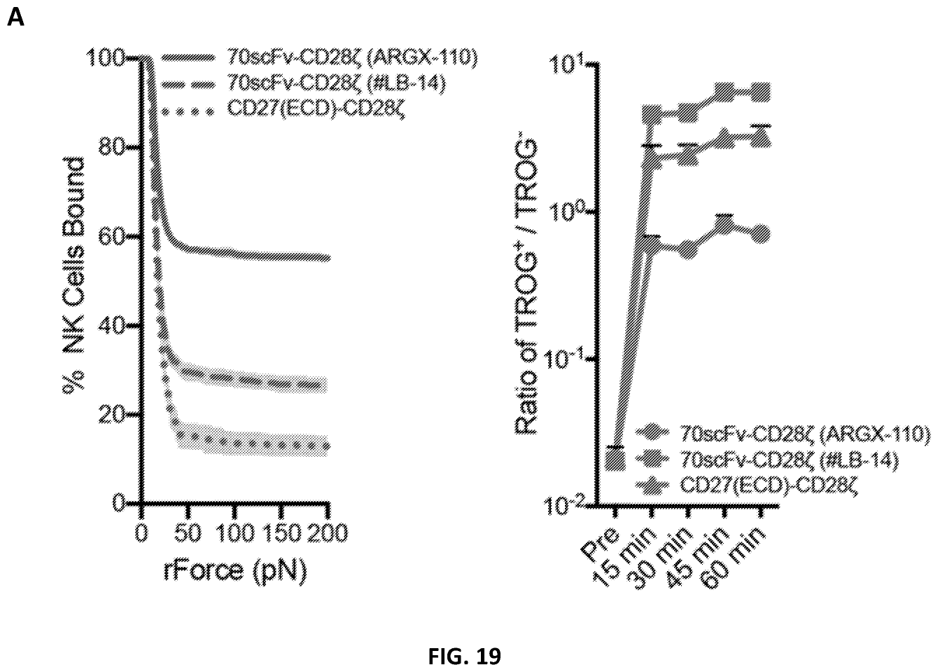

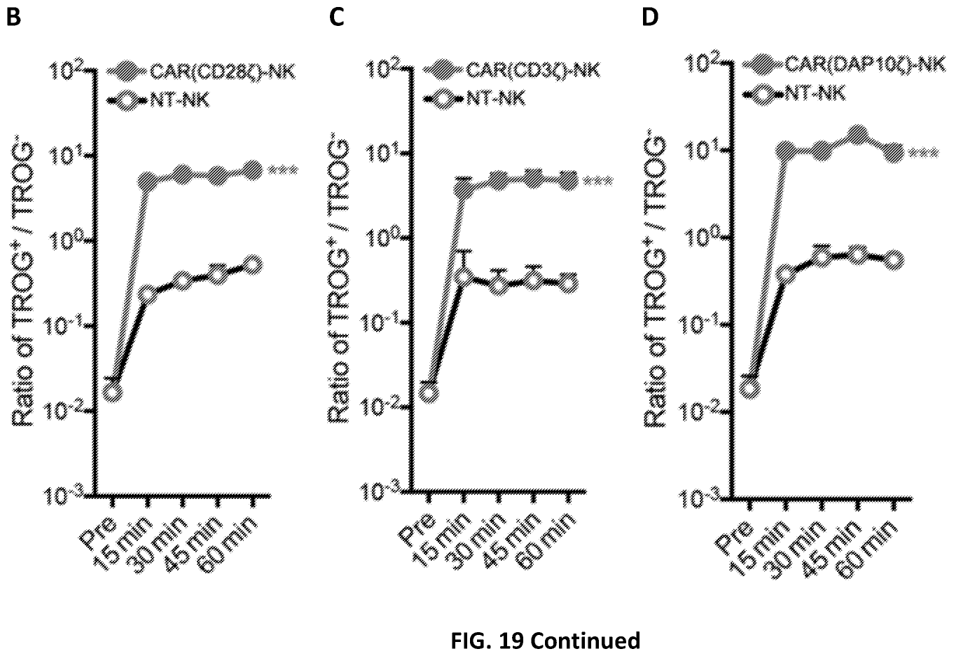

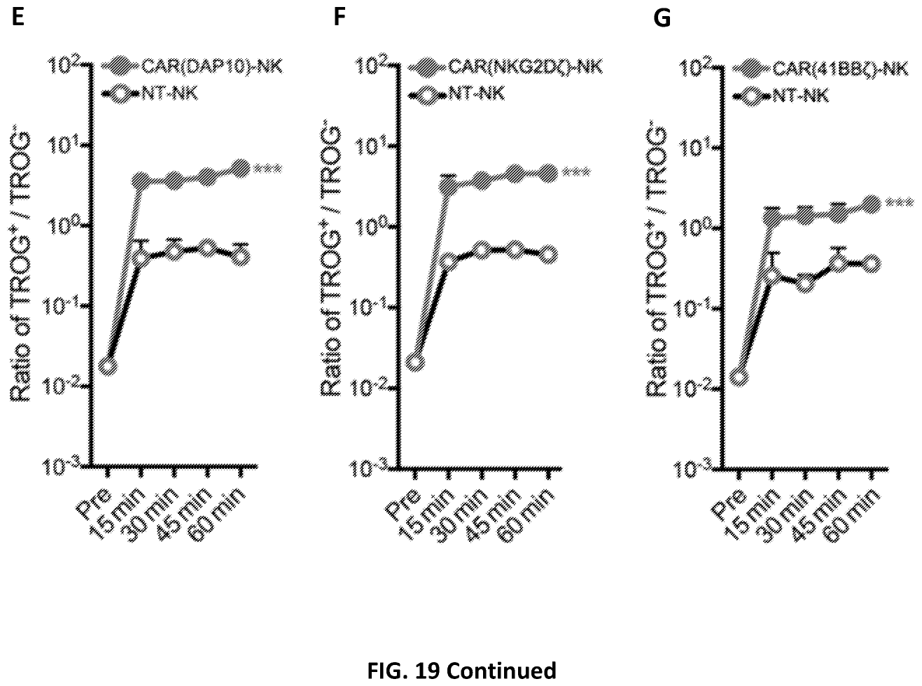

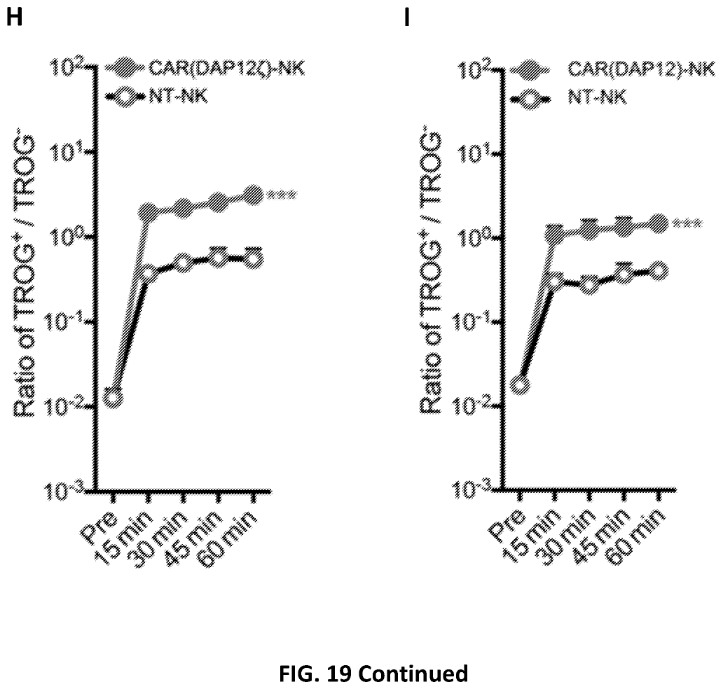

CAR19-NK cells also mediated strong trogocytic CD19 (tCD19) transfer after co-culture with different CD19⁺ targets such as NALM-6 cells, Ramos cells, healthy B cells, and ovarian cancer cells that were genetically engineered to express CD19 (SKOV3^{gCD19+}; see FIGS. 17 D and E). Importantly, CAR19-mediated trogocytosis was also observed when CAR19-NK cells were co-cultured with CD19⁺ primary tumor cells derived from patients with either Chronic lymphocytic leukemia (CLL) or Acute lymphocytic leukemia (ALL), concurrent with a reciprocal reduction in CD19 on the targets (FIG. 10 C, and FIG. 17 F). To validate this observation with different cognate antigens, CARs that recognized CD5, CD70, CD123, BCMA, or ROR1 were synthesized, and their ability to mediate trogocytosis was evaluated. A high level of cognate TROG-antigen was likewise detected on CAR-NK cells with the concurrent reduction of cognate antigen expression on co-cultured targets (FIGS. 18A-E). Notably, CAR-mediated trogocytosis appeared to be antigen-specific since cognate antigen transfer above baseline (seen with NT-NK control) was not observed when the CAR molecule was mismatched with the target antigen and was influenced by the affinity of the CAR molecule for its targeted antigen (FIG. 19A). Moreover, robust TROG-antigen transfer was not only observed with CD28/CD3ζ-based CAR-signaling, but also with NK cells transduced with CARs signaling through CD3ζ only, DAP10+/−/CD3′, DAP12+/−/CD3ζ, NKG2D/CD3ζ, and 41BB/CD3ζ (FIGS. 19A-I).

### Example 8

CAR-NK^{TROG+} cells are Highly Active but are Subject to Fratricide

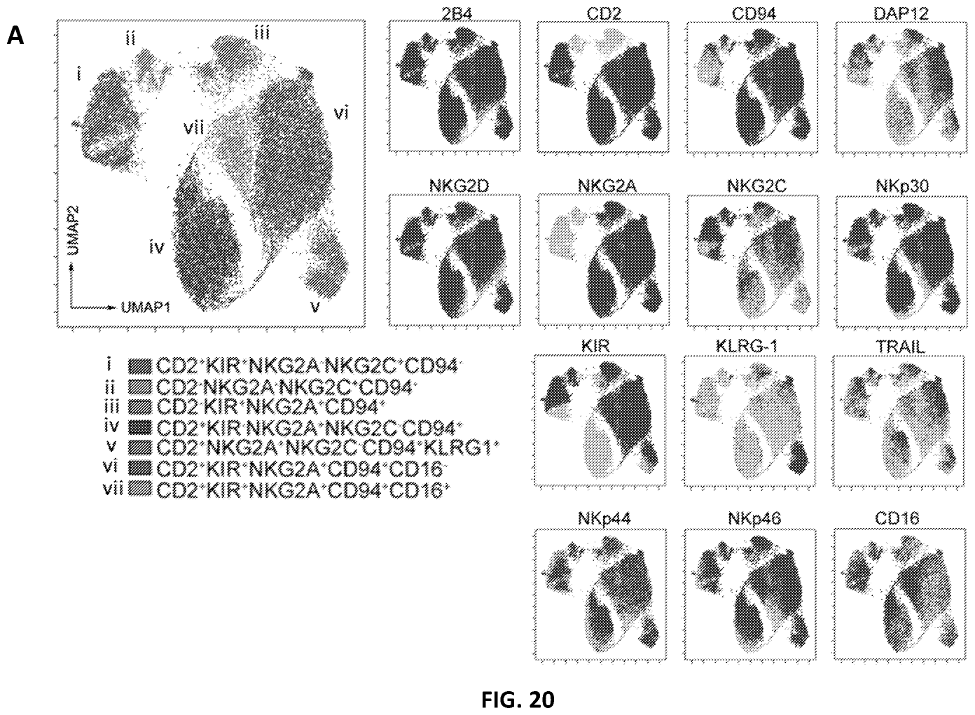

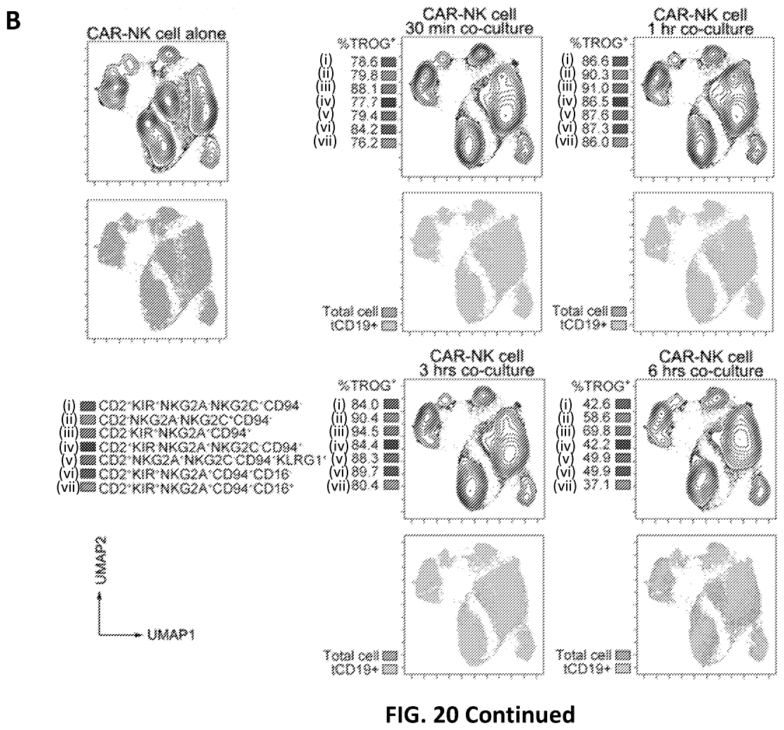

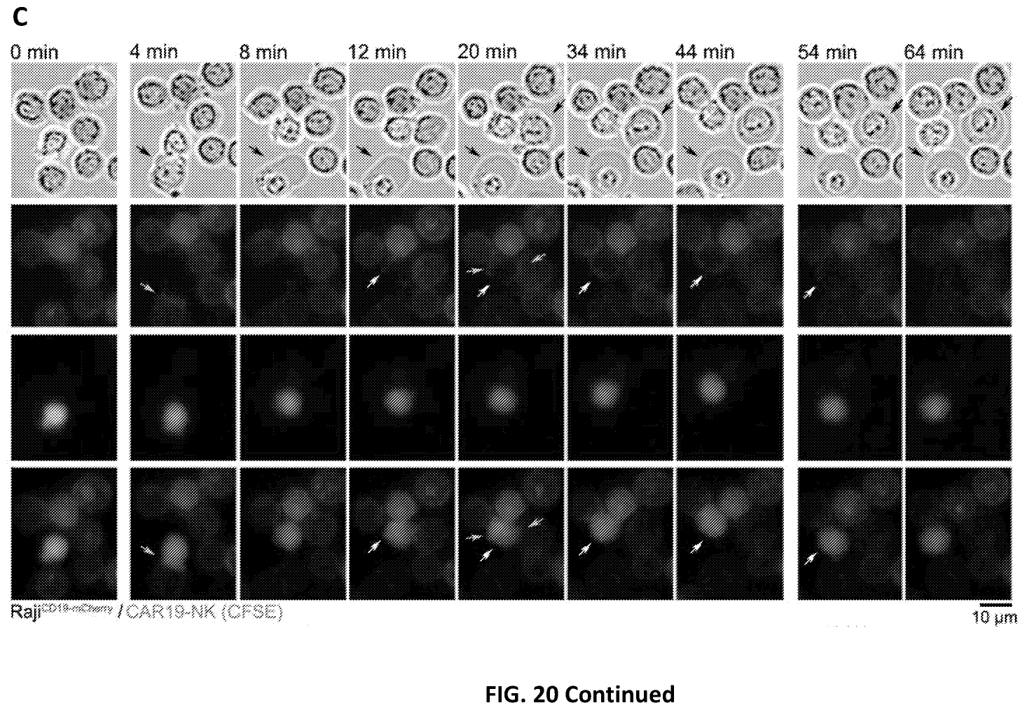

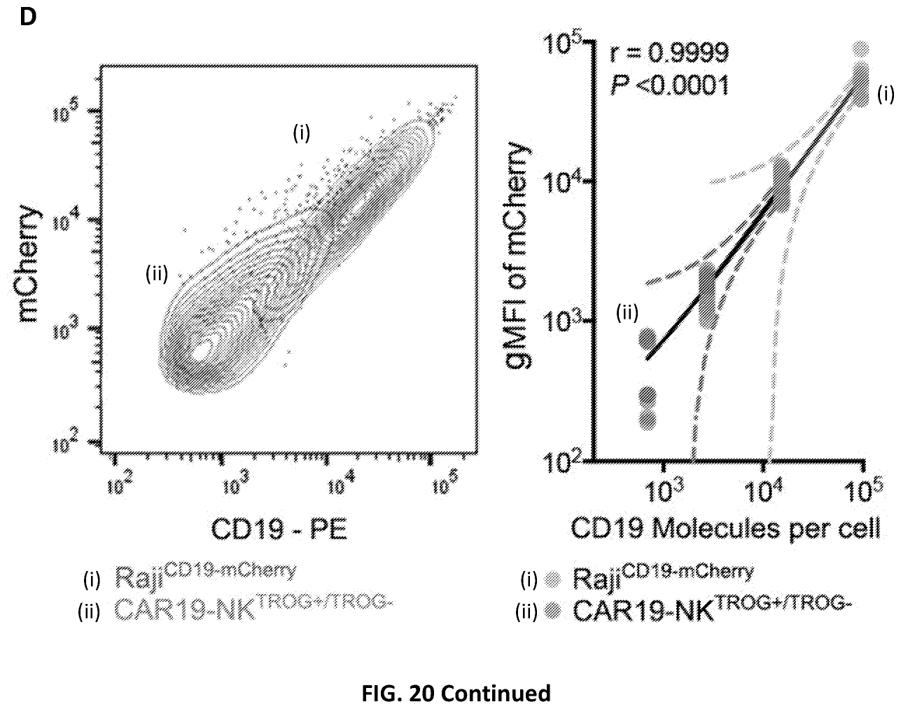

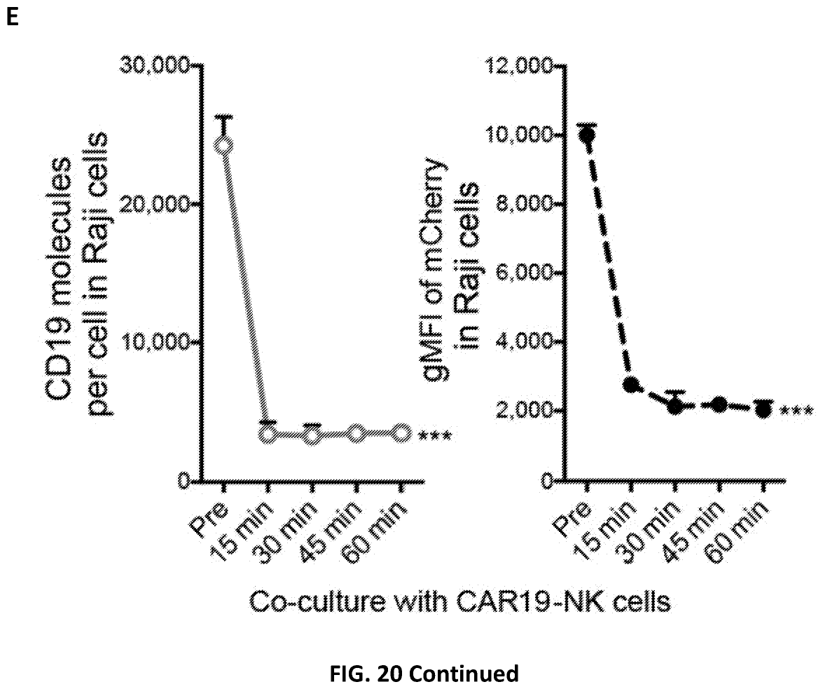

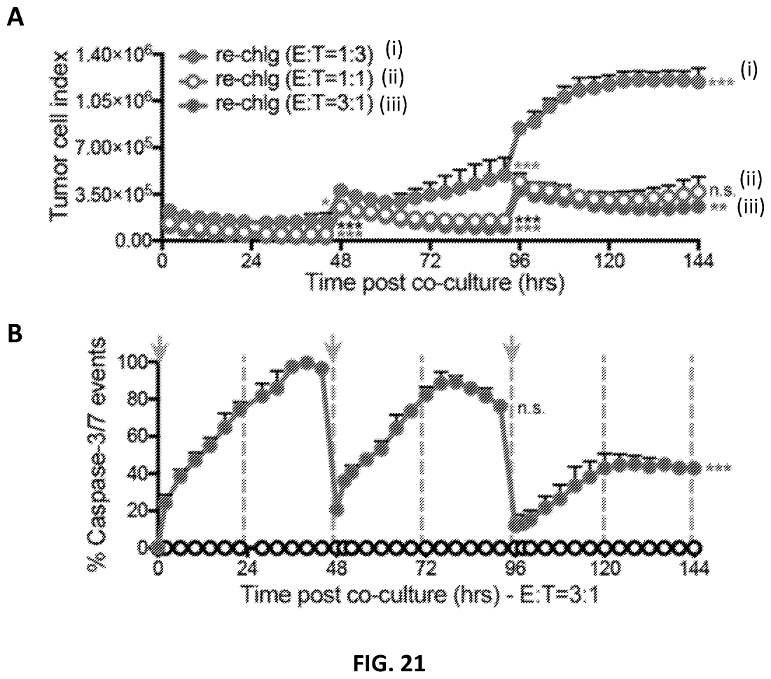

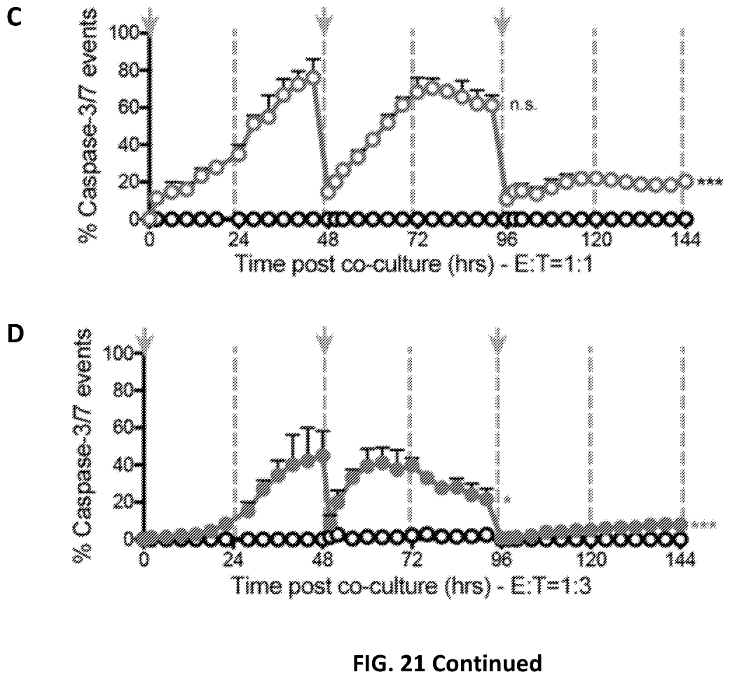

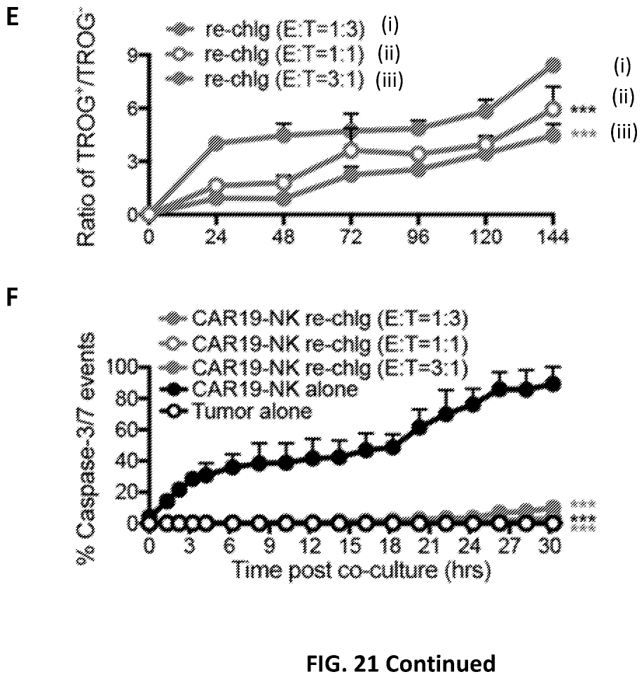

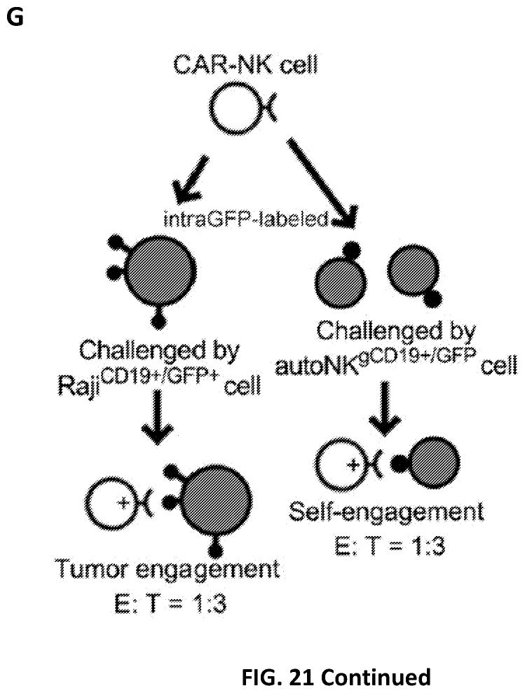

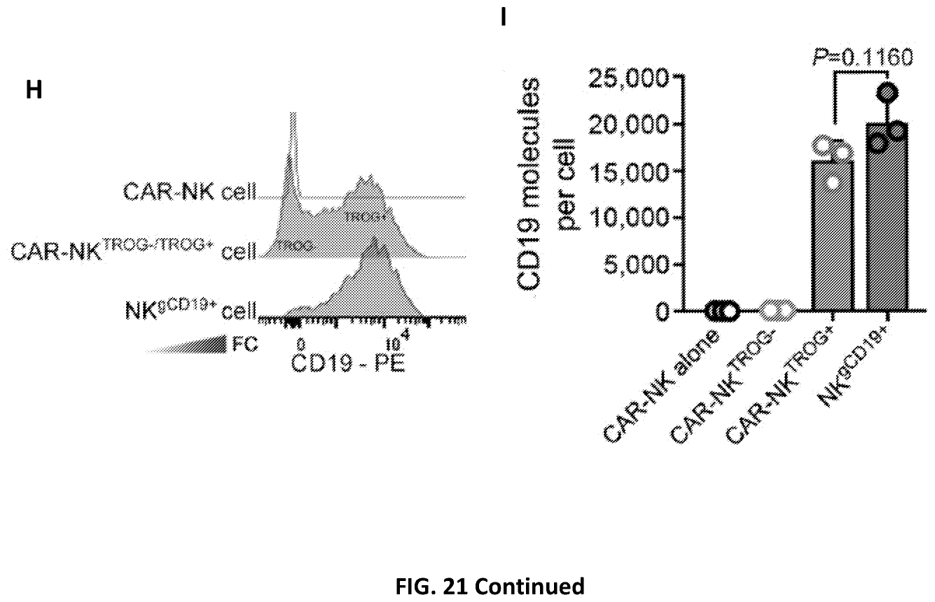

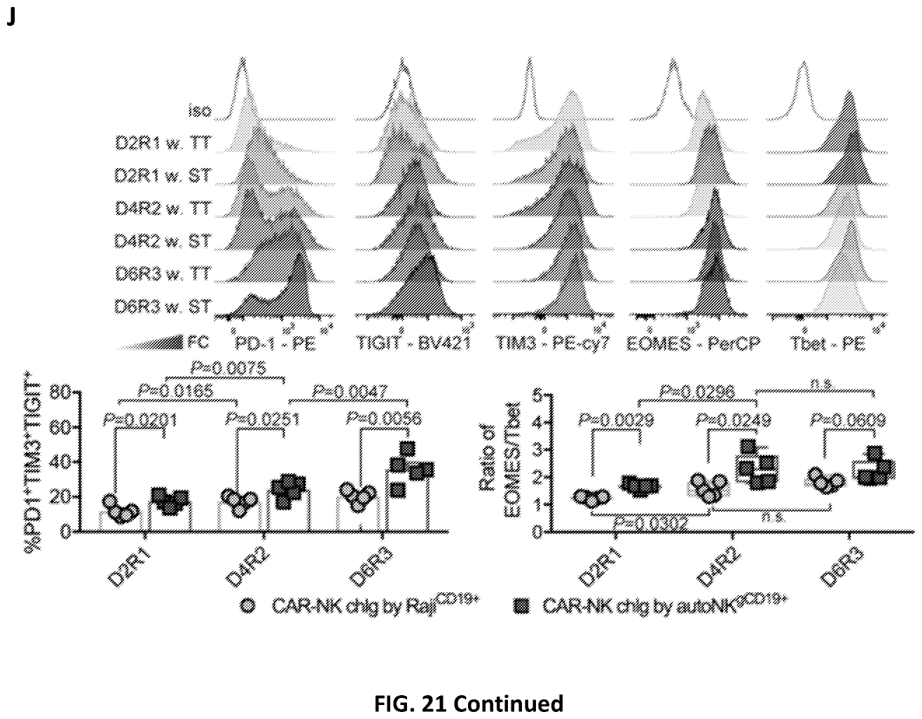

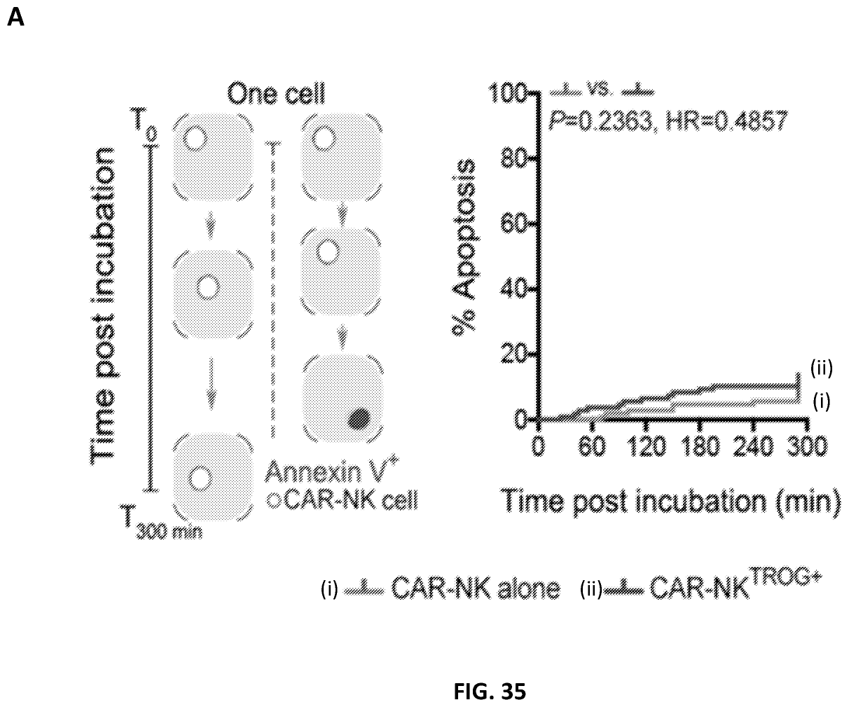

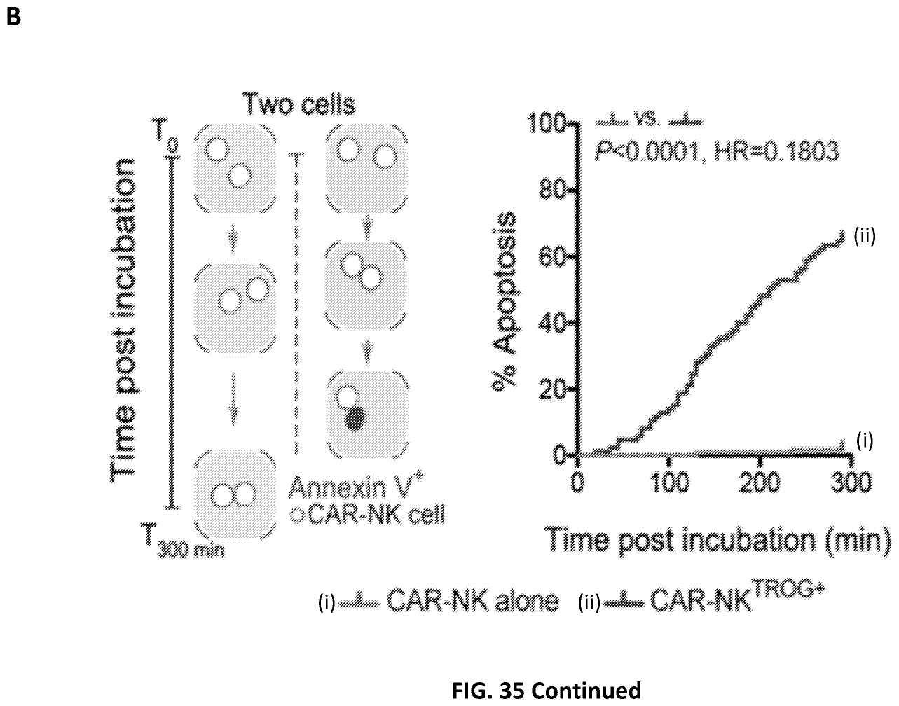

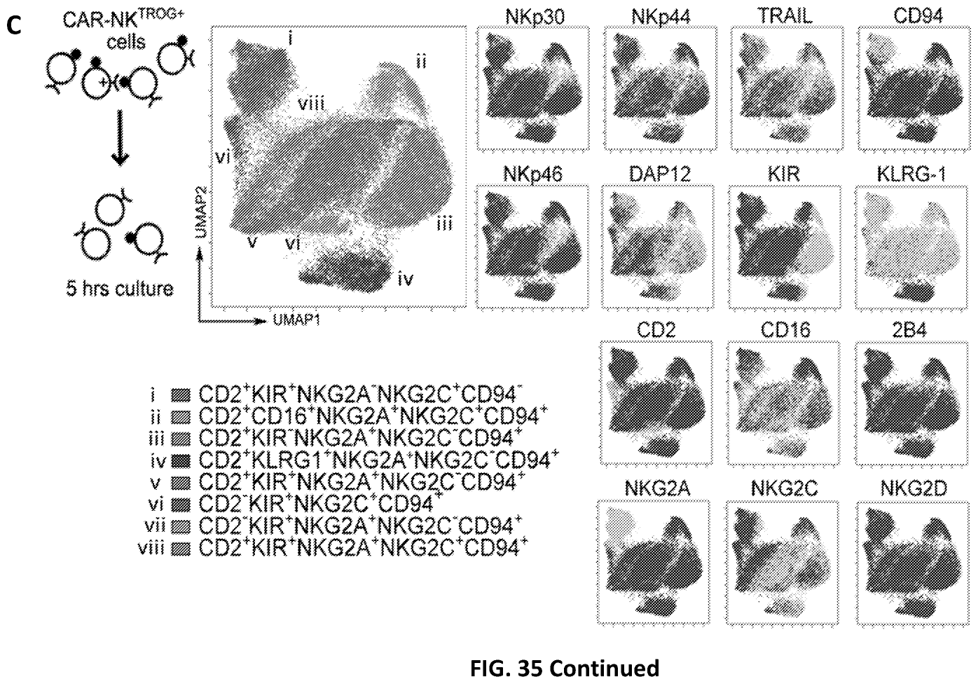

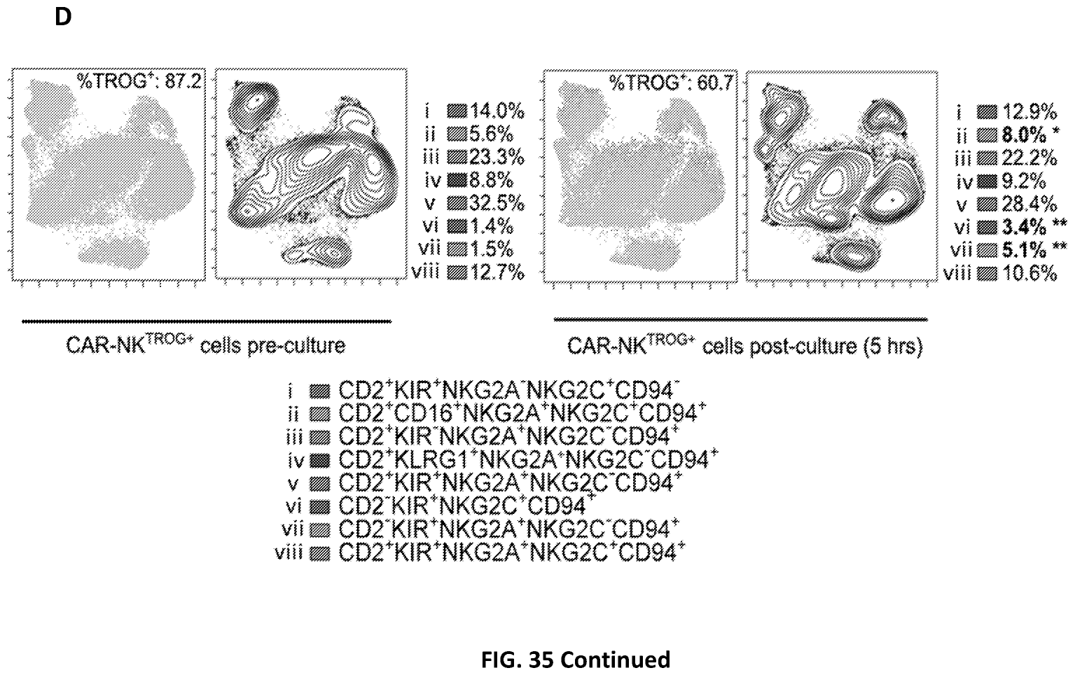

Trogocytosis has been reported to modulate NK cell function (see, e.g., Caumartin et al., 2007; Lu et al., 2021; Domaica et al., 2009; and Nakayama et al., 2011). Thus, whether TROG-antigen expression on CAR-NK cells could also modulate their effector function was determined, furthermore, whether this effect was positive or negative was also determined. It was observed that soon after co-culture with Raji targets, tCD19 was expressed on CAR19-NK cells (FIGS. 21A and B]) and those CAR-NK^{TROG+} cells displayed significantly higher levels of degranulation (CD107a) and IFN-γ accumulation in response to Raji targets when compared with their TROG⁻ counterparts (FIG. 10 D), consistent with greater activation and effector potential. Since CAR-activation is triggered by cognate antigen ligation (see, e.g., Sadelain et al., 2013), the inventors next investigated if CAR-NK cells could also mediate a cytotoxic response against their TROG⁺ NK cell siblings. To study this without the interference of using an antibody, the endogenous CD19 gene in Raji cells was knocked out followed by transduction with a CD19-mCherry fusion molecule that could be easily detected upon transfer to CAR-NK cells through trogocytosis (FIG. 10 E). The rapid transfer of both CD19 and mCherry on CAR-NK cells upon engagement with Raji^{CD19-mCherry} cells was confirmed (FIGS. 20 C-E). Notably, and in keeping with the previous findings, CAR-mediated CD19-mCherry transfer required the formation of an immunological synapse, since it was prevented by LatA pre-treatment (FIG. 10 F); and it also depended on CAR-activation as NK cells expressing 19scFv failed to transfer TROG-antigen to the same extent upon co-culture with Raji^{CD19-mCherry} cells (FIG. 10 F). The inventors also confirmed that fratricide was mediated by TROG-antigen recognition, since a significantly larger proportion of CAR-NK^{TROG+} cell populations underwent apoptosis upon co-culture with autologous CAR19-transduced NK cells compared with the control of 19scFv-NK^{TROG+} cells alone (FIG. 10 G, and FIGS. 35A-D). The addition of a CD19-blocking antibody to compete with CAR19-recognition and CD19 ligation reduced apoptosis of CAR19-NK^{TROG+} cells (FIG. 10 G), indicating that the fratricide of NK^{TROG+} population was mediated by on-target recognition, ligation and cytotoxic response by CAR-NK cells.

### Example 9

### Self-Engagement Mediated by CAR-Recognition of Cognate-Antigen Drives NK Cell Exhaustion and Metabolic Dysregulation

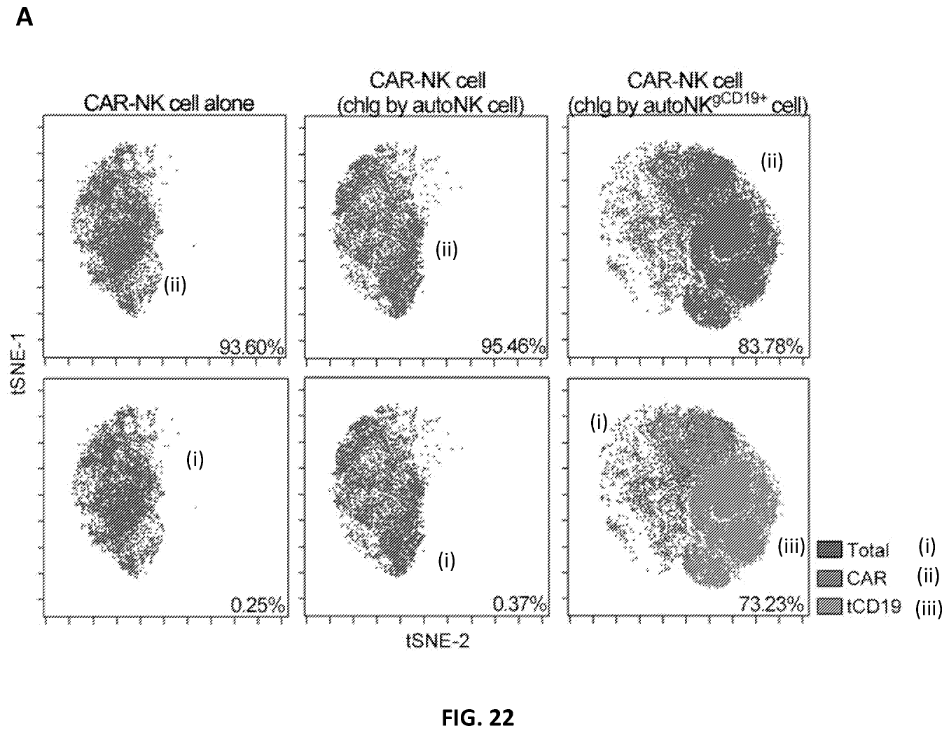

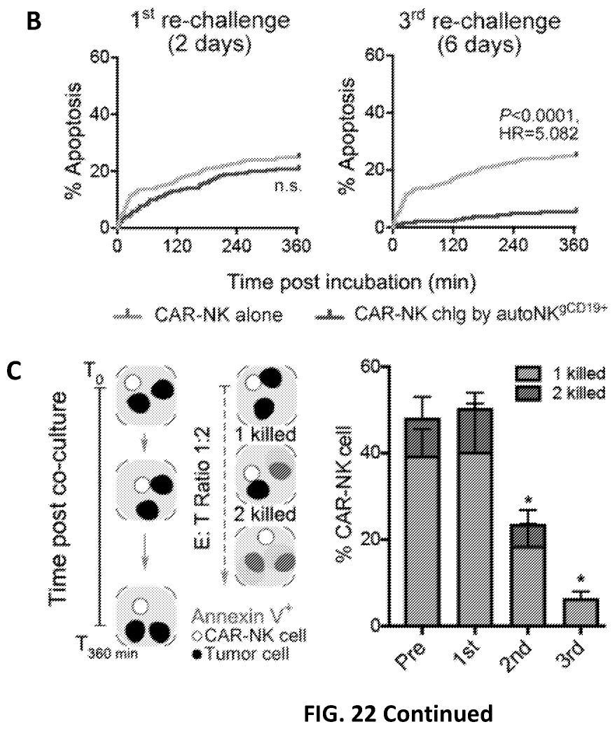

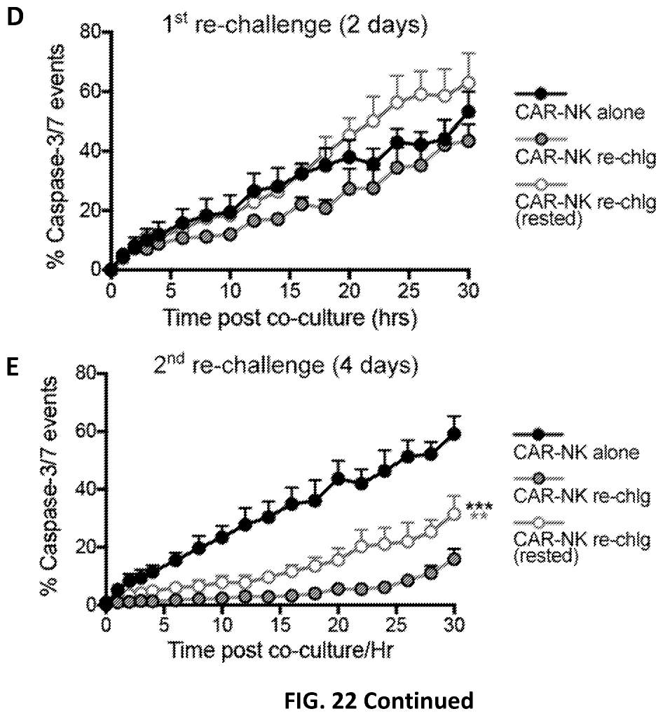

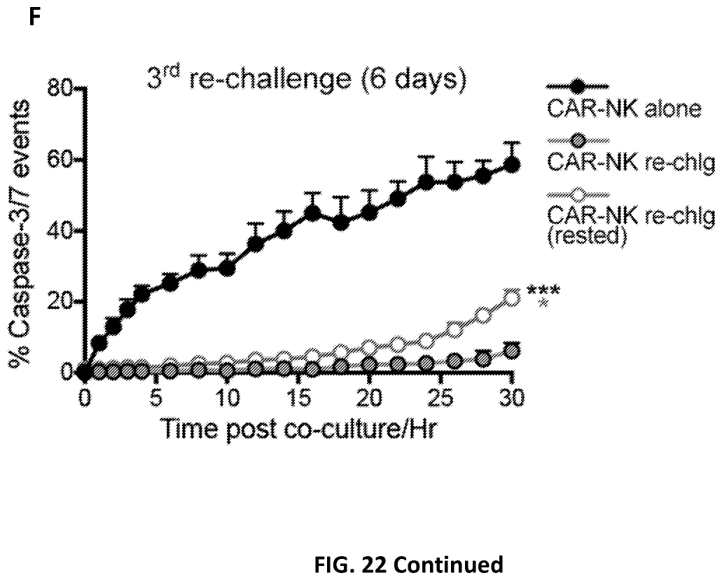

Repeated antigenic stimulation by tumor cells is known to drive exhaustion of immune effector cells (see, e.g., Poorebrahim et al., 2021; and Judge et al., 2020). Indeed, the results showed that while expression of TROG-antigen was initially associated with higher CAR-NK cell activation, it did not result in sustained anti-tumor activity in a repeated tumor challenge model (FIGS. 21A-F). Thus, it was determined whether TROG⁺ CAR-NK cells that did not succumb to fratricide were susceptible to functional exhaustion through repeated TROG-antigen-mediated CAR activation. CAR-NK cells were repeatedly challenged at a lower E:T ratio of 1:3 (to minimize fratricide) with either GFP-labeled Raji^{CD19+} cells or with autologous NK cells that were genetically modified to stably express CD19 on their surface and GFP intracellularly (autoNK^{gCD19+/GFP+} cells; FIG. 21 G). The level of CD19 expression on NK^{gCD19+} cells was approximately equivalent to that of tCD19 detected on CAR-NK^{TROG+} cells (FIGS. 21 H and I). CAR19-NK cells cultured alone or after co-culture with autoNK^{gGFP+} cells (lacking CD19 expression) were used as controls. In keeping with the rechallenge data with Raji tumor cells, CAR-NK effector cells (GFP-negative) that were repeatedly challenged with autoNK^{gCD19+/GFP} cells acquired an exhaustion phenotype with a notable percentage (21%±9%) of cells co-expressing multiple checkpoints (PD1, TIM3, and TIGIT) (see, e.g., Pesce et al., 2017; and Anderson et al., 2016), and a higher ratio of eomesodermin (EOMES) to T-bet (see, e.g., Gill et al., 2012; and Simonetta et al., 2016) (FIG. 21 J). Further phenotypic interrogation using mass cytometry (CyTOF) confirmed that over 65% of CAR-NK effector cells challenged with autoNK^{gCD19+/GFP+} targets (clusters EC3-EC5; FIGS. 11A and B) co-expressed PD1, TIGIT, LAG3, TIM3, with increased EOMES, and downregulation of Tbet and Ki67, when compared with CAR-NK cells cultured alone or with autoNK^{gGFP+} cells that did not express CD19 (FIG. 11 C). Acquisition of tCD19 from autoNK^{gCD19+/GFP+} cells by CAR-NK effector cells (FIG. 22A) was associated with down-regulation of NK cell activating receptors such as NKG2D, NCRs, CD16, 2B4, adaptor molecules (Syk, Zap70, DAP12, and SAP), as well as cytolytic proteins (granzymes and perforin; FIG. 11 C). Interestingly, nearly 35% of CAR-NK cells (EC4-EC5) co-cultured with autoNK^{gCD19+/GFP+} also expressed higher levels of co-activating receptors such as NKG2C, DNAM-1, OX40, and CS1, as well as CD25 and CD69 (FIG. 11 C), suggesting that CAR-NK cells only acquired their exhausted phenotype after CAR-antigen mediated self-engagement and activation.

Based on the phenotypic observation, the inventors anticipated that repeated antigenic challenge by sibling autoNK^{gCD19+/GFP+} cells drives the functional exhaustion of CAR-NK cells. To test this, CAR-NK effector cells (GFP-negative) were isolated after 4 days of co-culture with autoNK^{gCD19+/GFP+} cells (GFP-positive), and their cytotoxicity was compared to that of CAR-NK cells cultured alone or co-cultured with autoNK^{GFP+} cells. In each nanowell, only one CAR-NK effector cell was incubated with its target cell to rule out the possibility of fratricide (Methods; FIG. 11 D). At the single-cell level, CAR19-NK cells that were continuously activated through fratricide (vs. autoNK^{gCD19+/GFP+} cells) killed significantly fewer K562 targets (FIG. 11 E) and CD19⁺ Raji tumor targets (FIG. 11 F, and FIGS. 22 B and C) when compared with controls, and this was especially the case at lower E:T ratios and after multiple rounds of rechallenge with autoNK^{gCD19+/GFP+} cells (FIGS. 22 D-F). Together, these findings confirmed impaired CAR-NK effector function resulting from antigen-induced self-engagement.

Given the importance of cellular metabolic fitness for NK cell effector function (see e.g., Gardiner & Finlay 2017, and O'Brien & Finlay 2019), the inventors also evaluated whether CAR-NK cells co-cultured with autoNK^{gCD19+/GFP+} had dysregulated metabolic capacity. Compared to controls, CAR-NK cells previously challenged with autoNK^{gCD19+/GFP+} had significant impairment in their metabolic machinery at their baseline level (without stimulation) and in response to the maximum stimulation when compared to controls, with a significant reduction in their glycolytic capacity as measured by the extracellular acidification rate (ECAR) (FIG. 11 G), and oxygen consumption rate (OCR) and oxidative phosphorylation (OXPHOS; FIG. 11 H). Taken together, these data supported a model in which continuous CAR-activation, via self-engagement with the TROG antigen, drives the functional exhaustion and metabolic dysregulation of CAR-NK cells.

### Example 10

### TROG-Antigen Expression Reduces the Persistence of CAR-NK Cells In Vivo

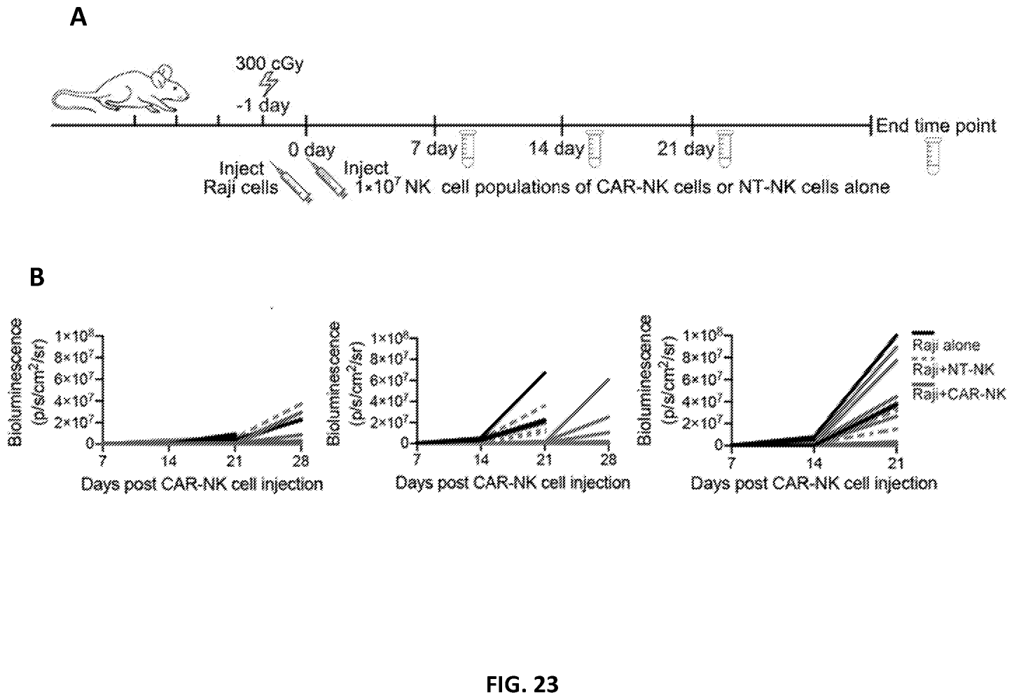

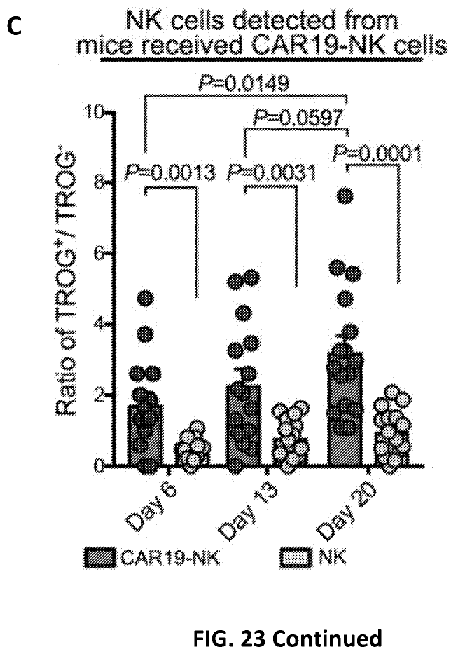

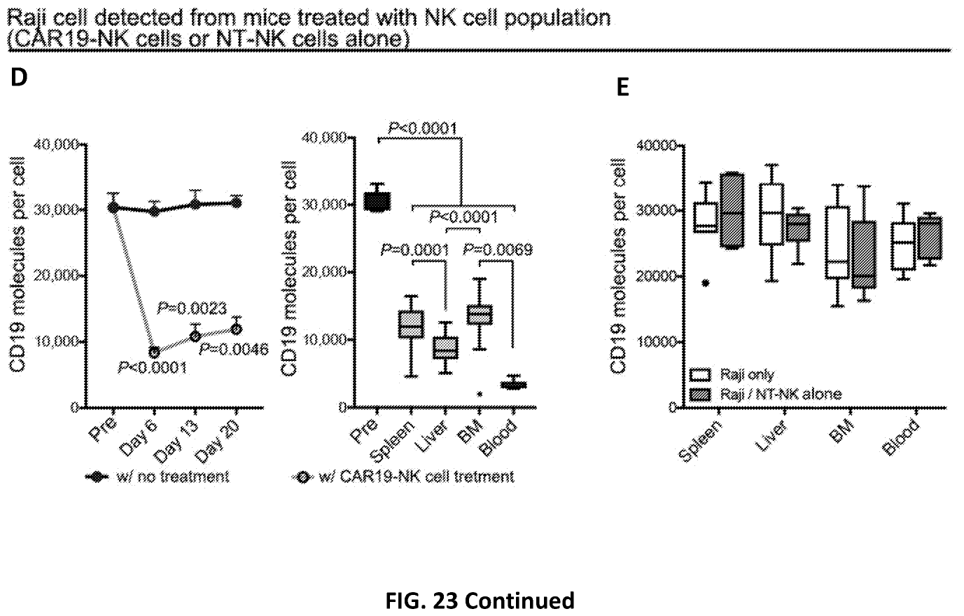

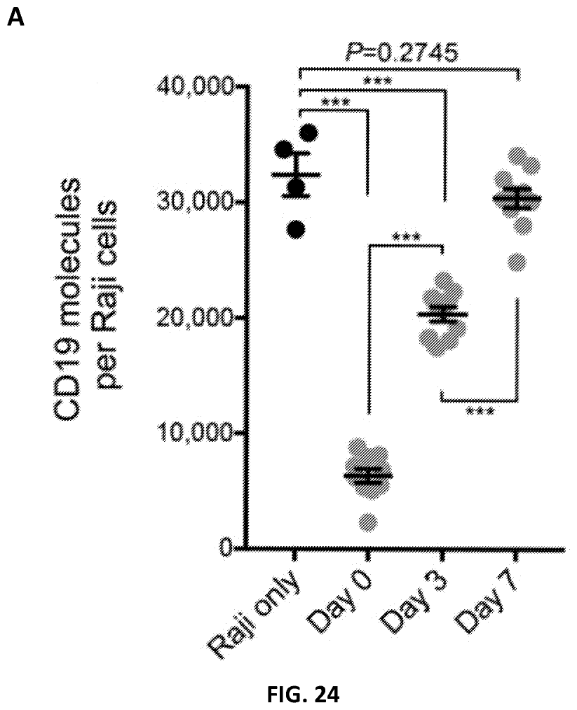

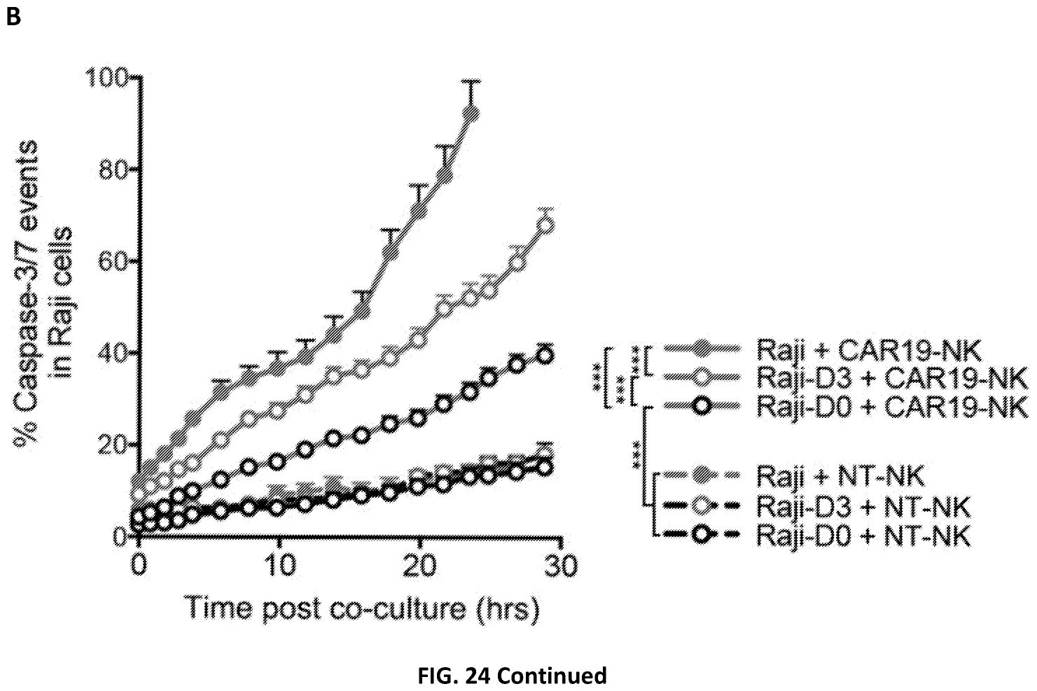

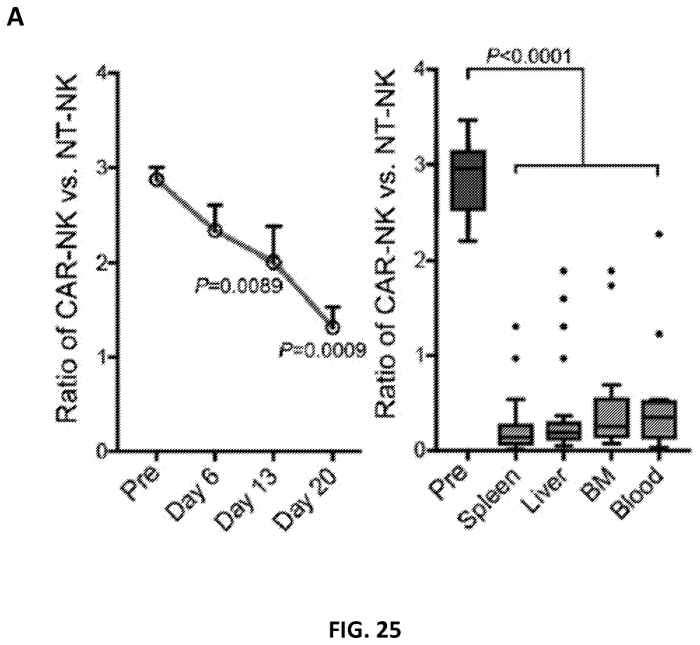

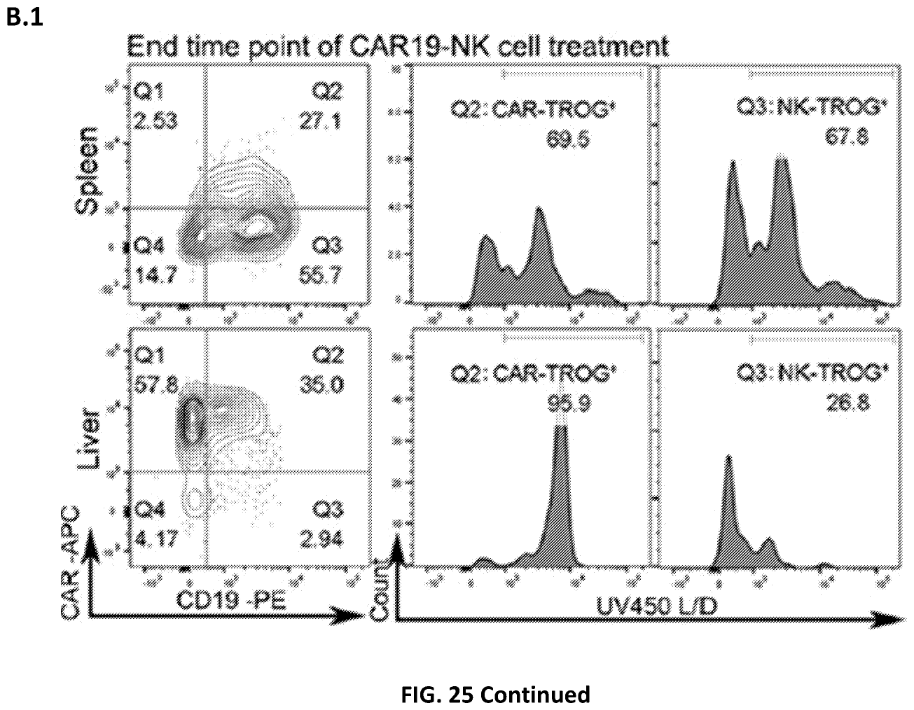

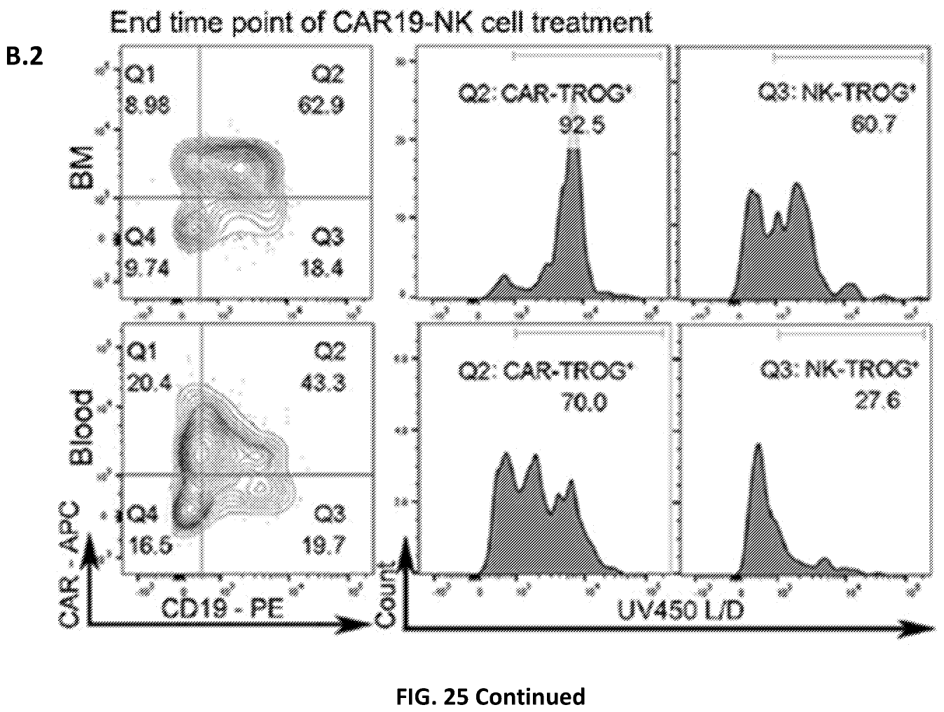

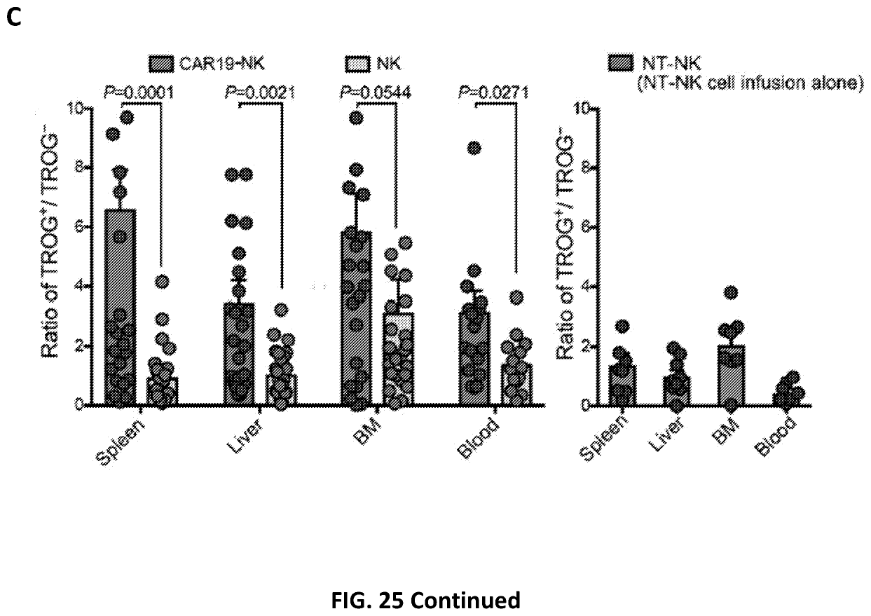

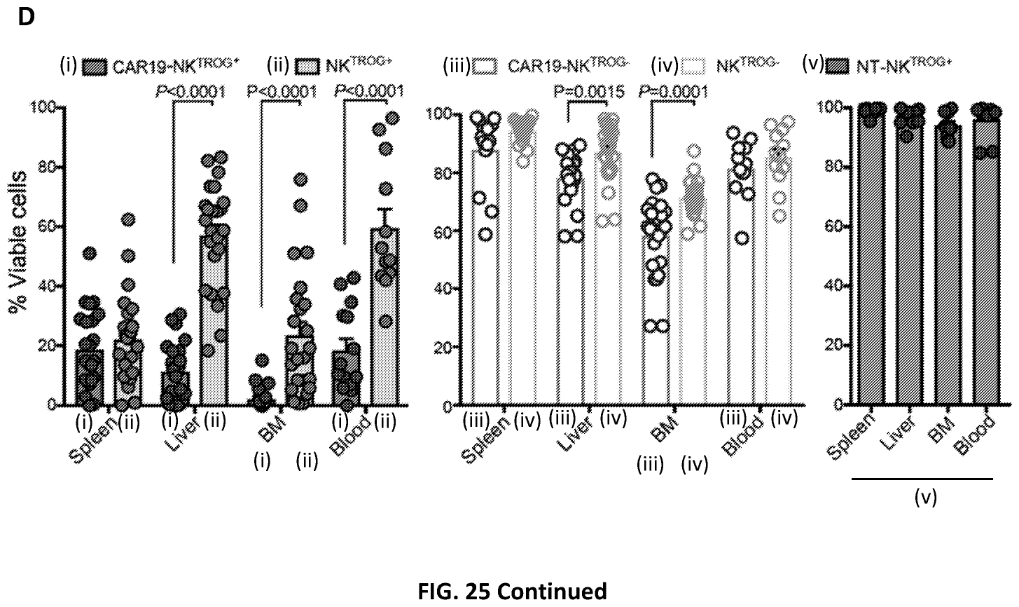

To investigate the in vivo kinetics of TROG-antigen acquisition on CAR-NK cells, mice were xenografted with 3 escalating dose levels of Raji cells (0.2×10⁵, 1×10⁵, or 0.5×10⁶), respectively, to cover the various levels of tumor burden, followed by a single infusion of CAR-NK cells (Methods; FIGS. 23A and B). In keeping with the in vitro data, significant fractions of CD19-expressing CAR19-NK cells (CAR19-NK^{tCD19+}) were detected as early as six days post-infusion, with the TROG⁺ population increasing over the treatment course, regardless of their initial tumor burden (FIG. 23 C). The transfer of tCD19 to CAR-NK cells was also associated with a decrease in CD19 expression on Raji cells (FIG. 23 D), but the same phenomenon was not observed in mice treated with ex vivo expanded non-transduced NK cells (NT-NK cells; FIG. 23 E), suggesting a robust in vivo CAR-mediated trogocytosis. Notably, CD19 expression in Raji cells harvested from the organs of the animals returned to baseline after a short-term period of ex vivo culture (FIG. 24A) with the restoration of their susceptibility to CAR-NK cell-mediated cytotoxicity in vitro (FIG. 24 B). Notably, in vivo acquisition of TROG-antigen by CAR-NK cells was associated with a reduction in their cell number and lower viability when compared with controls (FIGS. 25A-D), suggesting that trogocytosis of the CD19 antigen was associated with fratricide that contributed to the substantial in vivo loss of CAR19-NK cells.

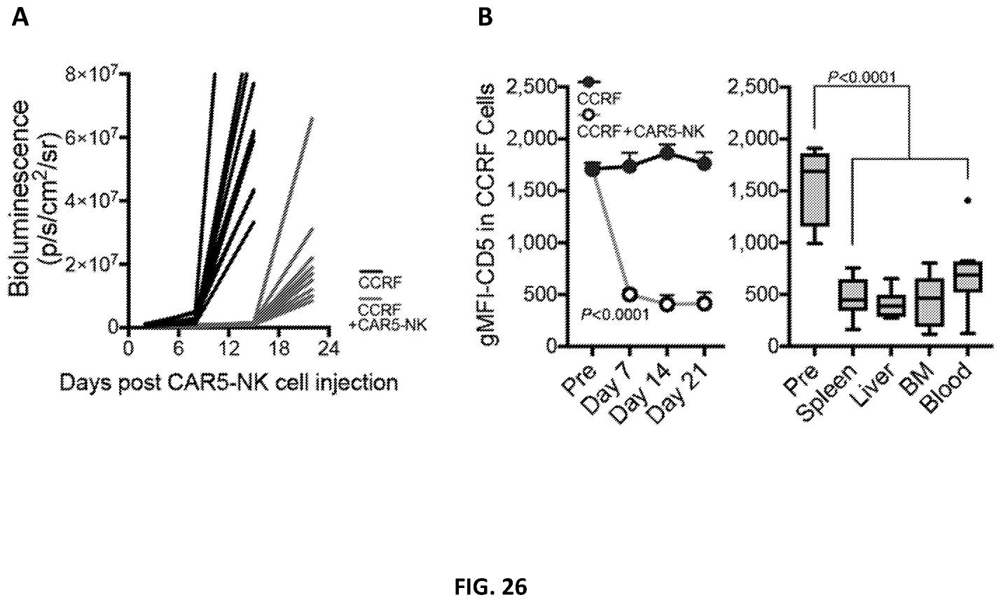

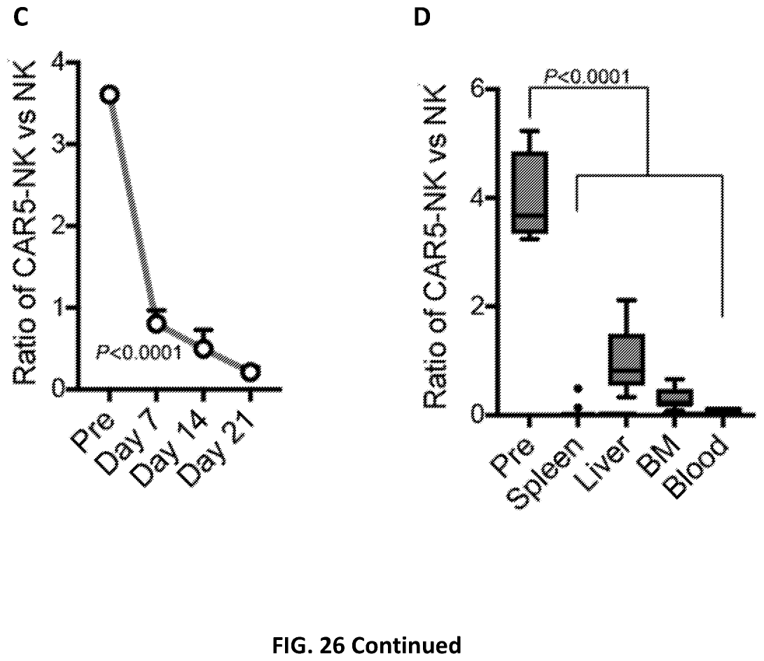

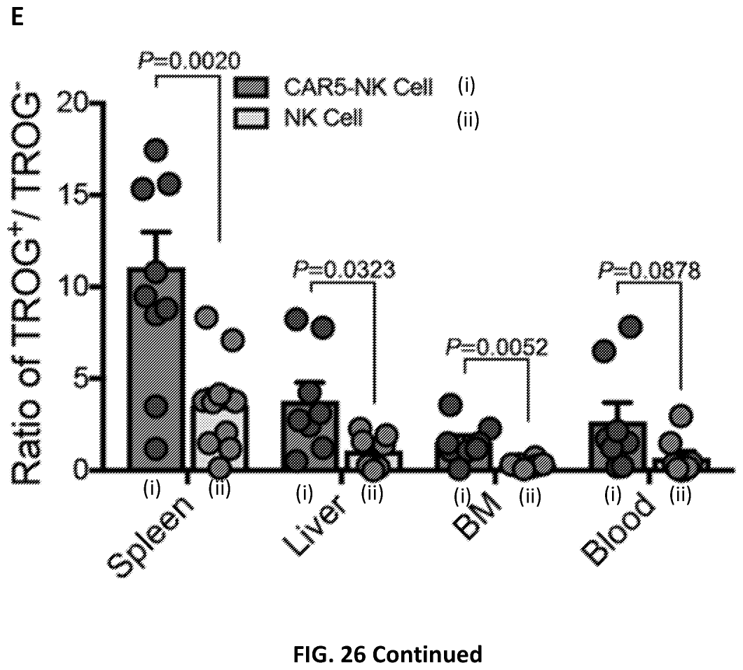

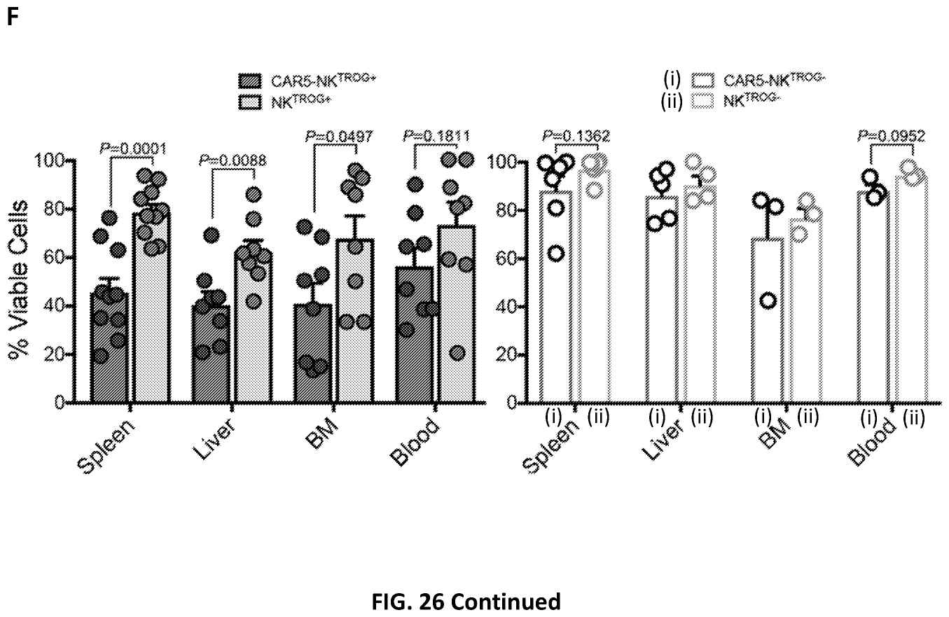

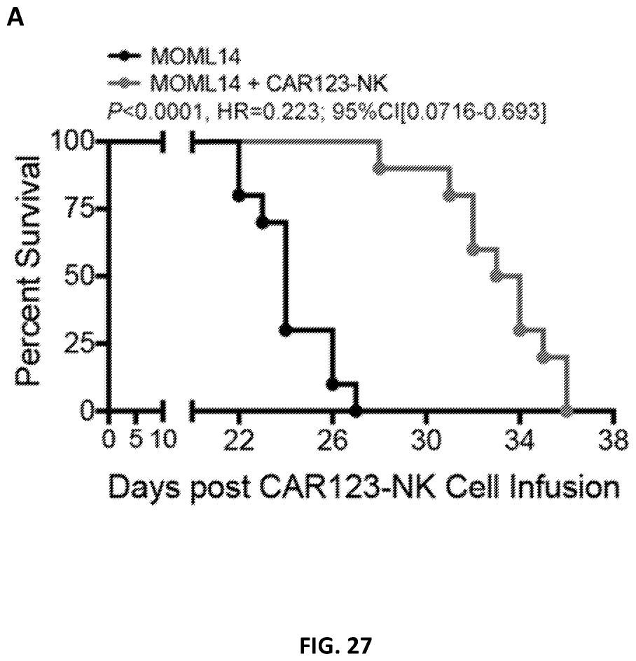

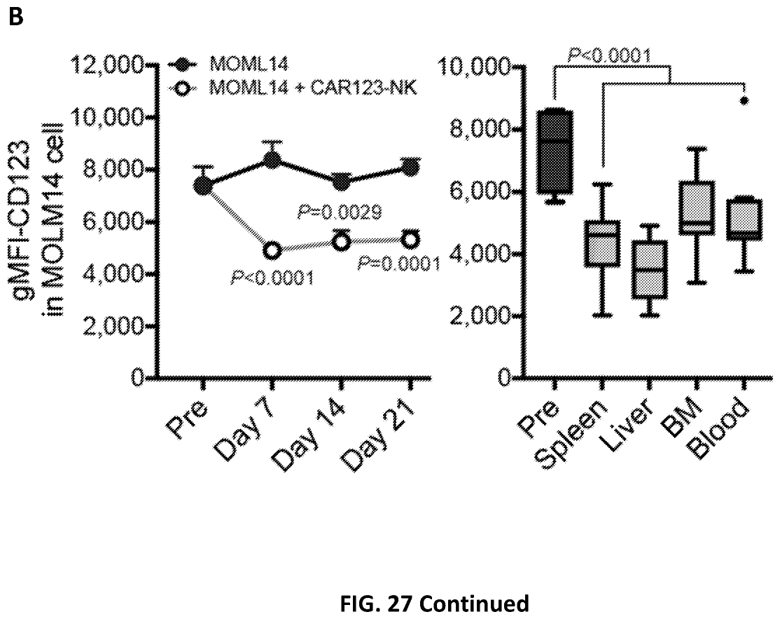

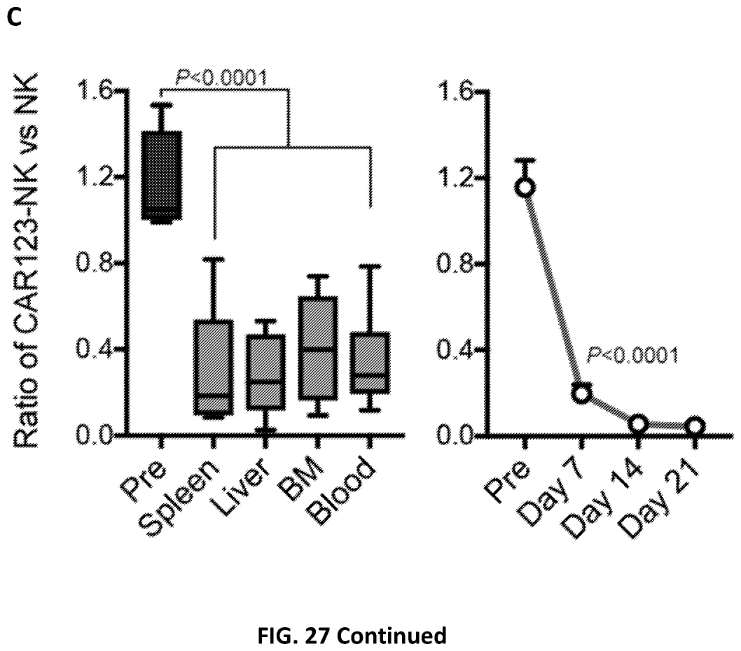

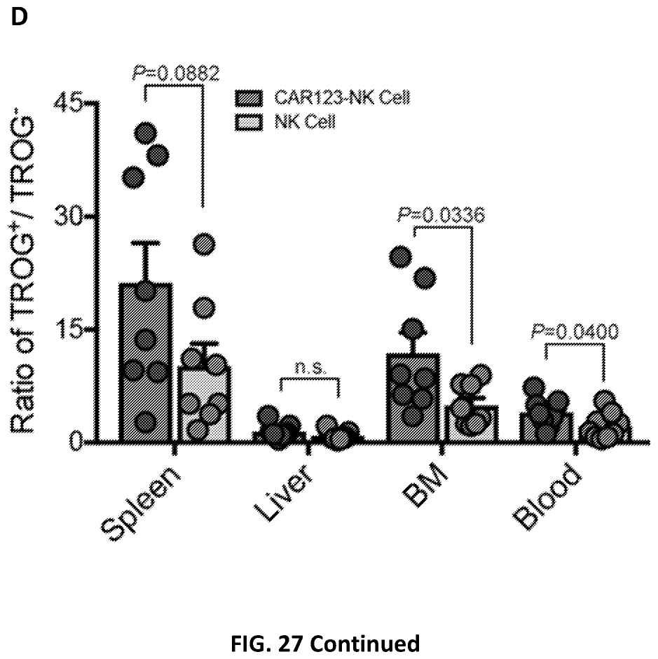

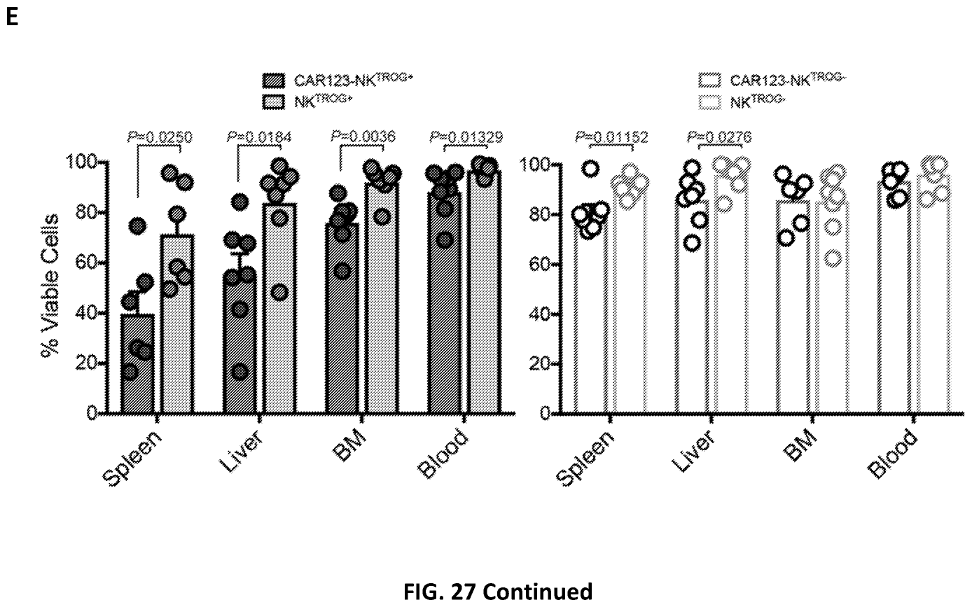

Similar observations were detected in mouse models treated with anti-CD5 CAR-NK cells (grafted with CCRF^{CD5+}; FIGS. 26A-F) and anti-CD123 CAR-NK cells (grafted with MOLM14^{CD123+}; FIGS. 27A-E). Together, these findings showed that TROG-antigen induced in vivo fratricide of CAR-NK cells was common and likely universal.

### Example 11

CAR-NK^{TROG+} Cells have an Exhausted Phenotype In Vivo and their Accumulation is Associated with Tumor Progression

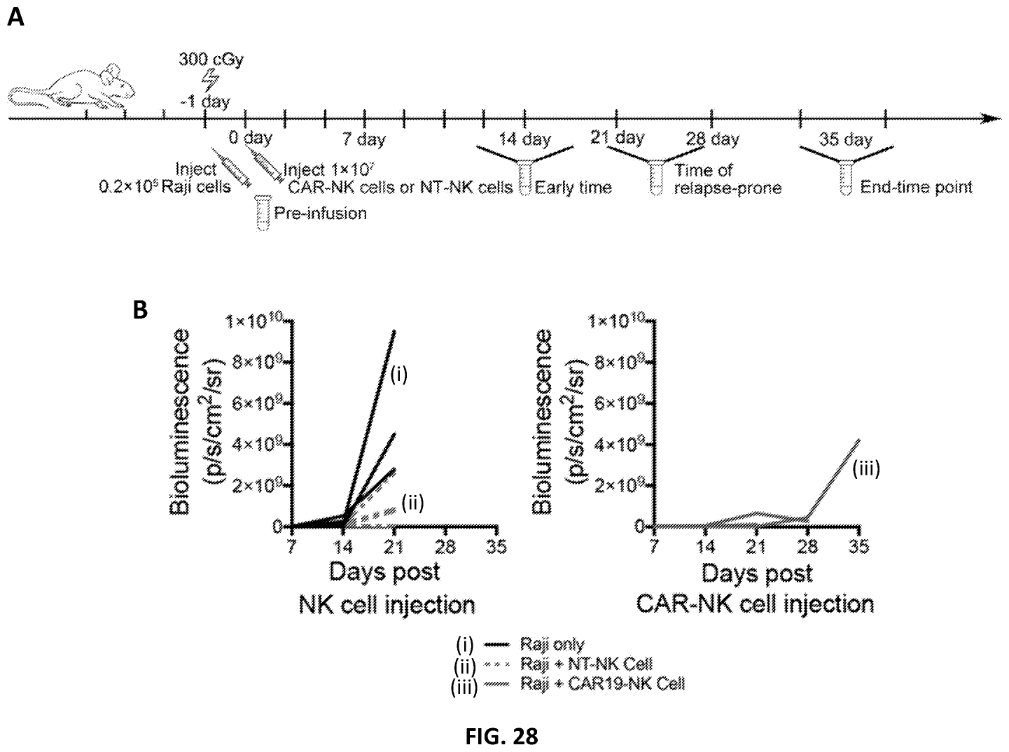

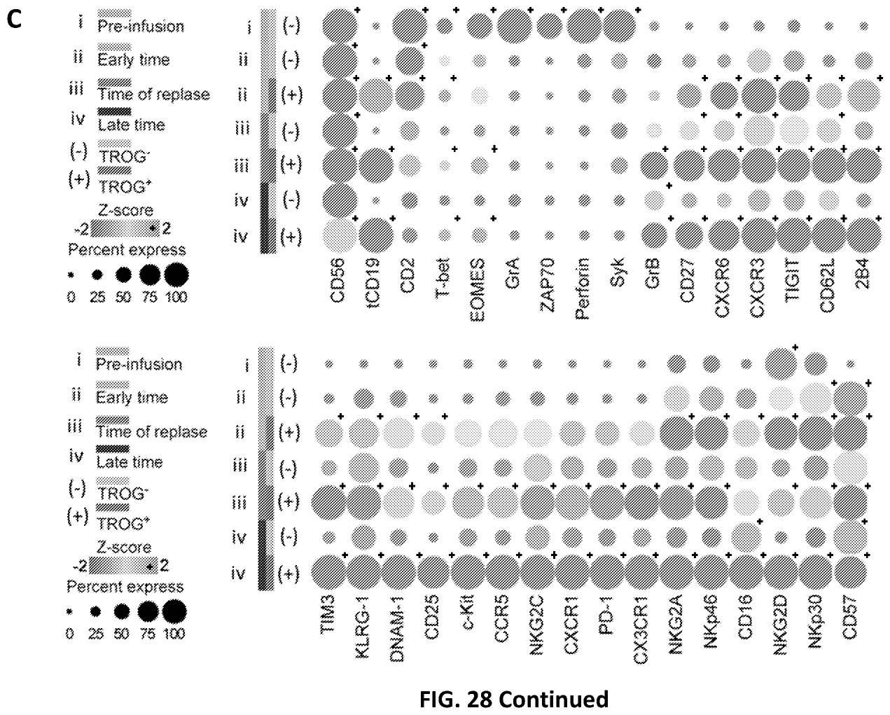

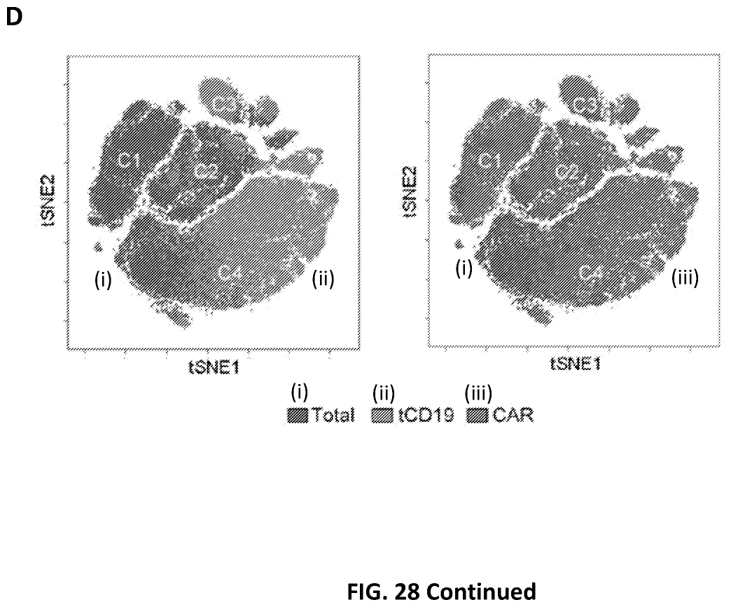

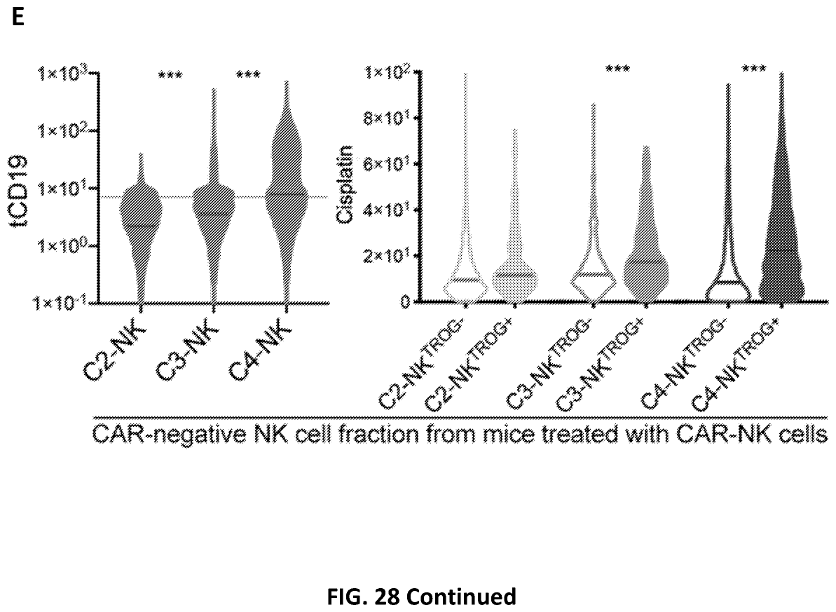

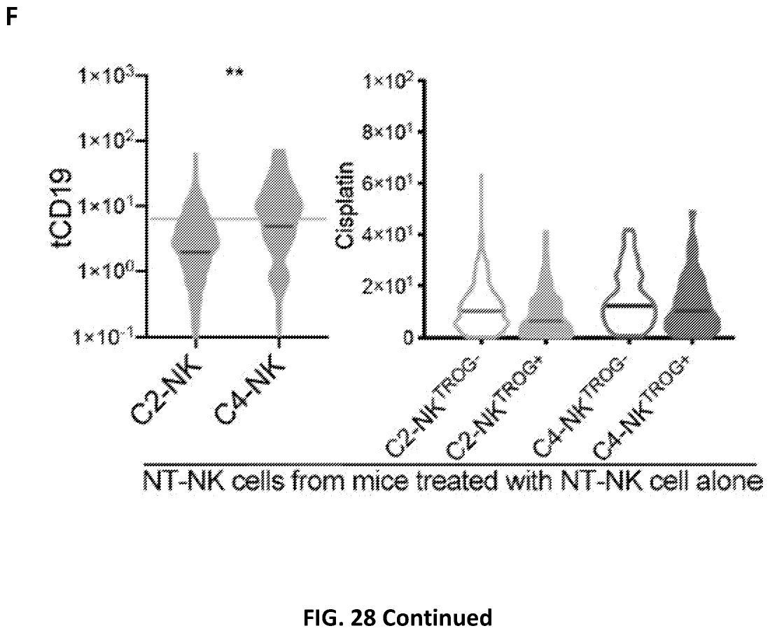

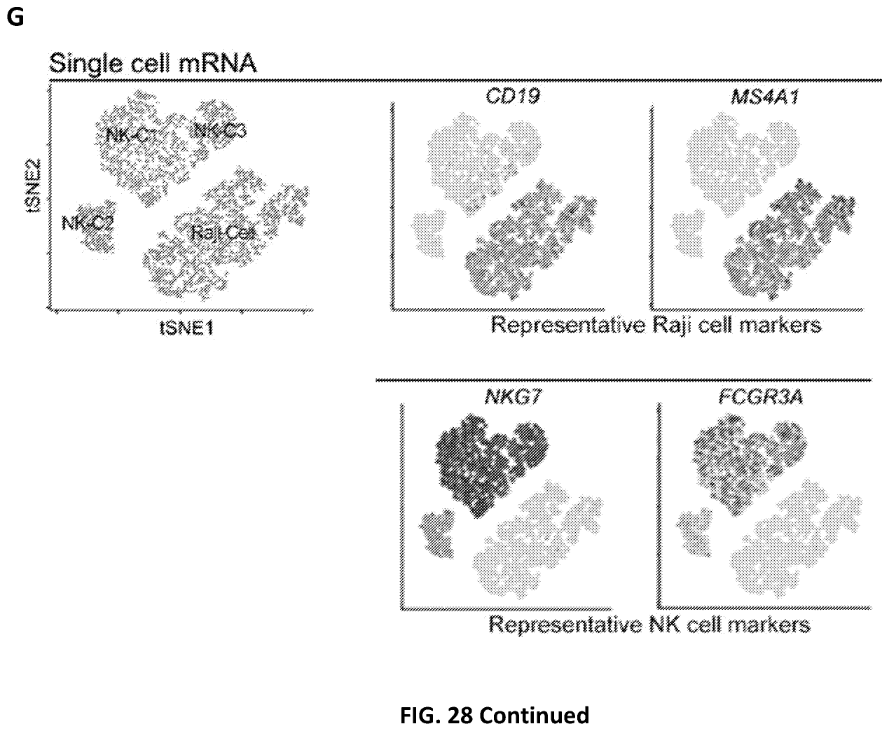

A non-curative mouse model of Raji tumor was then utilized. In this model, a single dose of CAR-NK cell infusion could initially control tumor progression, but was frequently followed by tumor relapse (see, e.g., Liu et al., 2018; and Daher et al., 2021(b); and FIGS. 28 A and B). Mice were sacrificed at regular intervals following CAR-NK cell infusion, with their blood and tissues harvested for comprehensive phenotypic analysis by CyTOF. Using a 1-SNE map, 4 major clusters of CAR-NK cells were observed (FIG. 12A). Pre-infusion CAR-NK cells were exclusively clustered in C1 (99%; FIG. 12 B), but cells from early time points post-infusion were predominantly found in C2 (66%; FIG. 12 B); at later time points and during the relapse phase, the majority of CAR-NK cells were segregated in C3 and C4 (73%-97%; FIG. 12 B), with higher expression of TROG-antigen (such as tCD19) when compared with their non-CAR expressing NK counterparts (FIG. 12 C). In vivo acquisition of TROG-antigen was associated with higher expression of both activation and inhibitory markers (FIGS. 28 C and D). Notably, CAR-NK cells in C3 and C4 showed higher expression of checkpoint markers (PD1, TIM3, TIGIT), lower expression of transcription factors, cytolytic proteins, and adaptor molecules, but upregulation of cytokine receptors IL-2R (CD25), SCF receptor (c-Kit), co-activating receptors (NKG2C, 2B4, DNAM-1), and chemokine receptors (FIG. 12 D), which is consistent with a previously activated phenotype and eventual exhaustion (see, e.g., Li et al., 2019; Judge et al., 2020; and Merino et al., 2019).

Cells were then remapped to metaclusters using FlowSOM, an unbiased gating method from the inbuilt algorithm of tSNE (see, e.g., Van Gassen et al., 2015), to compare the phenotypic signatures of TROG⁺ vs. TROG⁻ CAR-NK cells (FIG. 12 E). In keeping with the earlier results, expression of tCD19 on CAR-NK cells was associated with a reduction in their in vivo viability, as shown by the accumulation of cisplatin (FIGS. 12 F-H, and FIG. 28 E). However, this phenomenon was not observed in NK^{TROG+} cells from mice treated with NT-NK cells alone (FIG. 28 F), confirming the in vitro results that NK cell fratricide was antigen-specific and depended upon recognition and ligation of CAR with the TROG-antigen. In addition, when compared to the TROG⁻ CAR-NK cell population, TROG⁺ CAR-NK cells displayed higher expression of c-kit, Tbet, EOMES, activating adaptors, and cytolytic proteins (FIGS. 12 I-K), while also expressing checkpoint markers such as TIGIT, PD1, and TIM3 (FIG. 12 L), especially at late time points (exclusively in C4). These data showed that acquisition of TROG-antigen on CAR-NK cells was associated with activation first, followed by fratricide, functional exhaustion, and failure of NK cells to control the tumor in vivo.

At the mRNA level, B cell markers ((D)19, MSA41), were not detected in the NK cell clusters (defined by NKG7 and FCGR3A expression; FIG. 28 G). Taken together, these findings confirmed that TROG-antigen expression on CAR-NK cells is mediated by post-transcriptional transfer of protein from tumor targets that leads to activation, fratricide and eventual exhaustion of infused CAR-NK cells.

### Example 12

### Lower Car-Mediated Trogocytosis Favored the Therapeutic Efficacy of CAR-NK Cell Immunotherapy

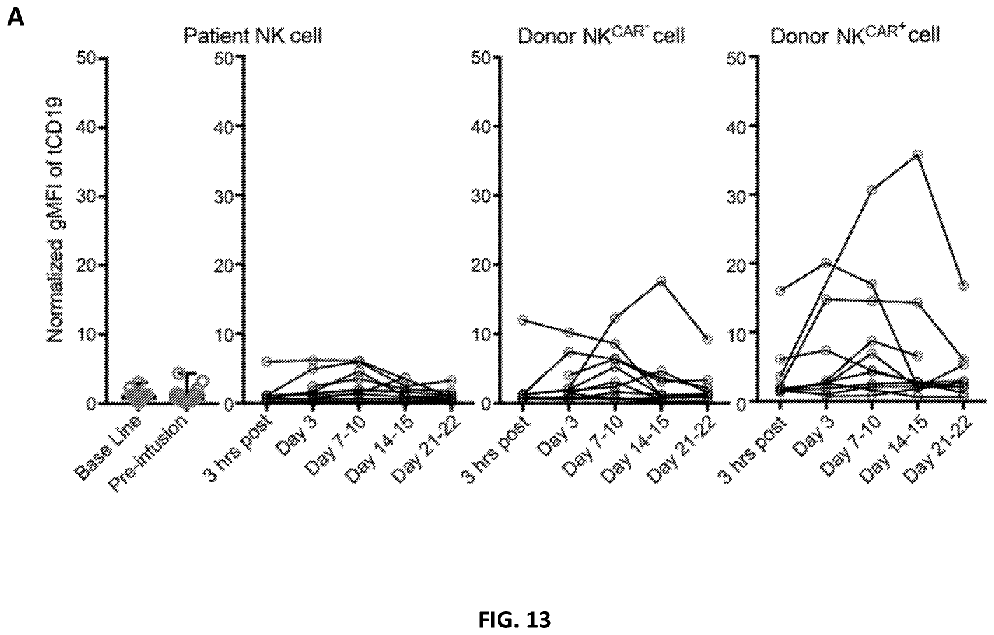

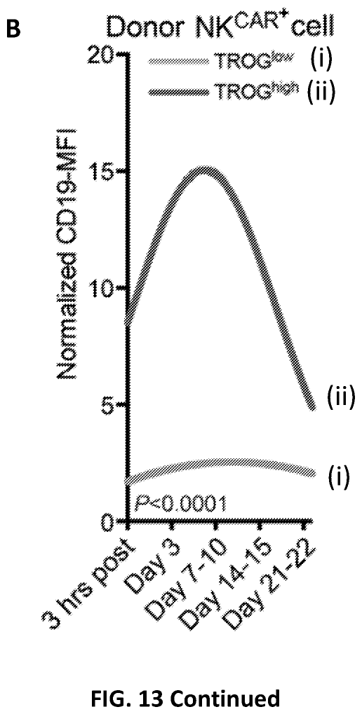

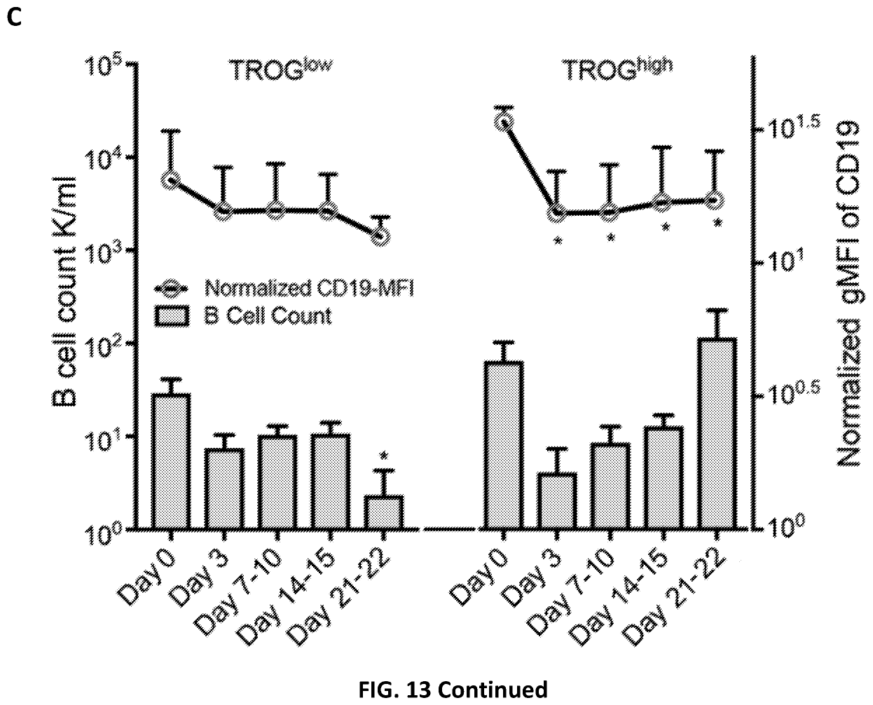

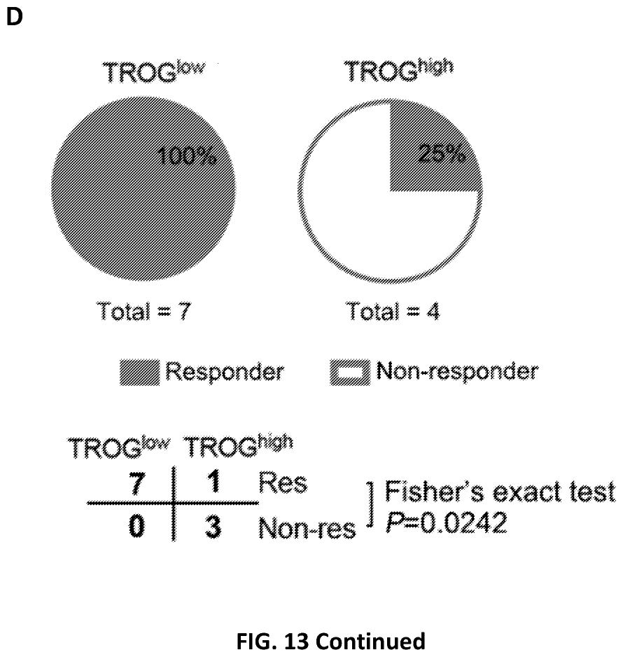

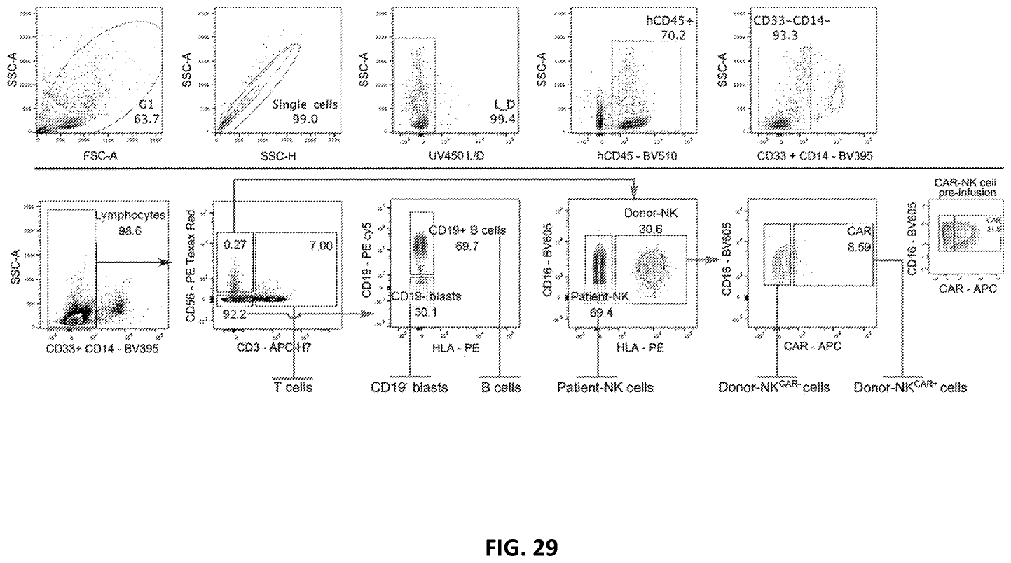

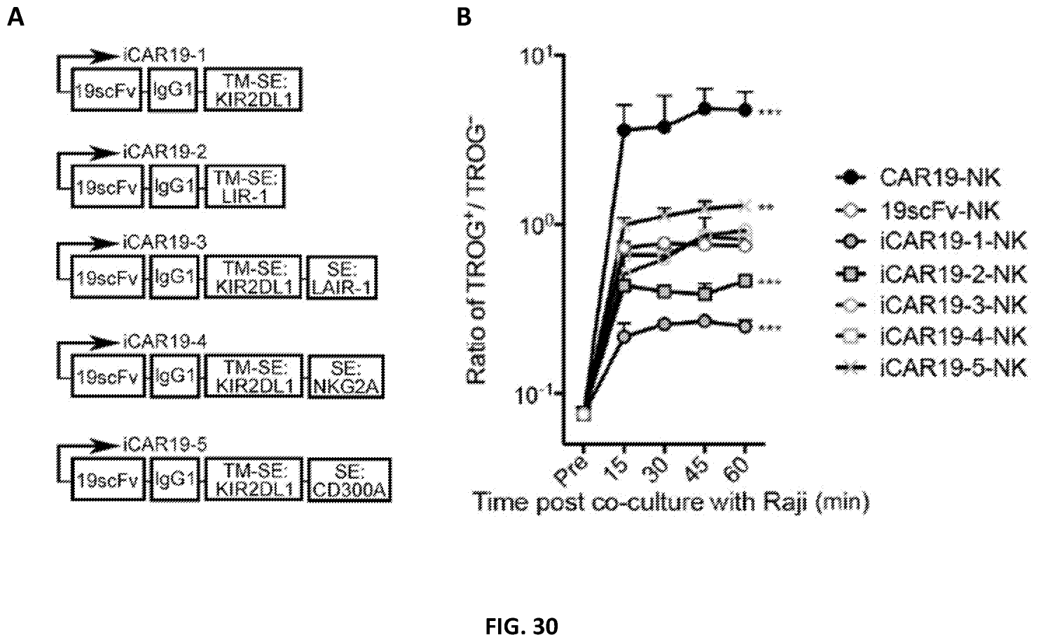

The contribution of trogocytosis to CAR-NK cell fratricide, and tumor progression in patients with lymphoid malignancies treated in the previously reported CAR-NK cell trial (NCT03056339) (see, e.g., Liu et al., 2020) was then determined. Using peripheral blood samples collected at multiple time points after CAR-NK cell infusion, patients were divided into two groups based on the overall tCD19 expression in CAR-NK cells (TROG^{high} (n=4) vs. TROG^{low} (n=7)); (FIGS. 13A and B, and FIG. 29). Acquisition of tCD19 expression on CAR-NK cells was associated with a reciprocal reduction in CD19 expression on B cells and a higher probability of relapse (3 of 4 patients) compared to patients in the TROG^{low} group (0 of 7 patients; FIGS. 13 C and D). Although this analysis was based on a small number of patients, the clinical observations further supported the unfavorable effect of CAR-mediated trogocytosis on the anti-tumor efficacy of CAR-NK cells.

### Example 13

An Inhibitory-CAR Against an NK-Specific Antigen Prevents Fratricide and Exhaustion Mediated by the on-Target Off-Tumor Effect of the Activating CAR

The dynamic balance of activating and inhibitory signals determines NK cell-mediated cytotoxicity and target lysis (see, e.g., Paul & Lal 2017; Wu & Lanier 2003; Bryceson & Long 2008; and Pegram et al., 2011). Thus, a genetic engineering strategy was utilized to exploit the physiological HLA class-I mediated NK cell inhibition in order to control NK cell activity in an antigen-specific manner (see, e.g., Elliott& Yokoyama 2011; Anfossi et al., 2006; and Bryceson & Long 2008). It was determined that an inhibitory CAR (iCAR), which incorporated an scFv directed to an NK-specific antigen linked to a powerful NK cell inhibitory signal, could limit NK cell responsiveness despite the simultaneous antigen-engagement of an activating CAR (aCAR), thus allowing for on-target/on-tumor recognition.

A series of iCAR constructs that fused an antigen-specific scFv with the transmembrane domain and immunoreceptor tyrosine-based inhibition motifs (ITIMs) derived from exemplary key NK cell inhibitory receptors, KIR2DL1 (see, e.g., Vivier et al., 2004), LIR-1 (see, e.g., Kirwan & Burshtyn 2005), CD300A (see, e.g., Zenarruzabeitia et al., 2015), NKG2A (see, e.g., Andre et al., 2018), and LAIR1 (see, e.g., Meyaard et al., 1997) were designed. As proof-of-principle, the inventors first tested an scFv specific for CD19 and confirmed that anti-CD19 iCARs (iCAR19s) were expressed on the surface of NK cells at similar levels to their aCAR-NK cell counterparts (CAR19: CD28/CD3ζ-based; FIGS. 30A and B, and FIG. 1). By culturing iCAR19-expressing NK cells with CD19⁺ targets and measuring the phosphorylation status of inhibitory signals vs. activating signals upon target engagement (see, e.g., Long et al., 2013; Bryceson & Long 2013; and Wu et al., 2021), the inventors determined if iCARs limited NK cell activity in an antigen-specific manner. Antigen stimulation resulted in higher phosphorylation of SHP1 in iCAR19-expressing NK cells (FIG. 3 A), without inducing phosphorylation in ITAM adapter proteins (Sky and Zap70; FIG. 3 B). Additionally, iCAR19-NK cells produced fewer effector cytokines with lower cytotoxicity against CD19⁺ targets (such as Raji cell, or autoNK^{gCD19+} cell), but not against CD19-negative targets such as K562 or CD19-KO Raji cells (FIGS. 4A-D, and FIGS. 5A-B). Of note, iCARs did not negatively impact the proliferation of NK cells cultured with an engineered K562 cell line (uAPC) and IL-2 (FIGS. 8A and 8B). Compared to the CD19-aCARs, trogocytosis was significantly less with iCARs, with the lowest level was observed when NK cells were transduced with an iCAR incorporating KIR2DL1 (iCAR1; FIG. 30 B). Thus, for subsequent experiments, the signaling endodomain for KIR2DL1 (iCAR1) was utilized.

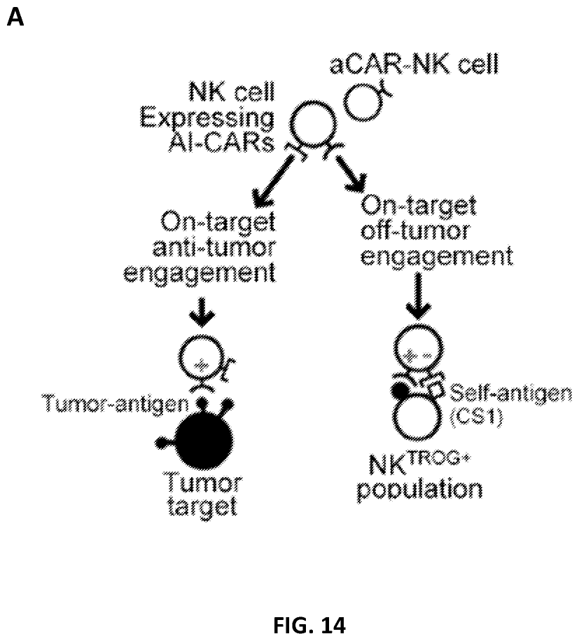

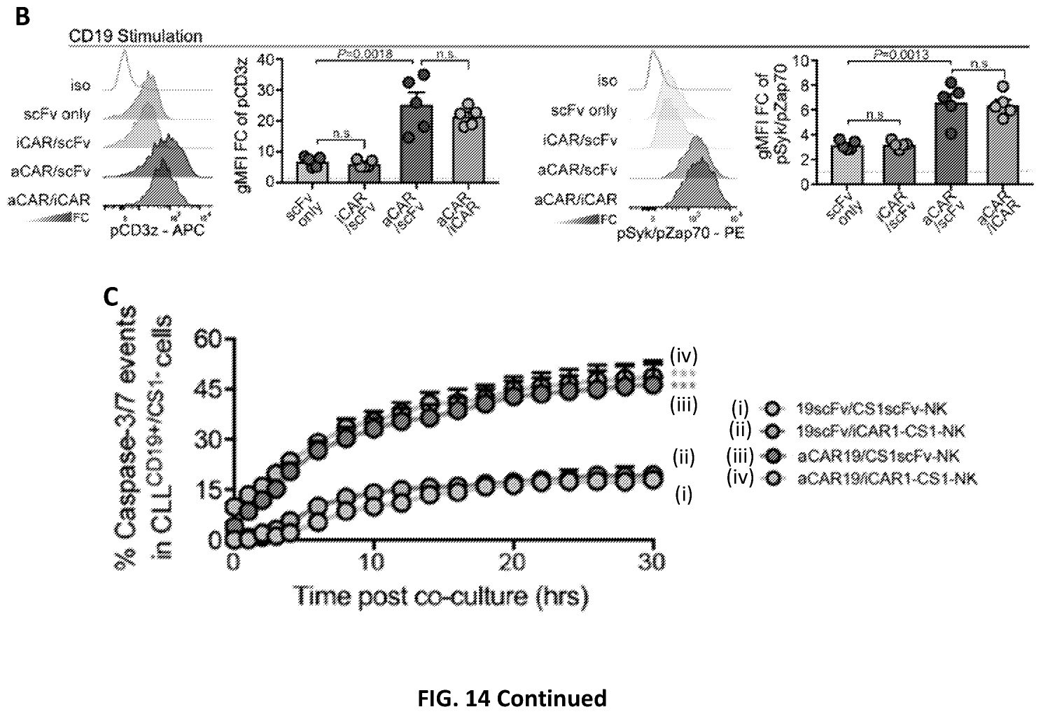

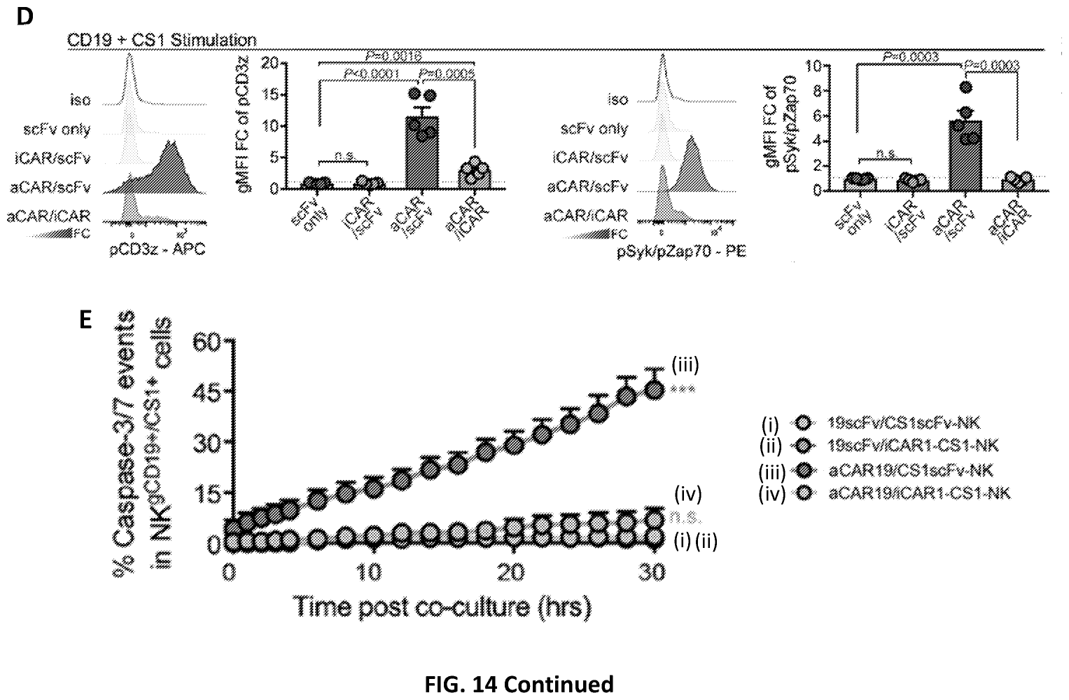

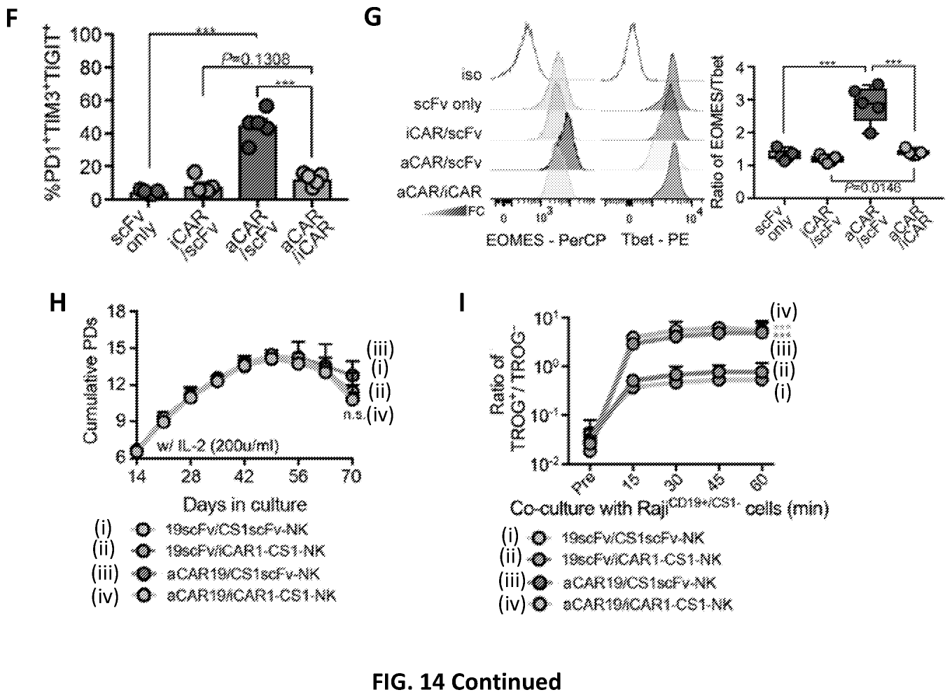

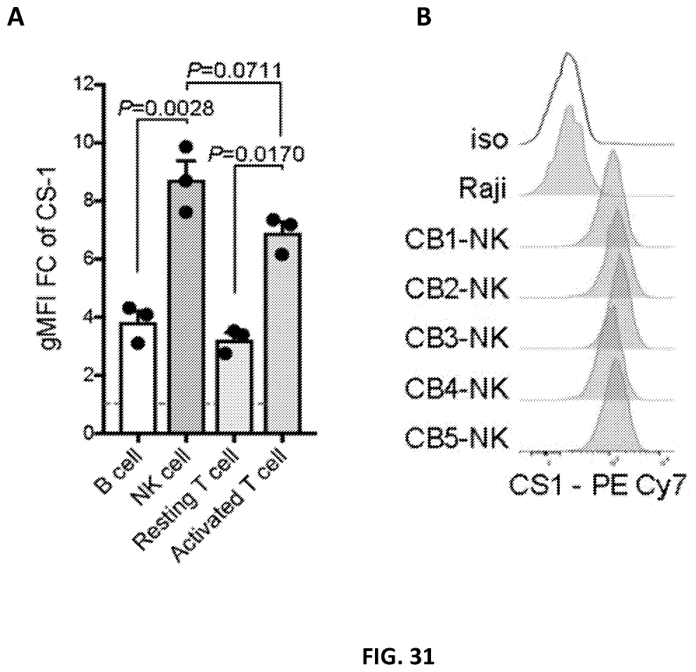

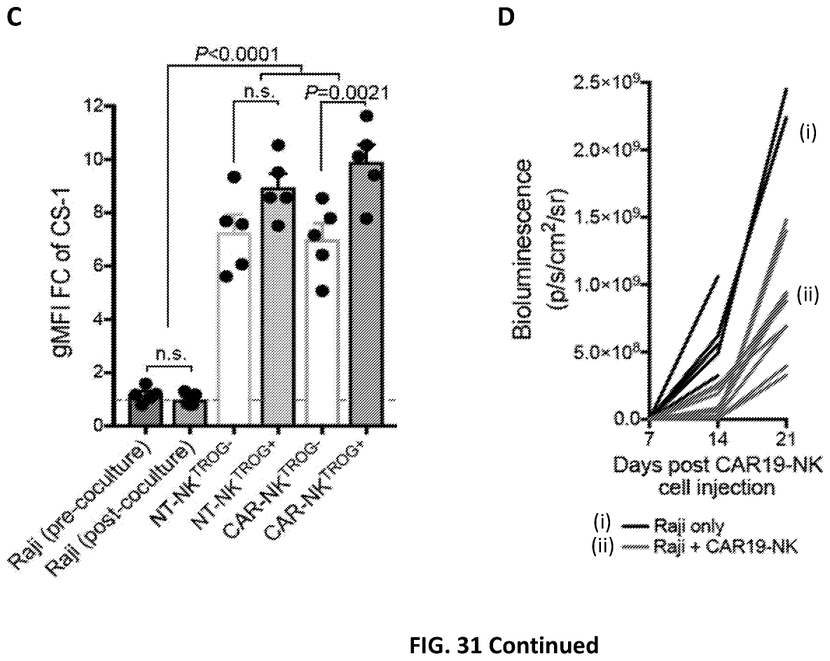

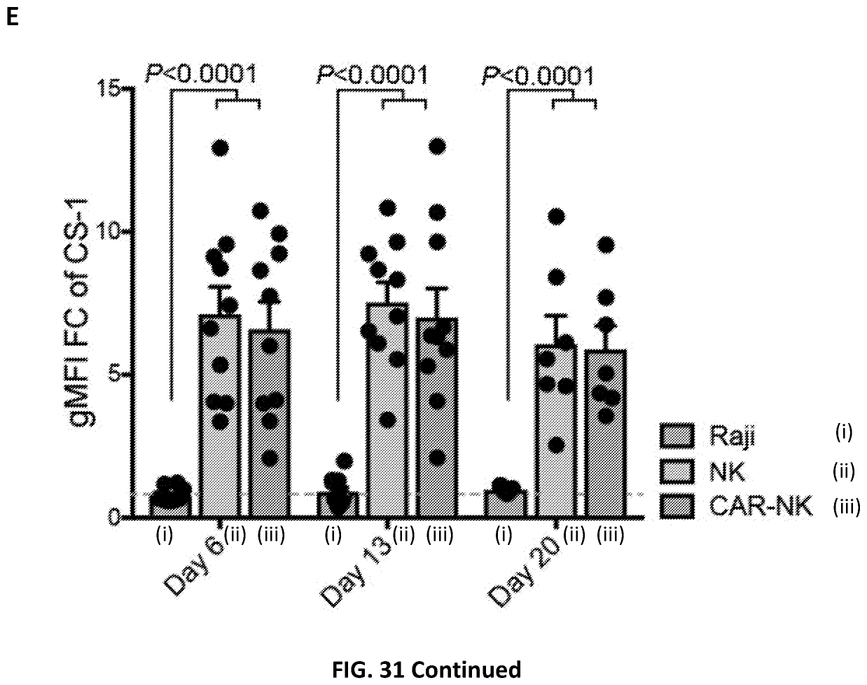

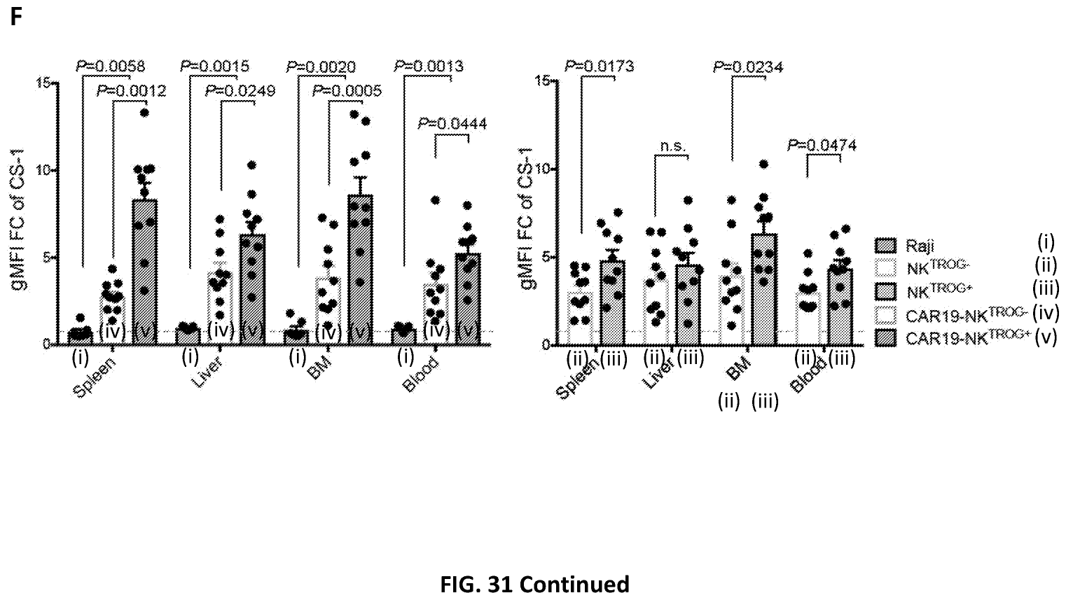

To determine if iCARs can inhibit the on-target off-tumor fratricide mediated through the aCAR, an iCAR was synthesized that recognized an NK self-antigen by fusing the transmembrane and intracellular domains of iCAR1 with an scFv targeting CS1, a co-receptor that is constitutively expressed on all normal NK cells (see, e.g., Tai et al., 2008; and Hsi et al., 2008), but absent on most CD19⁺ lymphoid-derived malignancies (see, e.g., Ma et al., 2018; Landau et al., 2013; and Lohr et al., 2012) (FIGS. 31A and B). The results confirmed that CS1 was expressed at high levels on NK^{TROG+} cells both in vitro and in vivo (FIGS. 31 B-F). Primary NK cells were transduced with the AI-CAR system (aCAR targeting CD19: aCAR19; and iCAR targeting CS1: iCAR1-CS1), and evaluated for their function against different targets (FIG. 2, and FIG. 14A). Controls included 19scFv and CS1-scFv without activating or inhibitory signaling endodomains, respectively. iCAR-CS1 expression spared the on-target anti-tumor activity of aCAR19 against CD19⁺ tumor targets (FIG. 14 B), including primary CLL samples from patients (FIG. 14 C and FIG. 7A), but inhibited the activity of aCAR-NK cells when both CD19 and CS1 were expressed on the target (FIG. 14 D, and FIG. 7 C). The ability of iCAR to restrict aCAR-mediated cytotoxicity against autoNK^{gCD19+/CS1+} cells was then investigated. NK cells expressing the AI-CARs displayed marked reduction in cytotoxicity and fratricide against autoNK^{gCD19+/CS1+} cells (FIG. 14 E, and FIG. 7 B), but maintained their effector function and cytokine response to CD19+CS1− target cells (FIGS. 7 D and E) with no evidence of exhaustion (FIGS. 14 F and G), or significant impairment in their in vitro proliferation capacity (FIG. 14 H). Notably, iCAR expression had little impact on aCAR-mediated cognate-antigen trogocytosis in co-cultures of NK cells with CD19 CS1-Raji targets (FIG. 14 I). These findings confirmed that the CS1-targeting iCAR selectively prevented NK cells from antigen-induced on-tumor/off-target effect, while preserving the therapeutic response of the aCAR against “on-target” CD19⁺ tumor cells.

### Example 14

### AI-CARS Limit In Vivo Fratricide, and Improve the Persistence and Anti-Tumor Activity of Nk Cells

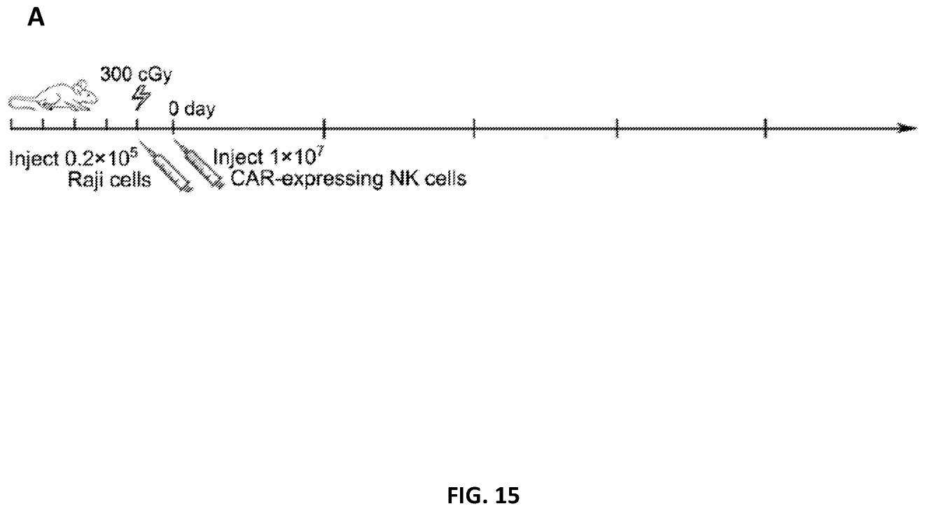

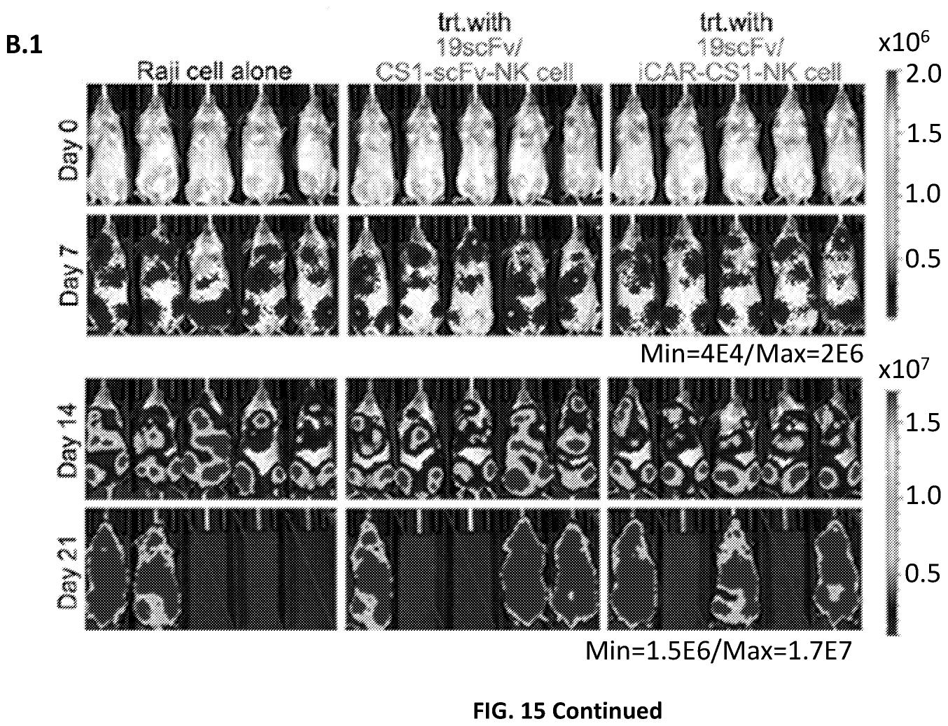

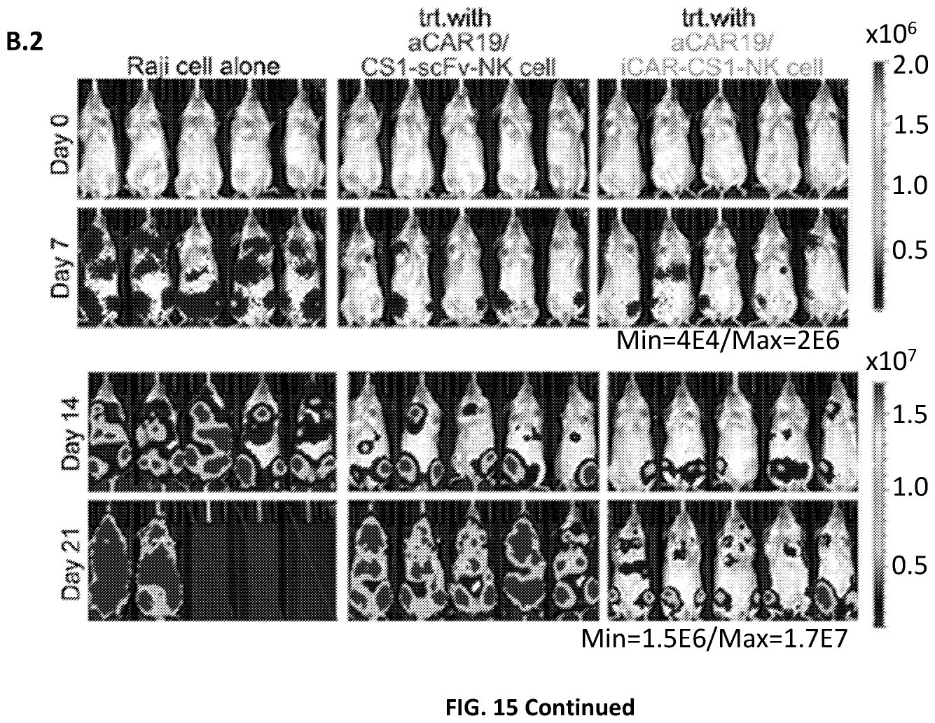

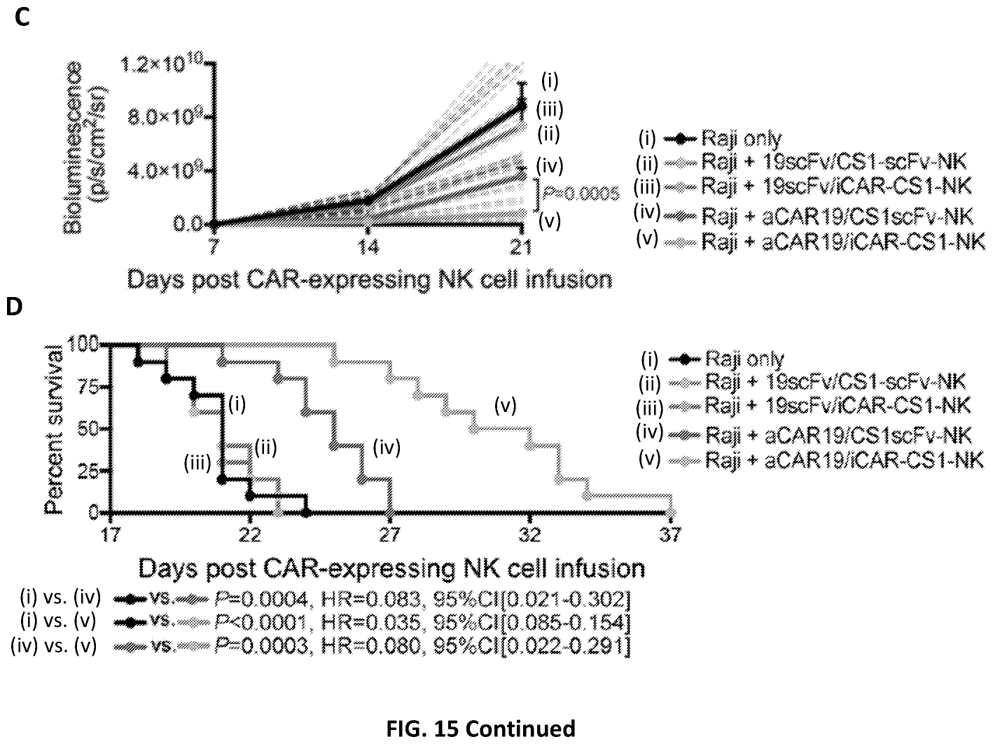

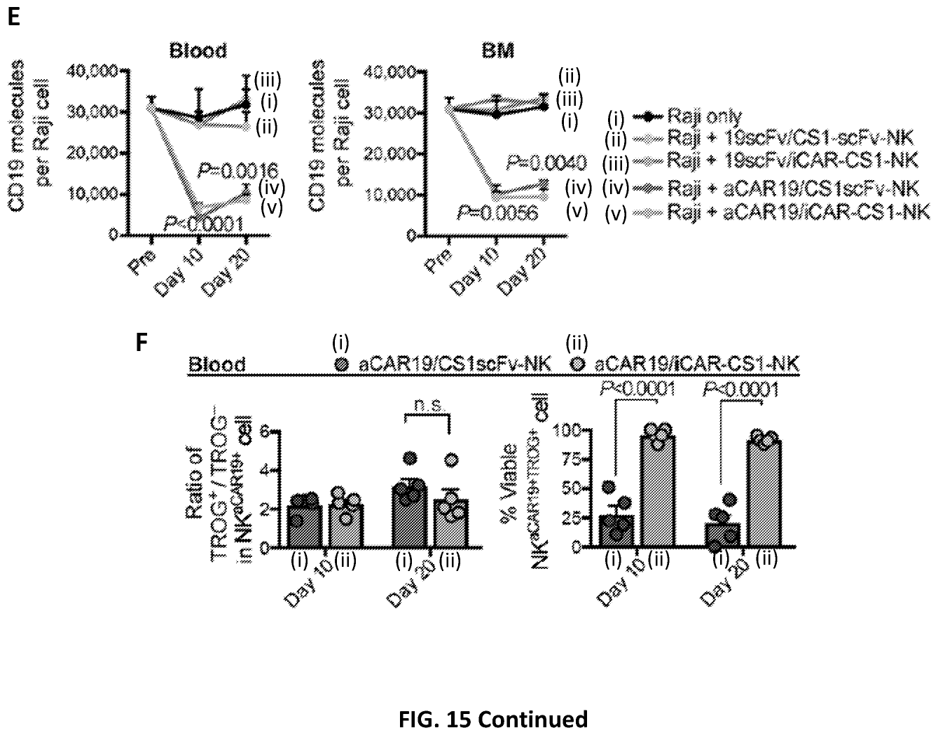

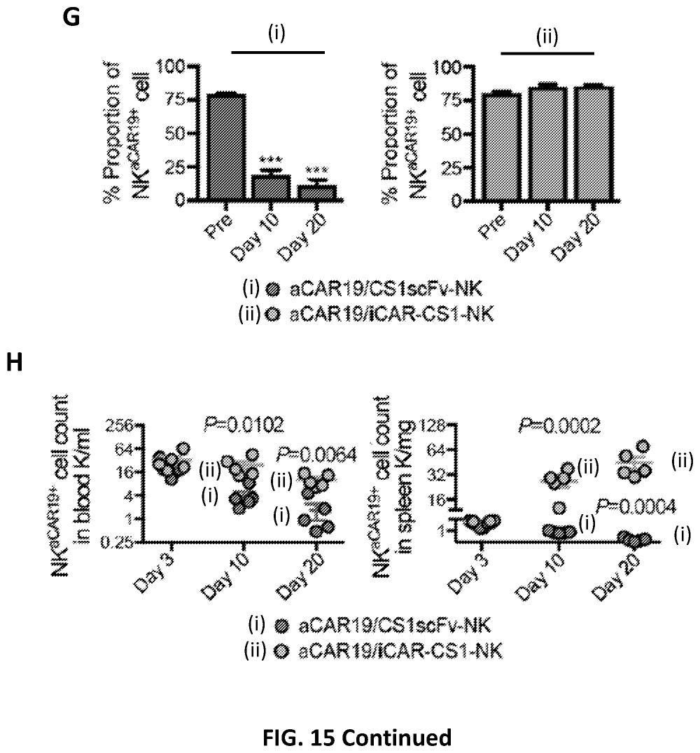

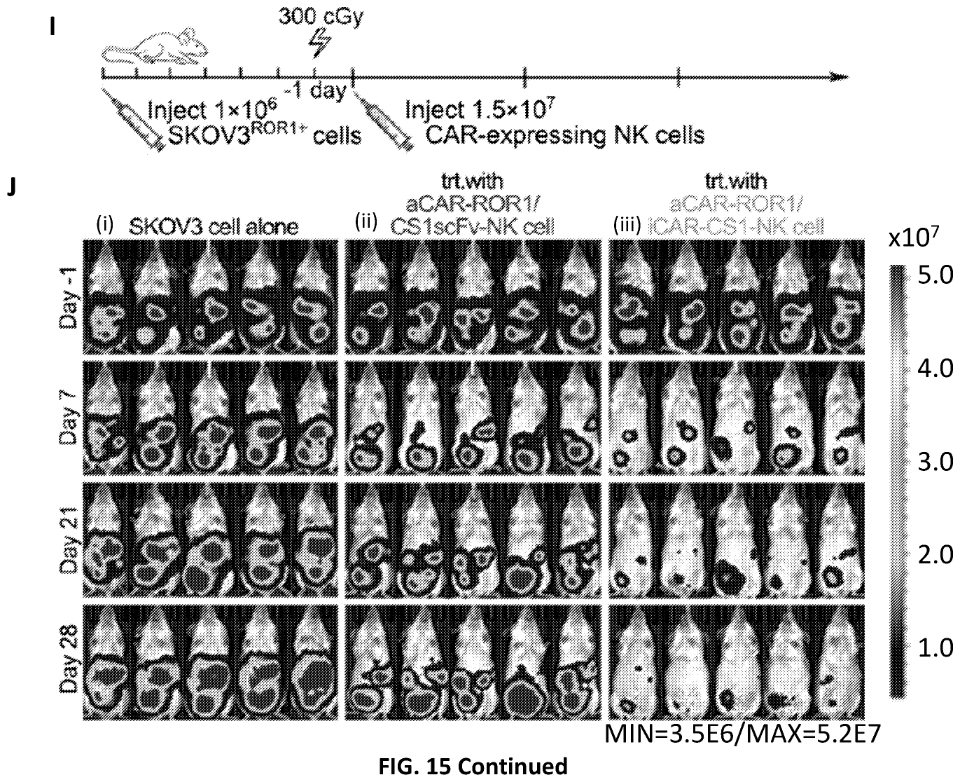

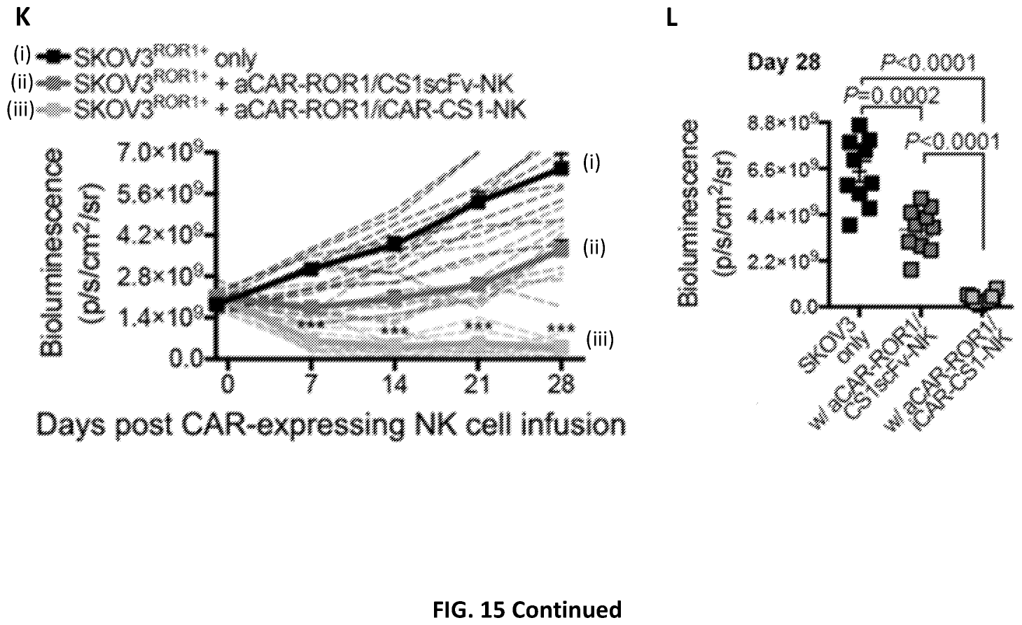

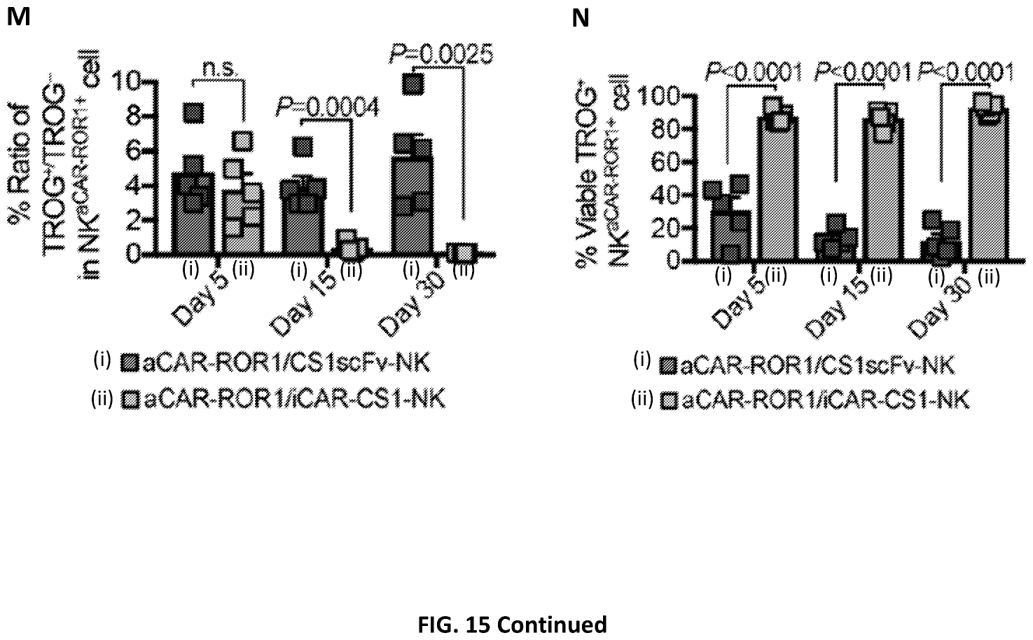

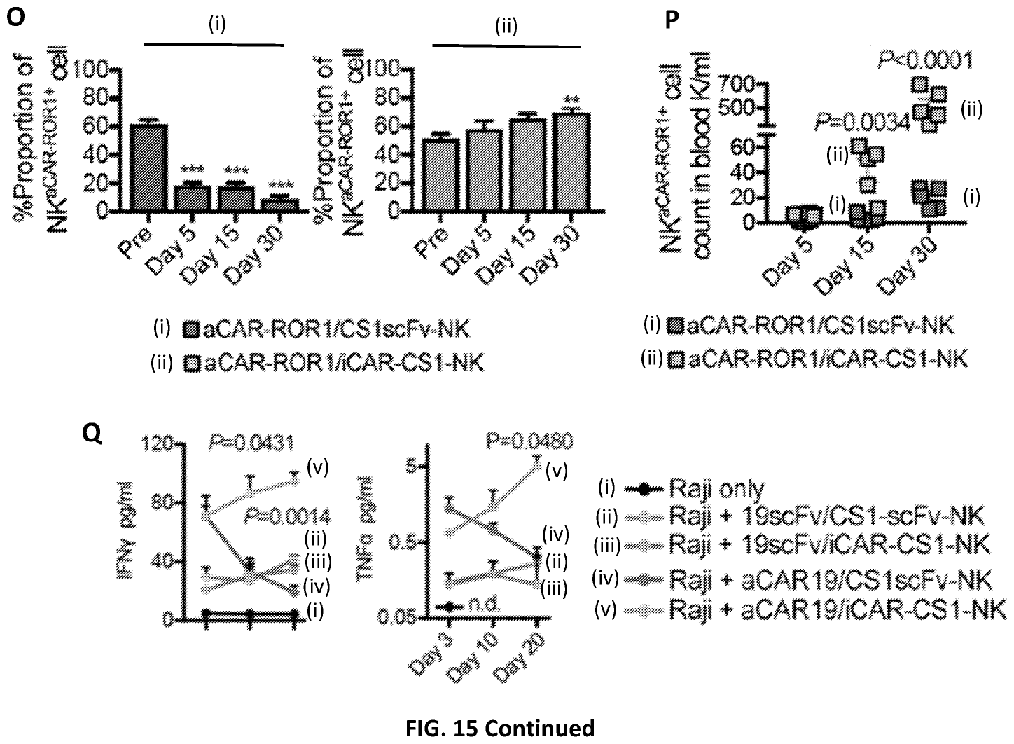

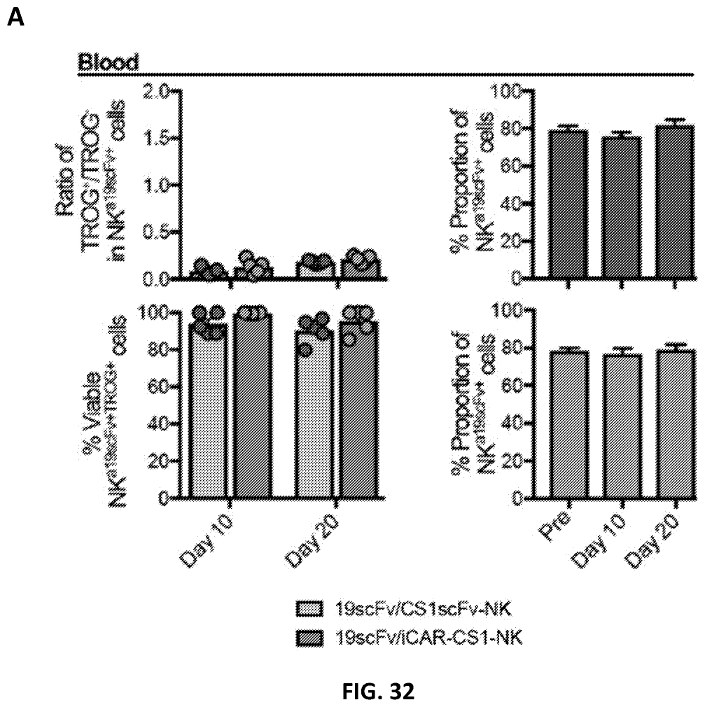

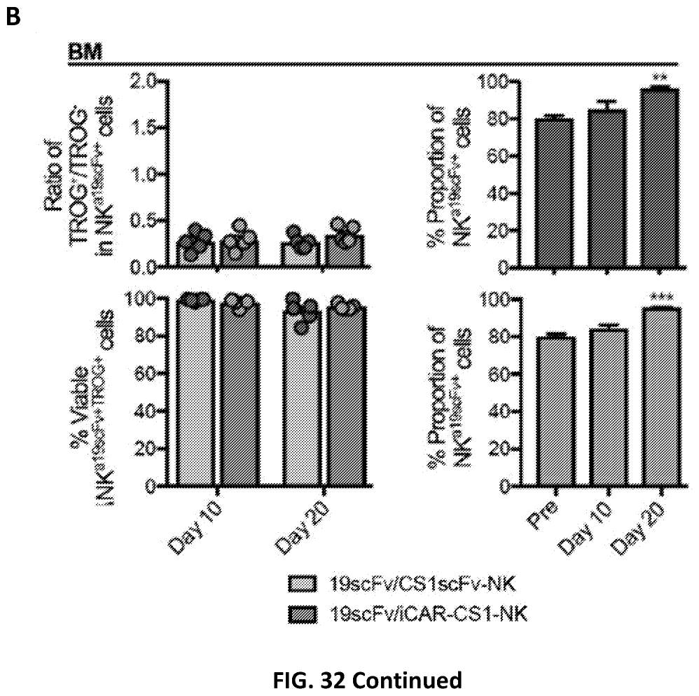

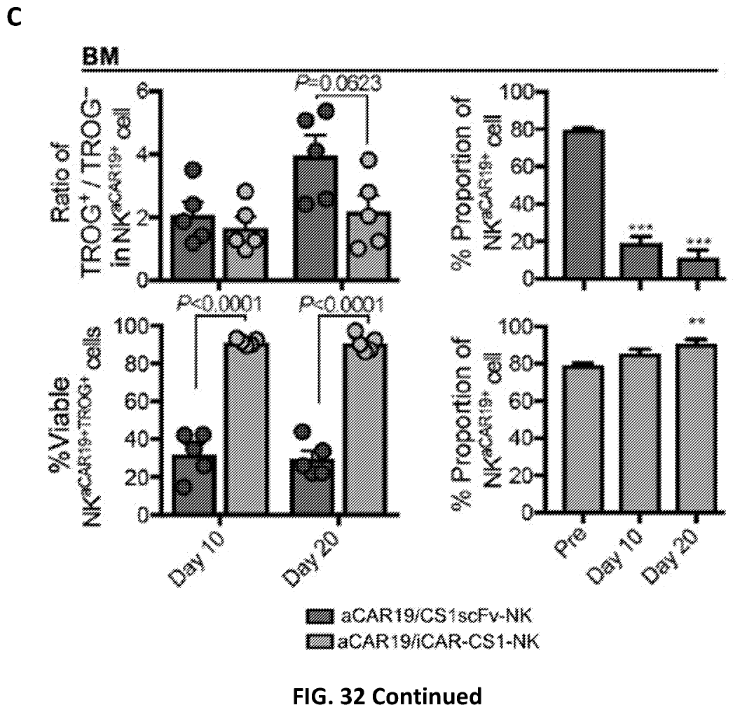

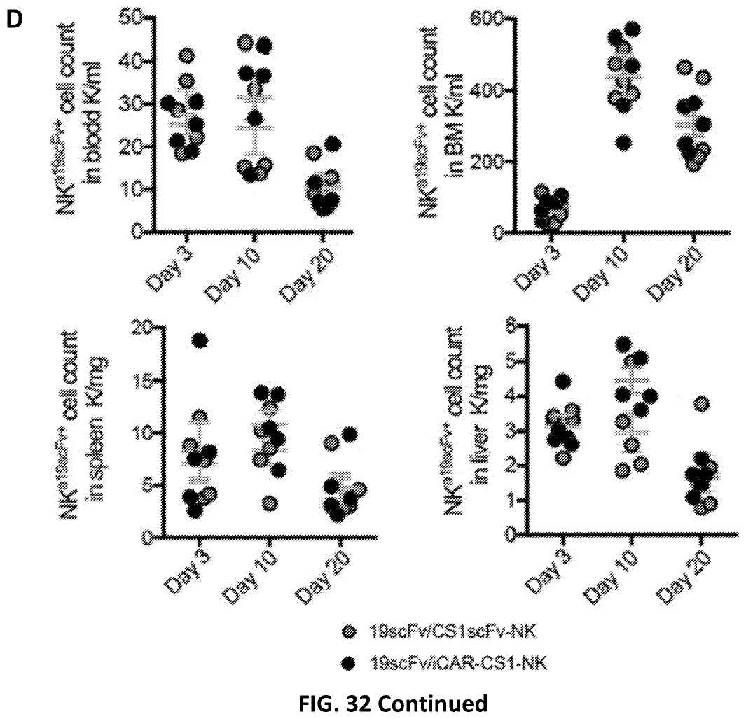

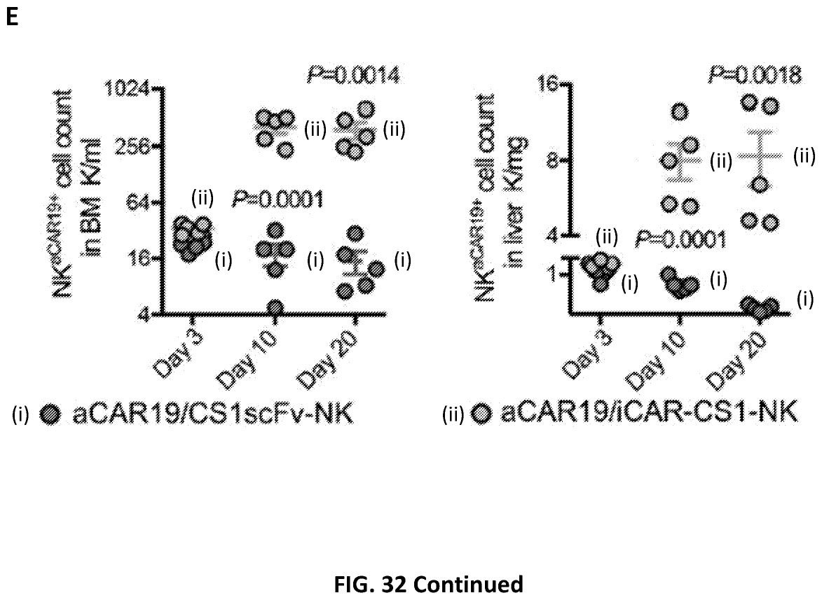

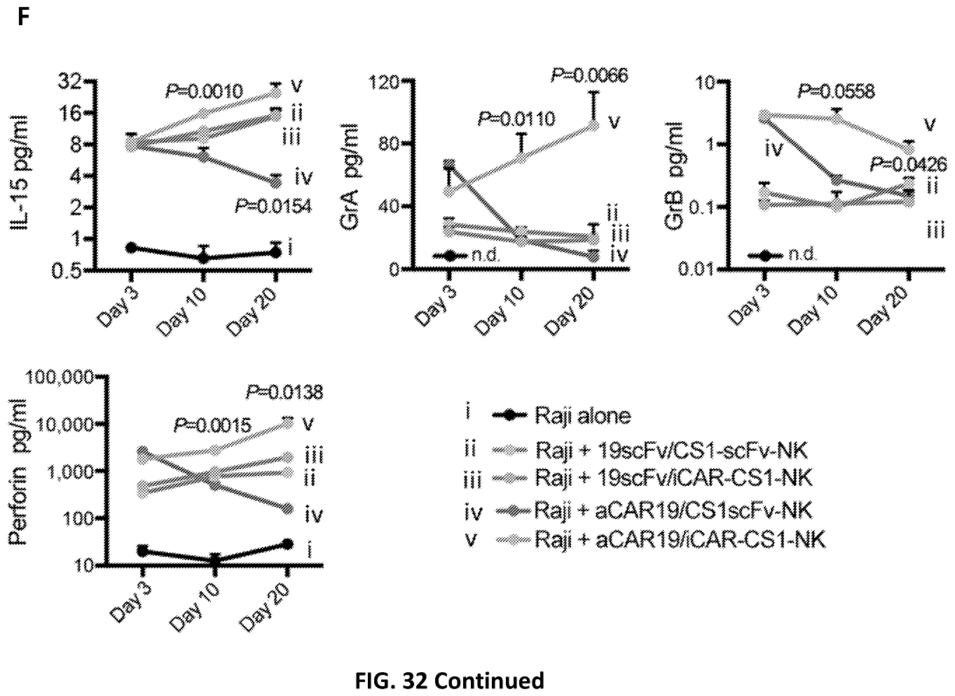

To investigate whether AI-CAR-NK cells could protect NK cells from aCAR-mediated fratricide and exhaustion in vivo, the well-established NOD/SCID/gamma-null (NSG) mouse model with Raji tumor (see, e.g., Liu et al., 2018; and Daher et al., 2021(b)) was utilized. Mice received one dose of 1×10⁷ NK cells expressing anti-CD19 aCAR/anti-CS1 iCAR1, with controls of NK cell expressing anti-CD19 scFv/anti-CS1 scFv, anti-CD19 scFv/anti-CS1 iCAR1, or anti-CD19 aCAR/anti-CS1 scFv (FIG. 15A). The anti-CD19 aCAR/anti-CS1 iCAR1-NK cells mediated the strongest anti-tumor response with a significantly lower bioluminescence imaging (BLI) signal and better overall survival compared to the control groups (FIGS. 15 B-D). To confirm that the improved in vivo anti-tumor response was mediated through a decrease in TROG-antigen induced fratricide and improved effector function by AI-CAR-NK cells, in a parallel experiment, mice were sacrificed at serial time points and blood and organs were collected for NK cell profiling. While AI-CAR-NK cells did not impact the extent of aCAR-mediated trogocytosis (FIG. 15 E), less trogocytosis-mediated in vivo fratricide was observed, as evidenced by the better viability, higher persistence (FIGS. 15 F-H, and FIGS. 32A-E), and improved effector function of AI-CAR-NK cells when compared with control groups (FIG. 15 Q, and FIG. 32 F).

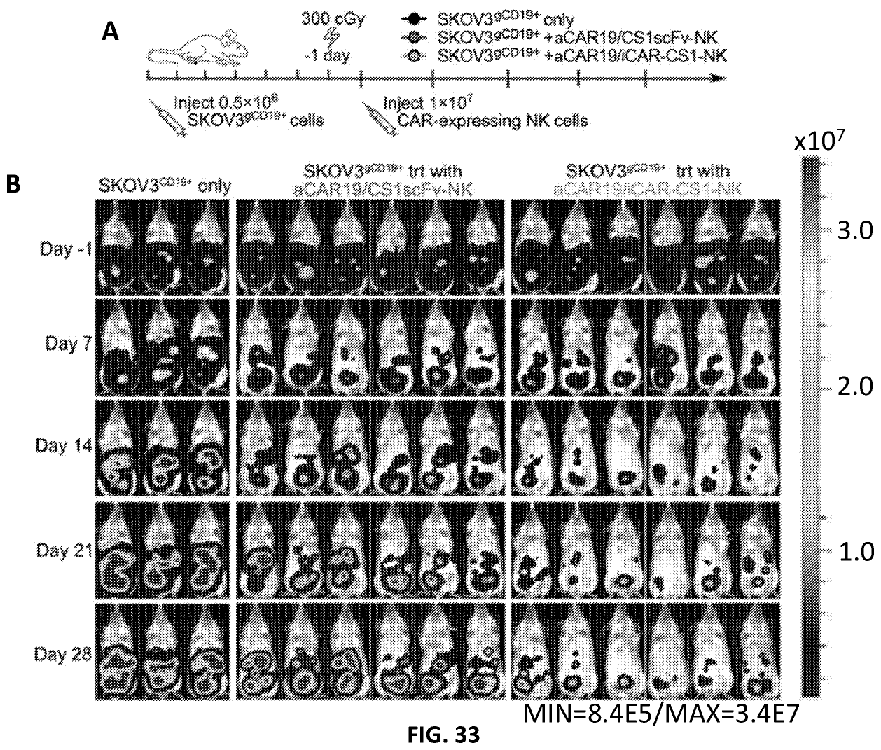

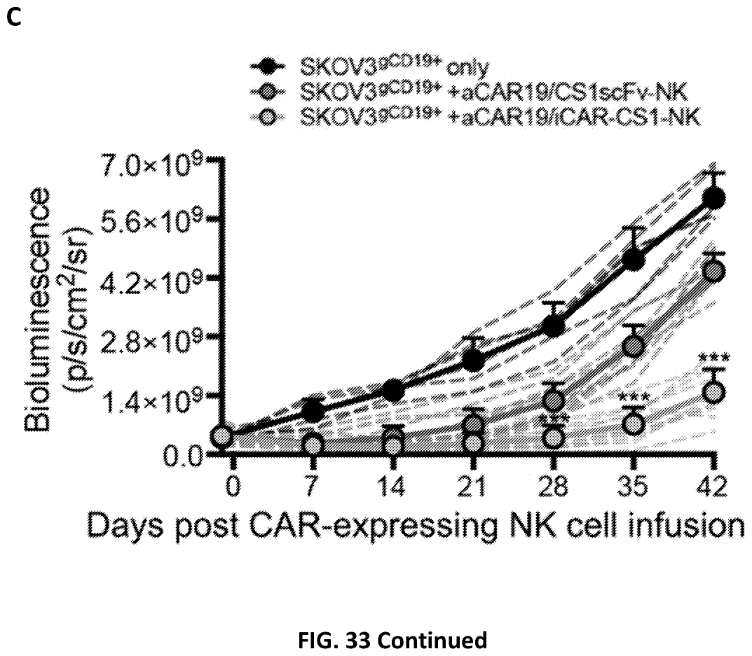

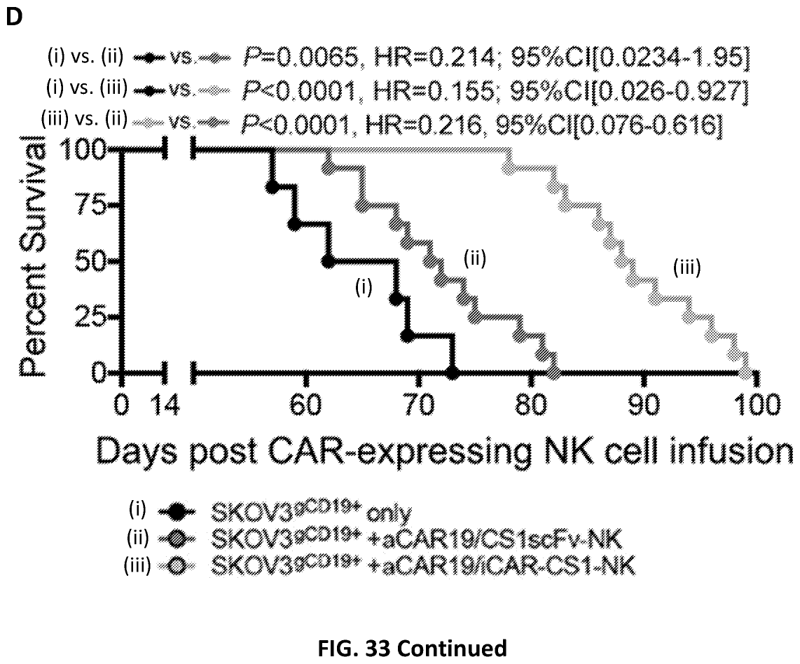

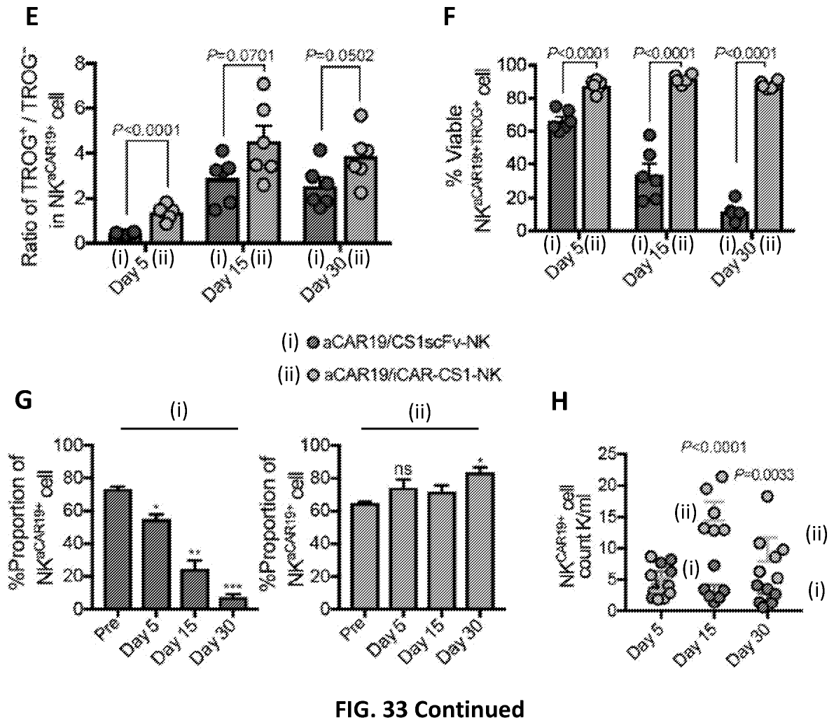

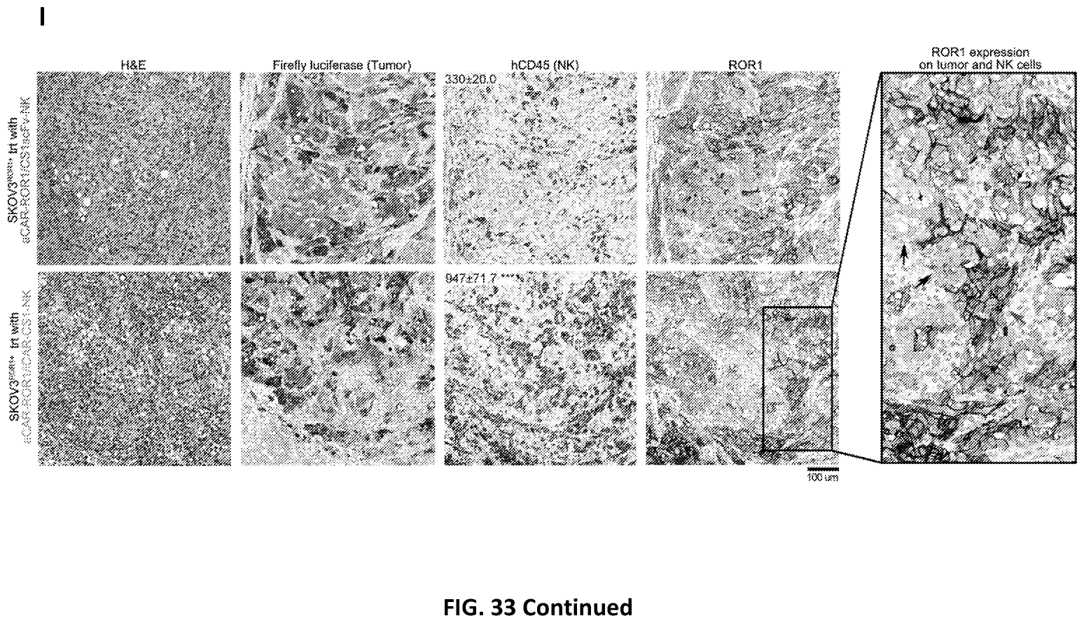

A solid tumor model of ovarian cancer was also tested. SKOV3 cells that were endogenously expressing ROR1, or genetically engineered to express CD19 (SKOV3^{gCD19+}) were intraperitoneally injected into NSG mice, followed by a single infusion of NK cells co-expressing antigen-specific AI-CARs or the relevant scFv controls (FIG. 15 I, and FIG. 33A). Again, superior anti-tumor control with AI-CAR-NK cells was observed (FIGS. 15 J-L, and FIGS. 33 B-D). This superior anti-tumor control was associated with improved NK cell viability, persistence, and infiltration within the tumor microenvironment with variable expression of the ROR1 TROG-antigen on their surface (FIGS. 15 M-P, and FIGS. 33 E-I). Taken together, these results provided evidence that iCAR limit the aCAR response to the TROG-antigen on NK cells in an antigen-specific fashion in vivo, reducing fratricide, preserving NK cell effector function, and NK cell persistence, but without abrogating the aCAR response to the tumor target, thus, resulting in improved NK cell anti-tumor activity.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### REFERENCES

Ahmed, K. A., Munegowda, M. A., Xie, Y. & Xiang, J. Intercellular trogocytosis plays an important role in modulation of immune responses. Cell Mol Immunol 5, 261-269 (2008).
Alhajjat, A. M., et al. Trogocytosis as a mechanistic link between chimerism and prenatal tolerance. Chimerism 4, 126-131 (2013).
Anderson, A. C., Joller, N. & Kuchroo, V. K. Lag-3, Tim-3, and TIGIT: Co-inhibitory Receptors with Specialized Functions in Immune Regulation. Immunity 44, 989-1004 (2016).
Andre, P., et al. Anti-NKG2A mAb Is a Checkpoint Inhibitor that Promotes Anti-tumor Immunity by Unleashing Both T and NK Cells. Cell 175, 1731-1743 e1713 (2018).
Andreas, A., Christophe, C. & Sarah, G. Sparse least trimmed squares regression for analyzing high-dimensional large data sets. The Annals of Applied Statistics 7, 226-248 (2013).
Anfossi, N., et al. Human NK cell education by inhibitory receptors for MHC class I. Immunity 25, 331-342 (2006).
Ausubel et al., Current Protocols in Molecular Biology, Greene Publishing Associates and John Wiley & Sons, N Y, 1996.
Bennekov T, Spector D, Langhoff E. Induction of immunity against human cytomegalovirus. Mt Sinai J Med. 2004 March; 71(2):86-93.
Bettadapur, A., Miller, H. W. & Ralston, K. S. Biting Off What Can Be Chewed: Trogocytosis in Health, Infection, and Disease. Infect Immun 88(2020).
Beum, P. V., Lindorfer, M. A. & Taylor, R. P. Within peripheral blood mononuclear cells, antibody-dependent cellular cytotoxicity of rituximab-opsonized Daudi cells is promoted by NK cells and inhibited by monocytes due to shaving. J Immunol 181, 2916-2924 (2008).
Brentjens, R. J., et al. Safety and persistence of adoptively transferred autologous CD19-targeted T cells in patients with relapsed or chemotherapy refractory B-cell leukemias. Blood 118, 4817-4828 (2011).
Brentjens, R. J., et al. CD19-targeted T cells rapidly induce molecular remissions in adults with chemotherapy-refractory acute lymphoblastic leukemia. Sci Transl Med 5, 177ra138 (2013).
Bryceson, Y. T. & Long, E. O. Line of attack: NK cell specificity and integration of signals. Curr Opin Immunol 20, 344-352 (2008).
Cameron, B. J., et al. Identification of a Titin-derived HLA-A1-presented peptide as a cross-reactive target for engineered MAGE A3-directed T cells. Sci Transl Med 5, 197ra103 (2013).
Carlsten, M., et al. Checkpoint Inhibition of KIR2D with the Monoclonal Antibody IPH2101 Induces Contraction and Hyporesponsiveness of NK Cells in Patients with Myeloma. Clin Cancer Res 22, 5211-5222 (2016).
Caumartin, J., et al. Trogocytosis-based generation of suppressive NK cells. EMBO J 26, 1423-1433 (2007).
Cheng, M., Chen, Y., Xiao, W., Sun, R. & Tian, Z. NK cell-based immunotherapy for malignant diseases. Cell Mol Immunol 10, 230-252 (2013).
Chothia et al., The outline structure of the T-cell αβ receptor. EMBO J. 7:3745-3755, 1988.
Cohen et al., Recognition of fresh human tumor by human peripheral blood lymphocytes transduced with a bicistronic retroviral vector encoding a murine anti-p53 TCR. J Immunol. 175:5799-5808, 2005.
Daher, M., Melo Garcia, L., Li, Y. & Rezvani, K. CAR-NK cells: the next wave of cellular therapy for cancer. Clin Transl Immunology 10, e1274 (2021). (Daher et al., 2021(a))
Daher, M., et al. Targeting a cytokine checkpoint enhances the fitness of armored cord blood CAR-NK cells. Blood 137, 624-636 (2021). (Daher et al., 2021(b))
Dance, A. Core Concept: Cells nibble one another via the under-appreciated process of trogocytosis. Proc Natl Acad Sci USA 116, 17608-17610 (2019).
Davila et al. CD19 CAR-targeted T cells induce long-term remission and B Cell Aplasia in an immunocompetent mouse model of B cell acute lymphoblastic leukemia. PLOS ONE 8(4): e61338, 2013.
Domaica, C. I., et al. Tumour-experienced T cells promote NK cell activity through trogocytosis of NKG2D and NKp46 ligands. EMBO Rep 10, 908-915 (2009).
Du, X., Ho, M. & Pastan, I. New immunotoxins targeting CD123, a stem cell antigen on acute myeloid leukemia cells. J Immunother 30, 607-613 (2007).
Elliott, J. M. & Yokoyama, W. M. Unifying concepts of MHC-dependent natural killer cell education. Trends Immunol 32, 364-372 (2011).
Fang, F., Xiao, W. & Tian, Z. NK cell-based immunotherapy for cancer. Semin Immunol 31, 37-54 (2017).
Fedorov V D, Themeli M, Sadelain M. PD-1- and CTLA-4-based inhibitory chimeric antigen receptors (iCARs) divert off-target immunotherapy responses. Sci Transl Med. 2013 Dec. 11; 5(215):215ra172.
Ford Mcintyre, M. S., Young, K. J., Gao, J., Joe, B. & Zhang, L. Cutting edge: in vivo trogocytosis as a mechanism of double negative regulatory T cell-mediated antigen-specific suppression. J Immunol 181, 2271-2275 (2008).
Frolet C, Beniazza M, Roux L, et al. New adhesin functions of surface-exposed pneumococcal proteins. BMC Microbiol. 2010; 10:190. Published 2010 Jul. 12.
Gardiner, C. M. & Finlay, D. K. What Fuels Natural Killers? Metabolism and NK Cell Responses. Front Immunol 8, 367 (2017).
Gill, S., et al. Rapid development of exhaustion and down-regulation of eomesodermin limit the antitumor activity of adoptively transferred murine natural killer cells. Blood 119, 5758-5768 (2012).
Griffiths, G. M., Tsun, A. & Stinchcombe, J. C. The immunological synapse: a focal point for endocytosis and exocytosis. J Cell Biol 189, 399-406 (2010).
Haas, A. R., et al. Phase I Study of Lentiviral-Transduced Chimeric Antigen Receptor-Modified T Cells Recognizing Mesothelin in Advanced Solid Cancers. Mol Ther 27, 1919-1929 (2019).
Hamieh, M., et al. CAR T cell trogocytosis and cooperative killing regulate tumour antigen escape. Nature 568, 112-116 (2019).
Heemskerk et al. Adoptive cell therapy for patients with melanoma, using tumor-infiltrating lymphocytes genetically engineered t secrete interleukin-2. Hum Gene Ther. 19:496-510, 2008
Hegde, M., et al. Tumor response and endogenous immune reactivity after administration of HER2 CAR T cells in a child with metastatic rhabdomyosarcoma. Nat Commun 11, 3549 (2020).
Hochreiter-Hufford, A. & Ravichandran, K. S. Clearing the dead: apoptotic cell sensing, recognition, engulfment, and digestion. Cold Spring Harb Perspect Biol 5, a008748 (2013).
Hoyos, V., et al. Engineering CD19-specific T lymphocytes with interleukin-15 and a suicide gene to enhance their anti-lymphoma/leukemia effects and safety. Leukemia 24, 1160-1170 (2010).
Hsi, E. D., et al. CS1, a potential new therapeutic antibody target for the treatment of multiple myeloma. Clin Cancer Res 14, 2775-2784 (2008).
Hudrisier, D., Aucher, A., Puaux, A. L., Bordier, C. & Joly, E. Capture of target cell membrane components via trogocytosis is triggered by a selected set of surface molecules on T or B cells. J Immunol 178, 3637-3647 (2007).
Janeway et al., Immunobiology: The Immune System in Health and Disease, 3^{rd} Ed., Current Biology Publications, p. 433, 1997.
Johnson et al. Gene therapy with human and mouse T-cell receptors mediates cancer regression and targets normal tissues expressing conate antigen. Blood 114:535-46, 2009.
Joly, E. & Hudrisier, D. What is trogocytosis and what is its purpose? Nat Immunol 4, 815 (2003).
Jores et al., PNAS U.S.A. 87:9138-9142, 1990
Judge, S. J., Murphy, W. J. & Canter, R. J. Characterizing the Dysfunctional NK Cell: Assessing the Clinical Relevance of Exhaustion, Anergy, and Senescence. Front Cell Infect Microbiol 10, 49 (2020).
June, C. H. & Sadelain, M. Chimeric Antigen Receptor Therapy. N Engl J Med 379, 64-73 (2018).
Kabat et al., “Sequences of Proteins of Immunological Interest, US Dept. Health and Human Services, Public Health Service National Institutes of Health, 1991, 5th ed.
Kalluri, R. & LeBleu, V. S. The biology, function, and biomedical applications of exosomes. Science 367(2020).
Kalos, M., et al. T cells with chimeric antigen receptors have potent antitumor effects and can establish memory in patients with advanced leukemia. Sci Transl Med 3, 95ra73 (2011).
Kieback, E., Charo, J., Sommermeyer, D., Blankenstein, T. & Uckert, W. A safeguard eliminates T cell receptor gene-modified autoreactive T cells after adoptive transfer. Proc Natl Acad Sci USA 105, 623-628 (2008).
Kirwan, S. E. & Burshtyn, D. N. Killer cell Ig-like receptor-dependent signaling by Ig-like transcript 2 (ILT2/CD85j/LILRB1/LIR-1). J Immunol 175, 5006-5015 (2005).
Koehl, U., et al. Advances in clinical NK cell studies: Donor selection, manufacturing and quality control. Oncoimmunology 5, e1115178 (2016).
Landau, D. A., et al. Evolution and impact of subclonal mutations in chronic lymphocytic leukemia. Cell 152, 714-726 (2013).
Lefranc et al., IMGT unique numbering for immunoglobuling and T cell receptor variable domains and Ig superfamily V-like domains. Dev. Comp. Immunol. 27:55, 2003.
Li, et al., Directed evolution of human T-cell receptors with picomolar affinities by phage display. Nat Biotechnol. 2005 March; 23(3):349-542005.
Li, Y., Hermanson, D. L., Moriarity, B. S. & Kaufman, D. S. Human iPSC-Derived Natural Killer Cells Engineered with Chimeric Antigen Receptors Enhance Anti-tumor Activity. Cell Stem Cell 23, 181-192 e185 (2018).
Li, L., et al. A novel immature natural killer cell subpopulation predicts relapse after cord blood transplantation. Blood Adv 3, 4117-4130 (2019).
Liadi, I., et al. Individual Motile CD4(+) T Cells Can Participate in Efficient Multikilling through Conjugation to Multiple Tumor Cells. Cancer Immunol Res 3, 473-482 (2015).
Linette, G. P., et al. Cardiovascular toxicity and titin cross-reactivity of affinity-enhanced T cells in myeloma and melanoma. Blood 122, 863-871 (2013).
Linnemann C, van Buuren M M, Bies L, Verdegaal E M, Schotte R, Calis J J, Behjati S, Velds A, Hilkmann H, Atmioui D E, Visser M, Stratton M R, Haanen J B, Spits H, van der Burg S H, Schumacher T N. High-throughput epitope discovery reveals frequent recognition of neo-antigens by CD4+ T cells in human melanoma. Nat Med. 2015 January; 21(1):81-5. Epub 2014 Dec. 22. Erratum in: Nat Med. 2016 Oct. 6; 22(10):1192.
Liu, E., et al. Cord blood NK cells engineered to express IL-15 and a CD19-targeted CAR show long-term persistence and potent antitumor activity. Leukemia 32, 520-531 (2018).
Liu, E., et al. Use of CAR-Transduced Natural Killer Cells in CD19-Positive Lymphoid Tumors. N Engl J Med 382, 545-553 (2020).
Liu, E., et al. GMP-Compliant Universal Antigen Presenting Cells (uAPC) Promote the Metabolic Fitness and Antitumor Activity of Armored Cord Blood CAR-NK Cells. Front Immunol 12, 626098 (2021).
Lockey et al., Epstein-Barr virus vaccine development: a lytic and latent protein cocktail. Front. Biosci. 13:5916-27, 2008.
Loewendorf A, Benedict C A. Modulation of host innate and adaptive immune defenses by cytomegalovirus: timing is everything. J Intern Med. 2010 May; 267(5):483-501.
Lohr, J. G., et al. Discovery and prioritization of somatic mutations in diffuse large B-cell lymphoma (DLBCL) by whole-exome sequencing. Proc Natl Acad Sci USA 109, 3879-3884 (2012).
Long, E. O., Kim, H. S., Liu, D., Peterson, M. E. & Rajagopalan, S. Controlling natural killer cell responses: integration of signals for activation and inhibition. Annu Rev Immunol 31, 227-258 (2013).
Lu, T., et al. Hijacking TYRO3 from tumor cells via trogocytosis enhances NK-cell effector functions and proliferation. Cancer Immunol Res (2021).
Lupo-Stanghellini, M. T., et al. Clinical impact of suicide gene therapy in allogeneic hematopoietic stem cell transplantation. Hum Gene Ther 21, 241-250 (2010).
Ma, X., et al. Pan-cancer genome and transcriptome analyses of 1,699 paediatric leukaemias and solid tumours. Nature 555, 371-376 (2018).
Marshall E E, Geballe A P. Multifaceted evasion of the interferon response by cytomegalovirus. J Interferon Cytokine Res. 2009; 29(9):609-619.
Merino, A., et al. Chronic stimulation drives human NK cell dysfunction and epigenetic reprograming. J Clin Invest 129, 3770-3785 (2019).
Merouane, A., et al. Automated profiling of individual cell-cell interactions from high-throughput time-lapse imaging microscopy in nanowell grids (TIMING). Bioinformatics 31, 3189-3197 (2015).
Meyaard, L., et al. LAIR-1, a novel inhibitory receptor expressed on human mononuclear leukocytes. Immunity 7, 283-290 (1997).
Miner, C. A., Giri, T. K., Meyer, C. E., Shabsovich, M. & Tripathy, S. K. Acquisition of activation receptor ligand by trogocytosis renders NK cells hyporesponsive. J Immunol 194, 1945-1953 (2015).
Miyake, K. & Karasuyama, H. The Role of Trogocytosis in the Modulation of Immune Cell Functions. Cells 10(2021).
Moreno, D. F. & Cid, J. Graft-versus-host disease. Med Clin (Barc) 152, 22-28 (2019).
Morgan, R. A., et al. Case report of a serious adverse event following the administration of T cells transduced with a chimeric antigen receptor recognizing ERBB2. Mol Ther 18, 843-851 (2010).
Myers, J. A. & Miller, J. S. Exploring the NK cell platform for cancer immunotherapy. Nat Rev Clin Oncol 18, 85-100 (2021).
Nakamura, K., et al. Fratricide of natural killer cells dressed with tumor-derived NKG2D ligand. Proc Natl Acad Sci USA 110, 9421-9426 (2013). (Nakamura et al., 2013(a))
Nakamura, K., et al. NK-cell fratricide: Dynamic crosstalk between NK and cancer cells. Oncoimmunology 2, e26529 (2013). (Nakamura et al., 2013(b))
Nakayama, M., et al. Natural killer (NK)-dendritic cell interactions generate MHC class II-dressed NK cells that regulate CD4+ T cells. Proc Natl Acad Sci USA 108, 18360-18365 (2011).
O'Brien, K. L. & Finlay, D. K. Immunometabolism and natural killer cell responses. Nat Rev Immunol 19, 282-290 (2019).
Parker, K. R., et al. Single-Cell Analyses Identify Brain Mural Cells Expressing CD19 as Potential Off-Tumor Targets for CAR-T Immunotherapies. Cell 183, 126-142 e117 (2020).
Parkhurst et al., Characterization of genetically modified T cell receptors that recognize the CEA:691-699 peptide in the context of HA-A2.1 on human colorectal cancer cells. Clin Cancer Res. 15: 169-180, 2009.
Paul, S. & Lal, G. The Molecular Mechanism of Natural Killer Cells Function and Its Importance in Cancer Immunotherapy. Front Immunol 8, 1124 (2017).
Pegram, H. J., Andrews, D. M., Smyth, M. J., Darcy, P. K. & Kershaw, M. H. Activating and inhibitory receptors of natural killer cells. Immunol Cell Biol 89, 216-224 (2011).
Pesce, S., et al. Identification of a subset of human natural killer cells expressing high levels of programmed death 1: A phenotypic and functional characterization. J Allergy Clin Immunol 139, 335-346 e333 (2017).
Poorebrahim, M., et al. Counteracting CAR T cell dysfunction. Oncogene 40, 421-435 (2021).
Przepiorka, D., et al. Evaluation of anti-CD5 ricin A chain immunoconjugate for prevention of acute graft-vs.-host disease after HLA-identical marrow transplantation. Ther Immunol 1, 77-82 (1994).
Rafiq, S., Hackett, C. S. & Brentjens, R. J. Engineering strategies to overcome the current roadblocks in CAR T cell therapy. Nat Rev Clin Oncol 17, 147-167 (2020).
Rieder et al., Rhabdovirus evasion of the interferon system. J. Interferon Cytokine Res. (9):499-509, 2009.
Rohaan, M. W., Wilgenhof, S. & Haanen, J. Adoptive cellular therapies: the current landscape. Virchows Arch 474, 449-461 (2019).
Ryckman B J, Jarvis M A, Drummond D D, Nelson J A, Johnson D C. Human cytomegalovirus entry into epithelial and endothelial cells depends on genes UL128 to UL150 and occurs by endocytosis and low-pH fusion. J Virol. 2006 January; 80(2):710-22. doi: 10.1128/JVI.80.2.710-722.2006.
Sadelain, M., Brentjens, R. & Riviere, I. The basic principles of chimeric antigen receptor design. Cancer Discov 3, 388-398 (2013).
Sadelain, M. CAR therapy: the CD19 paradigm. J Clin Invest 125, 3392-3400 (2015).
Sambrook et al., Molecular Cloning: A Laboratory Manual, 3rd ed., Cold Spring Harbor Press, Cold Spring Harbor, N.Y. 2001.
Shah N, Martin-Antonio B, Yang H, Ku S, Lee D A, Cooper L J, Decker W K, Li S, Robinson S N, Sekine T, Parmar S, Gribben J, Wang M, Rezvani K, Yvon E, Najjar A, Burks J, Kaur I, Champlin R E, Bollard C M, Shpall E J. Antigen presenting cell-mediated expansion of human umbilical cord blood yields log-scale expansion of natural killer cells with anti-myeloma activity. PLOS One. 2013 Oct. 18; 8(10):e76781.
Silence, K., et al. ARGX-110, a highly potent antibody targeting CD70, eliminates tumors via both enhanced ADCC and immune checkpoint blockade. MAbs 6, 523-532 (2014).
Singh H, Manuri P R, Olivares S, Dara N, Dawson M J, Huls H, Hackett P B, Kohn D B, Shpall E J, Champlin R E, Cooper L J. Redirecting specificity of T-cell populations for CD19 using the Sleeping Beauty system. Cancer Res. 2008 Apr. 15; 68(8):2961-71.
Singh H, Figliola M J, Dawson M J, Huls H, Olivares S, Switzer K, Mi T, Maiti S, Kebriaei P, Lee D A, Champlin R E, Cooper L J. Reprogramming CD19-specific T cells with IL-21 signaling can improve adoptive immunotherapy of B-lineage malignancies. Cancer Res. 2011 May 15; 71(10):3516-27. Epub 2011 May 10. Erratum in: Cancer Res. 2011 Jul. 1; 71(13):4734.
Simonetta, F., Pradier, A. & Roosnek, E. T-bet and Eomesodermin in NK Cell Development, Maturation, and Function. Front Immunol 7, 241 (2016).
Snyder J W, Munier G K, Heckman S A, Camp P, Overman T L. Failure of the B D GeneOhm StaphSR assay for direct detection of methicillin-resistant and methicillin-susceptible Staphylococcus aureus isolates in positive blood cultures collected in the United States. J Clin Microbiol. 2009 November; 47(11):3747-8.
Snyder J W, Munier G K, Johnson C L. Comparison of the B D GeneOhm methicillin-resistant Staphylococcus aureus (MRSA) PCR assay to culture by use of BBL CHROMagar MRSA for detection of MRSA in nasal surveillance cultures from intensive care unit patients. J Clin Microbiol. 2010; 48(4): 1305-1309.
Spanholtz J, Preijers F, Tordoir M, et al. Clinical-grade generation of active NK cells from cord blood hematopoietic progenitor cells for immunotherapy using a closed-system culture process. PLOS One. 2011; 6(6): e20740.
Tabiasco, J., et al. Active trans-synaptic capture of membrane fragments by natural killer cells. Eur J Immunol 32, 1502-1508 (2002).
Tabiasco, J., et al. Acquisition of viral receptor by NK cells through immunological synapse. J Immunol 170, 5993-5998 (2003).
Tai, Y. T., et al. Anti-CS1 humanized monoclonal antibody HuLuc63 inhibits myeloma cell adhesion and induces antibody-dependent cellular cytotoxicity in the bone marrow milieu. Blood 112, 1329-1337 (2008).
Taylor, R. P. & Lindorfer, M. A. Fcgamma-receptor-mediated trogocytosis impacts mAb-based therapies: historical precedence and recent developments. Blood 125, 762-766 (2015).
Turtle et al., Engineerd T cells for anti-cancer therapy. Curr. Opin. Immunol., 24(5): 633-39, 2012.
Van Gassen, S., et al. FlowSOM: Using self-organizing maps for visualization and interpretation of cytometry data. Cytometry A 87, 636-645 (2015).
Varela-Rohena et al., Genetic engineering of T cells for adoptive immunotherapy. Immunol Res. 2008; 42(1-3): 166-81.
Vera, J., et al. T lymphocytes redirected against the kappa light chain of human immunoglobulin efficiently kill mature B lymphocyte-derived malignant cells. Blood 108, 3890-3897 (2006).
Vivier, E., Nunes, J. A. & Vely, F. Natural killer cell signaling pathways. Science 306, 1517-1519 (2004).
Vogler, I., et al. An improved bicistronic CD20/tCD34 vector for efficient purification and in vivo depletion of gene-modified T cells for adoptive immunotherapy. Mol Ther 18, 1330-1338 (2010).
Wang, D., et al. CRISPR Screening of CAR T Cells and Cancer Stem Cells Reveals Critical Dependencies for Cell-Based Therapies. Cancer Discov 11, 1192-1211 (2021).
Wu, J. & Lanier, L. L. Natural killer cells and cancer. Adv Cancer Res 90, 127-156 (2003).
Wu et al., Adoptive T-cell therapy using autologous tumor-infiltrating lymphocytes for metastatic melanoma: current status and future outlook. Cancer, 18(2): 160-75, 2012.
Wu, Z., et al. Dynamic variability in SHP-1 abundance determines natural killer cell responsiveness. Sci Signal 14, eabe5380 (2021).
Zah, E., et al. Systematically optimized BCMA/CS1 bispecific CAR-T cells robustly control heterogeneous multiple myeloma. Nat Commun 11, 2283 (2020).
Zenarruzabeitia, O., Vitalle, J., Eguizabal, C., Simhadri, V. R. & Borrego, F. The Biology and Disease Relevance of CD300a, an Inhibitory Receptor for Phosphatidylserine and Phosphatidylethanolamine. J Immunol 194, 5053-5060 (2015).
Zysk G, Bejo L, Schneider-Wald B K, Nau R, Heinz H. Induction of necrosis and apoptosis of neutrophil granulocytes by Streptococcus pneumoniae. Clin Exp Immunol. 2000 October; 122(1):61-6.

What is claimed is:

1. A composition comprising an engineered immune effector cell, comprising:
(a) at least one inhibitory chimeric antigen receptor (iCAR) comprising:
(1) at least one extracellular antigen binding domain, wherein a first extracellular antigen binding domain binds a first antigen;
(2) a first transmembrane domain; and
(3) at least one natural killer (NK) cell inhibitory signaling domain and/or at least one co-inhibitory domain; and
(b) at least one activating chimeric antigen receptor (aCAR) comprising:
(1) at least one extracellular antigen binding domain, wherein a second extracellular binding domain binds a second antigen;
(2) a second transmembrane domain; and
(3) at least one activating endodomain with or without a costimulatory signaling domain.

2. The composition of claim 1, wherein the cell is a NK cell or T cell.

3. The composition of claim 1 or 2, wherein the first antigen and the second antigen are different antigens.

4. The composition of claim 1, 2, or 3, wherein the first extracellular antigen binding domain of (a)(1) binds an antigen on an NK cell.

5. The composition of claim 4, wherein the cell is an NK cell and the first extracellular antigen binding domain of (a)(1) binds an antigen on an NK cell.

6. The composition of claim 5, wherein the antigen on an NK cell is KIR2DL1, KIR2DL2, KIR2DL3, KIR2DL5, KIR3DL1, KIR3DL2, KIR3DL3, KIR2DL4, KIR2DS1, KIR2DS2, KIR2DS3, KIR2DS4, KIR2DS5, KIR3DS1, DNAX-activation protein 10 (DAP10), DNAX-activation protein 12 (DAP12), CD56, CD57, CD25, CD122, NKP30, NKP44, NKP46, NKG2-C type II integral membrane protein (NKG2C), NKG2-D type II integral membrane protein (NKG2D), NKG2-A/NKG2-B type II integral membrane protein (NKG2A), Cytotoxic and regulatory T-cell molecule (CRTAM), T cell immunoreceptor with Ig and ITIM domains (TIGIT), CD96, 2B4, CD16, CD27, CD100, CD160, ILT2, ILT4, Killer cell lectin-like receptor subfamily G member 1 (KLRG1), Leukocyte-associated immunoglobulin-like receptor 1/CD305 (LAIR1), CD161, CS1, an (Natural Cytotoxicity Receptor) NCR, KIR, and/or other NK-related antigen.

7. The composition of claim 5 or 6, wherein the iCAR has two antigen binding domains that each target different antigens.

8. The composition of any one of the preceding claims, wherein the second extracellular antigen binding domain of (b)(1) binds a cancer antigen or a pathogen antigen.

9. The composition of claim 8, wherein the second extracellular antigen binding domain of (b)(1) binds a cancer antigen on a solid tumor or on a hematological malignancy.

10. The composition of any one of the preceding claims, wherein the NK cell inhibitory signaling domain and/or co-inhibitory domain is from an NK cell inhibitory receptor.

11. The composition of any one of the preceding claims, wherein the NK cell inhibitory signaling domain and/or co-inhibitory domain are from leukocyte immunoglobulin-like receptor (LIR-1), CD300A, NKG2A, Siglec-7, CD96, cell immunoglobulin and mucin-domain containing-3 (TIM3), TIGIT, LAIR-1, KIR2DL1, KIR2DL2, KIR2DL3, KIR2DL4, KIR2DL5, KIR3DL1, KIR3DL2, KIR3DL3, KIR2DL5A, and/or KIR2DL5B

12. The composition of any one of the preceding claims, wherein the first transmembrane domain and the inhibitory signaling domain are from the same molecule.

13. The composition of claim 12, wherein the first transmembrane domain and the inhibitory signaling domain are from LIR-1 or KIR2DL1.

14. The composition of any one of claims 1-13, wherein the iCAR comprises at least one co-inhibitory domain.

15. The composition of claim 14, wherein the co-inhibitory domain is from LAIR-1, NKG2A, CD300A, or a combination thereof.

16. The composition of any one of the preceding claims, wherein the first and/or second extracellular antigen binding domain comprises an scFv or a natural ligand.

17. The composition of any one of the preceding claims, wherein the second extracellular antigen binding domain of (b)(1) comprises an scFv that binds an antigen selected from the group consisting of CD19, EBNA, CD123, HER2, CA-125, TRAIL/DR4, CD20, CD70, HLA-G, CD3δ, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases, MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, GAGE-2, GAGE-3, GAGE-4, GAGE-5, GAGE-6, GAGE-7A, GAGE-7B, GAGE-8, NA88-A, MCIR, MDA-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, a Phosphoinositide 3-kinase, a TRK receptor, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1), TACSTD2, a receptor tyrosine kinase, Epidermal Growth Factor receptor (EGFR), EGFRvIII, platelet derived growth factor receptor (PDGFR), vascular endothelial growth factor receptor (VEGFR), VEGFR2, a cytoplasmic tyrosine kinase, integrin-linked kinase (ILK), signal transducers and activators of transcription STAT3, STATS, and STATE, HIF-1, HIF-2, Nuclear Factor-Kappa B (NF-B), a Notch receptor NY ESO 1, c-Met, mammalian targets of rapamycin (mTOR), WNT, extracellular signal-regulated kinases (ERKs), PMSA, PR-3, MDM2, Mesothelin, renal cell carcinoma-5T4, SM22-alpha, carbonic anhydrases I (CAI), CAIX), STEAD, TEL/AML1, GD2, proteinase3, hTERT, sarcoma translocation breakpoints, EphA2, ML-IAP, EpCAM, TMPRSS2 ETS fusion gene, ERG, NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, and a combination thereof.

18. The composition of any one of the preceding claims, wherein the iCAR comprises:
a first extracellular antigen binding domain of (a)(1) that comprises an scFv that binds CS1;
an IgG1 hinge;
a first transmembrane domain of (a)(2) from KIR2DL1; and
an inhibitory signaling domain of (a)(3) from KIR2DL1.

19. The composition of any one of the preceding claims, wherein the iCAR comprises:
a first extracellular antigen binding domain of (a)(1) that comprises an scFv that binds CD19;
an IgG1 hinge;
a first transmembrane domain of (a)(2) from KIR2DL1; and
an inhibitory signaling domain of (a)(3) from KIR2DL1.

20. The composition of any one of the preceding claims, wherein the iCAR comprises:
a first extracellular antigen binding domain of (a)(1) that comprises an scFv that binds CD19;
an IgG1 hinge;
a first transmembrane domain of (a)(2) from LIR-1; and
an inhibitory signaling domain of (a)(3) from LIR-1.

21. The composition of any one of the preceding claims, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from LAIR-1.

22. The composition of any one of the preceding claims, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from NKG2A.

23. The composition of any one of the preceding claims, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from CD300A.

24. The composition of any one of the preceding claims, wherein the iCAR comprises SEQ ID NO:2, SEQ ID NO:4, SEQ ID NO:6, SEQ ID NO:8, or SEQ ID NO:10.

25. The composition of any one of the preceding claims, wherein at least part of the iCAR is encoded by sequence of SEQ ID NO: 1, SEQ ID NO:3, SEQ ID NO:5, SEQ ID NO:7, or SEQ ID NO:9.

26. The composition of any one of the preceding claims, further defined as comprising a plurality of the cells.

27. The composition of claim 26, said composition housed in a pharmaceutically acceptable carrier.

28. A method of enhancing an adoptive cell therapy for an individual in need thereof, comprising, administering to the individual a therapeutically effective amount of engineered immune effector cells, each comprising:
(a) at least one inhibitory chimeric antigen receptor (iCAR) comprising:
at least one extracellular antigen binding domain, wherein a first extracellular antigen binding domain binds a first antigen; and
at least one natural killer (NK) cell inhibitory signaling domain and/or at least one co-inhibitory domain; and
(b) at least one activating chimeric antigen receptor (aCAR) comprising:
at least one extracellular antigen binding domain, wherein a second antigen binding domain binds a second antigen; and
an activating endodomain and at least one costimulatory signaling domain, and wherein:
(I) when the first antigen and the second antigen are the same and when the engineered immune effector cell binds through the second extracellular antigen binding domain a cell that expresses the antigen, the iCAR inhibits the killing by the engineered immune effector cell of the cell that expresses the antigen; or
(II) when the first and second antigen are non-identical and are both expressed on a fellow engineered immune effector cell or on a non-engineered immune effector cell of the same type or on a non-diseased cell, when the engineered immune effector cell binds through the second extracellular antigen binding domain to the second antigen on the fellow engineered immune effector cell or the non-engineered immune effector cell of the same type or the non-diseased cell, respectively, the iCAR inhibits the killing by the engineered immune effector cell of the fellow engineered immune effector cell or the non-engineered immune effector cell of the same type or the non-diseased cell, respectively.

29. The method of claim 28, wherein in (I) the cell that expresses the antigen is a non-cancerous cell.

30. The method of claim 28, wherein in (I) the cell that expresses the antigen is a fellow engineered immune effector cell.

31. The method of claim 28, wherein in (II), the engineered immune effector cell binds through the second extracellular antigen binding domain to the second antigen on the fellow engineered immune effector cell.

32. The method of claim 31, wherein the second antigen is expressed by the fellow engineered immune effector cell as a result of trogocytosis.

33. The method of any one of claims 28-32, wherein engineered immune effector cells are obtained from storage.

34. The method of any one of claims 28-32, wherein engineered immune effector cells are produced without storage.

35. The method of any one of claims 28-34, wherein the engineered immune effector cells are engineered to express the iCAR prior to being engineered to express the aCAR.

36. The method of any one of claims 28-34, wherein the engineered immune effector cells are engineered to express the iCAR subsequent to being engineered to express the aCAR.

37. The method of any one of claims 28-36, wherein the engineered immune effector cells are NK cells and are engineered to express an iCAR that targets an NK cell self antigen.

38. The method of any one of claims 28-35, wherein the engineered immune effector cells are engineered to express an iCAR that targets an NK cell self antigen and are then engineered to express an aCAR that targets an antigen on cancer cells of the individual.

39. The method of any one of claims 28-38, wherein the engineered immune effector cells are derived from cells that are autologous with respect to the individual.

40. The method of any one of claims 28-38, wherein the engineered immune effector cells are derived from cells that are allogeneic with respect to the individual.

41. The method of any one of claims 28-40, wherein the engineered immune effector cells are NK cells or T cells.

42. The method of any one of claims 28-41, wherein the first extracellular antigen binding domain binds an antigen on an NK cell.

43. The method of any one of claims 28-42, wherein the engineered immune effector cells are NK cells and the first extracellular antigen binding domain binds an antigen on an NK cell.

44. The method of claim 43, wherein the antigen on the NK cell is CS1, CD56, NKG2D, an NCR, KIR, or other NK-related antigen.

45. The method of any one of claims 28-44, wherein the iCAR has two antigen binding domains that each target different antigens.

46. The method of any one of claims 28-45, wherein the second extracellular antigen binding domain binds a cancer antigen or a pathogen antigen.

47. The method of any one of claims 28-46, wherein the second extracellular antigen binding domain binds a cancer antigen on a solid tumor or on a hematological malignancy.

48. The method of any one of claims 28-47, wherein the NK cell inhibitory signaling domain and/or co-inhibitory domain is from an NK cell inhibitory receptor.

49. The method of any one of claims 28-47, wherein the first transmembrane domain and the inhibitory signaling domain are from the same molecule.

50. The method of claim 49, wherein the first transmembrane domain and the inhibitory signaling domain are from LIR-1 or KIR2DL1.

51. The method of any one of claims 28-50, wherein the iCAR comprises at least one co-inhibitory domain.

52. The method of claim 51, wherein the co-inhibitory domain is from LAIR-1, NKG2A, CD300A, or a combination thereof.

53. The method of any one of claims 28-52, wherein the first and/or second extracellular antigen binding domain comprises an scFv or a natural ligand.

54. The method of any one of the preceding claims, wherein the second extracellular antigen binding domain comprises an scFv that binds an antigen selected from the group consisting of CD19, EBNA, CD123, HER2, CA-125, TRAIL/DR4, CD20, CD70, HLA-G, CD3δ, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases, MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, GAGE-2, GAGE-3, GAGE-4, GAGE-5, GAGE-6, GAGE-7A, GAGE-7B, GAGE-8, NA88-A, MC1R, MDA-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, a Phosphoinositide 3-kinase, a TRK receptor, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1), TACSTD2, a receptor tyrosine kinase, Epidermal Growth Factor receptor (EGFR), EGFRvIII, platelet derived growth factor receptor (PDGFR), vascular endothelial growth factor receptor (VEGFR), VEGFR2, a cytoplasmic tyrosine kinase, integrin-linked kinase (ILK), signal transducers and activators of transcription STAT3, STATS, and STATE, HIF-1, HIF-2, Nuclear Factor-Kappa B (NF-B), a Notch receptor NY ESO 1, c-Met, mammalian targets of rapamycin (mTOR), WNT, extracellular signal-regulated kinases (ERKs), PMSA, PR-3, MDM2, Mesothelin, renal cell carcinoma-5T4, SM22-alpha, carbonic anhydrases I (CAI), CAIX), STEAD, TEL/AML1, GD2, proteinase3, hTERT, sarcoma translocation breakpoints, EphA2, ML-IAP, EpCAM, TMPRSS2 ETS fusion gene, ERG, NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, and a combination thereof.

55. The method of any one of claims 28-54, wherein the iCAR comprises:
a first extracellular antigen binding domain of (a)(1) that comprises an scFv that binds CS1;
an IgG1 hinge;
a first transmembrane domain of (a)(2) from KIR2DL1; and
an inhibitory signaling domain of (a)(3) from KIR2DL1.

56. The method of any one of claims 28-54, wherein the iCAR comprises:
a first extracellular antigen binding domain of (a)(1) that comprises an scFv that binds CD19;
an IgG1 hinge;
a first transmembrane domain of (a)(2) from KIR2DL1; and
an inhibitory signaling domain of (a)(3) from KIR2DL1.

57. The method of any one of claims 28-54, wherein the iCAR comprises:
a first extracellular antigen binding domain of (a)(1) that comprises an scFv that binds CD19;
an IgG1 hinge;
a first transmembrane domain of (a)(2) from LIR-1; and
an inhibitory signaling domain of (a)(3) from LIR-1.

58. The method of any one of claims 28-54, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from LAIR-1.

59. The method of any one of claims 28-54, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from NKG2A.

60. The method of any one of claims 28-54, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from CD300A.

61. The method of any one of claims 28-57, wherein the iCAR comprises SEQ ID NO:2, SEQ ID NO:4, SEQ ID NO:6, SEQ ID NO:8, or SEQ ID NO: 10.

62. The method of any one of claims 28-57, wherein at least part of the iCAR is encoded by sequence of SEQ ID NO:1, SEQ ID NO:3, SEQ ID NO:5, SEQ ID NO:7, or SEQ ID NO:9.

63. The method of any one of claims 28-62, wherein the individual has cancer and is administered a therapeutically effective amount of a second cancer therapy.

64. The method of claim 63, wherein the second cancer therapy is surgery, radiation, chemotherapy, drug therapy, hormone therapy, immunotherapy or a combination thereof.

65. The method of claim 63 or 64, wherein the second cancer therapy is delivered prior to, during, or after the engineered immune effector cells.

66. The method of any one of claims 28-65, wherein the individual has metastatic cancer.

67. A method for increasing the likelihood of an individual being classified as responding to an immune effector cell therapy when compared to a control population of individuals receiving an immune effector cell therapy, comprising: administering to the individual a therapeutically effective amount of engineered immune effector cells, each comprising:
(a) at least one inhibitory chimeric antigen receptor (iCAR) comprising:
at least one extracellular antigen binding domain, wherein a first extracellular antigen binding domain binds a first antigen; and
at least one natural killer (NK) cell inhibitory signaling domain and/or at least one co-inhibitory domain; and
(b) at least one activating chimeric antigen receptor (aCAR) comprising:
at least one extracellular antigen binding domain, wherein a second antigen binding domain binds a second antigen; and
an activating endodomain and at least one costimulatory signaling domain.

68. A method for increasing at least one of in vivo persistence, viability, or efficacy of effector cells comprised in an immune effector cell therapy when compared to control effector cells comprised in a control immune effector cell therapy, comprising:
administering to an individual a therapeutically effective amount of engineered immune effector cells, each comprising:
(a) at least one inhibitory chimeric antigen receptor (iCAR) comprising:
at least one extracellular antigen binding domain, wherein a first extracellular antigen binding domain binds a first antigen; and
at least one natural killer (NK) cell inhibitory signaling domain and/or at least one co-inhibitory domain; and
(b) at least one activating chimeric antigen receptor (aCAR) comprising:
at least one extracellular antigen binding domain, wherein a second antigen binding domain binds a second antigen; and
an activating endodomain and at least one costimulatory signaling domain.

69. A method for increasing the likelihood of survival of an individual following treatment with an immune effector cell therapy when compared to a control population of individuals receiving an immune effector cell therapy, comprising:
administering to the individual a therapeutically effective amount of engineered immune effector cells, each comprising:
(a) at least one inhibitory chimeric antigen receptor (iCAR) comprising:
at least one extracellular antigen binding domain, wherein a first extracellular antigen binding domain binds a first antigen; and
at least one natural killer (NK) cell inhibitory signaling domain and/or at least one co-inhibitory domain; and
(b) at least one activating chimeric antigen receptor (aCAR) comprising:
at least one extracellular antigen binding domain, wherein a second antigen binding domain binds a second antigen; and
an activating endodomain and at least one costimulatory signaling domain.

70. A method for increasing the circulating serum levels of at least one effector cell associated protein in an individual following treatment with an immune effector cell therapy when compared to a control population of individuals receiving an immune effector cell therapy, comprising: administering to the individual a therapeutically effective amount of engineered immune effector cells, each comprising:
(a) at least one inhibitory chimeric antigen receptor (iCAR) comprising:
at least one extracellular antigen binding domain, wherein a first extracellular antigen binding domain binds a first antigen; and
at least one natural killer (NK) cell inhibitory signaling domain and/or at least one co-inhibitory domain; and
(b) at least one activating chimeric antigen receptor (aCAR) comprising:
at least one extracellular antigen binding domain, wherein a second antigen binding domain binds a second antigen; and
an activating endodomain and at least one costimulatory signaling domain;
wherein the at least one effector cell associated protein is selected from Granzyme A (GrA), Granzyme B (GrB), Perforin, Interferon gamma (IFN-γ), and Tumor Necrosis Factor alpha (TNF-α).

71. The method of any one of claims 67-70, wherein:
(I) when the first antigen and the second antigen are the same and when the engineered immune effector cell binds through the second extracellular antigen binding domain a cell that expresses the antigen, the iCAR inhibits the killing by the engineered immune effector cell of the cell that expresses the antigen; or
(II) when the first and second antigen are non-identical and are both expressed on a fellow engineered immune effector cell or on a non-engineered immune effector cell of the same type or on a non-diseased cell, when the engineered immune effector cell binds through the second extracellular antigen binding domain to the second antigen on the fellow engineered immune effector cell or the non-engineered immune effector cell of the same type or the non-diseased cell, respectively, the iCAR inhibits the killing by the engineered immune effector cell of the fellow engineered immune effector cell or the non-engineered immune effector cell of the same type or the non-diseased cell, respectively.

72. The method of claim 71, wherein in (I) the cell that expresses the antigen is a non-cancerous cell.

73. The method of claim 71, wherein in (I) the cell that expresses the antigen is a fellow engineered immune effector cell.

74. The method of claim 71, wherein in (II), the engineered immune effector cell binds through the second extracellular antigen binding domain to the second antigen on the fellow engineered immune effector cell.

75. The method of claim 74, wherein the second antigen is expressed by the fellow engineered immune effector cell as a result of trogocytosis.

76. The method of any one of claims 71-75, wherein engineered immune effector cells are obtained from storage.

77. The method of any one of claims 71-75, wherein engineered immune effector cells are produced without storage.

78. The method of any one of claims 71-77, wherein the engineered immune effector cells are engineered to express the iCAR prior to being engineered to express the aCAR.

79. The method of any one of claims 71-77, wherein the engineered immune effector cells are engineered to express the iCAR subsequent to being engineered to express the aCAR.

80. The method of any one of claims 71-79, wherein the engineered immune effector cells are NK cells and are engineered to express an iCAR that targets an NK cell self antigen.

81. The method of any one of claims 71-78, wherein the engineered immune effector cells are engineered to express an iCAR that targets an NK cell self antigen and are then engineered to express an aCAR that targets an antigen on cancer cells of the individual.

82. The method of any one of claims 71-81, wherein the engineered immune effector cells are derived from cells that are autologous with respect to the individual.

83. The method of any one of claims 71-81, wherein the engineered immune effector cells are derived from cells that are allogeneic with respect to the individual.

84. The method of any one of claims 71-83, wherein the engineered immune effector cells are NK cells or T cells.

85. The method of any one of claims 71-84, wherein the first extracellular antigen binding domain binds an antigen on an NK cell.

86. The method of any one of claims 71-85, wherein the engineered immune effector cells are NK cells and the first extracellular antigen binding domain binds an antigen on an NK cell.

87. The method of claim 86, wherein the antigen on the NK cell is CS1, CD56, NKG2D, an NCR, KIR, or other NK-related antigen.

88. The method of any one of claims 71-87, wherein the iCAR has two antigen binding domains that each target different antigens.

89. The method of any one of claims 71-88, wherein the second extracellular antigen binding domain binds a cancer antigen or a pathogen antigen.

90. The method of any one of claims 71-89, wherein the second extracellular antigen binding domain binds a cancer antigen on a solid tumor or on a hematological malignancy.

91. The method of any one of claims 71-90, wherein the NK cell inhibitory signaling domain and/or co-inhibitory domain is from an NK cell inhibitory receptor.

92. The method of any one of claims 71-90, wherein the first transmembrane domain and the inhibitory signaling domain are from the same molecule.

93. The method of claim 92, wherein the first transmembrane domain and the inhibitory signaling domain are from LIR-1 or KIR2DL1.

94. The method of any one of claims 71-93, wherein the iCAR comprises at least one co-inhibitory domain.

95. The method of claim 94, wherein the co-inhibitory domain is from LAIR-1, NKG2A, CD300A, or a combination thereof.

96. The method of any one of claims 71-95, wherein the first and/or second extracellular antigen binding domain comprises an scFv or a natural ligand.

97. The method of any one of claims 71-96, wherein the second extracellular antigen binding domain comprises an scFv that binds an antigen selected from the group consisting of CD19, EBNA, CD123, HER2, CA-125, TRAIL/DR4, CD20, CD70, HLA-G, CD3δ, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases, MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, GAGE-2, GAGE-3, GAGE-4, GAGE-5, GAGE-6, GAGE-7A, GAGE-7B, GAGE-8, NA88-A, MCIR, MDA-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, a Phosphoinositide 3-kinase, a TRK receptor, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1), TACSTD2, a receptor tyrosine kinase, Epidermal Growth Factor receptor (EGFR), EGFRvIII, platelet derived growth factor receptor (PDGFR), vascular endothelial growth factor receptor (VEGFR), VEGFR2, a cytoplasmic tyrosine kinase, integrin-linked kinase (ILK), signal transducers and activators of transcription STAT3, STATS, and STATE, HIF-1, HIF-2, Nuclear Factor-Kappa B (NF-B), a Notch receptor NY ESO 1, c-Met, mammalian targets of rapamycin (mTOR), WNT, extracellular signal-regulated kinases (ERKs), PMSA, PR-3, MDM2, Mesothelin, renal cell carcinoma-5T4, SM22-alpha, carbonic anhydrases I (CAI), CAIX), STEAD, TEL/AML1, GD2, proteinase3, hTERT, sarcoma translocation breakpoints, EphA2, ML-IAP, EpCAM, TMPRSS2 ETS fusion gene, ERG, NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, and a combination thereof.

98. The method of any one of claims 71-97, wherein the iCAR comprises:
a first extracellular antigen binding domain of (a)(1) that comprises an scFv that binds CS1;
an IgG1 hinge;
a first transmembrane domain of (a)(2) from KIR2DL1; and
an inhibitory signaling domain of (a)(3) from KIR2DL1.

99. The method of any one of claims 71-97, wherein the iCAR comprises:
a first extracellular antigen binding domain of (a)(1) that comprises an scFv that binds CD19;
an IgG1 hinge;
a first transmembrane domain of (a)(2) from KIR2DL1; and
an inhibitory signaling domain of (a)(3) from KIR2DL1.

100. The method of any one of claims 71-97, wherein the iCAR comprises:
a first extracellular antigen binding domain of (a)(1) that comprises an scFv that binds CD19;
an IgG1 hinge;
a first transmembrane domain of (a)(2) from LIR-1; and
an inhibitory signaling domain of (a)(3) from LIR-1.

101. The method of any one of claims 71-97, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from LAIR-1.

102. The method of any one of claims 71-97, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from NKG2A.

103. The method of any one of claims 71-97, wherein the iCAR comprises:
a first extracellular antigen binding domain of (a)(1) that comprises an scFv that binds CD19;
an IgG1 hinge;
a first transmembrane domain of (a)(2) from KIR2DL1;
an inhibitory signaling domain of (a)(3) from KIR2DL1; and
a co-inhibitory domain from CD300A.

104. The method of any one of claims 71-100, wherein the iCAR comprises SEQ ID NO:2, SEQ ID NO:4, SEQ ID NO:6, SEQ ID NO:8, or SEQ ID NO: 10.

105. The method of any one of claims 71-100, wherein at least part of the iCAR is encoded by sequence of SEQ ID NO:1, SEQ ID NO:3, SEQ ID NO:5, SEQ ID NO:7, or SEQ ID NO:9.

106. The method of any one of claims 71-105, wherein the individual has cancer and is administered a therapeutically effective amount of a second cancer therapy.

107. The method of claim 106, wherein the second cancer therapy is surgery, radiation, chemotherapy, drug therapy, hormone therapy, immunotherapy or a combination thereof.

108. The method of claim 106 or 107, wherein the second cancer therapy is delivered prior to, during, or after the engineered immune effector cells.

109. The method of any one of claims 71-108, wherein the individual has metastatic cancer.


SEQUENCE LISTING (12 sequences)

SEQ ID NO: 1 - DNA, 309 bp, Artificial Sequence
  Synthetic Nucleotide
attctgattgggacctcagtggtcatcatcctcttcatcctcctcttctttctccttcatcgctggtgctccaacaaaaaaaatgctgcggtaatggaccaagagtctgcaggaaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgccca

SEQ ID NO: 2 - protein, 103 aa, Artificial Sequence
  Synthetic Polypeptides
ILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 3 - DNA, 573 bp, Artificial Sequence
  Synthetic Nucleotide
gttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggcagagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 4 - protein, 189 aa, Artificial Sequence
  Synthetic Polypeptides
VIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 5 - DNA, 612 bp, Artificial Sequence
  Synthetic Nucleotide
attctgattgggacctcagtggtcatcatcctcttcatcctcctcttctttctccttcatcgctggtgctccaacaaaaaaaatgctgcggtaatggaccaagagtctgcaggaaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccacatcgccagaatcagataaagcaggggccccccagaagcaaggacgaggagcagaagccacagcagaggcctgacctggctgttgatgttctagagaggacagcagacaaggccacagtcaatggacttcctgagaaggacagagagacggacacctcggccctggctgcagggagttcccaggaggtgacgtatgctcagctggaccactgggccctcacacagaggacagcccgggctgtgtccccacagtccacaaagcccatggccgagtccatcacgtatgcagccgttgccagacac

SEQ ID NO: 6 - protein, 204 aa, Artificial Sequence
  Synthetic Polypeptides
ILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCPHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 7 - DNA, 519 bp, Artificial Sequence
  Synthetic Nucleotide
attctgattgggacctcagtggtcatcatcctcttcatcctcctcttctttctccttcatcgctggtgctccaacaaaaaaaatgctgcggtaatggaccaagagtctgcaggaaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccaaaagaaccggcgagcccgctggataaatgccattataccaaagataacggccagtttgatcagagcgcgaaacagctgaacctggaagcgtataccattgaacaggaaaccgcgctgattagcaacaaaaacggcaaaccgaaacgccagcagcgcaaaccgaacccgccgctgaacctggatagctatattgtgggccagaacgatatg

SEQ ID NO: 8 - protein, 173 aa, Artificial Sequence
  Synthetic Polypeptides
ILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCPKEPASPLDKCHYTKDNGQFDQSAKQLNLEAYTIEQETALISNKNGKPKRQQRKPNPPLNLDSYIVGQNDM

SEQ ID NO: 9 - DNA, 603 bp, Artificial Sequence
  Synthetic Nucleotide
attctgattgggacctcagtggtcatcatcctcttcatcctcctcttctttctccttcatcgctggtgctccaacaaaaaaaatgctgcggtaatggaccaagagtctgcaggaaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccaaggatgtttcagaaatggatcaaagctggtgaccattcagagctgtcccagaaccccaagcaggctgccacgcagagtgagctgcactacgcaaatctggagctgctgatgtggcctctgcaggaaaagccagcaccaccaagggaggtggaggtggaatacagcactgtggcctcccccagggaagaacttcactatgcctcggtggtgtttgattctaacaccaacaggatagctgctcagaggcctcgggaggaggaaccagattcagattacagtgtgataaggaagaca

SEQ ID NO: 10 - protein, 201 aa, Artificial Sequence
  Synthetic Polypeptides
ILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCPRMFQKWIKAGDHSELSQNPKQAATQSELHYANLELLMWPLQEKPAPPREVEVEYSTVASPREELHYASVVFDSNTNRIAAQRPREEEPDSDYSVIRKT

SEQ ID NO: 11 - DNA, 23 bp, Artificial Sequence
  Synthetic crRNA
ctaggtccgaaacattccaccgg

SEQ ID NO: 12 - DNA, 23 bp, Artificial Sequence
  Synthetic crRNA
cgaggaacctctagtggtgaagg